(19) United States
(12) Patent Application Publication
(10) Pub. No.: US 2026/0216341 A1
(43) Pub. Date: Jul. 30, 2026

(54) KETO ACID BIOPOLYMERS AND POLYOL KETO ESTERS AND USES THEREOF

(71) Applicant: ATOSSA THERAPEUTICS, INC., Seattle, WA (US)

(72) Inventors: Natasha Mimosa Quay, Seattle, WA (US); Steven Carl Quay, Seattle, WA (US)

(21) Appl. No.: 19/145,308

(22) PCT Filed: Jan. 2, 2024
(86) PCT No.: PCT/US2024/010082

§ 371 Date: Jul. 2, 2025

Related U.S. Application Data
(60) Provisional application No. 63/436,791, filed on Jan. 3, 2023.
(60) Provisional application No. 63/522,982, filed on Jun. 23, 2023.

(51) Int. Cl. A61K 47/36; A61K 9/00; A61K 9/19; A61K 31/19; A61K 31/197; A61K 31/405; A61K 31/4172

(52) U.S. Cl. CPC A61K 47/36; A61K 9/0056; A61K 9/19; A61K 31/19; A61K 31/197; A61K 31/405; A61K 31/4172

(57) ABSTRACT

Described herein are compositions comprising alpha-keto acid analogues of amino acids. The alpha-keto acids may be in a composition also comprising a biopolymer such as chitosan. The alpha-keto acid analogues of amino acids may be bonded to a chitosan backbone. The alpha-keto acids may be in a composition with polyol compounds to form polyol keto esters. Also described herein are consumable compositions such as medical foods containing either the chitosan alpha-keto acid compositions or polyol keto ester compositions that may be used as protein supplements for patients with kidney disease or at risk of developing kidney disease. The compositions may be used to treat or prevent a kidney disease by increasing blood amino acid levels and suppressing blood ammonia and urea levels.

### CROSS-REFERENCE

This application claims the benefit of U.S. Provisional Application No. 63/436,791, titled “KETO ACID BIOPOLYMERS AND USES THEREOF,” filed Jan. 3, 2023, and U.S. Provisional Application No. 63/522,982, titled “KETO ACID BIOPOLYMERS AND POLYOL KETO ESTERS AND USES THEREOF,” filed Jun. 23, 2023, which applications are incorporated herein by reference in their entirety.

### BACKGROUND

Amino acids are an important class of biomolecules that serve as the building blocks for proteins and have vital roles in other metabolic processes. Specifically, there are 9 essential amino acids that humans cannot naturally synthesize, and which therefore must come from dietary sources. However, certain conditions limit the amount of protein which can be safely consumed. Patients with impaired kidney function can have heightened sensitivities to by-products of amino acid metabolism (such as ammonia and urea), which can build up in the blood stream instead of being excreted in the urine. Accordingly, patients with kidney diseases such as chronic kidney disease (CKD), diabetic kidney disease, polycystic kidney disease, and uremia often need to closely monitor their amino acid intake to keep healthy protein levels but minimize toxic nitrogen containing by-products.

Compounding the challenges of limited protein metabolism, amino acid deficiency is a common problem for patients with severe kidney disease. Treatment for low protein levels in kidney disease patients may involve administering a form of the amino acid without the nitrogen containing amino group termed the alpha-keto acid form. However, due to the acidity of these compounds, they are often administered as calcium and magnesium salts, which, even at moderate levels, can be harmful to kidney function. There is a need for providing the essential amino acids to kidney disease patients with low protein levels without calcium or magnesium ions for delivery, while mitigating ammonia and urea by-products.

### SUMMARY

In various aspects, the present disclosure provides a composition comprising: a polysaccharide, and an alpha-keto acid composition comprising one or more of an alpha-keto acid analogue of an amino acid.

In some aspects, the alpha-keto acid analogue of an amino acid is an alpha-keto acid analogue of an essential amino acid. In some aspects, the polysaccharide is a starch, a cellulose, an amino polysaccharide, an alginate, a carrageenan, a chitin, a chondroitin sulfate, a dextran, a galactomannan, a glycogen, a hyaluronic acid, a glycogen, a galactogen, an inulin, an arabinoxylan, or a pectin. In some aspects, the polysaccharide is an amino polysaccharide. In some aspects, the amino polysaccharide comprises an amino cellulose, an amino dextran, an amino galactose, an amino arabinose, a chitin, a chitosan, or a combination thereof. In some aspects, the polysaccharide is a chitosan.

In some aspects, the chitosan comprises Formula (I):

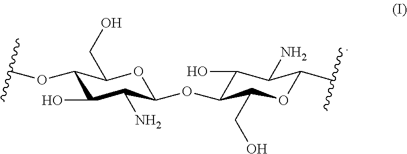

In some aspects, the polysaccharide comprises a positive charge at a neutral pH. In some aspects, the positive charge is a positively charged amino group. In some aspects, the alpha-keto acid analogue of an amino acid comprises a negative charge at a neutral pH. In some aspects, the negative charge is a negatively charged carboxylate group.

In some aspects, the alpha-keto acid analogue of an amino acid is:

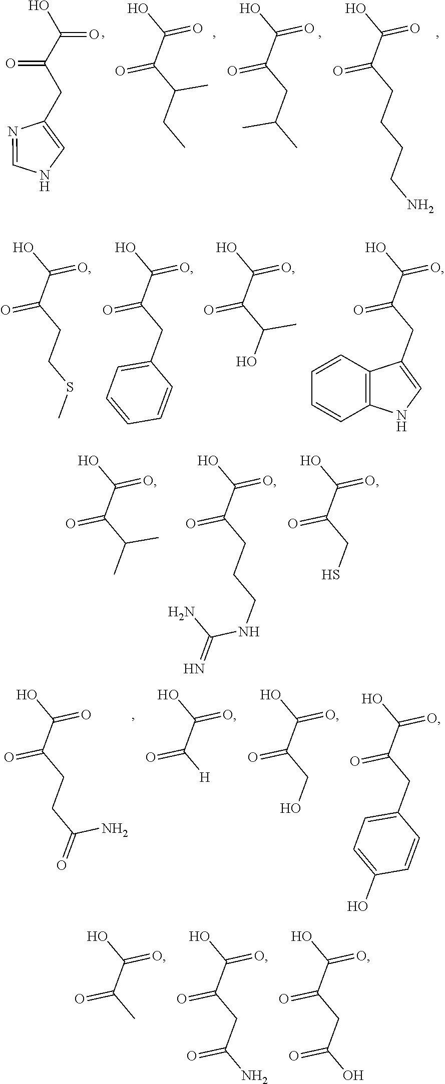

or a stereoisomer or pharmaceutically acceptable salt thereof.

In some aspects, the alpha-keto acid analogue of an amino acid is:

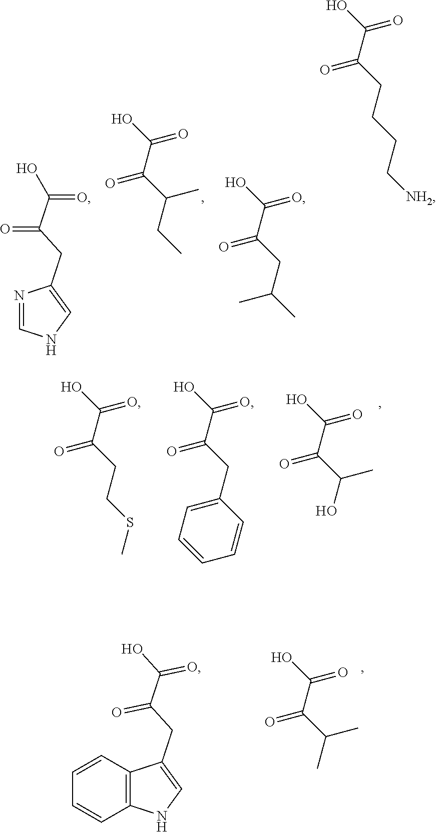

or a stereoisomer or pharmaceutically acceptable salt thereof.

In some aspects, the polysaccharide is ionically coupled to the alpha-keto acid analogue of an amino acid. In some aspects, the ionic coupling comprises an ionic bond between a positively charged amino group and a negatively charged carboxylate group. In some aspects, at least 10% and no more than 99% of the keto acid composition is bound to the polysaccharide. In some aspects, at least 15% and no more than 99%, at least 20% and no more than 99%, at least 25% and no more than 99%, at least 30% and no more than 99%, at least 35% and no more than 99%, at least 40% and no more than 99%, at least 45% and no more than 99%, at least 50% and no more than 99%, at least 55% and no more than 99%, at least 60% and no more than 99%, at least 65% and no more than 99%, at least 70% and no more than 99%, at least 75% and no more than 99%, at least 80% and no more than 99%, at least 85% and no more than 99%, at least 90% and no more than 99%, or at least 95% and no more than 99% of the keto acid composition is bound to the polysaccharide.

In some aspects, the composition comprises Formula (VI):

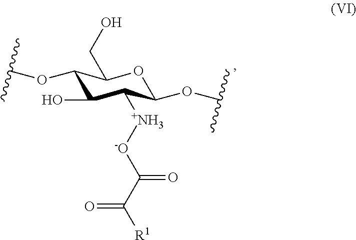

or a stereoisomer or pharmaceutically acceptable salt thereof, wherein each R¹ is independently a sidechain of a natural amino acid.

In some aspects, each R¹ is independently:

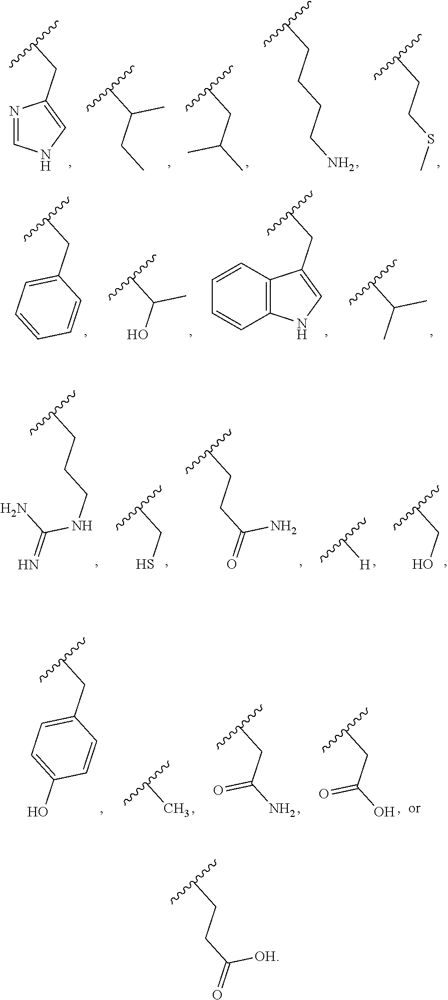

In some aspects, each R¹ is independently:

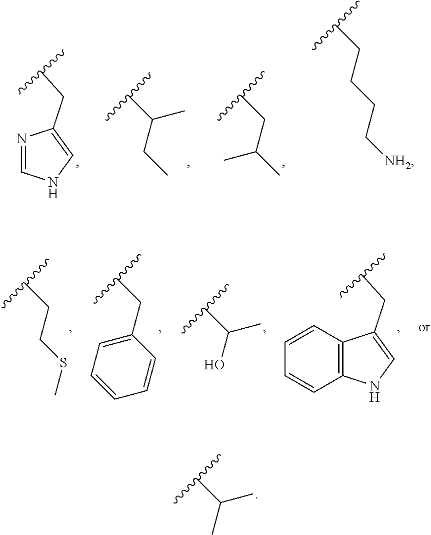

In some aspects, R¹ comprises:

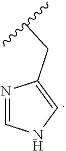 [SMILES: *Cc1c[nH]cn1]

In some aspects, R¹ comprises:

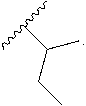 [SMILES: *C(C)CC]

In some aspects, R¹ comprises:

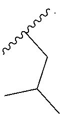 [SMILES: *CC(C)C]

In some aspects, R¹ comprises:

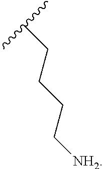 [SMILES: *CCCCN]

In some aspects, R¹ comprises:

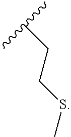 [SMILES: *CCSC]

In some aspects, R¹ comprises:

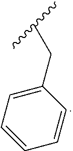 [SMILES: *Cc1ccccc1]

In some aspects, R¹ comprises:

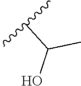 [SMILES: *C(C)O]

In some aspects, R¹ comprises:

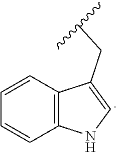 [SMILES: *Cc1c[nH]c2ccccc12]

In some aspects, R¹ comprises:

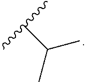 [SMILES: *C(C)C]

In some aspects, R¹ comprises:

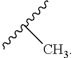

In some aspects, R¹ comprises:

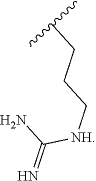 [SMILES: *CCCNC(=N)N]

In some aspects, R¹ comprises:

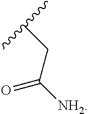 [SMILES: *CC(N)=O]

In some aspects, R¹ comprises:

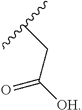 [SMILES: *CC(=O)O]

In some aspects, R¹ comprises:

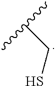 [SMILES: *CS]

In some aspects, R¹ comprises:

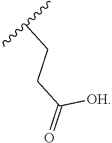 [SMILES: *CCC(=O)O]

In some aspects, R¹ comprises:

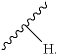

In some aspects, R¹ comprises:

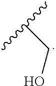 [SMILES: *CO]

In some aspects, R¹ comprises:

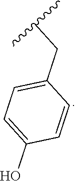 [SMILES: *Cc1ccc(O)cc1]

In some aspects, R¹ comprises:

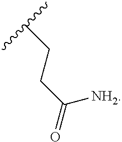 [SMILES: *CCC(N)=O]

In some aspects, the chitosan is not more than 50%, not more than 45%, not more than 40%, not more than 35%, not more than 30%, not more than 25%, not more than 20%, not more than 15%, not more than 10%, or not more than 5% acetylated. In some aspects, the chitosan comprises a copolymer. In some aspects, the copolymer is a linear copolymer, a block copolymer, an alternating copolymer, a periodic copolymer, a statistical copolymer, a stereoblock copolymer, a gradient copolymer, branched copolymer, a graft copolymer, a start copolymer, or a combination thereof. In some aspects, a stoichiometric ratio of the chitosan amine and the alpha-keto acid composition is from 1:1 to 1:10, from 1:1 to 1:100, from 1:1 to 1:1000, from 1:1 to 1:10,000, or from 1:1 to 1:100,000. In some aspects, a weight ratio of the chitosan to the alpha-keto acid composition is from 1:1 to 1:10, from 1:1 to 1:100, from 1:1 to 1:1000, from 1:1 to 1:10,000, or from 1:1 to 1:100,000. In some aspects, the alpha-keto acid composition comprises at least 0.01 mg/ml and no more than 20 mg/ml of the alpha-keto acid analogue of an amino acid. In some aspects, the alpha-keto acid composition comprises at least 0.01 mg/ml and no more than 20 mg/ml, at least 0.1 mg/ml and no more than 20 mg/ml, at least 0.1 mg/ml and no more than 15 mg/ml, at least 0.1 mg/ml and no more than 10 mg/ml, or at least 0.5 mg/ml and no more than 10 mg/ml of the alpha-keto acid analogue of an amino acid.

In various aspects, the present disclosure provides, a pharmaceutical composition comprising the composition as described herein and a pharmaceutically acceptable diluent or excipient.

In some aspects, the pharmaceutical composition is formulated for oral, topical, transdermal, rectal, intravenous, intra-arterial, intra-peritoneal, parenteral, or inhalation administration.

In various aspects, the present disclosure provides a medical food composition comprising the composition as described herein or the pharmaceutical composition as described herein and a food component.

In some aspects, the medical food composition is a liquid, a solid, a colloid, a gel, or a combination thereof. In some aspects, the medical food composition is formulated as a beverage, a drink mix, a solid food, a beverage, a bar, a cereal, a sports drink, a gel, a gelatin, a gelatinous gummy, a cracker, a chip, a puff, a granola cereal, a granola bar, a tablet, a powder, or an additive.

In some aspects, the medical food composition further comprises: a) a Cd content of less than 5 μg per daily serving or of less than 0.5 μg per g; and/or b) a Pb content of less than 5 μg per daily serving or less than 0.5 μg per g; and/or c) an As content of less than 15 μg per daily serving or less than 1.5 μg per g; and/or d) a Hg content of less than 30 μg per daily serving or less than 3 μg per g; and/or e) a Co content of less than 50 μg per daily serving or less than 5 μg per g; and/or f) a V content of less than 100 μg per daily serving or less than 10 μg per g; and/or g) a Ni content of less than 200 μg per daily serving or less than 20 μg per g; and/or h) a Tl content of less than 8 μg per daily serving or less than 0.8 μg per g; and/or i) an Au content of less than 300 μg per daily serving or less than 30 μg per g; and/or j) a Pd content of less than 100 μg per daily serving or less than 10 μg per g; and/or k) an Ir content of less than 100 μg per daily serving or less than 10 μg per g; and/or 1) an Os content of less than 100 μg per daily serving or less than 10 μg per g; and/or m) a Rh content of less than 100 μg per daily serving or less than 10 μg per g; and/or n) a Ru content of less than 100 μg per daily serving or less than 10 μg per g; and/or o) a Se content of less than 150 μg per daily serving or less than 15 μg per g; and/or p) an Ag content of less than 150 μg per daily serving or less than 15 μg per g; and/or q) a Pt content of less than 100 μg per daily serving or less than 10 μg per g; and/or r) a Li content of less than 550 μg per daily serving or less than 55 μg per g; and/or s) a Sb content of less than 1200 μg per daily serving or less than 120 μg per g; and/or t) a Ba content of less than 1400 μg per daily serving or less than 140 μg per g; and/or u) a Mo content of less than 3000 μg per daily serving or less than 300 μg per g; and/or v) a Cu content of less than 3000 μg per daily serving or less than 300 μg per g; and/or w) a Sn content of less than 6000 μg per daily serving or less than 600 μg per g; and/or x) a Cr content of less than 11000 μg per daily serving or less than 1100 μg per g; or any combination thereof.

In some aspects, the medical food composition comprises a microbial plate count of no more than 1000 colony forming per mL, no more than 10,000 colony forming per mL, or no more than 20,000 colony forming per mL.

In various aspects, the present disclosure provides a method of administering protein to a subject, the method comprising: administering a composition to the subject comprising: a polysaccharide, and an alpha-keto acid composition, wherein administering the composition to the subject increases a protein level in the subject as measured by a serum albumin test.

In various aspects, the present disclosure provides a method of treating a subject in need thereof, the method comprising: administering a composition to the subject comprising: a polysaccharide, and an alpha-keto acid composition, and increasing a protein level in the subject as measured by a serum albumin test, thereby treating the subject.

In some aspects, the composition is the composition as described herein, the pharmaceutical composition as described herein, or the medical food composition as described herein. In some aspects, the polysaccharide is chitosan. In some aspects, the alpha-keto acid composition comprises one or more alpha-keto acid analogues of an amino acid. In some aspects, the one or more alpha-keto acid analogues of an amino acid are alpha-keto acid analogues of an essential amino acid.

In some aspects, the subject has a higher than normal blood urea nitrogen level as measured by a serum blood urea nitrogen (BUN) test prior to administration of the composition. In some aspects, the higher than normal blood urea nitrogen level is caused by dehydration, burns, medicines, a high protein diet, or age. In some aspects, administering the composition to the subject results in a decrease of nitrogen by-products in the subject as compared to administering protein comprising amino acids to the subject. In some aspects, the nitrogen by-products comprise urea, uric acid, creatinine, ammonia, or a combination thereof. In some aspects, the decrease of nitrogen by-products is measured by a serum blood urea nitrogen (BUN) test.

In some aspects, administering the composition to the subject results in a decrease of a level of a salt in the subject as measured by a salt urine test as compared to administering protein comprising alpha-keto acid salts to the subject. In some aspects, the decrease of a level of a salt is measured by a urine test measuring sodium, potassium, and chloride. In some aspects, the salt comprises sodium, calcium, or magnesium. In some aspects, the subject does not have a kidney disease. In some aspects, the subject is at risk of developing a kidney disease. In some aspects, the subject has a condition that puts the subject at risk of a kidney disease. In some aspects, the condition is diabetes, high blood pressure, heart disease, obesity, a family history of chronic kidney disease, inherited kidney disorders, past damage to the kidneys, or old age.

In some aspects, the subject has a kidney disease. In some aspects, the kidney disease is chronic kidney disease, Fabry disease, cystinosis, glomerulonephritis, IgA nephropathy, lupus nephritis, atypical hemolytic uremic syndrome, polycystic kidney disease, diabetic kidney disease, or uremia. In some aspects, the subject is in an anabolic need. In some aspects, the anabolic need is characterized by a low protein-level as measured by a serum albumin test in the subject.

In various aspects, the present disclosure provides a method of synthesizing a chitosan keto acid composition comprising: combining a chitosan composition and an alpha-keto acid composition, adjusting the pH to promote binding of the alpha-keto acid composition to the chitosan composition, binding the alpha-keto acid composition to the chitosan composition thereby forming the chitosan keto acid composition.

In some aspects, the method further comprises lyophilizing the chitosan keto acid composition. In some aspects, the chitosan composition binds at least 15% and no more than 99%, at least 20% and no more than 99%, at least 25% and no more than 99%, at least 30% and no more than 99%, at least 35% and no more than 99%, at least 40% and no more than 99%, at least 45% and no more than 99%, at least 50% and no more than 99%, at least 55% and no more than 99%, at least 60% and no more than 99%, at least 65% and no more than 99%, at least 70% and no more than 99%, at least 75% and no more than 99%, at least 80% and no more than 99%, at least 85% and no more than 99%, at least 90% and no more than 99%, or at least 95% and no more than 99% of the alpha-keto acid composition. In some aspects, the alpha-keto acid composition comprises alpha-keto acid analogues of essential amino acids. In some aspects, the alpha-keto acid composition comprises at least 0.01 mg/ml and no more than 20 mg/ml, at least 0.1 mg/ml and no more than 20 mg/ml, at least 0.1 mg/ml and no more than 15 mg/ml, at least 0.1 mg/ml and no more than 10 mg/ml, or at least 0.5 mg/ml and no more than 10 mg/ml of one or more alpha-keto acid analogues of an amino acid.

In various aspects, the present disclosure provides a composition comprising a polysaccharide and an alpha-keto acid.

In various aspects, the present disclosure provides a composition comprising a polysaccharide and an alpha-keto acid analogue of an essential amino acid.

In various aspects, the present disclosure provides a composition comprising a polysaccharide non-covalently coupled to an alpha-keto acid.

In some aspects, the polysaccharide is a starch, a cellulose, an amino polysaccharide, an alginate, a carrageenan, a chitin, a chondroitin sulfate, a dextran, a galactomannan, a glycogen, a hyaluronic acid, a glycogen, a galactogen, an inulin, an arabinoxylan, or a pectin. In some aspects, the polysaccharide is an amino polysaccharide. In some aspects, the amino polysaccharide comprises an amino cellulose, an amino dextran, an amino galactose, an amino arabinose, a chitin, a chitosan, or a combination thereof. In some aspects, the polysaccharide is a chitosan. In some aspects, the chitosan comprises Formula (I):

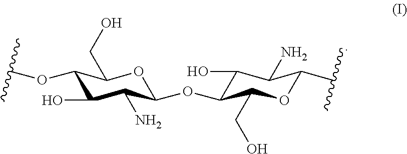

In some aspects, the alpha-keto acid comprises:

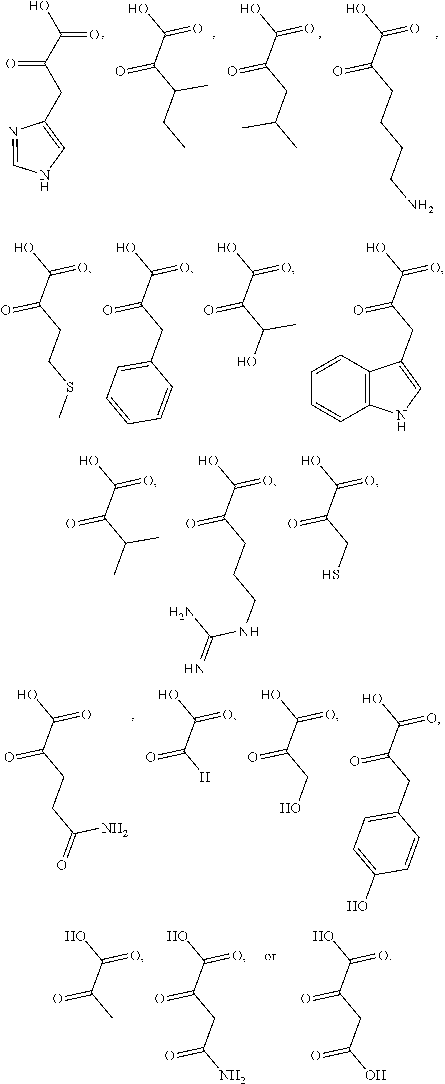

In some aspects, the alpha-keto acid comprises an alpha-keto acid analogue of an essential amino acid. In some aspects, the alpha-keto acid analogue of an essential amino acid comprises:

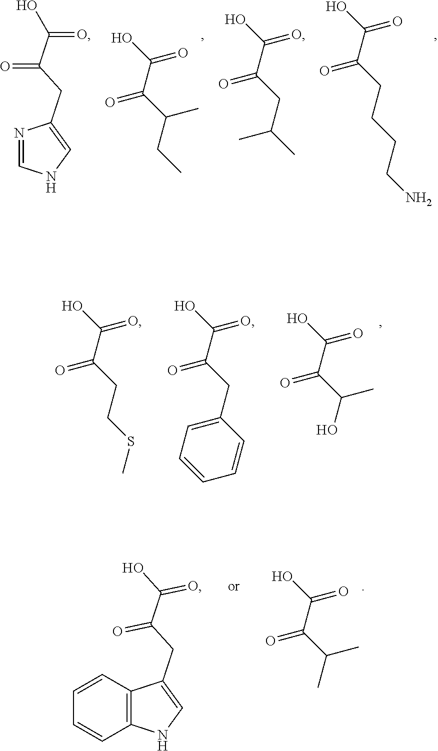

In some aspects, the alpha-keto acid is neutrally charged at pH 7. In some aspects, the alpha-keto acid is negatively charged at pH 7. In some aspects, the polysaccharide is neutrally charged at pH 7. In some aspects, the polysaccharide is positively charged at pH 7. In some aspects, the chitosan is neutrally charged at pH 7. In some aspects, the chitosan is positively charged at pH 7.

In some aspects, the polysaccharide is non-covalently coupled to the alpha-keto acid. In some aspects, the chitosan is ionically coupled to an alpha-keto acid through a positive charge on the chitosan and a negative charge on the alpha-keto acid. In some aspects, the chitosan is coupled to the alpha-keto acid through hydrogen bonding interactions. In some aspects, the chitosan is covalently coupled to the alpha-keto acid. In some aspects, the alpha-keto acid is covalently coupled to the polysaccharide as an amide. In some aspects, the alpha-keto acid is covalently coupled to the polysaccharide as an alpha-keto amide.

In some aspects, a plurality of alpha-keto acids is coupled to the chitosan. In some aspects, the plurality of alpha-keto acids comprises from 2 to 100,000, from 2 to 10,000, from 2 to 1000, from 10 to 100,000, from 10 to 10,000, from 10 to 1000, from 100 to 100,000, from 100 to 10,000, or from 100 to 1000 alpha-keto acids. In some aspects, between about 50% and 99.9%, about 60% and 99%, about 70% and 99%, about 80% and 99.9%, or about 90% and 99.9% of the plurality of alpha-keto acids are non-covalently coupled to the chitosan. In some aspects, between about 10% and 50%, about 20% and 60%, about 30% and 70%, about 40% and 80%, or about 50% and 90% of the plurality alpha-keto acids are covalently coupled to the chitosan. In some aspects, the plurality of alpha-keto acids comprises a first alpha-keto acid covalently coupled to the chitosan a second alpha-keto acid non-covalently coupled to the chitosan.

In some aspects, the composition comprises Formula (VI):

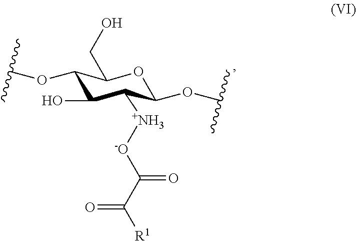

wherein each R¹ is independently a sidechain of a natural amino acid.

In some aspects, the composition comprises a chitosan cation and an alpha-keto acid anion.

In some aspects, the composition comprises Formula (VIII):

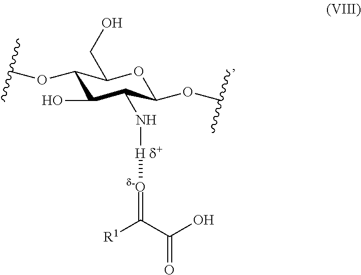

wherein each R¹ is independently a sidechain of a natural amino acid.

In some aspects, the composition comprises Formula (XII),

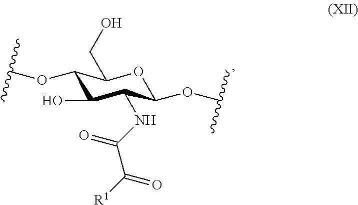

wherein each R¹ is independently a sidechain of a natural amino acid.

In some aspects, each R¹ is independently:

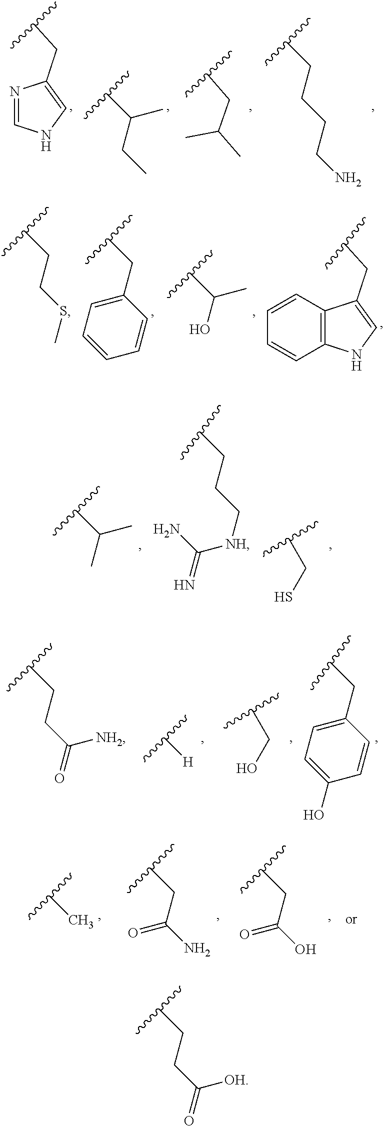

In some aspects, each R¹ is independently:

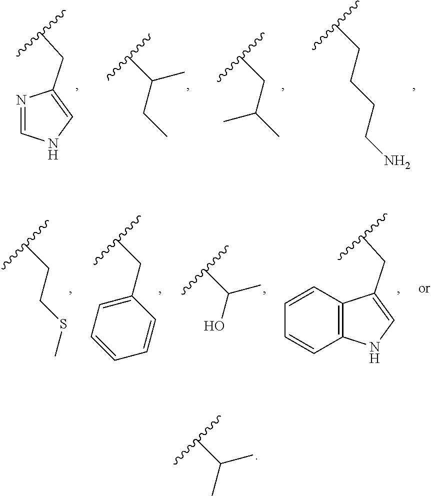

In some aspects, R¹ comprises:

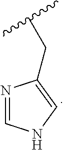 [SMILES: *Cc1c[nH]cn1]

In some aspects, R¹ comprises:

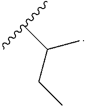 [SMILES: *C(C)CC]

In some aspects, R¹ comprises:

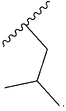 [SMILES: *CC(C)C]

In some aspects, R¹ comprises:

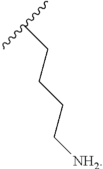 [SMILES: *CCCCN]

In some aspects, R¹ comprises:

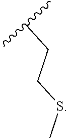 [SMILES: *CCSC]

In some aspects, R¹ comprises:

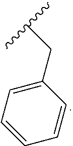 [SMILES: *Cc1ccccc1]

In some aspects, R¹ comprises:

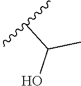 [SMILES: *C(C)O]

In some aspects, R¹ comprises:

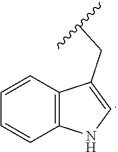 [SMILES: *Cc1c[nH]c2ccccc12]

In some aspects, R¹ comprises:

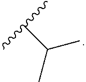 [SMILES: *C(C)C]

In some aspects, R¹ comprises:

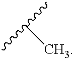

In some aspects, R¹ comprises:

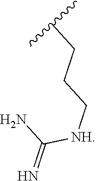 [SMILES: *CCCNC(=N)N]

In some aspects, R¹ comprises:

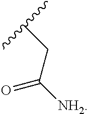 [SMILES: *CC(N)=O]

In some aspects, R¹ comprises:

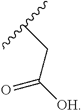 [SMILES: *CC(=O)O]

In some aspects, R¹ comprises:

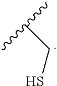 [SMILES: *CS]

In some aspects, R¹ comprises:

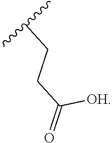 [SMILES: *CCC(=O)O]

In some aspects, R¹ comprises:

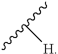

In some aspects, R¹ comprises:

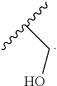 [SMILES: *CO]

In some aspects, R¹ comprises:

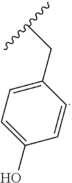 [SMILES: *Cc1ccc(O)cc1]

In some aspects, R¹ comprises:

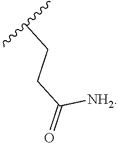 [SMILES: *CCC(N)=O]

In some aspects, not more than 50%, not more than 45%, not more than 40%, not more than 35%, not more than 30%, not more than 25%, not more than 20%, not more than 15%, not more than 10%, or not more than 5% of amines of the chitosan are acetylated.

In some aspects, the chitosan comprises a copolymer. In some aspects, the copolymer is a linear copolymer, a block copolymer, an alternating copolymer, a periodic copolymer, a statistical copolymer, a stereoblock copolymer, a gradient copolymer, branched copolymer, a graft copolymer, a start copolymer, or a combination thereof. In some aspects, the copolymer comprises Formula (I), Formula (II), Formula (III), Formula (IV), or combinations thereof:

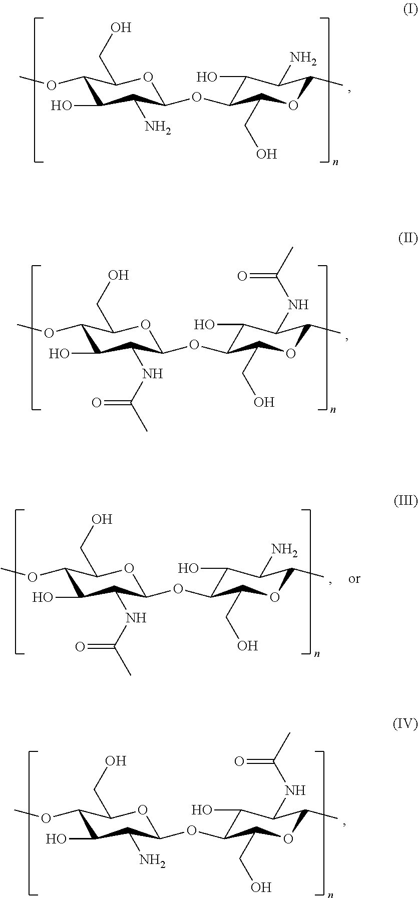

wherein each instance of n is independently an integer.

In some aspects, each instance of n is independently an integer of from 1 to 1000. In some aspects, the chitosan comprises two or more of Formula (VI), Formula (VIII), Formula (X), Formula (XII), or Formula (XIV). In some aspects, the chitosan comprises a ratio of Formula (VI) to Formula (VIII) of from 10:1 to 1:10, from 100:1 to 10:1, or from 1:10 to 1:100. In some aspects, the alpha-keto acid is non-stochastically distributed about the chitosan. In some aspects, a stoichiometric ratio of the chitosan amine and the alpha-keto acid is from 1:1 to 1:10, from 1:1 to 1:100, from 1:1 to 1:1000, from 1:1 to 1:10,000, or from 1:1 to 1:100,000. In some aspects, a weight ratio of the chitosan to the alpha-keto acid is from 1:1 to 1:10, from 1:1 to 1:100, from 1:1 to 1:1000, from 1:1 to 1:10,000, or from 1:1 to 1:100,000.

In various aspects, the present disclosure provides a composition comprising a compound of Formula (XVI),

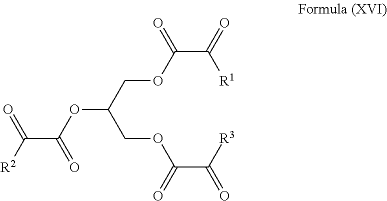

wherein R¹, R², and R³ comprise an amino acid sidechain each independently selected from TABLE 1, TABLE 2, or TABLE 3.

In some aspects, R¹, R², and R³ are the same. In some aspects, R¹, R², and R³ are different. In some aspects, one or more of R¹, R², and R³ is a histidine sidechain. In some aspects, one or more of R¹, R², and R³ is an isoleucine sidechain. In some aspects, one or more of R¹, R², and R³ is a leucine sidechain. In some aspects, one or more of R¹, R², and R³ is a lysine sidechain. In some aspects, one or more of R¹, R², and R³ is a methionine sidechain. In some aspects, one or more of R¹, R², and R³ is a phenylalanine sidechain. In some aspects, one or more of R¹, R², and R³ is a threonine sidechain. In some aspects, one or more of R¹, R², and R³ is a tryptophan sidechain. In some aspects, one or more of R¹, R², and R³ is a valine sidechain. In some aspects, one or more of R¹, R², and R³ is an alanine sidechain. In some aspects, one or more of R¹, R², and R³ is an arginine sidechain. In some aspects, one or more of R¹, R², and R³ is an asparagine sidechain. In some aspects, one or more of R¹, R², and R³ is an aspartate sidechain. In some aspects, one or more of R¹, R², and R³ is a cysteine sidechain. In some aspects, one or more of R¹, R², and R³ is a glutamate sidechain. In some aspects, one or more of R¹, R², and R³ is a glycine sidechain. In some aspects, one or more of R¹, R², and R³ is a serine sidechain. In some aspects, one or more of R¹, R², and R³ is a tyrosine sidechain. In some aspects, one or more of R¹, R², and R³ is a glutamine sidechain.

In various aspects, the present disclosure provides a composition comprising a compound of Formula (XVII),

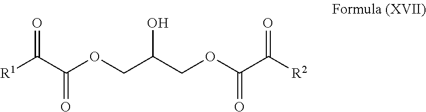

wherein R¹ and R² comprise an amino acid sidechain each independently selected from TABLE 1, TABLE 2, or TABLE 3.

In some aspects, R¹ and R² are the same. In some aspects, R¹ and R² are different. In some aspects, one or both of R¹ and R² is a histidine sidechain. In some aspects, one or both of R¹ and R² is an isoleucine sidechain. In some aspects, one or both of R¹ and R² is a leucine sidechain. In some aspects, one or both of R¹ and R² is a lysine sidechain. In some aspects, one or both of R¹ and R² is a methionine sidechain. In some aspects, one or both of R¹ and R² is a phenylalanine sidechain. In some aspects, one or both of R¹ and R² is a threonine sidechain. In some aspects, one or both of R¹ and R² is a tryptophan sidechain. In some aspects, one or both of R¹ and R² is a valine sidechain. In some aspects, one or both of R¹ and R² is an alanine sidechain. In some aspects, one or both of R¹ and R² is an arginine sidechain. In some aspects, one or both of R¹ and R² is an asparagine sidechain. In some aspects, one or both of R¹ and R² is an aspartate sidechain. In some aspects, one or both of R¹ and R² is a cysteine sidechain. In some aspects, one or both of R¹ and R² is a glutamate sidechain. In some aspects, one or both of R¹ and R² is a glycine sidechain. In some aspects, one or both of R¹ and R² is a serine sidechain. In some aspects, one or both of R¹ and R² is a tyrosine sidechain. In some aspects, one or both of R¹ and R² is a glutamine sidechain.

In various aspects, the present disclosure provides a composition comprising a compound of Formula (XVIII),

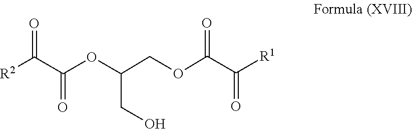

wherein R¹ and R² comprise an amino acid sidechain each independently selected from TABLE 1, TABLE 2, OR TABLE 3.

In some aspects, R¹ and R² are the same. In some aspects, R¹ and R² are different. In some aspects, one or both of R¹ and R² is a histidine sidechain. In some aspects, one or both of R¹ and R² is an isoleucine sidechain. In some aspects, one or both of R¹ and R² is a leucine sidechain. In some aspects, one or both of R¹ and R² is a lysine sidechain. In some aspects, one or both of R¹ and R² is a methionine sidechain. In some aspects, one or both of R¹ and R² is a phenylalanine sidechain. In some aspects, one or both of R¹ and R² is a threonine sidechain. In some aspects, one or both of R¹ and R² is a tryptophan sidechain. In some aspects, one or both of R¹ and R² is a valine sidechain. In some aspects, one or both of R¹ and R² is an alanine sidechain. In some aspects, one or both of R¹ and R² is an arginine sidechain. In some aspects, one or both of R¹ and R² is an asparagine sidechain. In some aspects, one or both of R¹ and R² is an aspartate sidechain. In some aspects, one or both of R¹ and R² is a cysteine sidechain. In some aspects, one or both of R¹ and R² is a glutamate sidechain. In some aspects, one or both of R¹ and R² is a glycine sidechain. In some aspects, one or both of R¹ and R² is a serine sidechain. In some aspects, one or both of R¹ and R² is a tyrosine sidechain. In some aspects, one or both of R¹ and R² is a glutamine sidechain.

In various aspects, the present disclosure provides a composition comprising a compound of Formula (XIX),

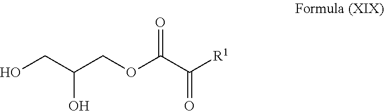

wherein R¹ comprises an amino acid sidechain selected from TABLE 1, TABLE 2, OR TABLE 3.

In some aspects, R¹ is a histidine sidechain. In some aspects, R¹ is an isoleucine sidechain. In some aspects, R¹ is a leucine sidechain. In some aspects, R¹ is a lysine sidechain. In some aspects, R¹ is a methionine sidechain. In some aspects, R¹ is a phenylalanine sidechain. In some aspects, R¹ is a threonine sidechain. In some aspects, R¹ is a tryptophan sidechain. In some aspects, R¹ is a valine sidechain. In some aspects, R¹ is an alanine sidechain. In some aspects, R¹ is an arginine sidechain. In some aspects, R¹ is an asparagine sidechain. In some aspects, R¹ is an aspartate sidechain. In some aspects, R¹ is a cysteine sidechain. In some aspects, R¹ is a glutamate sidechain. In some aspects, R¹ is a glycine sidechain. In some aspects, R¹ is a serine sidechain. In some aspects, R¹ is a tyrosine sidechain. In some aspects, R¹ is a glutamine sidechain.

In various aspects, the present disclosure provides a composition comprising a compound of Formula (XVI),

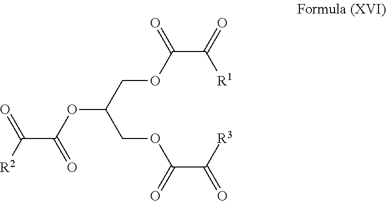

wherein R¹, R², and R³ comprise an amino acid sidechain each independently selected from the group consisting of

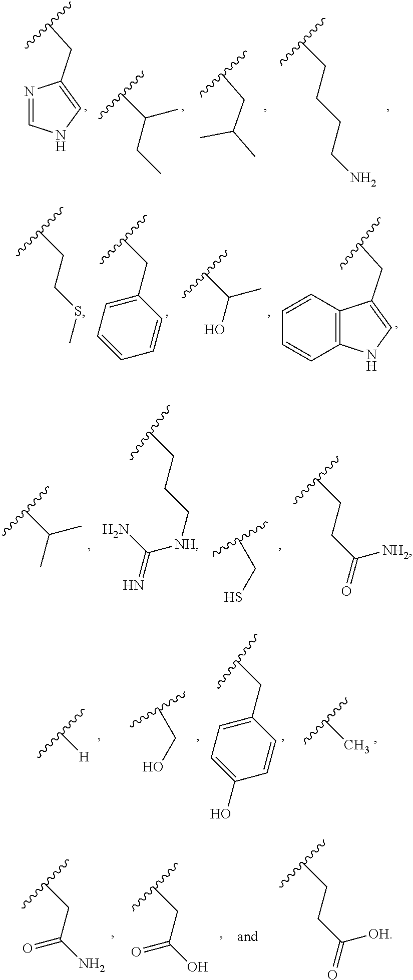

In some aspects, R¹, R², and R³ comprise an amino acid sidechain each independently selected from the group consisting of

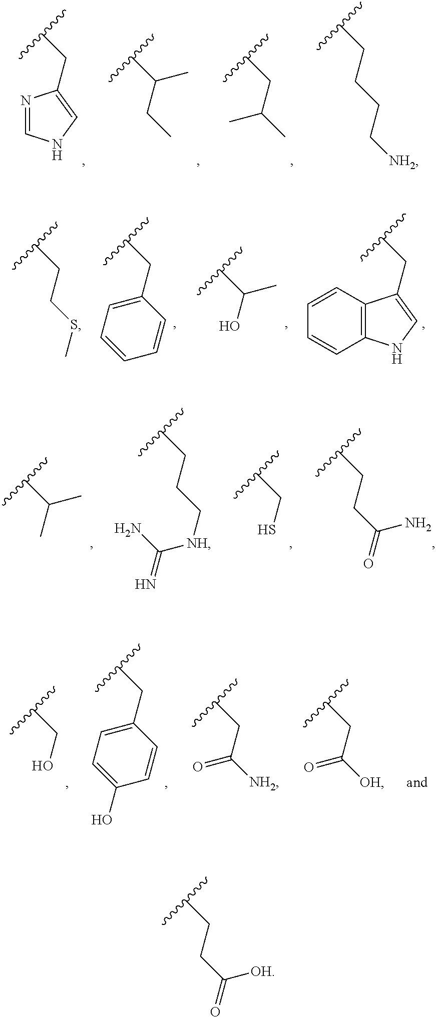

In some aspects, R¹, R², and R³ comprise an amino acid sidechain each independently selected from the group consisting of

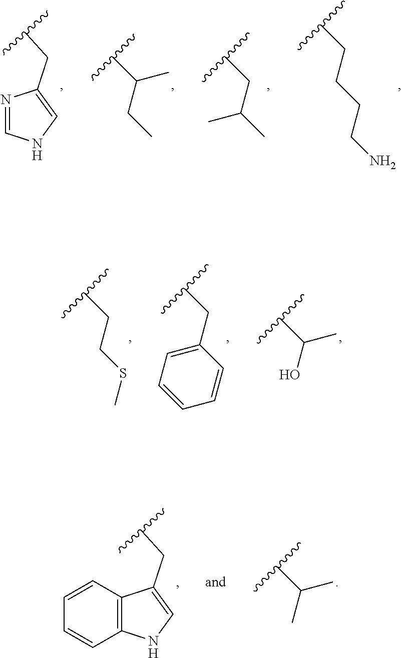

In some aspects, R¹, R², and R³ are the same. In some aspects, R¹, R², and R³ are different. In some aspects, one or more of R¹, R², and R³ is

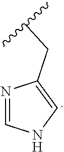 [SMILES: *Cc1c[nH]cn1]

In some aspects, one or more of R¹, R², and R³ is

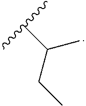 [SMILES: *C(C)CC]

In some aspects, one or more of R¹, R², and R³ is

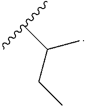 [SMILES: *C(C)CC]

In some aspects, one or more of R¹, R², and R³ is

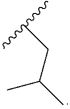 [SMILES: *CC(C)C]

In some aspects, one or more of R¹, R², and R³ is

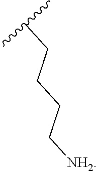 [SMILES: *CCCCN]

In some aspects, one or more of R¹, R², and R³ is

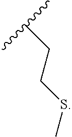 [SMILES: *CCSC]

In some aspects, one or more of R¹, R², and R³ is

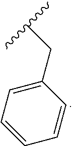 [SMILES: *Cc1ccccc1]

In some aspects, one or more of R¹, R², and R³ is

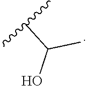 [SMILES: *C(C)O]

In some aspects, one or more of R¹, R², and R³ is

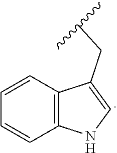 [SMILES: *Cc1c[nH]c2ccccc12]

In some aspects, one or more of R¹, R², and R³ is

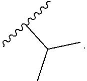 [SMILES: *C(C)C]

In some aspects, one or more of R¹, R², and R³ is

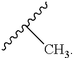

In some aspects, one or more of R¹, R², and R³ is

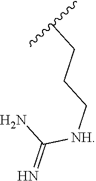 [SMILES: *CCCNC(=N)N]

In some aspects, one or more of R¹, R², and R³ is

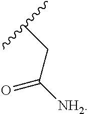 [SMILES: *CC(N)=O]

In some aspects, one or more of R¹, R², and R³ is

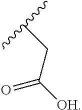 [SMILES: *CC(=O)O]

In some aspects, one or more of R¹, R², and R³ is

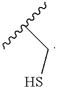 [SMILES: *CS]

In some aspects, one or more of R¹, R², and R³ is

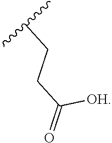 [SMILES: *CCC(=O)O]

In some aspects, one or more of R¹, R², and R³ is

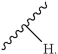

In some aspects, one or more of R¹, R², and R³ is

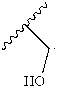 [SMILES: *CO]

In some aspects, one or more of R¹, R², and R³ is

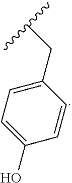 [SMILES: *Cc1ccc(O)cc1]

In some aspects, one or more of R¹, R², and R³ is

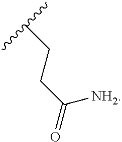 [SMILES: *CCC(N)=O]

In various aspects, the present disclosure provides a composition comprising a compound of Formula (XVII),

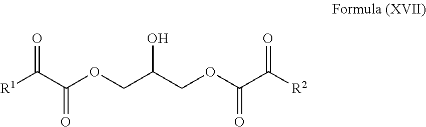

wherein R¹ and R² comprise an amino acid sidechain each independently selected from the group consisting of

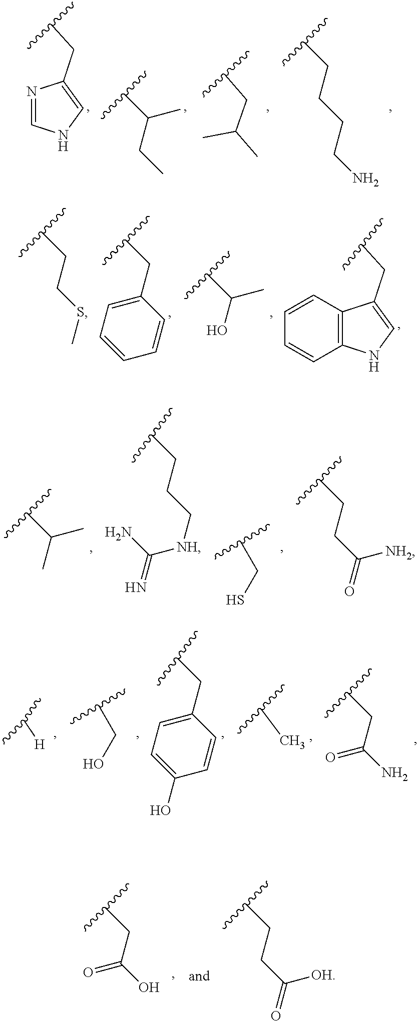

In some aspects, R¹ and R² comprise an amino acid sidechain each independently selected from the group consisting of

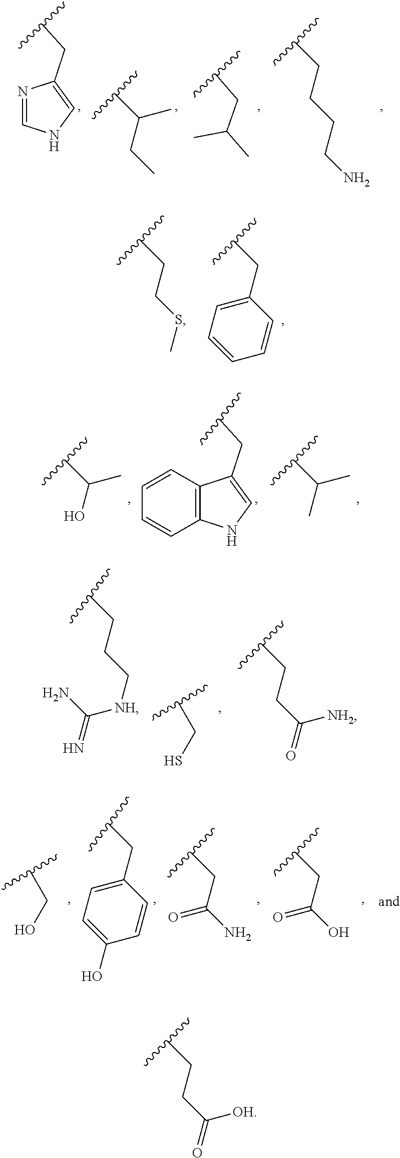

In some aspects, R¹ and R² comprise an amino acid sidechain each independently selected from the group consisting of

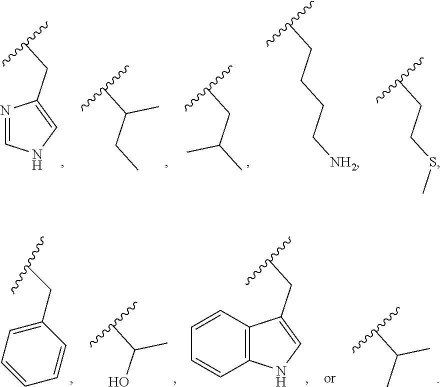

In some aspects, R¹ and R² are the same. In some aspects, R¹ and R² are different. In some aspects, one or both of R¹ and R² is

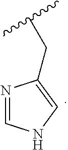 [SMILES: *Cc1c[nH]cn1]

In some aspects, one or both of R¹ and R² is

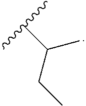 [SMILES: *C(C)CC]

In some aspects, one or both of R¹ and R² is

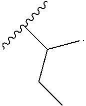 [SMILES: *C(C)CC]

In some aspects, one or both of R¹ and R² is

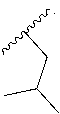 [SMILES: *CC(C)C]

In some aspects, one or both of R¹ and R² is

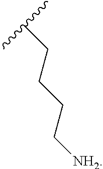 [SMILES: *CCCCN]

In some aspects, one or both of R¹ and R² is

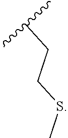 [SMILES: *CCSC]

In some aspects, one or both of R¹ and R² is

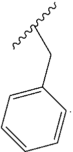 [SMILES: *Cc1ccccc1]

In some aspects, one or both of R¹ and R² is

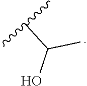 [SMILES: *C(C)O]

In some aspects, one or both of R¹ and R² is

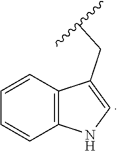 [SMILES: *Cc1c[nH]c2ccccc12]

In some aspects, one or both of R¹ and R² is

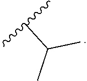 [SMILES: *C(C)C]

In some aspects, one or both of R¹ and R² is

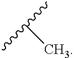

In some aspects, one or both of R¹ and R² is

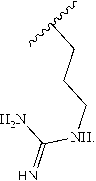 [SMILES: *CCCNC(=N)N]

In some aspects, one or both of R¹ and R² is

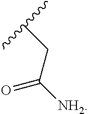 [SMILES: *CC(N)=O]

In some aspects, one or both of R¹ and R² is

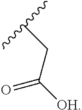 [SMILES: *CC(=O)O]

In some aspects, one or both of R¹ and R² is

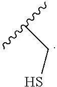 [SMILES: *CS]

In some aspects, one or both of R¹ and R² is

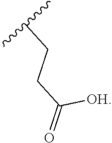 [SMILES: *CCC(=O)O]

In some aspects, one or both of R¹ and R² is

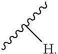

In some aspects, one or both of R¹ and R² is

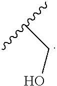 [SMILES: *CO]

In some aspects, one or both of R¹ and R² is

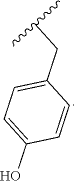 [SMILES: *Cc1ccc(O)cc1]

In some aspects, one or both of R¹ and R² is

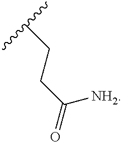 [SMILES: *CCC(N)=O]

In various aspects, the present disclosure provides a compositions comprising a compound of Formula (XVIII),

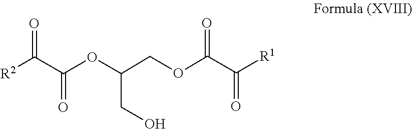

wherein R¹ and R² comprise an amino acid sidechain each independently selected from the group consisting of

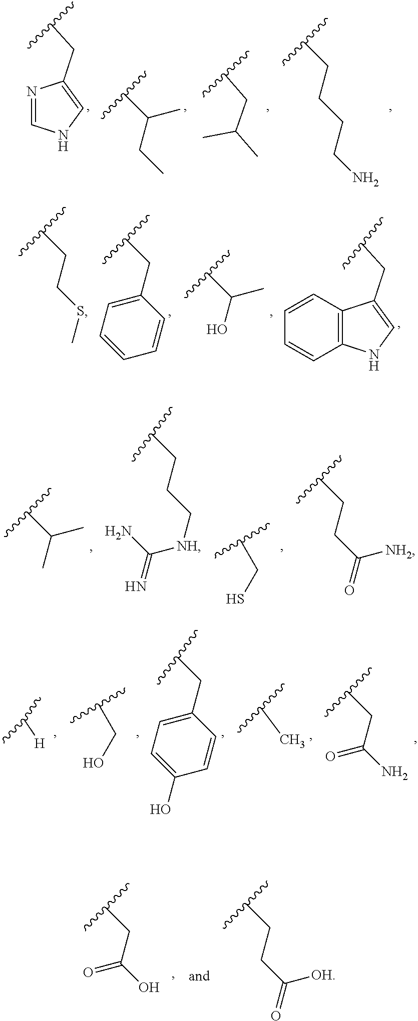

In some aspects, R¹ and R² comprise an amino acid sidechain each independently selected from the group consisting of

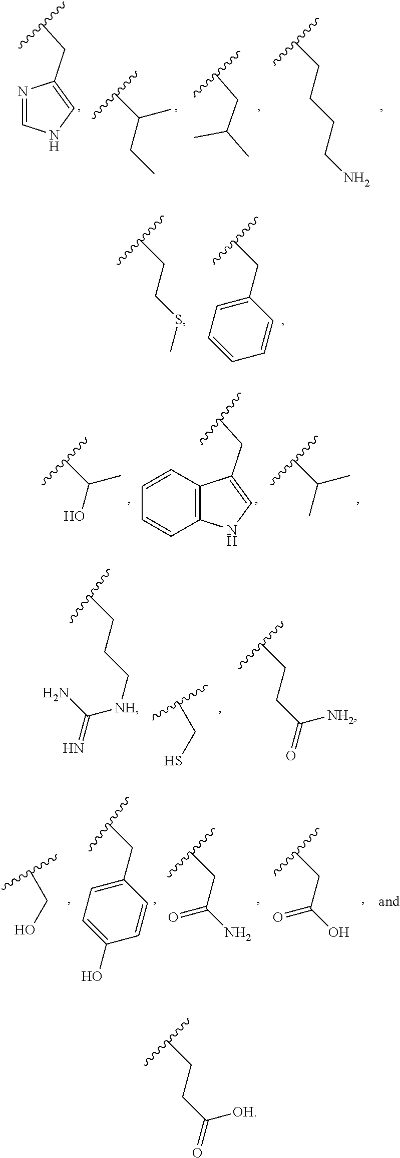

In some aspects, R¹ and R² comprise an amino acid sidechain each independently selected from the group consisting of

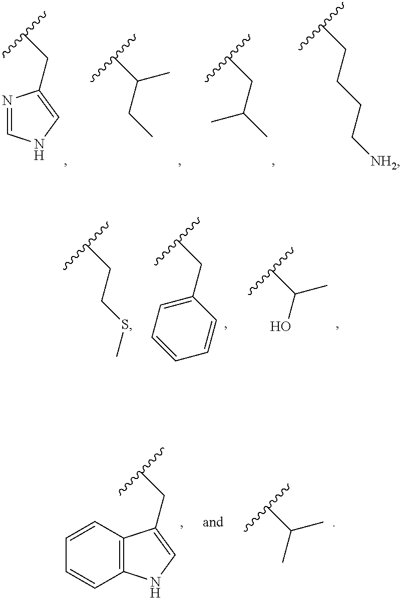

In some aspects, R¹ and R² are the same. In some aspects, R¹ and R² are different. In some aspects, one or both of R¹ and R² is

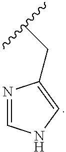 [SMILES: *Cc1c[nH]cn1]

In some aspects, one or both of R¹ and R² is

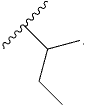 [SMILES: *C(C)CC]

In some aspects, one or both of R¹ and R² is

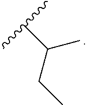 [SMILES: *C(C)CC]

In some aspects, one or both of R¹ and R² is

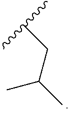 [SMILES: *CC(C)C]

In some aspects, one or both of R¹ and R² is

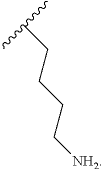 [SMILES: *CCCCN]

In some aspects, one or both of R¹ and R² is

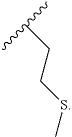 [SMILES: *CCSC]

In some aspects, one or both of R¹ and R² is

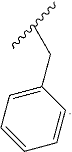 [SMILES: *Cc1ccccc1]

In some aspects, one or both of R¹ and R² is

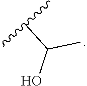 [SMILES: *C(C)O]

In some aspects, one or both of R¹ and R² is

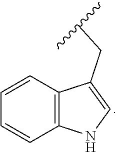 [SMILES: *Cc1c[nH]c2ccccc12]

In some aspects, one or both of R¹ and R² is

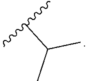 [SMILES: *C(C)C]

In some aspects, one or both of R¹ and R² is

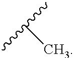

In some aspects, one or both of R¹ and R² is

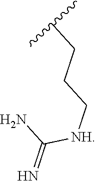 [SMILES: *CCCNC(=N)N]

In some aspects, one or both of R¹ and R² is

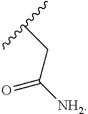 [SMILES: *CC(N)=O]

In some aspects, one or both of R¹ and R² is

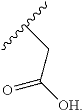 [SMILES: *CC(=O)O]

In some aspects, one or both of R¹ and R² is

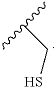 [SMILES: *CS]

In some aspects, one or both of R¹ and R² is

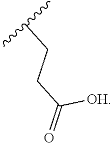 [SMILES: *CCC(=O)O]

In some aspects, one or both of R¹ and R² is

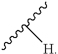

In some aspects, one or both of R¹ and R² is

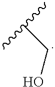 [SMILES: *CO]

In some aspects, one or both of R¹ and R² is

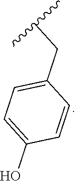 [SMILES: *Cc1ccc(O)cc1]

In some aspects, one or both of R¹ and R² is

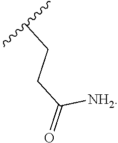 [SMILES: *CCC(N)=O]

In various aspects, the present disclosure provides a composition comprising a compound of Formula (XIX),

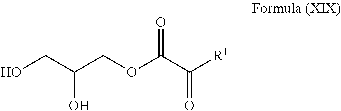

wherein R¹ comprises an amino acid sidechain selected from the group consisting of

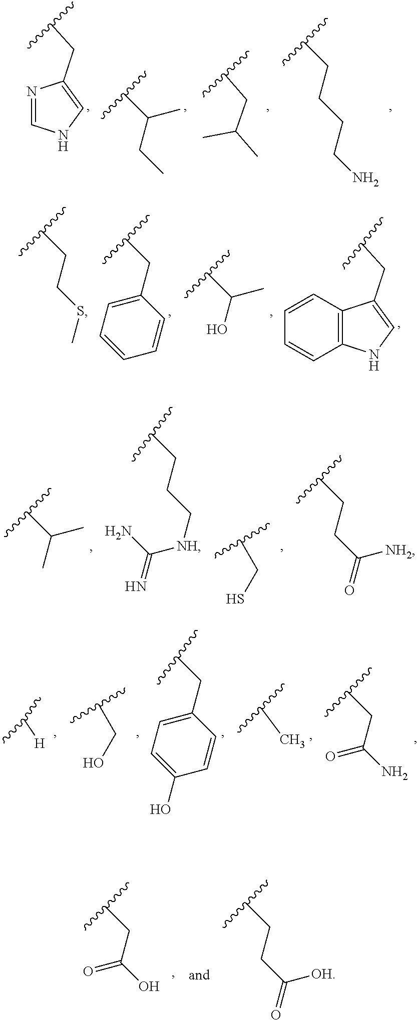

In various aspects, the present disclosure provides a composition comprising a compound of Formula (XX),

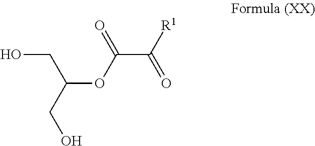

wherein R¹ comprises an amino acid sidechain selected from the group consisting of

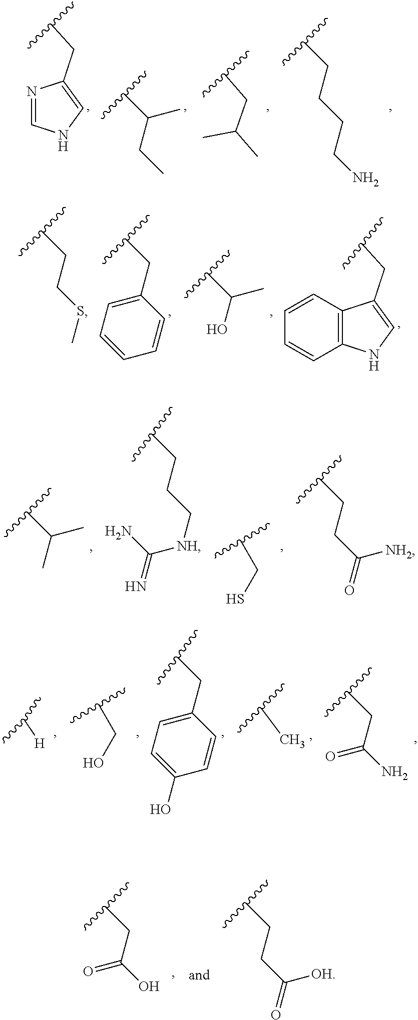

In some aspects, R¹ comprises an amino acid sidechain selected from the group consisting of

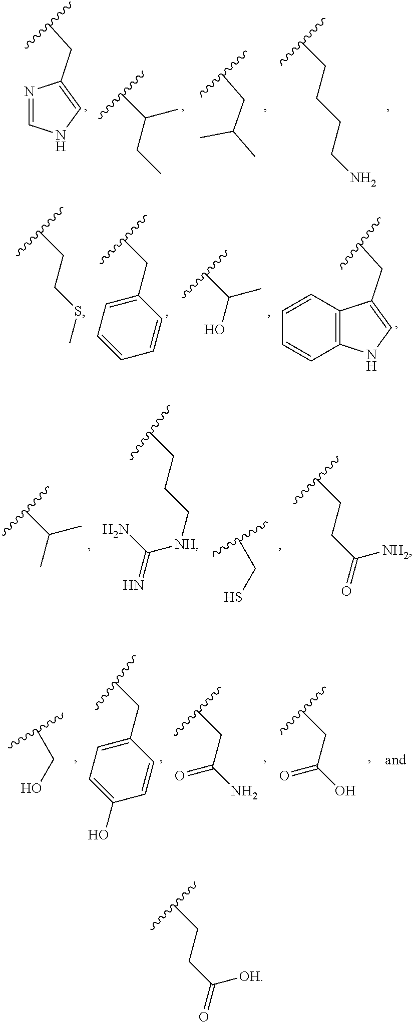

In some aspects, R¹ comprises an amino acid sidechain selected from the group consisting of

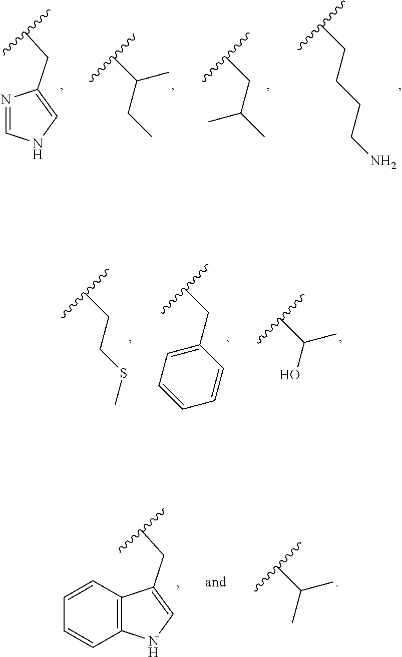

In some aspects, R¹ is

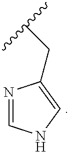 [SMILES: *Cc1c[nH]cn1]

In some aspects, R¹ is

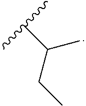 [SMILES: *C(C)CC]

In some aspects, R¹ is

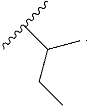 [SMILES: *C(C)CC]

In some aspects, R¹ is

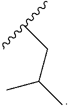 [SMILES: *CC(C)C]

In some aspects, R¹ is

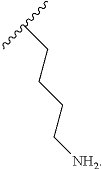 [SMILES: *CCCCN]

In some aspects, R¹ is

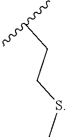 [SMILES: *CCSC]

In some aspects, R¹ is

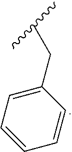 [SMILES: *Cc1ccccc1]

In some aspects, R¹ is

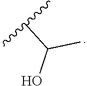 [SMILES: *C(C)O]

In some aspects, R¹ is

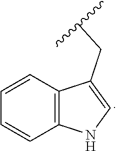 [SMILES: *Cc1c[nH]c2ccccc12]

In some aspects, R¹ is

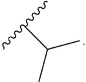 [SMILES: *C(C)C]

In some aspects, R¹ is

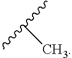

In some aspects, R¹ is

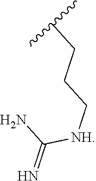 [SMILES: *CCCNC(=N)N]

In some aspects, R¹ is

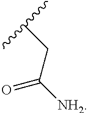 [SMILES: *CC(N)=O]

In some aspects, R¹ is

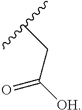 [SMILES: *CC(=O)O]

In some aspects, R¹ is

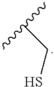 [SMILES: *CS]

In some aspects, R¹ is

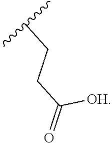 [SMILES: *CCC(=O)O]

In some aspects, R¹ is

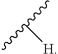

In some aspects, R¹ is

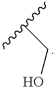 [SMILES: *CO]

In some aspects, R¹ is

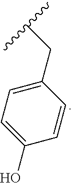 [SMILES: *Cc1ccc(O)cc1]

In some aspects, R¹ is

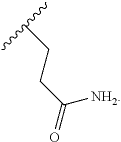 [SMILES: *CCC(N)=O]

In various aspects, the present disclosure provides a composition comprising a compound of Formula (XXI):

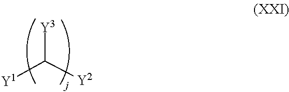

wherein: Y¹ and Y² are each independently selected from the group consisting of —CH₂Y³, —C(═O)H, —C(═O)OX₂, and —C(═O)NX²X³; each instance of Y³ is each independently selected from the group consisting of —H, —OX¹, —NX²X³, —C(═O)OX₂, and —C(═O)NX²X³; each instance of X¹ is independently selected from the group consisting of —H, —C₁₋₃ alkyl, —C(═O)CH₃, —C(═O)OX₂, —C(═O)NX²X³, —PO₃²⁻, —SO₃⁻, a C₄₋₉ monosaccharide, a C₈₋₁₈ disaccharide, and

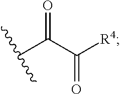

each instance of X² and X³ is independently selected from the group consisting of —H and —C₁-C₃ alkyl; each instance of R⁴ is independently selected from the group consisting of non-proline, non-alanine, and non-glycine natural amino acid sidechains, subscript j is an integer from 0 to 23; and at least one instance of Y¹, Y², and Y³ is —OX¹ of which X¹ is

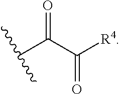

In some aspects, Y¹ and Y² are each —CH₂Y³. In some aspects, at most one instance of Y³ is

—C(═O)OX₂ and the remaining instances of Y³ are —OX¹.

In some aspects, the compound of Formula (XXI) is a compound of Formula (XXIa):

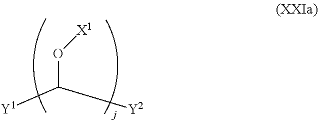

wherein: Y¹ and Y² are each independently selected from the group consisting of —CH₂Y³, —C(═O) H, —C(═O)OX₂, and —C(═O)NX²X³; each instance of Y³ is each independently selected from the group consisting of —H, —OX¹, —NX²X³, —C(═O)OX₂, and —C(═O)NX²X³; each instance of X¹ is independently selected from the group consisting of —H, —C₁₋₃ alkyl, —C(═O)CH₃, —C(═O)OX₂, —C(═O)NX²X³, —PO₃²⁻, —SO₃⁻, a C₄₋₉ monosaccharide, a C₈₋₁₈ disaccharide, and

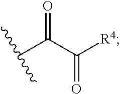

each instance of X² and X³ is independently selected from the group consisting of —H and —C₁-C₃ alkyl; each instance of R⁴ is independently selected from the group consisting of non-proline, non-alanine, and non-glycine natural amino acid sidechains, subscript j is an integer from 0 to 23; and at least one instance of X¹ is

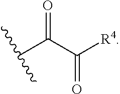

In some aspects, at most one instance of X¹ is —PO₃²⁻, —SO₃⁻, a C₄₋₉ monosaccharide, or a C₈₋₁₈ disaccharide, and the remaining instances are each independently selected from the group consisting of —H, C₁₋₃ alkyl, and

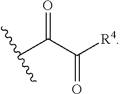

In some aspects, each instance of X¹ is independently selected from the group consisting of —H and

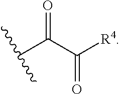

In some aspects, subscript j is an integer from 1 to 6. In some aspects, each instance of R⁴ is independently selected from the group consisting of

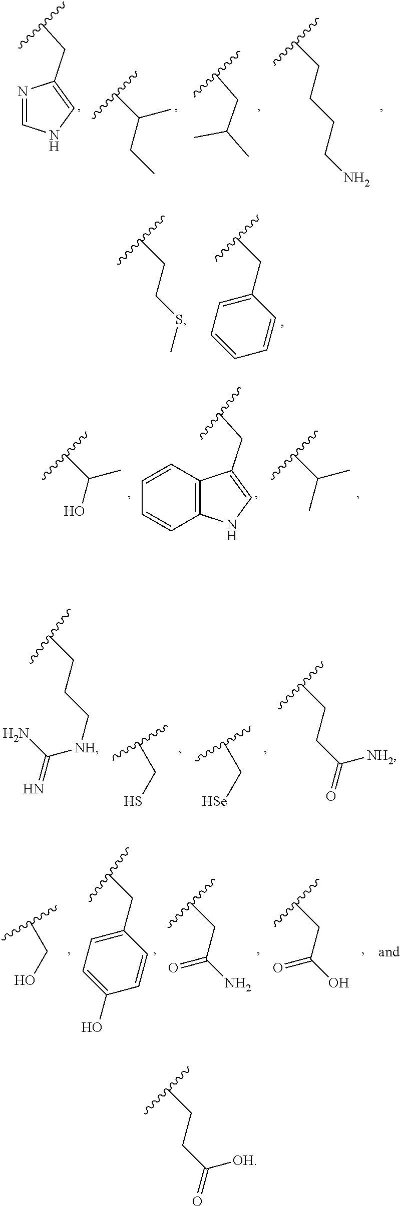

In some aspects, each instance of R⁴ is independently selected from the group consisting of

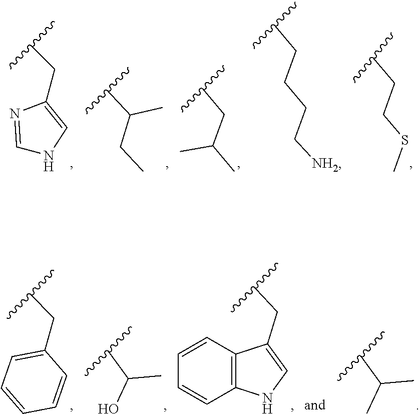

In various aspects, the present disclosure provides a composition comprising a compound of Formula (XXII):

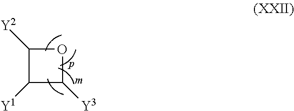

wherein: Y¹ is selected from the group consisting of —H, —OX¹, —C(═O)OX₂, —C(═O)NX²x³; Y² is selected from the group consisting of —H, —OX¹, —CH₂OX¹, —C(═O)OX₂, —C(═O)NX²X³, and

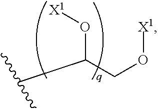

wherein subscript q is an integer from 1 to 3 and [symbol] denotes a point of attachment of Y² to the remainder of Formula (XXII); each instance of Y³ is each independently selected from the group consisting of —H, —OX¹, —C(═O)OX₂, and —C(═O)NX²X³; each instance of X¹ is independently selected from the group consisting of —H, —C₁₋₃ alkyl, —PO₃²⁻, —SO₃⁻, a C₄₋₉ monosaccharide, a C₈₋₁₈ disaccharide, and

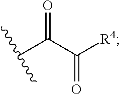

each instance of X² and X³ is independently selected from the group consisting of —H and C₁₋₃ alkyl; each instance of R⁴ is independently selected from the group consisting of non-proline, non-alanine, and non-glycine natural amino acid sidechains, and subscript m is an integer from 1 to 23, and subscript p is 0 or 1.

In some aspects, subscript m is an integer from 2 to 5. In some aspects, Y¹ is selected from the group consisting of —H and —OX¹. In some aspects, p is 0 and Y² is —OX¹. In some aspects, each instance of Y³ is each independently selected from the group consisting of —H and —OX¹.

In some aspects, the compound of Formula (XXII) is a compound of Formula (XXIIa):

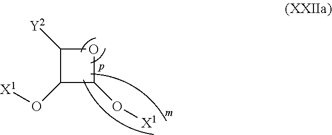

wherein: Y² is selected from the group consisting of —OX¹, —CH₂OX¹, and

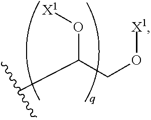

wherein subscript q is an integer from 1 to 3 and [symbol] denotes a point of attachment of Y² to the remainder of Formula (XXII); each instance of X¹ is independently selected from the group consisting of —H, —C₁₋₃ alkyl, —PO₃²⁻, —SO₃⁻, a C₄₋₉ monosaccharide, a C₈₋₁₈ disaccharide, and

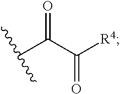

each instance of X² and X³ is independently selected from the group consisting of —H and C₁₋₃ alkyl; each instance of R⁴ is independently selected from the group consisting of non-proline, non-alanine, and non-glycine natural amino acid sidechains, subscript m is an integer from 1 to 23; and subscript p is 0 or 1.

In some aspects, one, two, or three instances of X¹ are

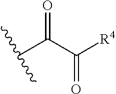

and the remaining instances are —H. In some aspects, at most one instance of X¹ is —PO₃²⁻, —SO₃⁻, a C₄₋₉ monosaccharide, or a C₈₋₁₈ disaccharide, and the remaining instances are each independently selected from the group consisting of —H and

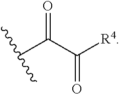

In some aspects, each instance of R⁴ is independently selected from the group consisting of

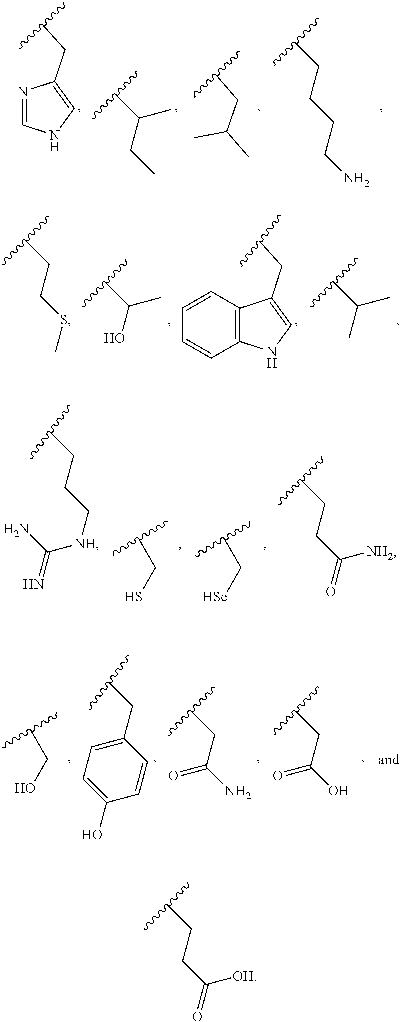

In some aspects, each instance of R⁴ is independently selected from the group consisting of

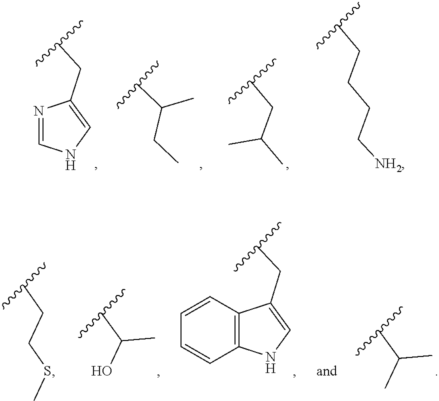

In some aspects, the composition further comprises a food component. In some aspects, the composition is formulated as a medical food. In some aspects, the medical food is a beverage, a drink mix, or a solid food.

In some aspects, the composition further comprises a pharmaceutically acceptable excipient. In some aspects, the composition is formulated for administration orally, topically, transdermally, rectally, intravenously, intra-arterially, intra-peritoneally, parenterally, or via inhalation.

In various aspects, the present disclosure provides a consumable formulation comprising a composition as described herein.

In some aspects, between about 30% and 90%, between about 50% and 80%, or between about 60% and 95% of dry weight of the consumable composition is carbohydrates, fats, protein, or a combination thereof. In some aspects, the consumable formulation is formulated as a liquid, a solid, a colloid, a gel, or a combination thereof. In some aspects, the consumable formulation is a medical grade food.

In various aspects, the present disclosure provides a medical food comprising a composition as described herein.

In some aspects, the medical food is formulated as a beverage, a drink mix, or a solid food. In some aspects, the medical food is formulated as a solid food. In some aspects, the medical food is formulated as a beverage. In some aspects, the medical food is a solid. In some aspects, the medical food is a liquid. In some aspects, the medical food is gelatinous. In some aspects, the medical food is formulated as a beverage, a bar, a cereal, a sports drink, a gel, a gelatin, a gelatinous gummy, a cracker, a chip, a puff, a granola cereal, a granola bar, a tablet, a powder, or an additive.

In some aspects, the medical food comprises a Cd content of less than 5 μg per daily serving or of less than 0.5 μg per g. In some aspects, the medical food comprises a Pb content of less than 5 μg per daily serving or less than 0.5 μg per g. In some aspects, the medical food comprises an As content of less than 15 μg per daily serving or less than 1.5 μg per g. In some aspects, the medical food comprises a Hg content of less than 30 μg per daily serving or less than 3 μg per g. In some aspects, the medical food comprises a Co content of less than 50 μg per daily serving or less than 5 μg per g. In some aspects, the medical food comprises a V content of less than 100 μg per daily serving or less than 10 μg per g. In some aspects, the medical food comprises a Ni content of less than 200 μg per daily serving or less than 20 μg per g. In some aspects, the medical food comprises a Tl content of less than 8 μg per daily serving or less than 0.8 μg per g. In some aspects, the medical food comprises an Au content of less than 300 μg per daily serving or less than 30 μg per g. In some aspects, the medical food comprises a Pd content of less than 100 μg per daily serving or less than 10 μg per g. In some aspects, the medical food comprises an Ir content of less than 100 μg per daily serving or less than 10 μg per g. In some aspects, the medical food comprises an Os content of less than 100 μg per daily serving or less than 10 μg per g. In some aspects, the medical food comprises a Rh content of less than 100 μg per daily serving or less than 10 μg per g. In some aspects, the medical food comprises a Ru content of less than 100 μg per daily serving or less than 10 μg per g. In some aspects, the medical food comprises a Se content of less than 150 μg per daily serving or less than 15 μg per g. In some aspects, the medical food comprises an Ag content of less than 150 μg per daily serving or less than 15 μg per g. In some aspects, the medical food comprises a Pt content of less than 100 μg per daily serving or less than 10 μg per g. In some aspects, the medical food comprises a Li content of less than 550 μg per daily serving or less than 55 μg per g. In some aspects, the medical food comprises a Sb content of less than 1200 μg per daily serving or less than 120 μg per g. In some aspects, the medical food comprises a Ba content of less than 1400 μg per daily serving or less than 140 μg per g. In some aspects, the medical food comprises a Mo content of less than 3000 μg per daily serving or less than 300 μg per g. In some aspects, the medical food comprises a Cu content of less than 3000 μg per daily serving or less than 300 μg per g. In some aspects, the medical food comprises a Sn content of less than 6000 μg per daily serving or less than 600 μg per g. In some aspects, the medical food comprises a Cr content of less than 11000 μg per daily serving or less than 1100 μg per g.

In some aspects, the medical food comprises a microbial plate count of no more than 1000 colony forming per mL, no more than 10,000 colony forming per mL, or no more than 20,000 colony forming per mL.

In various aspects, the present disclosure provides a method of increasing a protein level in a subject in need thereof, the method comprising administering to the subject a composition as described herein, a consumable composition as described herein, or a medical food as described herein, thereby increasing the protein level in the subject.

In some aspects, the subject does not have kidney disease. In some aspects, the subject is at risk of developing kidney disease. In some aspects, the subject has a condition that puts the subject at risk of kidney disease. In some aspects, the condition is diabetes, high blood pressure, heart disease, obesity, a family history of chronic kidney disease, inherited kidney disorders, past damage to the kidneys, or old age. In some aspects, the subject has a kidney disease.

In various aspects, the present disclosure provides a method of treating disease in a subject in need thereof, the method comprising administering to the subject a composition as described herein, a consumable composition as described herein, or a medical food as described herein, thereby treating the disease in the subject.

In some aspects, the subject has kidney disease. In some aspects, the subject is at risk of developing kidney disease. In some aspects, the kidney disease is chronic kidney disease, Fabry disease, cystinosis, glomerulonephritis, IgA nephropathy, lupus nephritis, atypical hemolytic uremic syndrome, polycystic kidney disease, diabetic kidney disease, or uremia. In some aspects, the subject is in an anabolic need.

### INCORPORATION BY REFERENCE

All publications, patents, and patent applications mentioned in this specification are herein incorporated by reference to the same extent as if each individual publication, patent, or patent application was specifically and individually indicated to be incorporated by reference.

### BRIEF DESCRIPTION OF THE DRAWINGS

The novel features of the invention are set forth with particularity in the appended claims. A better understanding of the features and advantages of the present invention will be obtained by reference to the following detailed description that sets forth illustrative embodiments, in which the principles of the invention are utilized, and the accompanying drawings of which:

FIG. 1 illustrates a theoretical plot of alpha-keto acid detection as a function of alpha-keto acid added to solution.

FIG. 2A illustrates a proton NMR spectrum of a chitosan composition with two alpha-keto acids, 3-methyl-2-oxo-butanoic acid (alpha-keto acid analogue of valine) and 3-methyl-2-oxopentanoic acid (alpha-keto acid analogue of isoleucine).

FIG. 2B illustrates an overlay of two proton NMR spectra including a chitosan composition with a single keto acid, 3-methyl-2-oxo-butanoic acid (alpha-keto acid analogue of valine), top, and a chitosan composition with two alpha-keto acids, 3-methyl-2-oxo-butanoic acid (alpha-keto acid analogue of valine) and 3-methyl-2-oxopentanoic acid (alpha-keto acid analogue of isoleucine), bottom.

FIG. 2C illustrates the assigned peaks for a chitosan composition with two alpha-keto acids, 3-methyl-2-oxo-butanoic acid (alpha-keto acid analogue of valine) and 3-methyl-2-oxopentanoic acid (alpha-keto acid analogue of isoleucine) as determined by the overlay in FIG. 2B.

FIG. 3 shows HPLC spectra of a keto acid (KA) control mixture, Sample 80 (0.5% CS+0.1% Mg ST+1% ALG), Sample 81 (0.5% CS+0.025% DSPE-PEG+0.1% Mg ST+1% ALG), and Sample 82 (0.5% CS+0.025% DSPE-PEG+5% mannitol) measured at an absorbance of 210 nm.

FIG. 4 shows an HPLC spectrum of Sample 80 (0.5% CS+0.1% Mg ST+1% ALG) measured at an absorbance of 210 nm (top) and 280 nm (bottom).

FIG. 5 shows an HPLC spectrum of Sample 81 (0.5% CS+0.025% DSPE-PEG+0.1% Mg ST+1% ALG) measured at an absorbance of 210 nm (top) and 280 nm (bottom).

FIG. 6 shows an HPLC spectrum of Sample 82 (0.5% CS+0.025% DSPE-PEG+5% mannitol) measured at an absorbance of 210 nm (top) and 280 nm (bottom).

FIG. 7 is a bar graph of the percent bound (% Bound) keto acid analogues for glycine (Gly), valine (Val), methionine (Met), isoleucine (Ile), tyrosine (Tyr), leucine (Leu), phenylalanine (Phe), and tryptophan (Trp) for liquid formulations of a control sample that did not comprise chitosan (Control), Sample 80 (0.5% CS+0.1% Mg ST+1% ALG), Sample 81 (0.5% CS+0.025% DSPE-PEG+0.1% Mg ST+1% ALG), and Sample 82 (0.5% CS+0.025% DSPE-PEG+5% mannitol).

FIG. 8 is a bar graph of the percent bound (% Bound) keto acid analogues for glycine (Gly), valine (Val), methionine (Met), isoleucine (Ile), tyrosine (Tyr), leucine (Leu), phenylalanine (Phe), and tryptophan (Trp) for lyophilized formulations of a control sample that did not comprise chitosan (Control), Sample 80 (0.5% CS+0.1% Mg ST+1% ALG), Sample 81 (0.5% CS+0.025% DSPE-PEG+0.1% Mg ST+1% ALG), and Sample 82 (0.5% CS+0.025% DSPE-PEG+5% mannitol).

FIG. 9A provides a graph of the pressure profile during the lyophilization of the chitosan and keto acid compositions. The pressure profile includes the set lyophilizer pressure (“Trace 1”) as well as the measured Pirani pressure (“Trace 2”).

FIG. 9B provides a graph of the temperature profile during the lyophilization of the chitosan and keto acid compositions. The temperature profile includes the set temperature in the lyophilizer (“Trace 1”) and the measured temperature in the lyophilizer (“Trace 2”)

FIG. 10A provides a photograph of Sample 80 (0.5% CS+0.1% Mg ST+1% ALG), Sample 81 (0.5% CS+0.025% DSPE-PEG+0.1% Mg ST+1% ALG), and Sample 82 (0.5% CS+0.025% DSPE-PEG+5% mannitol) before lyophilization.

FIG. 10B provides a photograph of Sample 80 (0.5% CS+0.1% Mg ST+1% ALG), Sample 81 (0.5% CS+0.025% DSPE-PEG+0.1% Mg ST+1% ALG), and Sample 82 (0.5% CS+0.025% DSPE-PEG+5% mannitol) after lyophilization.

FIG. 10C provides a photograph of Sample 80 (0.5% CS+0.1% Mg ST+1% ALG), Sample 81 (0.5% CS+0.025% DSPE-PEG+0.1% Mg ST+1% ALG), and Sample 82 (0.5% CS+0.025% DSPE-PEG+5% mannitol) after grinding.

FIG. 10D provides a photograph of Sample 80 (0.5% CS+0.1% Mg ST+1% ALG), Sample 81 (0.5% CS+0.025% DSPE-PEG+0.1% Mg ST+1% ALG), and Sample 82 (0.5% CS+0.025% DSPE-PEG+5% mannitol) after reconstitution.

### DETAILED DESCRIPTION

The present disclosure provides biocompatible biopolymer keto acid complexes and compositions of keto ester polyols which, upon administration, can release anabolic amino acid precursors in the form of alpha-keto acids. As alpha-keto acids are readily converted to amino acids in vivo, these complexes can be used to supplement amino acids for subjects with limited protein intakes. Following administration, the compositions of biocompatible biopolymer keto acid complexes and compositions of keto ester polyols may be converted to amino acids without producing or with limited production of nitrogen by-products, such as ammonia or urea, which can otherwise create further complications for kidney disease patients. The production of nitrogen by-products may be lower following ingestion of a composition of the present disclosure compared to production of nitrogen by-products following ingestion of a comparable amount of an amino acid composition. While amino acid administration can lead to a spike in blood ammonia levels, and alpha-keto acid salts (e.g., sodium pyruvate) can generate buildups of inorganic counter ions, the complexes can generate low amounts of ammonia and can be administered with minimal inorganic cation (e.g., Na²⁺, Ca²⁺, or Mg²⁺) content. Accordingly, the compositions of biocompatible biopolymer keto acid complexes and compositions of keto ester polyols of the present disclosure can provide a low hepatic stress alternative for administering amino acid nutrients.

The hepatic stress can also be referred to as a metabolic burden on the kidneys. Hepatic stress (e.g., metabolic burden on the kidneys) may be increased by amino acid administration due to the nitrogen content from the amino group or by alpha keto acid salt administration due to the salt (e.g., Na²⁺, Ca²⁺, or Mg²⁺) content. Accordingly, there is a need for protein administration with decreased hepatic stress (e.g., metabolic burden on the kidneys). The administration of the compositions described herein (e.g., biopolymer keto acid compositions) for protein administration may result in a decrease in hepatic stress (e.g., metabolic burden on the kidneys) as compared to other routes of protein administration (e.g., amino acid administration or alpha keto acid salt administration). The decrease in hepatic stress (e.g., metabolic burden on the kidneys) from the administration of the compositions herein (e.g., biopolymer keto acid compositions) as compared to other routes of protein administration (e.g., amino acid administration or alpha keto acid salt administration) may be measured by comparing markers of kidney function. Markers of kidney function may be measured by the blood urea nitrogen (BUN) level, the urine-creatinine ratio (uACR), the estimated glomerular filtration rate (eGFR), or a combination thereof.

The compositions as described herein (e.g., biopolymer keto acid compositions) may have a lower nitrogen content per kg of protein than other compositions for protein administration (e.g., amino acids compositions). The nitrogen content in the compositions described herein (e.g., biopolymer keto acid compositions) or other compositions may be measured by USP method 461. The administration of the compositions described herein (e.g., biopolymer keto acid compositions) for protein administration may result in a decrease in nitrogen intake per kg of protein administered as compared to other routes of protein administration (e.g., amino acid administration). The decrease in nitrogen intake may result in a decrease in nitrogen metabolism by-products (e.g., urea, ammonia, or creatinine). The decrease in nitrogen intake may be measured by a decrease in nitrogen metabolism by-products (e.g., urea, ammonia, or creatinine) by blood or urine tests.

The administration of the compositions described herein (e.g., biopolymer keto acid compositions) for protein administration may result in a decrease in salt (e.g., Na²⁺, Ca²⁺, or Mg²⁺) intake per kg of protein administered as compared to other routes of protein administration (e.g., amino acid administration or alpha keto acid salt administration). The decrease in salt (e.g., Na²⁺, Ca²⁺, or Mg²⁺) intake per kg of protein administered may be measured by a salt (e.g., Na²⁺, Ca²⁺, or Mg²⁺) level in blood or urine.

### Biopolymer Keto Acid Compositions

A composition of the present disclosure can include a biopolymer and an alpha-keto acid (also referred to as an α-keto acid). A broad range of biopolymers are amenable for use in the compositions of the present disclosure. In many embodiments, the biopolymer is a polysaccharide (e.g., a chitosan, a starch, a cellulose, an amino polysaccharide, an alginate, a carrageenan, a chitin, a chondroitin sulfate, a dextran, a galactomannan, a glycogen, a hyaluronic acid, a glycogen, a galactogen, an inulin, an arabinoxylan, or a pectin). The biopolymer may be coupled to a keto acid (e.g., an alpha-keto acid analogue of an amino acid), forming what may be referred to herein as a biopolymer-keto acid composition. For example, the composition may be formulated as a salt wherein the keto acid is anionic and the biopolymer is cationic. In some embodiments, the biopolymer is non-covalently coupled to the keto acid. In some embodiments, the biopolymer is covalently coupled to the keto acid.

In some embodiments, the biopolymer is an amino polysaccharide. In some embodiments, the biopolymer is an amino polysaccharide such as an amino cellulose, an amino dextran, an amino galactose, an amino arabinose, a chitin, a chitosan, or a combination thereof. In some cases, the biopolymer comprises chitosan. In such cases, the composition can be referred to as a chitosan keto acid composition. For cases in which the biopolymer comprises chitosan and the keto acid is an alpha-keto acid, the composition can be referred to as a chitosan alpha-keto acid composition.

The compositions described herein (e.g., alpha-keto acid compositions) may be formulated for consumption, for example as a food (e.g., a medical grade food), an inhalable powder, or an intravenous formulation, and may be administered for the treatment of protein deficiency in kidney disease patients.

The compositions of the present disclosure (e.g., alpha-keto acid compositions) may be administered in the form of a medical grade food or as a food supplement to treat or prevent protein deficiency in kidney disease patients. There are several different classes of amino acids including essential, conditionally essential, and non-essential amino acids. Essential amino acids are amino acids that cannot be synthesized in vivo at the level needed for metabolism and are obtained from diet. Conditionally essential amino acids (also referred to as quasi-essential amino acids) are amino acids that might have their synthesis limited under certain conditions or pathophysiologies. Non-essential amino acids are amino acids that are readily synthesized in vivo in sufficient quantities for metabolism. Essential amino acids, conditionally essential amino acids, and non-essential amino acids can be generated through bioconversion of the chitosan alpha-keto acid compositions disclosed herein.

The compositions described herein may comprise a polysaccharide and an alpha-keto acid composition comprising one or more of an alpha-keto acid analogues of an amino acid. The alpha-keto acid composition may comprise any of the alpha-keto acids as described herein. In some embodiments, the alpha-keto acid composition comprises an alpha-keto acid analogue of Histidine, Isoleucine, Leucine, Lysine, Methionine, Phenylalanine, Threonine, Tryptophan, Valine, Arginine, Cysteine, Glutamine, Glycine, Serine, Tyrosine, Alanine, Asparagine, Aspartic Acid, Glutamic Acid, or any combination thereof. In some embodiments, the alpha-keto acid composition comprises an alpha-keto acid analogue of Histidine, Isoleucine, Leucine, Lysine, Methionine, Phenylalanine, Threonine, Tryptophan, Valine, or any combination thereof. In some embodiments, the alpha-keto acid analogue of an amino acid is an alpha-keto acid analogue of an essential amino acid. In some embodiments, the alpha-keto acid composition comprises an alpha-keto acid analogue selected from TABLE 1, TABLE 2, TABLE 3, or any combination thereof.

### Chitosan Alpha-Keto Acid Compositions

In one embodiment, the present disclosure provides a composition comprising a chitosan and an alpha-keto acid. In some cases, the chitosan is covalently coupled to the alpha-keto acid. In some cases, the chitosan is non-covalently coupled to the alpha-keto acid. In some cases, the alpha-keto acid is intercalated within the chitosan. In some cases, alpha-keto acid is coupled to a surface of the chitosan. Following administration, the composition can be readily metabolized to form alpha-keto acids, amino acids, and other anabolically beneficial species in vivo. The composition can contain low bioavailable ammonia and inorganic cation (e.g., Ca²⁺, Mg²⁺, Na⁺, K⁺) and therefore can serve as hepatically-compatible nutrient sources for subjects with amino acid deficiencies or low dietary protein tolerances.

### Chitosan

Chitosan is a biopolymer comprising monomers of D-glucosamine and N-acetylglucosamine. Chitosan may be isolated from shells, shellfish, exoskeletons of shellfish or insects, and fungi. Chitosan may be characterized by a degree of deacetylation. Chitosan may be also known as poly(D-glucosamine) and can have varying molecular weight dependent on the number of repeating monomers and degree of deacetylation. In some embodiments, chitosan may have very low molecular weight less than 50,000 Da. In some embodiments, the compositions of the present disclosure may comprise low molecular weight chitosan (50,000-190,000 Da). In some embodiments, the compositions of the present disclosure may comprise medium molecular weight chitosan (190,000-310,000 Da). In some embodiments, the compositions of the present disclosure may comprise high molecular weight chitosan (310,000-375,000 Da). In some embodiments, the compositions of the present disclosure may comprise high molecular weight chitosan greater than 375,000 Da.

Chitosan is often generated through deacetylation of chitin, which can vary in terms of purity, non-saccharide inclusions (such as covalently crosslinked quinones), crosslinking, crystallinity, and acetylation degree. Reflecting this variation in source material, chitosan can similarly vary in terms of chemical and physical properties. Chitosan can be substantially homogenous, or can include protein, quinone, and non-amino saccharide inclusions. Chitosan can be cross-linked or non-cross-linked, as well as crystalline or non-crystalline.

In some embodiments, the chitosan is a copolymer. In some cases, the chitosan copolymer is a linear copolymer, a block copolymer, an alternating copolymer, a periodic copolymer, a statistical copolymer, a stereoblock copolymer, a gradient copolymer, branched copolymer, a graft copolymer, a start copolymer, or a combination thereof. In some cases, the chitosan is branched. In some cases, the chitosan is linear. In some cases, the chitosan is crosslinked. In some cases, the chitosan is not crosslinked. In some cases, the chitosan is crystalline. In some cases, the chitosan is noncrystalline.

In some cases, the composition comprises a plurality of chitosan polymers or oligomers. The chitosan can have an average molecular weight of between about 1 kiloDalton (kDa) and 20 megaDaltons (MDa), between about 1 kDa and about 25 kDa, between about 10 and 100 kDa, between about 50 and 500 kDa, between about 100 kDa and 1 MDa, or between about 1 and 20 mDa.

In some embodiments, the chitosan has a monomeric unit which comprises two D-glucosamines linked by a β-(1→4) glycosidic bond. In some cases, the chitosan comprises Formula (I), wherein n is an integer. In some embodiments, n is an integer from 1 to 1000.

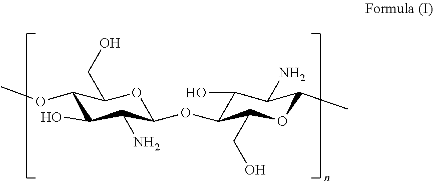

In some embodiments, the chitosan has a monomeric unit which comprises two N-acetylglucosamines linked by a β-(1→4) glycosidic bond. In some cases, the chitosan comprises Formula (II), wherein n is an integer. In some embodiments, n is an integer from 1 to 1000.

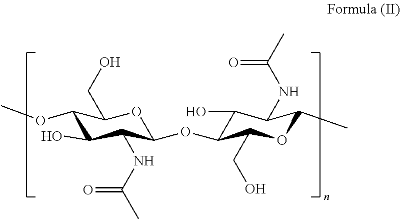

In some embodiments, the chitosan has a monomeric unit which comprises a mixture of D-glucosamine and N-acetylglucosamine linked by a β-(1→4) glycosidic bond. In some cases, the chitosan comprises Formula (III) wherein n is an integer. In some cases, the chitosan comprises Formula (IV) wherein n is an integer. In some embodiments, n is an integer from 1 to 1000.

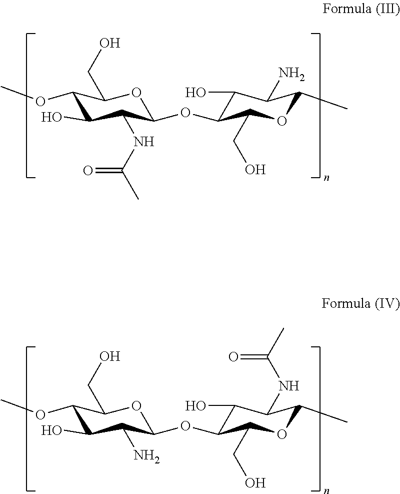

In some embodiments, the chitosan comprises a mixture of monomers selected from Formula (I), Formula (II), Formula (III), and Formula (IV). Formula (I)-Formula (IV) can be distributed in a random or ordered fashion throughout the chitosan. Multiple instances of Formula (I)-Formula (IV) can be interspersed by a non-D-glucosamine or N-acetylglucosamine moiety, such as a non-amino saccharide (e.g., glucose), a phenol, a quinone, or a peptide. In some embodiments, the chitosan has monomer repeats of any one of Formula (I), Formula (II), Formula (III), or Formula (IV) followed by any number of repeats of different monomers of any one of Formula (I), Formula (II), Formula (III), or Formula (IV). In some embodiments, the chitosan is a liner copolymer, a block copolymer, an alternating copolymer, a periodic copolymer, a statistical copolymer, a stereoblock copolymer, a gradient copolymer, branched copolymer, a graft copolymer, a start copolymer, or a combination thereof. In some embodiments, the chitosan is a linear copolymer comprises Formula (I), Formula (II), Formula (III), Formula (IV), or a combination thereof. In some embodiments, the chitosan is a block copolymer comprising Formula (I), Formula (II), Formula (III), Formula (IV), or a combination thereof. In some embodiments, the chitosan is an alternating copolymer comprising Formula (I), Formula (II), Formula (III), Formula (IV), or a combination thereof. In some embodiments, the chitosan is a periodic copolymer comprising Formula (I), Formula (II), Formula (III), Formula (IV), or a combination thereof. In some embodiments, the chitosan is a statistical copolymer comprising Formula (I), Formula (II), Formula (III), Formula (IV), or a combination thereof. In some embodiments, the chitosan is a stereoblock copolymer comprising Formula (I), Formula (II), Formula (III), Formula (IV), or a combination thereof. In some embodiments, the chitosan is a gradient copolymer comprising Formula (I), Formula (II), Formula (III), Formula (IV), or a combination thereof. In some embodiments, the chitosan is a branched copolymer comprising Formula (I), Formula (II), Formula (III), Formula (IV), or a combination thereof. In some embodiments, the chitosan is a graft copolymer comprising Formula (I), Formula (II), Formula (III), Formula (IV), or a combination thereof. In some embodiments, the chitosan is a star copolymer comprising Formula (I), Formula (II), Formula (III), Formula (IV), or a combination thereof.

In some embodiments, the monomer of the chitosan comprises two D-glucosamines with a monomer molecular formula of C₁₂H₂₂N₂O₈. In some cases, an oligomeric or polymeric portion of the chitosan contains a unit with the molecular formula (C₁₂H₂₂N₂O₈)ₙ, wherein n is an integer. In some embodiments, the monomer of the chitosan comprises two N-acetylglucosamines with a monomer molecular formula of C₁₆H₂₆N₂O₁₀. In some cases, an oligomeric or polymeric portion of the chitosan contains a unit with the molecular formula (C₁₆H₂₆N₂O₁₀)ₙ, wherein n is an integer. In some embodiments, the monomer of the chitosan comprises a combination of one D-glucosamine and one N-acetylglucosamine with a monomer molecular formula of C₁₄H₂₄N₂O₉. In some cases, an oligomeric or polymeric portion of the chitosan contains a unit with the molecular formula (C₁₄H₂₄N₂O₉)ₙ, wherein n represents an integer designating the number of repeating units. In some embodiments, the chitosan comprises a mixture of monomeric units with molecular formulas of C₁₂H₂₂N₂O₈, C₁₆H₂₆N₂O₁₀, and C₁₄H₂₄N₂O₉. In some embodiments, the chitosan is a copolymer with a mixture of monomeric units selected from C₁₂H₂₂N₂O₈, C₁₆H₂₆N₂O₁₀, and C₁₄H₂₄N₂O₉. In some embodiments, the chitosan has at least two of (C₁₂H₂₂N₂O₈)ₙ, (C₁₆H₂₆N₂O₁₀)ₙ, or (C₁₄H₂₄N₂O₉)ₙ. In some embodiments, the chitosan is linear copolymer comprising (C₁₂H₂₂N₂O₈)ₙ, (C₁₆H₂₆N₂O₁₀)ₙ, (C₁₄H₂₄N₂O₉)ₙ, or a combination thereof. In some embodiments, the chitosan is a block copolymer comprising (C₁₂H₂₂N₂O₈)ₙ, (C₁₆H₂₆N₂O₁₀)ₙ, (C₁₄H₂₄N₂O₉)ₙ, or a combination thereof. In some embodiments, the chitosan is an alternating copolymer comprising (C₁₂H₂₂N₂O₈)ₙ, (C₁₆H₂₆N₂O₁₀)ₙ, (C₁₄H₂₄N₂O₉)ₙ, or a combination thereof. In some embodiments, the chitosan is a periodic copolymer comprising (C₁₂H₂₂N₂O₈)ₙ, (C₁₆H₂₆N₂O₁₀)ₙ, (C₁₄H₂₄N₂O₉)ₙ, or a combination thereof. In some embodiments, the chitosan is a statistical copolymer comprising (C₁₂H₂₂N₂O₈)ₙ, (C₁₆H₂₆N₂O₁₀)ₙ, (C₁₄H₂₄N₂O₉)ₙ, or a combination thereof. In some embodiments, the chitosan is a stereoblock copolymer comprising (C₁₂H₂₂N₂O₈)ₙ, (C₁₆H₂₆N₂O₁₀)ₙ, (C₁₄H₂₄N₂O₉)ₙ, or a combination thereof. In some embodiments, the chitosan is a gradient copolymer comprising (C₁₂H₂₂N₂O₈)ₙ, (C₁₆H₂₆N₂O₁₀)ₙ, (C₁₄H₂₄N₂O₉)ₙ, or a combination thereof. In some embodiments, the chitosan is a branched copolymer comprising (C₁₂H₂₂N₂O₈)ₙ, (C₁₆H₂₆N₂O₁₀)ₙ, (C₁₄H₂₄N₂O₉)ₙ, or a combination thereof. In some embodiments, the chitosan is a graft copolymer comprising (C₁₂H₂₂N₂O₈)ₙ, (C₁₆H₂₆N₂O₁₀)ₙ, (C₁₄H₂₄N₂O₉)ₙ, or a combination thereof. In some embodiments, the chitosan is a star copolymer comprising (C₁₂H₂₂N₂O₈)ₙ, (C₁₆H₂₆N₂O₁₀)ₙ, (C₁₄H₂₄N₂O₉)ₙ, or a combination thereof.

The chitosan can have varying degrees of amine acylation. In the context of an amino saccharide, amine acylation can refer to the presence of an acetyl group on a saccharide amine on the amine of glucosamine. As used herein, “percent acylation” can refer to the percentage of amines which are acylated in a saccharide such as chitosan. In some cases, less than about 50%, less than about 45%, less than about 40%, less than about 35%, less than about 30%, less than about 25%, less than about 20%, less than about 15%, less than about 10%, less than about 5%, less than about 4%, less than about 3%, less than about 2%, or less than about 1% of amines of the chitosan are acetylated. In some cases, between about 1% and 50%, between about 1% and 10%, between about 3% and 15%, between about 5% and 20%, between about 10% and 35%, or between about 20% and 50% of amines of the chitosan are acylated.

The chitosan may have varying protonation states depending on pH, temperature, alpha-keto acid density, and local environment. In some embodiments, the chitosan is a cation. In the following examples “ [symbol] ” represents an attachment to the monomer structure as shown in Formula (I), Formula (II), Formula (III), and Formula (IV). In an example, the amino group on a monomer comprising N- or D-glucosamine may be protonated as [symbol] or a de-protonated as [symbol] -NH₂. In another example, the hydroxyl group on a monomer comprising N- or D-glucosamine may be protonated as [symbol] -OH or may be deprotonated as [symbol] -O⁻.

### Alpha-Keto Acids

An alpha-keto acid of the present disclosure may be an analogue of a natural or non-naturally occurring amino acid. An alpha-keto acid analogue of an amino acid may be an analogue of the amino acid in which a backbone amine is replaced by an oxo group. For example, pyruvate may be an alpha-keto acid analogue of alanine. Numerous alpha-keto acid forms of amino acids are present in vivo, and can be generated from amino acids by transaminases, which catalyze the transfer of the amino group of the amino acid to a different alpha-keto acid (e.g., alpha-keto glutarate), as well as numerous oxidases. Conversely, in vivo, alpha-keto acids can be converted to amino acids through transamination to convert the alpha-keto group to a hydride and an amine. Keto acids (which may be also referred to as oxo acids) comprise both a ketone functional group and a carboxylic acid group. Depending on the arrangement of the ketone functional group and the carboxylic acid functional group, keto acids can be characterized as alpha-keto acids, beta-keto acids, gamma-keto acids, or higher order keto acids. Keto acids can be substrates for transaminase enzymes, which may catalyze the conversion of a keto acid to an amino acid. For example, a general alpha-keto acid analogue of an amino acid may be represented by Formula (V), wherein the R¹ group may represent an amino acid sidechain.

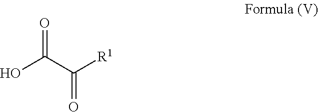

Alpha-keto acids of compounds, complexes, and compositions of the present disclosure may be analogues of essential amino acids. The alpha-keto acids may be analogues of conditionally essential amino acids. The alpha-keto acids may be analogues of non-essential amino acids. The alpha-keto acids may be selected from:

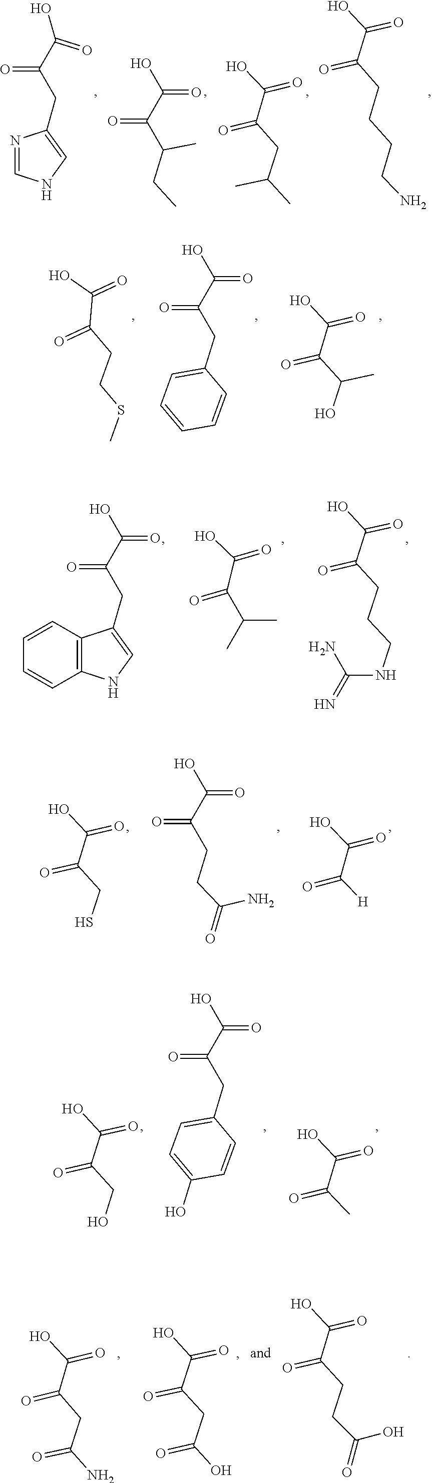

An alpha-keto acid of the present disclosure may be an alpha-keto acid analogue of a natural amino acid. While only 21 amino acids have been determined to be proteinogenic in eukaryotes, many of the approximately 500 amino acids identified in nature are actively metabolized in humans, rendering them as valuable nutrients. Furthermore, many non-proteinogenic amino acids may be direct precursors to proteinogenic amino acids, which can make them effective sources for proteinogenic amino acids and may be useful for treating specific amino acid deficiencies. Examples of natural, non-proteinogenic amino acids include citrulline, ornithine, and arginosuccinate, which are intermediates in the urea cycle; 1-3,4-dihydroxyphenylalanine (DOPA), which is a downstream oxidation product of tyrosine and a precursor to the neurotransmitter dopamine; and numerous hydroxylated amino acids, including 3-hydroxyproline and 5-hydroxylysine, which are typically formed post-translationally, but which can be funneled into amino acid biosynthesis pathways. Non-limiting examples of alpha-keto acid analogues of natural amino acids consistent with the present disclosure include proteinogenic alpha-keto acid analogues of amino acids and their sidechains (e.g., those outlined in TABLE 1, TABLE 2, and TABLE 3),

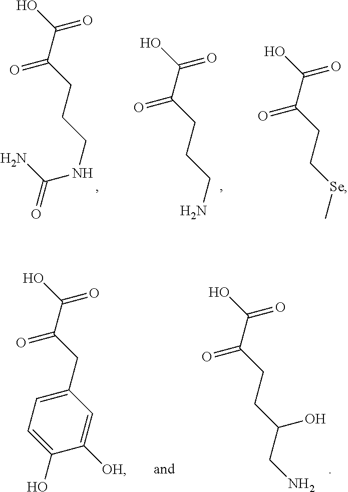

The alpha-keto acid can be an alpha-keto acid analogue of a non-proline proteinogenic amino acid. In some cases, the alpha-keto acid is selected from:

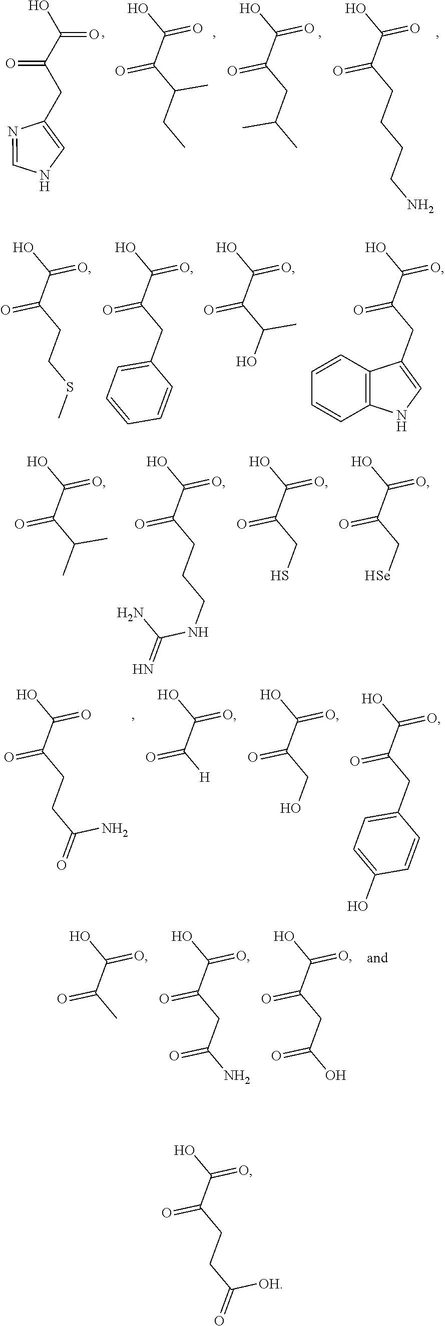

In some cases, the alpha-keto acid is selected from:

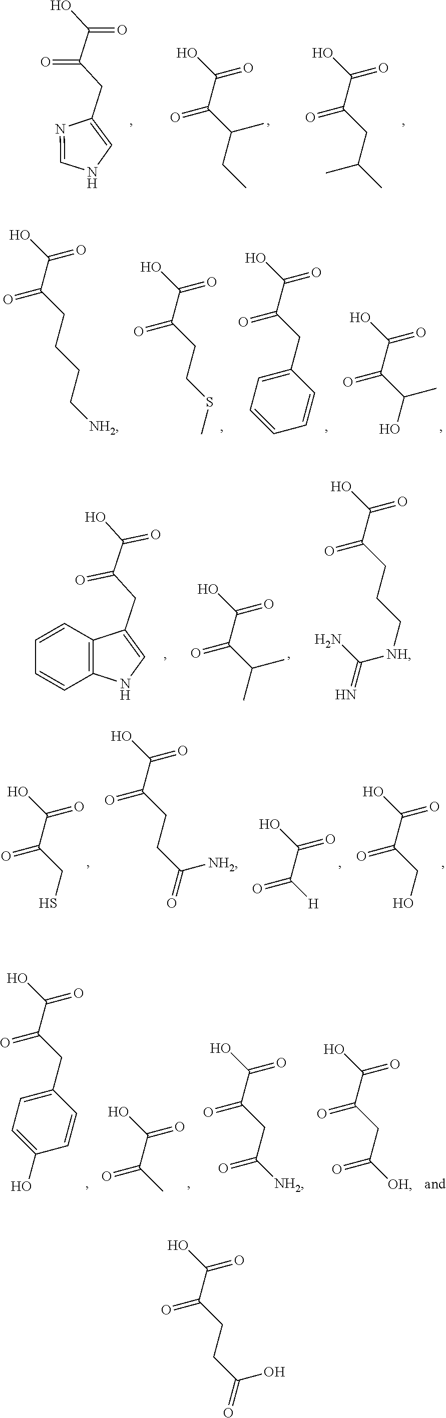

(e.g., does not include selenocysteine).

The alpha-keto acid may be an alpha-keto acid analogue of an essential amino acid. For example, the alpha-keto acid may comprise an alpha-keto acid analogue of histidine, an alpha-keto acid analogue of isoleucine, an alpha-keto acid analogue of leucine, an alpha-keto acid analogue of lysine, an alpha-keto acid analogue of methionine, an alpha-keto acid analogue of phenylalanine, an alpha-keto acid analogue of threonine, an alpha-keto acid analogue of tryptophan, or an alpha-keto acid analogue of valine. Examples of alpha-keto acid analogues of essential amino acids that may be included in the chitosan composition of the present disclosure are provided in TABLE 1.

TABLE 1
Alpha-Keto Acids Analogues of Essential Amino Acids

| | Alpha-Keto Acid Analogue | |
|---|---|---|
| | Structure and IUPAC Name | |
| Amino Acid (3-Letter Code) | (Common Name) | Side Chain Structure |
| Histidine (His) | 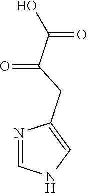 [SMILES: O=C(O)C(=O)Cc1c[nH]cn1] | 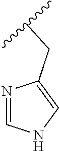 [SMILES: *Cc1c[nH]cn1] |
| | 3-(1H-imidazol-4-yl)-2-oxopropanoic | |
| | acid | |
| | (a-oxo-1H-imidazole-5-propanoic | |
| | acid) | |
| Isoleucine (Ile) | 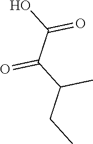 | 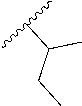 [SMILES: *C(C)CC] |
| | 3-methyl-2-oxopentanoic acid | |
| | ((±)-3-methyl-2-oxovaleric acid) | |
| Leucine (Leu) | 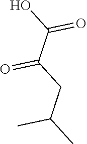 | 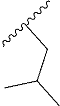 [SMILES: *CC(C)C] |
| | 4-methyl-2-oxopentanoic acid | |
| | (4-methyl-2-oxovaleric acid) | |
| Lysine (Lys) | 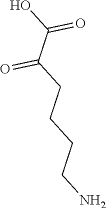 | 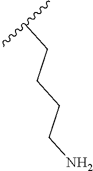 [SMILES: *CCCCN] |
| | 6-amino-2-oxohexanoic acid | |
| Methionine (Met) | 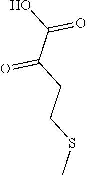 | 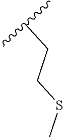 [SMILES: *CCSC] |
| | 4-(methylthio)-2-oxobutyric acid | |
| | (α-Keto-γ-(methylthio)butyric | |
| | acid) | |
| Phenylalanine (Phe) | 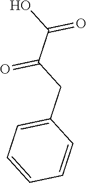 [SMILES: O=C(O)C(=O)Cc1ccccc1] | 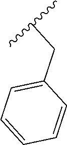 [SMILES: *Cc1ccccc1] |
| | 2-oxo-3-phenylpropanoic acid | |
| | (phenylpyruvic acid) | |
| Threonine (Thr) | 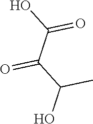 | 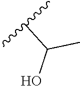 [SMILES: *C(C)O] |
| | 3-hydroxy-2-oxobutanoic acid | |
| Tryptophan (Trp) | 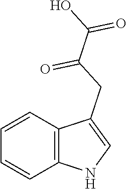 [SMILES: O=C(O)C(=O)Cc1c[nH]c2ccccc12] | 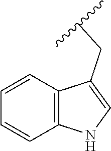 [SMILES: *Cc1c[nH]c2ccccc12] |
| | 3-(1H-indol-3-yl)-2-oxopropanoic acid | |
| | (indole-3-pyruvic acid) | |
| Valine (Val) | 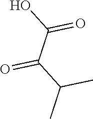 | 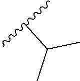 [SMILES: *C(C)C] |
| | 3-methyl-2-oxobutanoic acid | |
| | (α-Ketoisovaleric acid) | |

The alpha-keto acid may be an alpha-keto acid analogue of a conditionally essential amino acid. As used herein, the term “conditionally essential amino acids” can refer to the set amino acids which includes arginine, cysteine, glutamine, glycine, serine, and tyrosine, and can be used interchangeably with the term “quasi-essential amino acids.” For example, the alpha-keto acid may comprise an alpha-keto acid analogue of arginine, an alpha-keto acid analogue of cysteine, an alpha-keto acid analogue of glutamine, an alpha-keto acid analogue of tyrosine, an alpha-keto acid analogue of glycine, or an alpha-keto acid analogue of serine. Examples of alpha-keto acid analogues of conditionally essential amino acids that may be included in a composition of the present disclosure are provided in TABLE 2.

TABLE 2
Alpha-Keto Acid Analogues of Conditionally Essential Amino Acids

| | Alpha-Keto Acid Analogue | |
|---|---|---|
| Amino | Structure and IUPAC Name | Side Chain |
| Acid | (Common Name) | Structure |
| Arginine (Arg) | 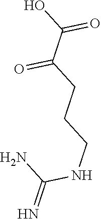 [SMILES: N=C(N)NCCCC(=O)C(=O)O] | 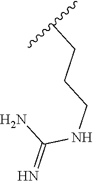 [SMILES: *CCCNC(=N)N] |
| | 5-guanidino-2-oxopentanoic acid | |
| | (δ-guanido-α-ketovaleric acid) | |
| Cysteine (Cys) | 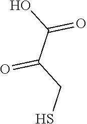 | 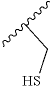 [SMILES: *CS] |
| | 3-mercapto-2-oxopropanoic acid | |
| | (3-mercaptopyruvic acid) | |
| Glutamine (Gln) | 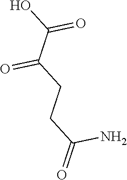 | 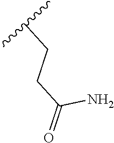 [SMILES: *CCC(N)=O] |
| | 5-amino-2,5-dioxopentanoic acid | |
| Glycine (Gly) | 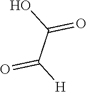 | 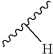 [SMILES: *[H]] |
| | 2-oxoacetic acid | |
| | (glyoxylic acid) | |
| Serine (Ser) | 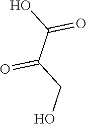 | 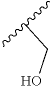 [SMILES: *CO] |
| | 3-hydroxy-2-oxopropanoic acid | |
| Tyrosine (Tyr) | 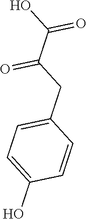 [SMILES: O=C(O)C(=O)Cc1ccc(O)cc1] | 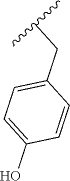 [SMILES: *Cc1ccc(O)cc1] |
| | 3-(4-hydroxyphenyl)-2-oxopropanoic acid | |
| | (4-hydroxyphenylpyruvic acid) | |

The alpha-keto acid may be an alpha-keto acid analogue of a non-essential amino acid. For example, an alpha-keto acid analogue of a non-essential amino acid may comprise an alpha-keto acid analogue of alanine (also known as pyruvate), an alpha-keto acid analogue of asparagine, an alpha-keto acid analogue of aspartate, or an alpha-keto acid analogue of glutamate (α-ketoglutarate). Examples of alpha-keto acid analogues of non-essential amino acids that may be included in the composition of the present disclosure are provided in TABLE 3.

TABLE 3
Alpha-Keto Acid Analogues of Non-Essential Amino Acids

| | Alpha-Keto Acid Analogue | Side |
|---|---|---|
| Amino Acid | Structure and IUPAC Name | Chain Structure |
| Alanine (Ala) | 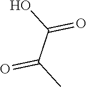 | 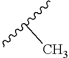 |
| | 2-oxopropanoic acid | |
| Asparagine (Asn) | 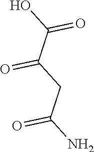 | 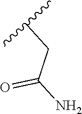 [SMILES: *CC(N)=O] |
| | 4-amino-2,4-dioxobutanoic acid | |
| Aspartic acid (Asp) | 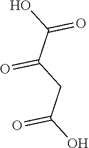 | 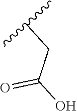 [SMILES: *CC(=O)O] |
| | 2-oxosuccinic acid | |
| Glutamic Acid (Glu) | 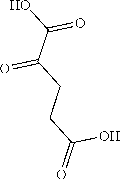 | 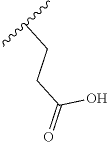 [SMILES: *CCC(=O)O] |
| | 2-oxopentanedioic acid | |

While the alpha-keto acids in TABLE 1, TABLE 2, and TABLE 3 are shown in their non-ionized forms, an alpha-keto acid of the present disclosure may comprise an ionized form of the alpha-keto acid (e.g., a protonated or deprotonated amino acid side chain, protonated or deprotonated carboxylic acid group). For example, an alpha-keto acid analogue of an amino acid may comprise a deprotonated glutamic acid sidechain or aspartic acid sidechain, a protonated arginine or lysine side chain, or a deprotonated carboxylic acid group.

### Polysaccharide Alpha-Keto Acid Compositions

Aspects of the present disclosure provide biopolymers with alpha-keto acid analogues of amino acids. In some embodiments, the biopolymer is a polysaccharide. In some embodiments, the polysaccharide is selected from a starch, a cellulose, an amino polysaccharide, an alginate, a carrageenan, a chitin, a chondroitin sulfate, a dextran, a galactomannan, a glycogen, a hyaluronic acid, a glycogen, a galactogen, an inulin, an arabinoxylan, or a pectin. In some embodiments, the polysaccharide comprises an amino group (e.g., an amino polysaccharide). In some embodiments, the polysaccharide is a chitosan. In some embodiments, the polysaccharide is a cation. In some embodiments, the polysaccharide is a polycation. A polysaccharide polycation may form a plurality of ionic bonds with a plurality of alpha-keto acid anions. In some embodiments, the average ratio of the alpha-keto acid anions associated to a polysaccharide polycation is at least 5:1 and no greater than 10,000:1. In some embodiments, the average ratio of the alpha-keto acid anions associated to a polysaccharide polycation is least 3:1 and no greater than 10,000:1, at least 4:1 and no greater than 10,000:1, at least 5:1 and no greater than 10,000:1, at least 10:1 and no greater than 10,000:1, at least 25:1 and no greater than 10,000:1, at least 50:1 and no greater than 10,000:1, at least 75:1 and no greater than 10,000:1, at least 100:1 and no greater than 10,000:1, at least 200:1 and no greater than 10,000:1, at least 300:1 and no greater than 10,000:1, at least 500:1 and no greater than 10,000:1, or at least 1,000:1 and no greater than 10,000:1. In some embodiments, the average ratio of the alpha-keto acid anions associated to a polysaccharide polycation is at least 3:1 and no greater than 1,000:1, at least 4:1 and no greater than 1,000:1, at least 5:1 and no greater than 1,000:1, at least 10:1 and no greater than 1,000:1, at least 25:1 and no greater than 1,000:1, at least 50:1 and no greater than 1,000:1, at least 75:1 and no greater than 1,000:1, at least 100:1 and no greater than 1,000:1, at least 200:1 and no greater than 1,000:1, at least 300:1 and no greater than 1,000:1, at least 500:1 and no greater than 1,000:1, or at least 750:1 and no greater than 1,000:1.

In some embodiments, the composition comprises a polysaccharide and an alpha-keto acid analogue of a natural amino acid. In some embodiments, the composition comprises a polysaccharide and an alpha-keto acid analogue of an essential amino acid. In some embodiments, the polysaccharide is non-covalently coupled to an alpha-keto acid.

While the forgoing primarily details chitosan-alpha-keto acid compositions, it would be understood that, in some embodiments, the chitosan is substituted for another amino polysaccharide (e.g., amino cellulose, an amino dextran, an amino galactose, an amino arabinose, a chitin, or a combination thereof).

Multiple alpha-keto acid-chitosan coupling modalities enable alpha-keto acid lability and thus bioavailability for amino acid biosynthesis and a source of protein intake when administered to a subject. The alpha-keto acid can be coupled to the chitosan through ionic bonding, hydrogen bonding, van der Waals interactions, covalent bonding, or a combination thereof. By combining the alpha-keto acid (Formula (V)) with the chitosan (Formula (I), Formula (II), Formula (III), or Formula (IV)), a positively charged amino group (pKₐ~6.5) of the chitosan can form an ionic bond with a negatively charged carboxylate group on the alpha-keto acid (pKa~2.5), thereby forming a salt. The chitosan may have a plurality of positively charged amino groups referred to as chitosan polycation. The chitosan polycation may form a plurality of ionic bonds with a plurality of alpha-keto acid anions. In some embodiments, the average ratio of the alpha-keto acid anions associated to a chitosan polycation is at least 5:1 and no greater than 10,000:1. In some embodiments, the average ratio of the alpha-keto acid anions associated to a chitosan polycation is least 3:1 and no greater than 10,000:1, at least 4:1 and no greater than 10,000:1, at least 5:1 and no greater than 10,000:1, at least 10:1 and no greater than 10,000:1, at least 25:1 and no greater than 10,000:1, at least 50:1 and no greater than 10,000:1, at least 75:1 and no greater than 10,000:1, at least 100:1 and no greater than 10,000:1, at least 200:1 and no greater than 10,000:1, at least 300:1 and no greater than 10,000:1, at least 500:1 and no greater than 10,000:1, or at least 1,000:1 and no greater than 10,000:1. In some embodiments, the average ratio of the alpha-keto acid anions associated to a chitosan polycation is at least 3:1 and no greater than 1,000:1, at least 4:1 and no greater than 1,000:1, at least 5:1 and no greater than 1,000:1, at least 10:1 and no greater than 1,000:1, at least 25:1 and no greater than 1,000:1, at least 50:1 and no greater than 1,000:1, at least 75:1 and no greater than 1,000:1, at least 100:1 and no greater than 1,000:1, at least 200:1 and no greater than 1,000:1, at least 300:1 and no greater than 1,000:1, at least 500:1 and no greater than 1,000:1, or at least 750:1 and no greater than 1,000:1.

In some cases, the composition comprises a chitosan cation and an alpha-keto acid anion. In many such cases, the chitosan comprises a positively charged ammonium group and the alpha-keto acid comprises a negatively charged carboxylate. In some cases, the alpha-keto acid is negatively charged. In some cases, the chitosan is neutral. In some cases, the chitosan cation and alpha-keto acid anion are in the form of a complex, wherein the chitosan cation and alpha-keto acid anion are associated through an ionic bonding interaction. In some embodiments, the chitosan is ionically coupled to an alpha-keto acid through the positive charge on the chitosan and a negative charge on the alpha-keto acid. The chitosan cation and alpha-keto acid anion can be provided in dry form as a salt. A complex of a chitosan cation and alpha-keto acid anion can be hydrated. A complex of a chitosan cation and alpha-keto acid anion can be charge balanced.

In some cases, the composition contains less than about 1000 ppm (e.g., on a weight-by-weight basis), less than about 750 ppm, less than about 500 ppm, less than about 400 ppm, less than about 300 ppm, less than about 250 ppm, less than about 200 ppm, less than about 150 ppm, less than about 100 ppm, or less than about 50 ppm of inorganic cations (e.g., Na⁺, K⁺, Mg²⁺, Ca²⁺, etc.). In some cases, a ratio of the alpha-keto acid to inorganic cations in the composition is at least about 1:1, at least about 3:2, at least about 5:2, at least about 5:1, at least about 10:1, at least about 20:1, at least about 50:1, at least about 100:1, at least about 200:1, or at least about 500:1.

In some cases, the alpha-keto acid is intercalated within the chitosan. In some cases, the alpha-keto acid is coupled to a surface of the chitosan. For example, at least about 50%, at least about 60%, at least about 70%, at least about 80%, at least about 90%, at least about 95%, or at least about 99% of instances of the alpha-keto acid can be coupled to the surface of the chitosan.

A chitosan complex of the present disclosure can include Formula (VI), Formula (VII), or a combination thereof. Formula (VI) is a D-glucosamine monomeric unit in which the C2-amine is positively charged and is ionically coupled to a negatively charged alpha-keto acid carboxylate. Formula (VII) is a D-glucosamine dimer in which both C2-amines are positively charged and are ionically coupled to negatively charged alpha-keto acid carboxylates. In some embodiments, charge (e.g., positive charge, negative charge, or neutral charge) may be determined at pH 7.0. In some instances of Formula (VI) and Formula (VII), each instance of R¹ can independently a sidechain of a natural amino acid (e.g., the side chain of an amino acid biosynthesized by a non-recombinant organism). Formula (VI) or Formula (VII) may be synthesized by combining an alpha-keto acid (e.g., an alpha-keto acid analogue of an amino acid listed in TABLE 1, TABLE 2, or TABLE 3) and a chitosan in a buffered aqueous solution at neutral pH. For example, Formula (VI) or Formula (VII) may be synthesized by adding chitosan with a keto acid mixture comprising a mixture of alpha-keto acids (e.g., alpha-keto acid analogues of amino acids listed in TABLE 1, TABLE 2, or TABLE 3). In some embodiments, a carboxylic acid in the R¹ group of an alpha-keto acid (e.g., a carboxylate of an alpha-keto acid analogue of glutamic acid or aspartic acid) can be ionically coupled to the chitosan. Coupling between the chitosan and the alpha-keto acid (e.g., an alpha-keto acid analogue of an amino acid) may be monitored gravimetrically, or by quantifying un-coupled alpha-keto acid content with high performance liquid chromatography (HPLC).

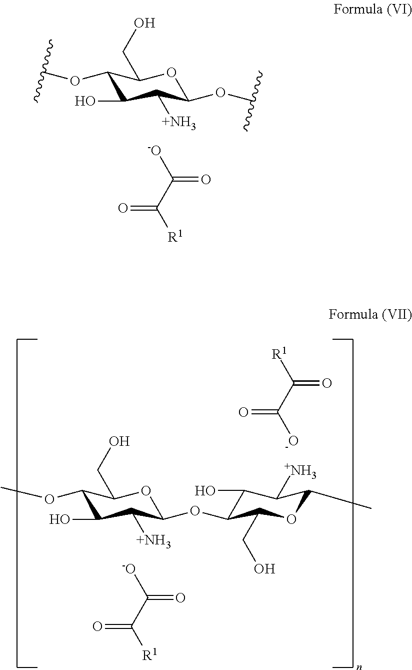

The chitosan may be coupled to the alpha-keto acid (e.g., an alpha-keto acid analogue of an amino acid) through hydrogen bonding interactions. The alpha-keto acid (e.g., Formula (V)) can have an alpha-ketone functional group that may act as a hydrogen bond acceptor for an amine, ammonium, or hydroxyl proton on the chitosan. In some cases, the composition comprises Formula (VIII), Formula (IX), or a combination thereof. Formula (VIII) is a D-glucosamine monomeric unit in which the C2-amine hydrogen bonded to the alpha-carbon ketone of the alpha-keto acid. Formula (IX) is a D-glucosamine monomeric unit in which the C2-amine is hydrogen bonded to the alpha-carbon ketone of the alpha-keto acid. Each instance of R¹ is independently a sidechain of a natural amino acid (e.g., the side chain structures listed in TABLE 1, TABLE 2, or TABLE 3). Formula (VIII) and Formula (IX) may be synthesized by combining an alpha-keto acid (e.g., an alpha-keto acid analogue of an amino acid listed in TABLE 1, TABLE 2, or TABLE 3) with a chitosan in a buffered aqueous solution at a basic pH.

In some embodiments, the synthesis of Formula (VI) or Formula (VII) may also comprise the addition of other chemical compounds for desired properties of the keto acid chitosan compositions such as lipids (e.g., DSPE-PEG, magnesium stearate), bases (e.g., NaOH), polysaccharides (e.g., sodium alginate or mannitol), tripoly phosphate (TPP), (hydroxypropyl)methyl cellulose phthalate (HPMCP), polymers (e.g., PEG, PEG3350, PS80, or P188), citrate, or any combination thereof. In some embodiments, the composition may be lyophilized.

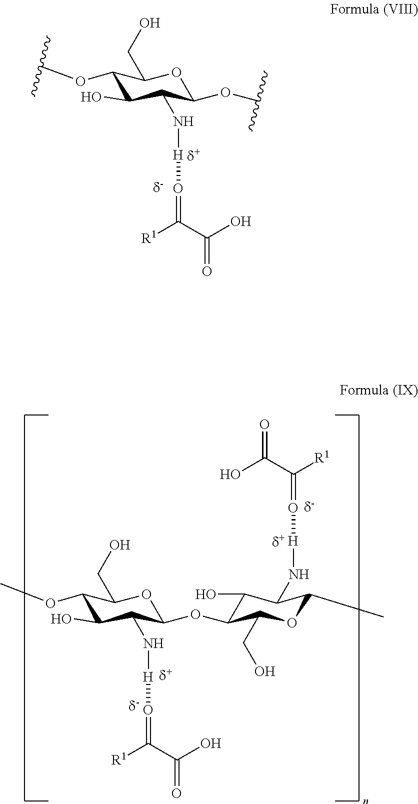

In some cases, the composition contains one or more instances of Formula (VI), Formula (VII), Formula (VIII), Formula (IX), or a combination thereof. In some cases, the composition contains additional non-covalent chitosan-alpha-keto acid units in addition to the one or more instances of Formula (VI)-Formula (IX).

The chitosan may also be covalently coupled to the alpha-keto acid. For example, a chitosan amine may be coupled to a carbonyl of the alpha-keto acid (e.g., the carboxylate or alpha-carbon ketone of an alpha-keto acid moiety of the alpha-keto acid) through nucleophilic substitution or decarboxylative addition to form an amide linkage. In some embodiments, the alpha-keto acid may be covalently coupled to the chitosan through an amide. An example of an alpha-keto acid bound to chitosan through covalent bonding is provided in monomeric form in Formula (X) and as a homodimer in Formula (XI), wherein each instance of R¹ is independently a sidechain of a natural amino acid (e.g., the side chain structures listed in TABLE 1, TABLE 2, or TABLE 3). Formula (X) and Formula (XI) may be synthesized by combining an alpha-keto acid (e.g., an alpha-keto acid analogue of an amino acid listed in TABLE 1, TABLE 2, or TABLE 3) with a chitosan in the presence of a coupling agent (e.g., dicyclohexyl carboiimide) or heat.

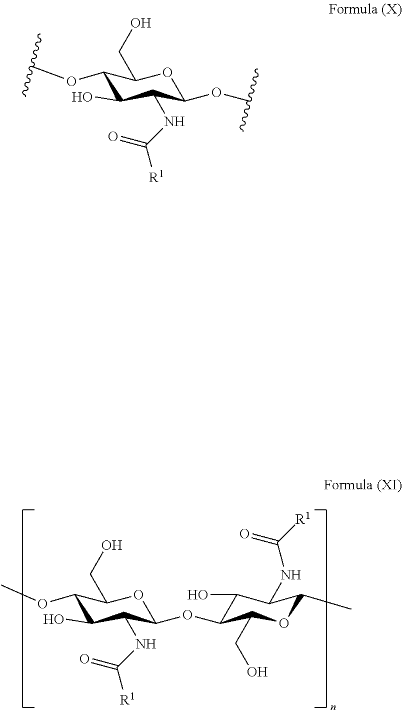

As decarboxylative amide formation is irreversible under many physiological conditions, Formula (X) and Formula (XI) typically provide low alpha-keto acid bioavailabilities upon administration. Accordingly, the present disclosure provides methods for generating low prevalence of Formula (X) and Formula (XI) among chitosan-complexed alpha-keto acids. In many cases, less than about 10%, less than about 8%, less than about 6%, less than about 5%, less than about 4%, less than about 3%, less than about 2%, less than about 1%, less than about 0.5%, less than about 0.2%, or less than about 0.1% of alpha-keto acids and chitosan coupled-products thereof in a composition are present as Formula (X) or Formula (XI).

In another example, the amine group on the chitosan (Formula (I), Formula (III), or Formula (IV)) may react with the carbonyl of the carboxylic acid group of an alpha-keto acid (e.g., an alpha-keto acid analogue of an amino acid) to form a keto-amide linkage in the presence of a coupling agent (e.g., dicyclohexyl carboiimide). In some embodiments, the alpha-keto acid may be covalently coupled to the polysaccharide (e.g., a chitosan) as a keto-amide. Examples of the alpha-keto acid (e.g., an alpha-keto acid analogue of an amino acid) covalently bound to the chitosan are provided in Formula (XII) and Formula (XIII), wherein each instance of R¹ is independently a sidechain of a natural amino acid (e.g., the side chain structures listed in TABLE 1, TABLE 2, or TABLE 3). Formula (XII) and Formula (XIII) may be synthesized by combining the alpha-keto acid (e.g., an alpha-keto acid analogue of an amino acid listed in TABLE 1, TABLE 2, or TABLE 3) with the chitosan in the presence of a coupling agent (e.g., dicyclohexyl carboiimide) or heat.

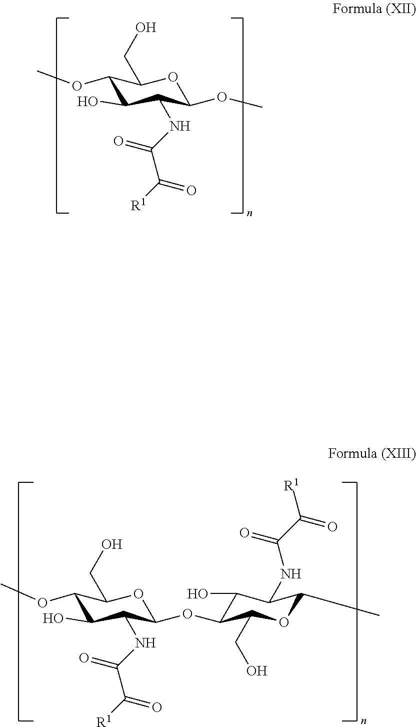

In another example, the chitosan is covalently coupled to an alpha carbon of the alpha-keto acid. For example, the alpha-keto acid can couple to the chitosan through a reaction in which a chitosan amine couples to an alpha-ketone of the alpha-keto acid to form an imine (e.g., Schiff base) intermediate and then is reduced to an amine. An example of the alpha-keto acid (e.g., an alpha-keto acid analogue of an amino acid) bound to the chitosan through covalent bonding is provided in monomeric form in Formula (XIV) and as a homodimer in Formula (XV), wherein each instance of R¹ is independently a sidechain of a natural amino acid (e.g., the side chain structures listed in TABLE 1, TABLE 2, or TABLE 3). Formula (XIV) and Formula (XV) may be synthesized by combining the alpha-keto acid (e.g., an alpha-keto acid analogue of an amino acid listed in TABLE 1, TABLE 2, or TABLE 3) with the chitosan in a buffered aqueous solution at acidic pH in the presence of a reducing agent (e.g., sodium borohydride). However, many of the compositions disclosed herein do not include Formula (XIV) or Formula (XV).

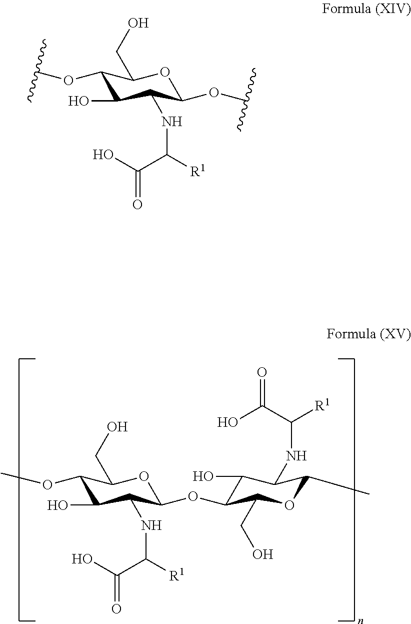

In some cases, the alpha-keto acid can be coupled to the polysaccharide in a manner which is irreversible under physiological conditions. In particular instances, irreversible covalent coupling may denote that the alpha-keto acid and the polysaccharide are coupled through an unhydrolyzable bond. For example, an irreversibly covalently coupled alpha-keto acid may be an amide formed through decarboxylative amidation between an alpha-keto acid and a polysaccharide amine.

While certain conditions may permit alpha-keto acid decoupling and regeneration from Formula (XIV) and Formula (XV), these species may provide lower alpha-keto acid bioavailability than Formula (VI)-Formula (IX) and Formula (XII)-Formula (XIII). Accordingly, in many cases, less than about 10%, less than about 8%, less than about 6%, less than about 5%, less than about 4%, less than about 3%, less than about 2%, less than about 1%, less than about 0.5%, less than about 0.2%, or less than about 0.1% of alpha-keto acids and chitosan-coupled products thereof in a composition are present as Formula (XIV) or Formula (XV).

In the context of an alpha-keto acid and a polysaccharide, covalently coupled can denote that a portion of the alpha-keto acid was cleaved in forming a bond with the polysaccharide. In one embodiment, a covalently coupled alpha-keto acid is an alpha-keto amide formed by condensation between a carboxylic acid of the alpha-keto acid with a polysaccharide derived amine. In another embodiment, the alpha-ketone of the alpha-keto acid is replaced with a hydride and a bond to an amine of the chitosan. In many cases, a covalently coupled alpha-keto acid can be liberated through hydrolysis to form a free alpha-keto acid.

In some embodiments, the composition as disclosed herein may have multiple types of bonds between the chitosan and the alpha-keto acid varying based on local structure and environment. In some embodiments, between about 50% and 99.9%, about 60% and 99%, about 70% and 99%, about 80% and 99.9%, or about 90% and 99.9% of alpha-keto acids are non-covalently coupled to the chitosan. In some embodiments, between about 10% and 50%, about 20% and 60%, about 30% and 70%, about 40% and 80%, or about 50% and 90% of alpha-keto acids are covalently coupled to the chitosan. In some embodiments, the chitosan is covalently coupled to a first instance of the alpha-keto acid and non-covalently coupled to a second instance of the alpha-keto acid. In some embodiments, the composition may comprise two or more of Formula (VI), Formula (VIII), Formula (X), Formula (XII), and Formula (XIV). In some embodiments, the chitosan comprises instances of Formula (VI), Formula (VIII), Formula (X), Formula (XII), and Formula (XIV). In some embodiments, the chitosan comprises one or more aminosaccharide monomer or dimer units which does not comprise (e.g., is not coupled to) the alpha-keto acid. In some embodiments, the chitosan comprises a ratio of Formula (VI) to Formula (VIII) of between about 10:1 and 1:10, about 100:1 and 10:1, or about 1:10 and 1:100.

Following administration (e.g., ingestion), the composition can be hydrolyzed (e.g., in vivo) to liberate the alpha-keto acid from the chitosan to yield a free alpha-keto acid and a chitosan polymer.

In some embodiments, the composition includes an alpha-keto acid analogue of a non-proline amino acid. In some embodiments, the composition includes an alpha-keto acid analogue of a non-proline natural amino acid. In some embodiments, the composition includes an alpha-keto acid analogue of a non-proline proteinogenic amino acid. In some embodiments, the composition includes an alpha-keto acid analogues of one or more essential amino acids. For example, the composition may include alpha-keto acid analogues of 9 essential amino acids. In some embodiments, the composition includes alpha-keto acid analogues of one or more conditionally essential amino acids. For example, the composition may include alpha-keto acid analogues of 6 conditionally essential amino acids. In some embodiments, the composition includes alpha-keto acid analogues of one or more non-essential amino acids. For example, the composition may include alpha-keto acid analogues of 4 non-essential amino acids. In some embodiments, the composition may include alpha-keto acid analogues of one or more essential amino acids, conditionally essential amino acids, or non-essential amino acids. For example, the composition may include alpha-keto acid analogues of 9 essential amino acids and 6 conditionally essential amino acids. In another example, the composition may include alpha-keto acid analogues of 9 essential amino acids, 6 conditionally essential amino acids, and 4 non-essential amino acids. In another example, the composition may include alpha-keto acid analogues of at least one essential amino acid, alpha-keto acid analogues of at least two essential amino acids, alpha-keto acid analogues of at least three essential amino acids, alpha-keto acid analogues of at least four essential amino acids, alpha-keto acid analogues of at least five essential amino acids, alpha-keto acid analogues of at least six essential amino acids, alpha-keto acid analogues of at least seven essential amino acids, or alpha-keto acid analogues of at least eight essential amino acids. In another example, the composition may include alpha-keto acid analogues of 9 essential amino acids and an alpha-keto acid analogue of a conditionally essential amino acid, alpha-keto acid analogues of at least two conditionally essential amino acids, alpha-keto acid analogues of at least three conditionally essential amino acids, alpha-keto acid analogues of at least four conditionally essential amino acids, or alpha-keto acid analogues of at least five conditionally essential amino acids. In another example, a composition may be formulated to include alpha-keto acid analogues of 9 of the essential amino acids, 6 of the conditionally essential amino acids and may further comprise alpha-keto acid analogues of at least one non-essential amino acid, alpha-keto acid analogues of at least two non-essential amino acids, or alpha-keto acid analogues of at least three non-essential amino acids. In some embodiments, the composition may include alpha-keto acid analogues of essential amino acids and alpha-keto acid analogues of non-proline conditionally essential amino acids. In some embodiments, the composition may include alpha-keto acid analogues of essential amino acids, alpha-keto acid analogues of non-proline conditionally essential amino acids, and alpha-keto acid analogues of non-essential amino acids.

Since the chitosan may comprise monomers of both D-glucosamine and N-acetylglucosamine, for example as shown in Formula (I), Formula (II), Formula (III), and Formula (IV), the chitosan may be characterized by a degree of deacetylation. As used herein in reference to chitosan, degree of deacetylation may quantify how many monomers are N-acetylglucosamine (i.e., have acetylated amines) and how many monomers are D-glucosamine (e.g., are deacetylated). The compositions of the present disclosure can utilize chitosan with a wide range of degrees of deacetylation. In some embodiments, the chitosan has a degree of deacetylation between about 1 and 55%. In some embodiments, the chitosan has a degree of deacetylation between about 55 and 70%. In some embodiments, the chitosan has a degree of deacetylation between about 70 and 85%. In some embodiments, the chitosan has a degree of deacetylation between about 85 and 95%. In some embodiments, the chitosan has a degree of deacetylation between about 95 and 100%. In some embodiments, the chitosan has a degree of deacetylation greater than about 50%, greater than about 55%, greater than about 60%, greater than about 65%, greater than about 70%, greater than about 75%, greater than about 80%, greater than about 85%, greater than about 90%, or greater than about 95%. In some embodiments, less than about 50%, less than about 45%, less than about 40%, less than about 35%, less than about 30%, less than about 25%, less than about 20%, less than about 15%, less than about 10%, less than about 5%, less than about 4%, less than about 3%, less than about 2%, or less than about 1% of amines of the chitosan are acetylated.

The viscosity of the composition may be varied to optimize suitability for different dosage forms. In some cases, the amount of hydrogen bonding or ionic bonding between the chitosan and the alpha-keto acid and the degree of deacetylation of the chitosan affect the viscosity of the composition. Similarly, the conformation of the chitosan may vary depending on hydrogen bonding, ionic bonding, solvent type, temperature, degree of deacetylation of a chitosan, and other factors. For example, the chitosan may have a spiral shape, an elongated conformation, or a quasi-globular conformation. The melting temperature of the composition can similarly vary based on characteristics of the chitosan, the alpha-keto acid, and additional constituents. The melting temperature of the composition can be the temperature at which the composition goes from a solid to liquid and can be dependent on a variety of factors such as hydrogen bonding, ionic bonding, degree of deacetylation of a chitosan, and others. In some embodiments, the melting temperature of the composition is from about 100° C. to about 130° C., from about 130° C. to 160° C., from about 160° C. to 190° C., from about 170° C. to 200° C., from about 180° C. to 200° C., from about 180° C. to 210° C., from about 190° C. to 220° C., or from about 220° C. to 250° C.

In some embodiments, the chitosan is esterified, etherified, oxidized, cross-linked, aminated, or partially degraded.

The chitosan can be isolated or derived from natural sources or can be synthetic. In some embodiments the chitosan is isolated from a marine source (e.g., brine shrimp, marine shrimp shells, crab female and crab male shells, cuttlefish pens, and lobster shells). In some embodiments the chitosan is isolated from fungi. For example, the chitosan is isolated from a species from the genus Aspergillus. In another example, the chitosan is isolated from a species of fungi such as Benjaminiella poitrasii (Zygomycetes, dimorphic), Hanseniaspora guilliermondii, Issatchenkia orientalis, Pichia membranifaciens, Saccharomyces cerevisiae (Ascomycetes, yeasts), Agaricus bisporus, or Pleurotus sajor-caju (Basidiomycetes)). Compositions and Formulations of Chitosan and Alpha-Keto Acids

The compositions described herein comprising a polysaccharide (e.g., a chitosan) and an alpha-keto acid composition may also comprise other chemical compounds. In some embodiments, the keto acid chitosan compositions such as lipids (e.g., DSPE-PEG, magnesium stearate), bases (e.g., NaOH), polysaccharides (e.g., sodium alginate or mannitol), tripoly phosphate (TPP), (hydroxypropyl)methyl cellulose phthalate (HPMCP), polymers (e.g., PEG, PEG3350, PS80, or P188), citrate, or any combination thereof. In some embodiments, the chitosan alpha-keto acid compositions as described herein may comprise magnesium stearate and alginate. In some embodiments, the chitosan alpha-keto acid compositions as described herein may comprise DSPE-PEG, magnesium stearate, and alginate. In some embodiments, the chitosan alpha-keto acid compositions as described herein may comprise DSPE-PEG, and mannitol.

The compositions of chitosan and alpha-keto acids as described herein may be prepared from a solution comprising chitosan, alpha-keto acids, and optionally additional chemical compounds (e.g., lipids (e.g., DSPE-PEG, magnesium stearate), bases (e.g., NaOH), polysaccharides (e.g., sodium alginate or mannitol), tripoly phosphate (TPP), (hydroxypropyl)methyl cellulose phthalate (HPMCP), polymers (e.g., PEG, PEG3350, PS80, or P188), citrate, or any combination thereof). In some embodiments, the compositions of chitosan and alpha-keto acids as described herein may be prepared by lyophilization. In some embodiments, the compositions of chitosan and alpha-keto acids as described herein may be prepared by reconstitution in liquid formulations. In some embodiments, the compositions of chitosan and alpha-keto acids as described herein may be prepared by reconstitution in gel formulations.

The compositions as described herein may be prepared from solutions that comprise at least 0.001% and no more than 99.99% chitosan, at least 0.01% and no more than 25% chitosan, at least 0.01% and no more than 15% chitosan, at least 0.01% and no more than 10% chitosan, at least 0.1% and no more than 15% chitosan, at least 0.1% and no more than 10% chitosan, or at least 0.1% and no more than 5% chitosan. In some embodiments, the compositions as described herein may comprise at least 0.001% and no more than 99.99% chitosan, at least 0.01% and no more than 25% chitosan, at least 0.01% and no more than 15% chitosan, at least 0.01% and no more than 10% chitosan, at least 0.1% and no more than 15% chitosan, at least 0.1% and no more than 10% chitosan, or at least 0.1% and no more than 5% chitosan. In some embodiments, the compositions as described herein may comprise at least 0.1% and no more than 10% chitosan. In some embodiments, the compositions as described herein may comprise at least 0.1% and no more than 5% chitosan. In some embodiments, the compositions as described herein may comprise at least 0.1% and no more than 1% chitosan. In some embodiments, the compositions as described herein may comprise 0.25%, 0.5%, 0.75%, 1%, 1.5%, 2.0%, 2.5%, 3.0%, 3.5%, 4.0%, 4.5%, 5.0%, 5.5%, 6.0%, 7.0%, 10%, 15%, or 20% chitosan. In some embodiments, the compositions as described herein may comprise 0.5% chitosan. In some embodiments, the compositions as described herein may comprise 1% chitosan. In some embodiments, the compositions as described herein may comprise 2% chitosan. In some embodiments, the compositions as described herein may comprise 3% chitosan. In some embodiments, the compositions as described herein may comprise 4% chitosan. In some embodiments, the compositions as described herein may comprise 6% chitosan. In some embodiments, the compositions as described herein may comprise 8% chitosan. In some embodiments, the compositions as described herein may comprise 10% chitosan.

The compositions of chitosan and alpha-keto acids as described herein may comprise at least 0.01 mg/ml and no more than 20 mg/ml of any individual alpha-keto acid analogue of an amino acid. In some embodiments, the compositions of chitosan and alpha-keto acids as described herein may comprise at least 0.01 mg/ml and no more than 20 mg/ml, at least 0.1 mg/ml and no more than 20 mg/ml, at least 0.1 mg/ml and no more than 15 mg/ml, at least 0.1 mg/ml and no more than 10 mg/ml, or at least 0.5 mg/ml and no more than 10 mg/ml of any individual alpha-keto acid analogue of an amino acid.

In some embodiments, the compositions of chitosan and alpha-keto acids as described herein may comprise at least 0.01 mg/ml and no more than 20 mg/ml, at least 0.1 mg/ml and no more than 20 mg/ml, at least 0.1 mg/ml and no more than 15 mg/ml, at least 0.1 mg/ml and no more than 10 mg/ml, or at least 0.5 mg/ml and no more than 10 mg/ml of the alpha-keto acid analogue of Tryptophan. In some embodiments, the compositions of chitosan and alpha-keto acids as described herein may comprise at least 0.1 mg/ml and no more than 10 mg/ml of the alpha-keto acid analogue of Tryptophan. In some embodiments, the compositions of chitosan and alpha-keto acids as described herein may comprise 0.8 mg/ml of the alpha-keto acid analogue of Tryptophan.

In some embodiments, the compositions of chitosan and alpha-keto acids as described herein may comprise at least 0.01 mg/ml and no more than 20 mg/ml, at least 0.1 mg/ml and no more than 20 mg/ml, at least 0.1 mg/ml and no more than 15 mg/ml, at least 0.1 mg/ml and no more than 10 mg/ml, or at least 0.5 mg/ml and no more than 10 mg/ml of the alpha-keto acid analogue of Glycine. In some embodiments, the compositions of chitosan and alpha-keto acids as described herein may comprise at least 0.1 mg/ml and no more than 10 mg/ml of the alpha-keto acid analogue of Glycine. In some embodiments, the compositions of chitosan and alpha-keto acids as described herein may comprise 4.4 mg/ml of the alpha-keto acid analogue of Glycine.

In some embodiments, the compositions of chitosan and alpha-keto acids as described herein may comprise at least 0.01 mg/ml and no more than 20 mg/ml, at least 0.1 mg/ml and no more than 20 mg/ml, at least 0.1 mg/ml and no more than 15 mg/ml, at least 0.1 mg/ml and no more than 10 mg/ml, or at least 0.5 mg/ml and no more than 10 mg/ml of the alpha-keto acid analogue of Methionine. In some embodiments, the compositions of chitosan and alpha-keto acids as described herein may comprise at least 0.1 mg/ml and no more than 10 mg/ml of the alpha-keto acid analogue of Methionine. In some embodiments, the compositions of chitosan and alpha-keto acids as described herein may comprise 1.5 mg/ml of the alpha-keto acid analogue of Methionine.

In some embodiments, the compositions of chitosan and alpha-keto acids as described herein may comprise at least 0.01 mg/ml and no more than 20 mg/ml, at least 0.1 mg/ml and no more than 20 mg/ml, at least 0.1 mg/ml and no more than 15 mg/ml, at least 0.1 mg/ml and no more than 10 mg/ml, or at least 0.5 mg/ml and no more than 10 mg/ml of the alpha-keto acid analogue of Isoleucine. In some embodiments, the compositions of chitosan and alpha-keto acids as described herein may comprise at least 0.1 mg/ml and no more than 10 mg/ml of the alpha-keto acid analogue of Isoleucine. In some embodiments, the compositions of chitosan and alpha-keto acids as described herein may comprise 4.0 mg/ml of the alpha-keto acid analogue of Isoleucine.

In some embodiments, the compositions of chitosan and alpha-keto acids as described herein may comprise at least 0.01 mg/ml and no more than 20 mg/ml, at least 0.1 mg/ml and no more than 20 mg/ml, at least 0.1 mg/ml and no more than 15 mg/ml, at least 0.1 mg/ml and no more than 10 mg/ml, or at least 0.5 mg/ml and no more than 10 mg/ml of the alpha-keto acid analogue of Histidine. In some embodiments, the compositions of chitosan and alpha-keto acids as described herein may comprise at least 0.1 mg/ml and no more than 10 mg/ml of the alpha-keto acid analogue of Histidine. In some embodiments, the compositions of chitosan and alpha-keto acids as described herein may comprise 2.0 mg/ml of the alpha-keto acid analogue of Histidine.

In some embodiments, the compositions of chitosan and alpha-keto acids as described herein may comprise at least 0.01 mg/ml and no more than 20 mg/ml, at least 0.1 mg/ml and no more than 20 mg/ml, at least 0.1 mg/ml and no more than 15 mg/ml, at least 0.1 mg/ml and no more than 10 mg/ml, or at least 0.5 mg/ml and no more than 10 mg/ml of the alpha-keto acid analogue of Valine. In some embodiments, the compositions of chitosan and alpha-keto acids as described herein may comprise at least 0.1 mg/ml and no more than 10 mg/ml of the alpha-keto acid analogue of Valine. In some embodiments, the compositions of chitosan and alpha-keto acids as described herein may comprise 5.2 mg/ml of the alpha-keto acid analogue of Valine.

In some embodiments, the compositions of chitosan and alpha-keto acids as described herein may comprise at least 0.01 mg/ml and no more than 20 mg/ml, at least 0.1 mg/ml and no more than 20 mg/ml, at least 0.1 mg/ml and no more than 15 mg/ml, at least 0.1 mg/ml and no more than 10 mg/ml, or at least 0.5 mg/ml and no more than 10 mg/ml of the alpha-keto acid analogue of Phenylalanine. In some embodiments, the compositions of chitosan and alpha-keto acids as described herein may comprise at least 0.1 mg/ml and no more than 10 mg/ml of the alpha-keto acid analogue of Phenylalanine. In some embodiments, the compositions of chitosan and alpha-keto acids as described herein may comprise 2.5 mg/ml of the alpha-keto acid analogue of Phenylalanine.

In some embodiments, the compositions of chitosan and alpha-keto acids as described herein may comprise at least 0.01 mg/ml and no more than 20 mg/ml, at least 0.1 mg/ml and no more than 20 mg/ml, at least 0.1 mg/ml and no more than 15 mg/ml, at least 0.1 mg/ml and no more than 10 mg/ml, or at least 0.5 mg/ml and no more than 10 mg/ml of the alpha-keto acid analogue of Leucine. In some embodiments, the compositions of chitosan and alpha-keto acids as described herein may comprise at least 0.1 mg/ml and no more than 15 mg/ml of the alpha-keto acid analogue of Leucine. In some embodiments, the compositions of chitosan and alpha-keto acids as described herein may comprise 7.8 mg/ml of the alpha-keto acid analogue of Leucine.

In some embodiments, the compositions of chitosan and alpha-keto acids as described herein may comprise at least 0.01 mg/ml and no more than 20 mg/ml, at least 0.1 mg/ml and no more than 20 mg/ml, at least 0.1 mg/ml and no more than 15 mg/ml, at least 0.1 mg/ml and no more than 10 mg/ml, or at least 0.5 mg/ml and no more than 10 mg/ml of the alpha-keto acid analogue of Cysteine. In some embodiments, the compositions of chitosan and alpha-keto acids as described herein may comprise at least 0.1 mg/ml and no more than 10 mg/ml of the alpha-keto acid analogue of Cysteine. In some embodiments, the compositions of chitosan and alpha-keto acids as described herein may comprise 0.8 mg/ml of the alpha-keto acid analogue of Cysteine.

In some embodiments, the compositions of chitosan and alpha-keto acids as described herein may comprise at least 0.01 mg/ml and no more than 20 mg/ml, at least 0.1 mg/ml and no more than 20 mg/ml, at least 0.1 mg/ml and no more than 15 mg/ml, at least 0.1 mg/ml and no more than 10 mg/ml, or at least 0.5 mg/ml and no more than 10 mg/ml of the alpha-keto acid analogue of Tyrosine. In some embodiments, the compositions of chitosan and alpha-keto acids as described herein may comprise at least 0.1 mg/ml and no more than 10 mg/ml of the alpha-keto acid analogue of Tyrosine. In some embodiments, the compositions of chitosan and alpha-keto acids as described herein may comprise 2.5 mg/ml of the alpha-keto acid analogue of Tyrosine.

The compositions of chitosan and alpha-keto acids as described herein may comprise at least 0.001% and no more than 10% of an additional chemical compound (e.g., lipids (e.g., DSPE-PEG, magnesium stearate), bases (e.g., NaOH), polysaccharides (e.g., sodium alginate or mannitol), tripoly phosphate (TPP), (hydroxypropyl)methyl cellulose phthalate (HPMCP), polymers (e.g., PEG, PEG3350, PS80, or P188), citrate, or any combination thereof). In some embodiments, compositions of chitosan and alpha-keto acids as described herein may comprise at least 0.001% and no more than 0.1% of DSPE-PEG. In some embodiments, compositions of chitosan and alpha-keto acids as described herein may comprise 0.025% of DSPE-PEG. In some embodiments, compositions of chitosan and alpha-keto acids as described herein may comprise at least 0.1% and no more than 10% of mannitol. In some embodiments, compositions of chitosan and alpha-keto acids as described herein may comprise 5% of mannitol. In some embodiments, compositions of chitosan and alpha-keto acids as described herein may comprise at least 0.1% and no more than 10% of magnesium stearate. In some embodiments, compositions of chitosan and alpha-keto acids as described herein may comprise 1% of magnesium stearate. In some embodiments, compositions of chitosan and alpha-keto acids as described herein may comprise at least 0.1% and no more than 10% of alginate. In some embodiments, compositions of chitosan and alpha-keto acids as described herein may comprise 1% of alginate.

The compositions of chitosan and alpha-keto acids as described herein may be formulated as a lyophilized formulation from a solution of the chitosan and alpha-keto acids as described herein. In some embodiments, the compositions of chitosan and alpha-keto acids as described herein are lyophilized for at least 1 and no more than 30 hours. In some embodiments, the compositions of chitosan and alpha-keto acids as described herein are lyophilized for at least 10 and no more than 30 hours. In some embodiments, the compositions of chitosan and alpha-keto acids as described herein are lyophilized for at least 15 and no more than 25 hours.

### Methods for Synthesizing Chitosan Alpha-Keto Acid Compositions

A composition of a polysaccharide (e.g., chitosan) and one or more alpha-keto acids (e.g., alpha-keto acid analogues of amino acids) may be synthesized for use in a medical food or other composition. The composition can be synthesized by combining the alpha-keto acid (e.g., alpha-keto acid analogues of amino acids) with the polysaccharide (e.g., the chitosan). In some embodiments, the compositions may be synthesized by combining the alpha-keto acid (e.g., alpha-keto acid analogues of amino acids) with the polysaccharide (e.g., the chitosan) and optionally additional chemical compounds (e.g., lipids (e.g., DSPE-PEG, magnesium stearate), bases (e.g., NaOH), polysaccharides (e.g., sodium alginate or mannitol), tripoly phosphate (TPP), (hydroxypropyl)methyl cellulose phthalate (HPMCP), polymers (e.g., PEG, PEG3350, PS80, or P188), citrate, or any combination thereof). The alpha-keto acid may intercalate into the polysaccharide. The alpha-keto acid may associate with the polysaccharide backbone. For example, the amine group on a chitosan may associate with a carboxylate on the alpha-keto acid. In some embodiments the polysaccharide (e.g., chitosan) comprises a positive charge at a neutral pH. In some embodiments, the positive charge is a positively charged amino group on the polysaccharide (e.g., chitosan). In some embodiments, the alpha-keto acid analogue of an amino acid comprises a negative charge at a neutral pH. In some embodiments, the negative charge is a negatively charged carboxylate group on the alpha-keto acid analogue of an amino acid. In some embodiments, the negative charge is a negatively charged carboxylate group of the keto acid group of the alpha-keto acid analogue of an amino acid. In some embodiments, the polysaccharide (e.g., chitosan) and the alpha-keto acid analogue of an amino acid ionically bond through a positive charge on the polysaccharide (e.g., chitosan) and a negative charge on the alpha-keto acid analogue of an amino acid. In some embodiments, the polysaccharide (e.g., chitosan) is ionically coupled to the alpha-keto acid analogue of an amino acid and the alpha-keto acid analogue of an amino acid is considered to be bound to the polysaccharide (e.g., chitosan). In some embodiments, the ionic coupling comprises an ionic bond between a positively charged amino group and a negatively charged carboxylate group.

A method of making the composition can include varying degree of association between the chitosan and the alpha-keto acid. In some embodiments, between about 10% and 100% of alpha-keto acids combined with the chitosan associate with the chitosan. The association of the alpha-keto acids to the chitosan may be monitored through changes in chitosan molecular weight as alpha-keto acids replace coordinated water molecules. The association of the alpha-keto acids to the polysaccharide (e.g., chitosan) may also be characterized by high performance liquid chromatography. The association of the alpha-keto acids to the polysaccharide (e.g., chitosan) may also be represented as the percentage of the alpha-keto acid analogues of amino acids bound to the polysaccharide (e.g., chitosan), referred to as “percent bound”.

In some embodiments, at least 10% and no more than 99% of the keto acid composition (e.g., compositions comprising alpha-keto acid analogues of amino acids) is bound to the polysaccharide (e.g., chitosan). In some embodiments, at least 15% and no more than 99%, at least 20% and no more than 99%, at least 25% and no more than 99%, at least 30% and no more than 99%, at least 35% and no more than 99%, at least 40% and no more than 99%, at least 45% and no more than 99%, at least 50% and no more than 99%, at least 55% and no more than 99%, at least 60% and no more than 99%, at least 65% and no more than 99%, at least 70% and no more than 99%, at least 75% and no more than 99%, at least 80% and no more than 99%, at least 85% and no more than 99%, at least 90% and no more than 99%, or at least 95% and no more than 99% of the keto acid composition (e.g., compositions comprising alpha-keto acid analogues of amino acids) is bound to the polysaccharide (e.g., chitosan). In some embodiments, alpha-keto acid analogues of amino acids may have different percentages bound. In some embodiments, at least 15% and no more than 99%, at least 20% and no more than 99%, at least 25% and no more than 99%, at least 30% and no more than 99%, at least 35% and no more than 99%, at least 40% and no more than 99%, at least 45% and no more than 99%, at least 50% and no more than 99%, at least 55% and no more than 99%, at least 60% and no more than 99%, at least 65% and no more than 99%, at least 70% and no more than 99%, at least 75% and no more than 99%, at least 80% and no more than 99%, at least 85% and no more than 99%, at least 90% and no more than 99%, or at least 95% and no more than 99% of any individual alpha-keto acid analogue of an amino acids is bound to the polysaccharide (e.g., chitosan). For example, a keto acid composition (e.g., compositions comprising alpha-keto acid analogues of amino acids) bound to the polysaccharide (e.g., chitosan) may have at least 15% and no more than 99% bound Histidine, at least 15% and no more than 99% bound Glycine, at least 15% and no more than 99% bound Valine, at least 15% and no more than 99% bound Methionine, at least 15% and no more than 99% bound Isoleucine, at least 15% and no more than 99% bound Tyrosine, at least 15% and no more than 99% bound Leucine, at least 15% and no more than 99% bound Phenylalanine, at least 15% and no more than 99% bound Tryptophan, or any combination thereof. In some embodiments, a keto acid composition (e.g., compositions comprising alpha-keto acid analogues of amino acids) bound to the polysaccharide (e.g., chitosan) may have at least 15% and no more than 99% bound Histidine, at least 15% and no more than 99% bound Glycine, at least 15% and no more than 99% bound Valine, at least 15% and no more than 99% bound Methionine, at least 15% and no more than 99% bound Isoleucine, at least 15% and no more than 99% bound Tyrosine, at least 15% and no more than 99% bound Leucine, at least 15% and no more than 99% bound Phenylalanine, and at least 15% and no more than 99% bound Tryptophan. In some embodiments, a keto acid composition (e.g., compositions comprising alpha-keto acid analogues of amino acids) bound to the polysaccharide (e.g., chitosan) may have at least 15% and no more than 99% bound Histidine, at least 1% and no more than 99% bound Glycine, at least 10% and no more than 99% bound Valine, at least 50% and no more than 99% bound Methionine, at least 20% and no more than 99% bound Isoleucine, at least 80% and no more than 99% bound Tyrosine, at least 10% and no more than 99% bound Leucine, at least 40% and no more than 99% bound Phenylalanine, and at least 20% and no more than 99% bound Tryptophan. In some embodiments, a keto acid composition (e.g., compositions comprising alpha-keto acid analogues of amino acids) bound to the polysaccharide (e.g., chitosan) may have at least 1% and no more than 99% bound Glycine, at least 10% and no more than 99% bound Valine, at least 50% and no more than 99% bound Methionine, at least 20% and no more than 99% bound Isoleucine, at least 80% and no more than 99% bound Tyrosine, at least 10% and no more than 99% bound Leucine, at least 40% and no more than 99% bound Phenylalanine, and at least 20% and no more than 99% bound Tryptophan. In some embodiments, a keto acid composition (e.g., compositions comprising alpha-keto acid analogues of amino acids) bound to the polysaccharide (e.g., chitosan) may have at least 10% and no more than 99% bound Valine, at least 50% and no more than 99% bound Methionine, at least 20% and no more than 99% bound Isoleucine, at least 80% and no more than 99% bound Tyrosine, at least 10% and no more than 99% bound Leucine, at least 40% and no more than 99% bound Phenylalanine, and at least 20% and no more than 99% bound Tryptophan. In some embodiments, a keto acid composition (e.g., compositions comprising alpha-keto acid analogues of amino acids) bound to the polysaccharide (e.g., chitosan) may have at least 15% and no more than 99% bound Histidine, at least 10% and no more than 99% bound Valine, at least 50% and no more than 99% bound Methionine, at least 20% and no more than 99% bound Isoleucine, at least 80% and no more than 99% bound Tyrosine, at least 10% and no more than 99% bound Leucine, at least 40% and no more than 99% bound Phenylalanine, and at least 20% and no more than 99% bound Tryptophan.

In some embodiments, a stoichiometric ratio of the alpha-keto acid and chitosan amines may be between about 1:1 and 1:10, about 1:1 and 1:100, about 1:1 and 1:1000, about 1:1 and 1:10,000, or about 1:1 and 1:100,000. A stoichiometric ratio of alpha-keto acids to a chitosan compound may refer to the stoichiometric ratio of alpha-keto acids to the amine groups on the chitosan. Another ratio that may be used for the number of alpha-keto acids to a chitosan may be a weight ratio. In some embodiments, a weight ratio of the chitosan to the alpha-keto acid may be between about 1:1 and 1:10, about 1:1 and 1:100, about 1:1 and 1:1000, about 1:1 and 1:10,000, or about 1:1 and 1:100,000.

### Consumable Compositions of Chitosan Keto Acids

The present disclosure provides consumable compositions comprising a composition of the present disclosure (e.g., a composition comprising a chitosan and one or more alpha-keto acid analogues of an amino acid). In some cases, the consumable composition comprises a chitosan and one or more alpha-keto acid analogues of amino acids described herein. In some cases, the consumable composition is a food. In some cases, the consumable composition may be a medical grade composition. In some cases, the consumable composition is a dietary supplement. As disclosed herein, a composition with a polysaccharide (e.g., a chitosan) and an alpha-keto acid (e.g., an alpha-keto acid analogue of an amino acid) can be used to supplement amino acid intake. Following consumption, the alpha-keto acid may disassociate from the polysaccharide to provide free alpha-keto acids, which may be absorbed and converted into amino acids. In some embodiments, a consumable composition may comprise a chitosan and one or more alpha-keto acid analogues of amino acids at concentrations similar to the daily requirements of the corresponding amino acids. In some embodiments, a unit dose or a daily dose of the compositions may comprise 5 mg tryptophan, 22 mg glycine, 19 mg methionine, 19 mg isoleucine, 14 mg histidine, 100 mg tyrosine, 20 mg threonine, 55 mg arginine, 24 mg valine, 33 mg phenylalanine, 42 mg leucine, 4 mg cysteine, 30 mg lysine, or any combination thereof, per kg of the body weight of a subject. In some embodiments, a unit dose or a daily dose of the compositions may comprise at least 5 mg tryptophan, at least 22 mg glycine, at least 19 mg methionine, at least 19 mg isoleucine, at least 14 mg histidine, at least 100 mg tyrosine, at least 20 mg threonine, at least 55 mg arginine, at least 24 mg valine, at least 33 mg phenylalanine, at least 42 mg leucine, at least 4 mg cysteine, at least 30 mg lysine, or any combination thereof, per kg of the body weight of a subject. In some embodiments, a unit dose or a daily dose of the compositions may comprise at least 2.5 mg and no more than 30 mg tryptophan per kg of the body weight of a subject. In some embodiments, a unit dose or a daily dose of the compositions may comprise at least 20 mg and no more than 50 mg glycine per kg of the body weight of a subject. In some embodiments, a unit dose or a daily dose of the compositions may comprise at least 15 mg and no more than 50 methionine per kg of the body weight of a subject. In some embodiments, a unit dose or a daily dose of the compositions may comprise at least 15 mg and no more than 50 mg isoleucine per kg of the body weight of a subject. In some embodiments, a unit dose or a daily dose of the compositions may comprise at least 10 mg and no more than 40 mg histidine per kg of the body weight of a subject. In some embodiments, a unit dose or a daily dose of the compositions may comprise at least 80 mg and no more than 200 mg tyrosine per kg of the body weight of a subject. In some embodiments, a unit dose or a daily dose of the compositions may comprise at least 15 mg and no more than 50 mg threonine per kg of the body weight of a subject. In some embodiments, a unit dose or a daily dose of the compositions may comprise at least 50 mg and no more than 100 mg arginine per kg of the body weight of a subject. In some embodiments, a unit dose or a daily dose of the compositions may comprise at least 20 mg and no more than 50 mg valine per kg of the body weight of a subject. In some embodiments, a unit dose or a daily dose of the compositions may comprise at least 25 mg and no more than 60 mg phenylalanine per kg of the body weight of a subject. In some embodiments, a unit dose or a daily dose of the compositions may comprise at least 30 mg and no more than 70 mg leucine per kg of the body weight of a subject. In some embodiments, a unit dose or a daily dose of the compositions may comprise at least 2.5 mg and no more than 30 mg cysteine per kg of the body weight of a subject. In some embodiments, a unit dose or a daily dose of the compositions may comprise at least 25 mg and no more than 60 mg lysine per kg of the body weight of a subject.

In some cases, the consumable composition may comprise of a liquid, a solid, a colloid, a gel, or a combination thereof. In some cases, the consumable composition may comprise a food or a beverage. For example, the consumable composition (e.g., a medical grade food) can comprise a sports drink, a bar, a cereal, a gel, a gelatin, a gummy, a cracker, a spread, a chip, a granola, a liquid, a tablet, a powder, a suspension, or a combination thereof. In some embodiments, the food may be portioned into a single serving or a plurality of servings. The food can provide one or more amino acids in the form of a composition comprising a chitosan bound to alpha-keto acid analogues of amino acids, and may further provide additional nutrients, such as vitamins, minerals, fats, carbohydrates, proteins, or free amino acids.

In some embodiments, the food may have a lower nitrogen content per kg of protein (e.g., amino acid or alpha-keto acid) than other protein supplements or protein sources. In some embodiments, the food may have a nitrogen content less than 16% relative to total food weight. In some embodiments, the food may have a nitrogen content less than 10% relative to total food weight. In some embodiments, the food may have a nitrogen content less than 5% relative to total food weight. In some embodiments, the food may have a nitrogen content less than 1% relative to total food weight. In some embodiments, the food may have a nitrogen content less than 0.1% relative to total food weight. The nitrogen content of the food may be measured by USP method 461.

In some embodiments, the food may have a lower salt (e.g., sodium, calcium, or magnesium) content per kg of alpha-keto acid content than other protein supplements or protein sources (e.g., alpha-keto acid salts).

The food compositions as described herein may have a lower salt content than other protein supplements or protein sources that may be beneficial in reducing overall salt intake. The food compositions as described herein may have a salt level that is less than the daily recommended value for salt intake as recommended by the Food & Drug Administration (FDA). For example, the FDA has a daily recommended value of 2,300 mg sodium per day for adults, 1,300 mg calcium per day for adults, and 420 mg of magnesium per day for adults. In some embodiments, the food may have a salt (e.g., sodium, calcium, or magnesium) content less than 20% of the recommended daily value of the salt. In some embodiments, the food may have a salt (e.g., sodium, calcium, or magnesium) content less than 10% of the recommended daily value of the salt. In some embodiments, the food may have a salt (e.g., sodium, calcium, or magnesium) content less than 5% of the recommended daily value of the salt. For example, the food may comprise less than 10% of the recommended daily value of sodium. In another example, the food may comprise less than 10% of the recommended daily value of calcium. example, the food may comprise less than 10% of the recommended daily value of magnesium. For example, the food may comprise less than 5% of the recommended daily value of sodium. In another example, the food may comprise less than 5% of the recommended daily value of calcium. example, the food may comprise less than 5% of the recommended daily value of magnesium. For example, the food may comprise less than 1% of the recommended daily value of sodium. In another example, the food may comprise less than 1% of the recommended daily value of calcium. example, the food may comprise less than 1% of the recommended daily value of magnesium.

In some embodiments, the food may provide one or more amino acids in the form of a composition comprising a chitosan bound to alpha-keto acid analogues of amino acids and may further provide the free amino acid of proline. In some embodiments, a composition may comprise a polysaccharide and a proline. In some embodiments, a composition may comprise a chitosan and a proline.

The consumable composition can comprise a chitosan and an alpha-keto acid. The consumable composition can comprise a plurality of chitosans and alpha-keto acids. In some embodiments, the consumable composition (e.g., a medical food) includes the chitosan and (i) an alpha-keto acid analogue of a non-proline natural amino acid, (ii) an alpha-keto acid analogue of a non-proline proteinogenic amino acid, (iii) an alpha-keto acid analogue of an essential amino acid, (iv) an alpha-keto acid analogue of a non-proline quasi-essential amino acid, (v) an alpha-keto acid analogue of a non-essential amino acid, or (vi) a combination thereof. In some embodiments, the consumable composition includes the chitosan and a plurality of alpha-keto acid analogues of essential amino acids. In some embodiments, the consumable composition includes the chitosan and a plurality of alpha-keto acid analogues of essential amino acids and non-proline quasi-essential amino acids. In some embodiments, the consumable composition includes the chitosan and a plurality of alpha-keto acid analogues of essential amino acids, non-proline conditionally essential amino acids, and non-essential amino acids.

In some cases, the consumable composition comprises a plurality of chitosans with varying degrees of deacetylation. In some cases, the consumable composition comprises a plurality of chitosans with varying sizes. In some such cases, the mixture can be stochastic, for example containing a combination of chitosan alpha-keto acid complexes with a statistical distribution of alpha-keto acid-to-chitosan amine ratios or a random mixture of types of alpha-keto acids. In other cases, the mixture can be non-stochastic, instead reflecting a mixture of multiple alpha-keto acid analogues of amino acids in controlled ratios (e.g., a collection of chitosan combined with a 5:5:5:4:1 mixture of

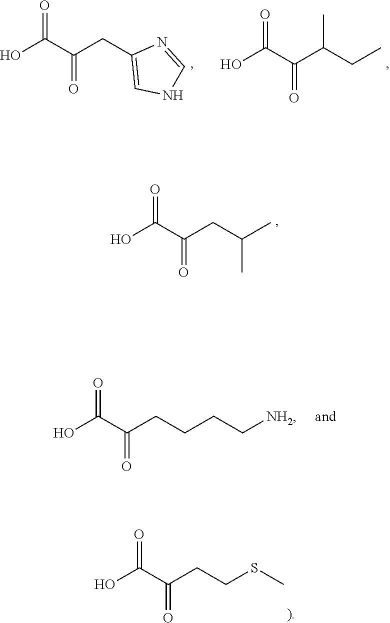

In such cases, the distributions of the chitosan alpha-keto acid compositions can be stochastic, but the ratios of alpha-keto acids and chitosan can be controlled through reagent stoichiometry. In some cases, the consumable composition comprises approximately equal amounts of two compounds (e.g., from a 5:4 ratio to a 4:5 ratio of two different alpha-keto acids, or from an 11:10 ratio to a 10:11 ratio of two different alpha-keto acids).

In some cases, the consumable composition comprises a plurality of chitosan-alpha-keto acid complexes comprising different alpha-keto acid analogues of non-proline natural amino acids (e.g., a first compound of chitosan comprising one or more alpha-keto acid analogues of alanine, a second compound of chitosan comprising one or more alpha-keto acid analogues of histidine, etc.). In some cases, the consumable composition comprises a plurality of compounds of chitosan comprising alpha-keto acid analogues of all non-proline natural amino acids.

In some cases, the consumable composition comprises a plurality of compounds of chitosan comprising alpha-keto acid analogues of all essential amino acids.

In some cases, the consumable composition comprises a plurality of compounds of chitosan comprising alpha-keto acid analogues of at least 3 essential amino acids.

The polysaccharide and alpha-keto acid compositions (e.g., a chitosan and alpha-keto acid analogues of one or more amino acids) described herein may be used as a treatment or nutritional supplement. In such cases, the composition may be provided with other composition that may improve its bioavailability, digestibility, or a different desirable feature. The consumable composition described herein may be present as a powder, liquid, or mixture that can be combined with a nutritional supplement. In some cases, the consumable composition comprises a polysaccharide, an alpha-keto acid, and a pharmaceutically acceptable excipient. In some cases, the consumable composition comprises a polysaccharide, an alpha-keto acid analogue of an essential amino acid, and a pharmaceutically acceptable excipient. In some cases, between about 30% and 90%, between about 50% and 80%, or between about 60% and 95% of dry weight of the consumable composition is carbohydrates, fats, protein, or a combination thereof.

### Polyol Keto Esters

The present disclosures provide the compositions comprising polyol keto ester (e.g., glyceride keto ester) compounds. These compounds may include at least one amino acid sidechain with an alpha-keto ester linkage to a polyol backbone. For example, these compounds can include a glycerol functionalized with one or more alpha-keto acyl groups to form a glyceride keto ester. Often, the compounds can be readily metabolized to form amino acids and other catabolically beneficial species in vivo, and therefore can serve as nutrient sources for subjects in need thereof, for example subjects with amino acid deficiencies or low dietary protein tolerances.

The alpha-keto acid forms of many amino acids are made in vivo from transaminases, which catalyze the transfer of the amino group of the amino acid to a different alpha-keto acid (e.g., alpha-keto glutarate). Keto acids contain both a ketone functional group, and a carboxylic acid group. Like the keto acid forms, keto esters (e.g., glyceride keto esters) comprise a ketone functional group. Unlike the keto acids, the keto esters (e.g., keto esters of Formula (XVI)-Formula (XXII)) described herein can further comprise a polyol backbone covalently bonded to the carboxylic acid carbon of the alpha-keto acid, thereby transforming the alpha-keto acid into an alpha-keto ester. Following administration (e.g., ingestion), alpha-keto ester compounds can be hydrolyzed (e.g., in vivo) to liberate alpha-keto substituents from polyol backbones to yield a polyol and an alpha-keto acid.

### Polyol Keto Ester Compounds

Aspects of the present disclosure provide polyol compounds with keto ester functionalizations bearing amino acid side chains. For many of the compounds disclosed herein, the keto ester side chains are alpha-keto esters, rendering them highly hydrolysable and promoting conversion to the corresponding alpha-keto acid and free polyol upon cleavage. The amino acid sidechain can be coupled directly to the keto group, such that transamination of the keto ester or a liberated keto acid thereof generates an amino acid or an analogue thereof.

As non-limiting examples, the polyol can be selected from the group consisting of:

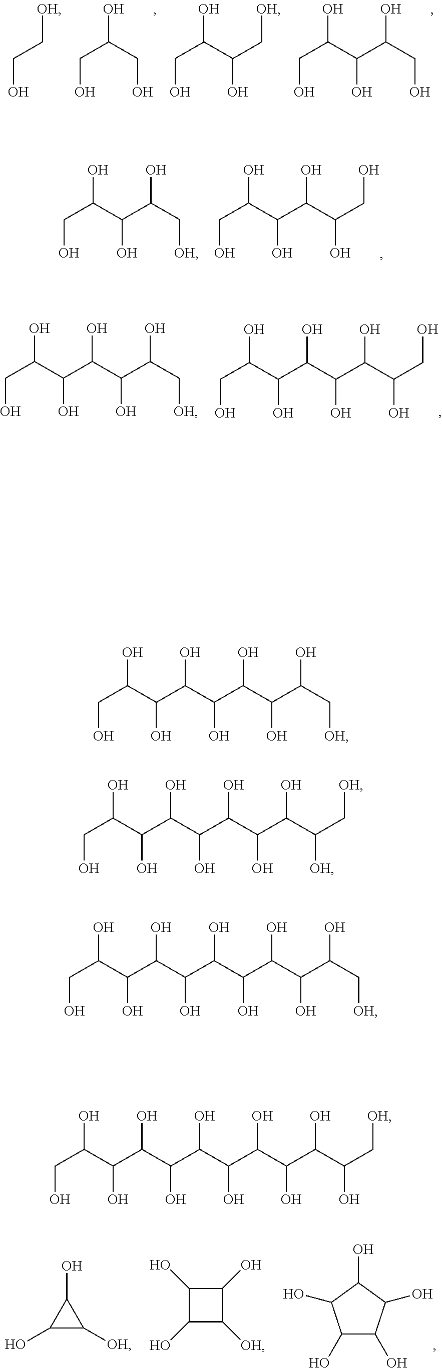

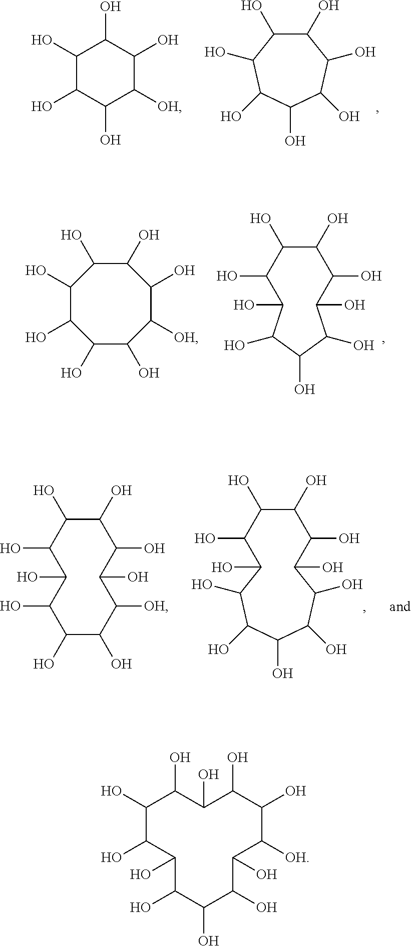

In some cases, the polyol is selected from the group consisting of:

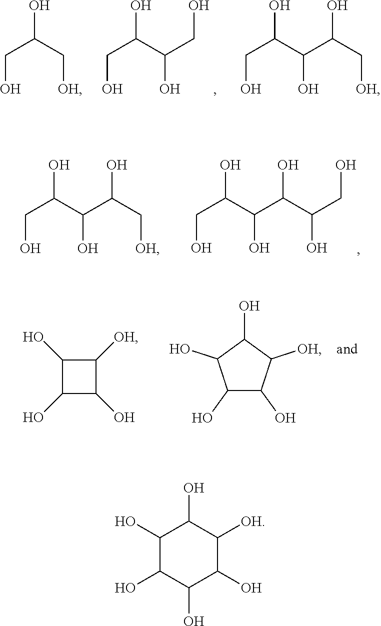

In some cases, the polyol is a sugar, for example,

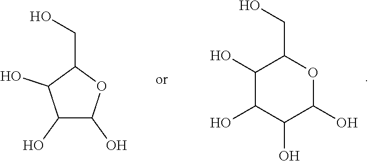 [SMILES: OCC1OC(O)C(O)C1O ; "or" ; OCC1OC(O)C(O)C(O)C1O ; "."]

In some cases, the polyol comprises absolute stereochemistry. For example, the polyol can be erythritol, threitol, arabitol, ribitol, xylitol, allitol, altritol, galactitol, glucitol, iditol, inositol, mannitol, sorbitol, perseitol, volemitol, isomalt, lactitol, maltitol, maltotriitol, maltotetraitol, polyglycitol, or a combination thereof. In some cases, the polyol includes a mixture of enantiomers. In some cases, the polyol is racemic. In some cases, the polyol is cyclic, linear, or branched. In some cases, the polyol is cyclic or linear. In some cases, the polyol is linear. In some cases, the polyol is cyclic.

The polyol can have a low glycemic index. As used herein, “glycemic index” can denote the degree to which a substance increases blood glucose levels following consumption. Glycemic index scales typically range from 0 to 100, with 100 corresponding to glucose, while unrefined carbohydrates (e.g., those present in most vegetables, grains, and fruits) have values of less than about 50. As high glycemic index foods can increase certain forms of lipogenesis and hepatic stress, a compound of the present disclosure (e.g., a compound of Formula (XVI)-Formula (XXII)) can utilize a polyol backbone with a low glycemic index, such that polyol liberation through keto ester hydrolysis affects minimal increases in blood glucose levels. For example, glycerol's low glycemic index of 3 (more than 22-times lower than that of sucrose) renders Formula (XVI)-Formula (XX) as suitable for administration to subjects with liver disease, diabetes, and other diseases related to hyperglycemia and insulin intolerance. Many of the polyol backbones of the compounds disclosed herein have similarly low glycemic indices, for example maltitol (glycemic index of 35), xylitol (glycemic index of 13), isomalt (glycemic index of 9), sorbitol (glycemic index of 9), lactitol (glycemic index of 6), erythritol (glycemic index of 0), and mannitol (glycemic index of 0). In some cases, the polyol of a compound disclosed herein has a glycemic index of not less than 0 and not greater than 50. In some cases, the polyol of a compound disclosed herein has a glycemic index of not less than 0 and not greater than 30. In some cases, the polyol of a compound disclosed herein has a glycemic index of not less than 0 and not greater than 20. In some cases, the polyol of a compound disclosed herein has a glycemic index of not less than 0 and not greater than 15. In some cases, the polyol of a compound disclosed herein has a glycemic index of not less than 0 and not greater than 10.

### Linear Keto Esters

In some embodiments, a compound of the present disclosure comprises a structure according to Formula (XXI):

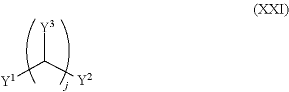

wherein:
Y¹ and Y² are each independently selected from the group consisting of —CH₂Y³, —C(═O) H, —C(═O)OX₂, and —C(═O)NX²X³;
each instance of Y³ is each independently selected from the group consisting of —H, —OX¹, —NX²X³, —C(═O)OX₂, and —C(═O)NX²X³;
each instance of X¹ is independently selected from the group consisting of —H, —C₁₋₃ alkyl, —C(═O)CH₃, —C(═O)OX₂, —C(═O)NX²X³, —PO₃²⁻, —SO₃⁻, a C₄₋₉ monosaccharide, a C₈₋₁₈ disaccharide, and

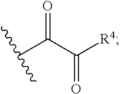

each instance of X² and X³ is independently selected from the group consisting of —H and —C₁-C₃ alkyl;
each instance of R⁴ is independently selected from the group consisting of non-proline, non-alanine, and non-glycine natural amino acid sidechains, subscript j is an integer from 0 to 23; and
at least one instance of Y¹, Y², and Y³ is —OX¹ of which X¹ is

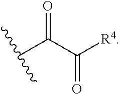

In some embodiments, a compound of the present disclosure comprises a structure according to Formula (XXIa):

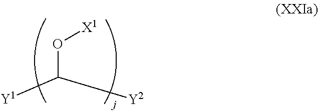

wherein:
Y¹ and Y² are each independently selected from the group consisting of —CH₂Y³, —C(═O) H, —C(═O)OX₂, and —C(═O)NX²X³;
each instance of Y³ is each independently selected from the group consisting of —H, —OX¹, —NX²X³, —C(═O)OX₂, and —C(═O)NX²X³;
each instance of X¹ is independently selected from the group consisting of —H, —C₁₋₃ alkyl, —C(═O)CH₃, —C(═O)OX₂, —C(═O)NX²X³, —PO₃²⁻, —SO₃⁻, a C₄₋₉ monosaccharide, a C₈₋₁₈ disaccharide, and

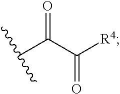

each instance of X² and X³ is independently selected from the group consisting of —H and —C₁-C₃ alkyl;
each instance of R⁴ is independently selected from the group consisting of non-proline, non-alanine, and non-glycine natural amino acid sidechains, subscript j is an integer from 0 to 23; and
at least one instance of X¹ is

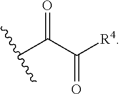

In some cases, a compound of the present disclosure comprises a structure according to Formula (XXIb):

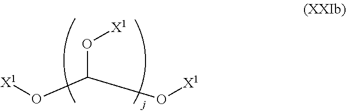

wherein:
each instance of X¹ is independently selected from the group consisting of —H, —C₁₋₃ alkyl, —C(═O)CH₃, —C(═O)OX₂, —C(═O)NX²X³, —PO₃²⁻, —SO₃⁻, a C₄₋₉ monosaccharide, a C₈₋₁₈ disaccharide, and

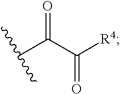

each instance of X² and X³ is independently selected from the group consisting of —H and —C₁-C₃ alkyl;
each instance of R⁴ is independently selected from the group consisting of non-proline natural amino acid sidechains, and
subscript j is an integer from 0 to 23; and
at least one instance of X¹ is

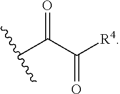

In some cases of Formulae (XXI) and (XXIa), Y¹ is —CH₂Y³. In some cases of Formulae (XXI) and (XXIa), Y¹ is —CH₂Y³ and Y² is selected from the group consisting of —CH₂Y³ and —C(═O)OX₂. In some cases of Formulae (XXI) and (XXIa), Y¹ and Y² are each —CH₂Y³.

In some cases of Formulae (XXI) and (XXIa), each instance of Y³ is independently selected from the group consisting of —H, —OX¹, and —C(═O)OX₂. In some cases of Formulae (XXI) and (XXIa), at most one instance of Y³ is —C(═O)OX₂ and the remaining instances of Y³ are —OX¹. In some cases of Formulae (XXI) and (XXIa), at most one instance of Y³ is —C(═O) OH and the remaining instances of Y³ are —OX¹. In some cases of Formulae (XXI) and (XXIa), each instance of Y³ is —OX¹.

In some cases of Formulae (XXI), (XXIa), and (XXIb), each instance of X¹ is independently selected from the group consisting of —H, C₁₋₃ alkyl, —PO₃²⁻, —SO₃⁻, a C₄₋₉ monosaccharide, and

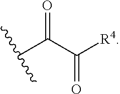

In some cases of Formulae (XXI), (XXIa), and (XXIb), at most one instance of X¹ is —PO₃²⁻, —SO₃⁻, a C₄₋₉ monosaccharide, or a C₈₋₁₈ disaccharide, and the remaining instances are each independently selected from the group consisting of —H, C₁₋₃ alkyl, and

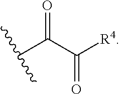

In some cases of Formulae (XXI), (XXIa), and (XXIb), each instance of X¹ is independently selected from the group consisting of —H, methyl, and

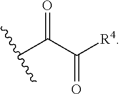

In some cases of Formulae (XXI), (XXIa), and (XXIb), each instance of X¹ is independently selected from the group consisting of —H and

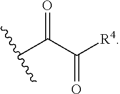

In some cases of Formulae (XXI), (XXIa), and (XXIb), at most two instances of X¹ are —H and the remaining instances of X¹ are

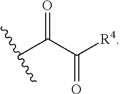

In some cases of Formulae (XXI), (XXIa), and (XXIb), at most one instance of X¹ is-H and the remaining instances of X¹ are

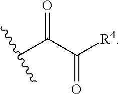

In some cases of Formulae (XXI), (XXIa), and (XXIb), 1, 2, or 3 instances of X¹ are

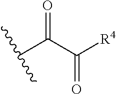

and the remaining instances are —H. In some cases of Formulae (XXI), (XXIa), and (XXIb), 1 or 2 instances of X¹ are and

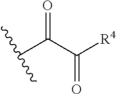

the remaining instances are —H. In some cases of Formulae (XXI), (XXIa), and (XXIb), one instance of X¹ is

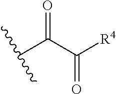

and the remaining instances are —H.

In some cases of Formulae (XXI), (XXIa), and (XXIb), subscript j is an integer from 1 to 10. In some cases of Formulae (XXI), (XXIa), and (XXIb), subscript j is an integer from 4 to 10. In some cases of Formulae (XXI), (XXIa), and (XXIb), subscript j is an integer from 6 to 12. In some cases of Formulae (XXI), (XXIa), and (XXIb), subscript j is an integer from 0 to 6. In some cases of Formulae (XXI), (XXIa), and (XXIb), subscript j is an integer from 1 to 6. In some cases of Formulae (XXI), (XXIa), and (XXIb), subscript j is an integer from 1 to 4. In some cases of Formulae (XXI), (XXIa), and (XXIb), subscript j is an integer from 1 to 3. In some cases of Formulae (XXI), (XXIa), and (XXIb), subscript j is 0. In some cases of Formulae (XXI), (XXIa), and (XXIb), subscript j is 1. In some cases of Formulae (XXI), (XXIa), and (XXIb), subscript j is 2. In some cases, subscript j is 3.

In some cases of Formulae (XXI), (XXIa), and (XXIb), each instance of R⁴ is independently selected from the group consisting of non-proline, non-alanine, and non-glycine natural amino acid sidechains. In some cases of Formulae (XXI), (XXIa), and (XXIb), each instance of R⁴ is independently selected from the group consisting of non-proline, non-alanine, and non-glycine proteinogenic amino acid sidechains. In some cases of Formulae (XXI), (XXIa), and (XXIb), each instance of R⁴ is independently selected from the group consisting of:

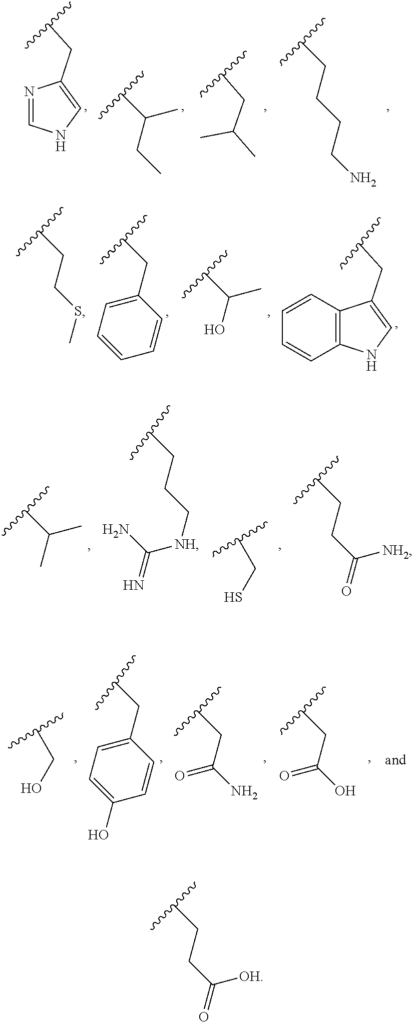

In some cases of Formulae (XXI), (XXIa), and (XXIb), each instance of R⁴ is independently selected from the group consisting of essential amino acid sidechains. In some cases of Formulae (XXI), (XXIa), and (XXIb), each instance of R⁴ is identical. In some cases of Formulae (XXI), (XXIa), and (XXIb), at least two instances of R⁴ are not identical.

### Cyclic Polyols

In some cases, a polyol keto ester of the present disclosure comprises a compound of Formula (XXII):

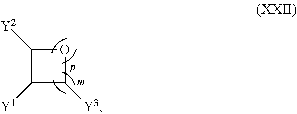

wherein:
Y¹ is selected from the group consisting of —H, —OX¹, —C(═O)OX₂, —C(═O)NX²X³;
Y² is selected from the group consisting of —H, —OX¹, —CH₂OX¹, —C(═O)OX₂, —C(═O)NX²X³, and

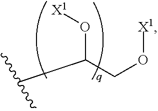

wherein subscript q is an integer from 1 to 3 and 3 and [symbol] denotes a point of attachment of Y² to the remainder of Formula (XXII);
each instance of Y³ is each independently selected from the group consisting of —H, —OX¹, —C(═O)OX₂, and —C(═O)NX²X³;
each instance of X¹ is independently selected from the group consisting of —H, —C₁₋₃ alkyl, —PO₃²⁻, —SO₃⁻, a C₄₋₉ monosaccharide, a C₈₋₁₈ disaccharide, and

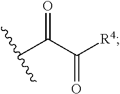

each instance of X² and X³ is independently selected from the group consisting of —H and —C₁₋₃ alkyl;
each instance of R⁴ is independently selected from the group consisting of non-proline, non-alanine, and non-glycine natural amino acid sidechains, and
subscript m is an integer from 1 to 23, and
subscript p is 0 or 1.

In some cases, a polyol keto ester of the present disclosure comprises a compound of Formula (XXIIa):

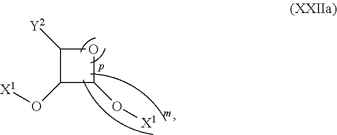

wherein:
Y² is selected from the group consisting of —OX¹, —CH₂OX¹, and

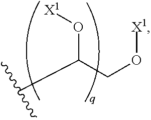

wherein subscript q is an integer from 1 to 3 and [symbol] denotes a point of attachment of Y² to the remainder of Formula (XXII);
each instance of X¹ is independently selected from the group consisting of —H, —C₁₋₃ alkyl,
—PO₃²⁻, —SO₃⁻, a C₄₋₉ monosaccharide, a C₈₋₁₈ disaccharide, and

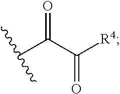

each instance of X² and X³ is independently selected from the group consisting of —H and —C₁₋₃ alkyl;
each instance of R⁴ is independently selected from the group consisting of non-proline, non-alanine, and non-glycine natural amino acid sidechains,
subscript m is an integer from 1 to 23; and
subscript p is 0 or 1.

In some cases, a polyol keto ester of the present disclosure comprises a compound of Formula (XXIIb):

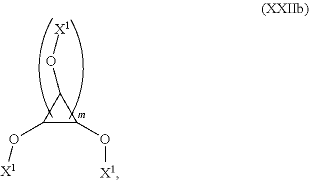

wherein:
each instance of X¹ is independently selected from the group consisting of —H, —C₁₋₃ alkyl, —PO₃²⁻, —SO₃⁻, a C₄₋₉ monosaccharide, a C₈₋₁₈ disaccharide, and

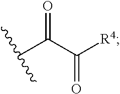

each instance of X² and X³ is independently selected from the group consisting of —H and —C₁₋₃ alkyl;
each instance of R⁴ is independently selected from the group consisting of non-proline, non-alanine, and non-glycine natural amino acid sidechains; and
subscript m is an integer from 1 to 23.

In some cases of Formulae (XXII), (XXIIa), and (XXIIb), subscript m is an integer from 2 to 8. In some cases of Formulae (XXII), (XXIIa), and (XXIIb), subscript m is an integer from 2 to 5. In some cases of Formulae (XXII), (XXIIa), and (XXIIb), subscript m is 3 or 4. In some cases, subscript m is 3. In some cases, subscript m is 4.

In some cases of Formulae (XXII) and (XXIIa), subscript p is 0. In some cases of Formulae (XXII) and (XXIIa), subscript p is 0 and Y² is —OX¹. In some cases of Formulae (XXII) and (XXIIa), subscript p is 1. In some cases of Formulae (XXII) and (XXIIa), subscript p is 1 and Y² is selected from the group consisting of —CH₂OX¹, and

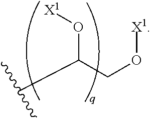

In some cases of Formula (XXII), Y¹ is selected from the group consisting of —H, —OX¹, and —C(═O)OX₂. In some cases of Formula (XXII), Y¹ is selected from the group consisting of —H and —OX¹. In some cases of Formula (XXII), Y¹ is —OX¹.

In some cases of Formulae (XXII) and (XXIIa), Y² is —OX¹. In some cases of Formulae (XXII) and (XXIIa), Y² is selected from the group consisting of —OX¹, —CH₂OX¹, and

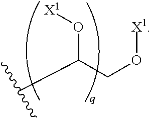

In some cases of Formulae (XXII) and (XXIIa), subscript q is 0 and Y² is —OX¹. In some cases of Formulae (XXII) and (XXIIa), subscript q is 1 and Y¹ is —CH₂OX¹.

In some cases of Formula (XXII), each instance of Y³ is each independently selected from the group consisting of —H, —OX¹, and —C(═O)OX₂. In some cases of Formula (XXII), each instance of Y³ is each independently selected from the group consisting of —H and —OX¹. In some cases of Formula (XXII), each instance of Y³ is —OX¹.

In some cases of Formulae (XXII), (XXIIa), and (XXIIb), each instance of X¹ is independently selected from the group consisting of —H, C₁₋₃ alkyl, —PO₃²⁻, —SO₃⁻, a C₄₋₉ monosaccharide, and

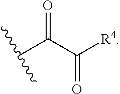

In some cases of Formulae (XXII), (XXIIa), and (XXIIb), at most one instance of X¹ is —PO₃²⁻, —SO₃⁻, a C₄₋₉ monosaccharide, or a C₈₋₁₈ disaccharide, and the remaining instances are each independently selected from the group consisting of —H, C₁₋₃ alkyl, and

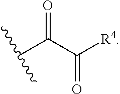

In some cases of Formulae (XXII), (XXIIa), and (XXIIb), at most one instance of X¹ is —PO₃²⁻, —SO₃⁻, a C₄₋₉ monosaccharide, or a C₈₋₁₈ disaccharide, and the remaining instances are each independently selected from the group consisting of —H and

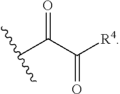

In some cases of Formulae (XXII), (XXIIa), and (XXIIb), each instance of X¹ is independently selected from the group consisting of —H, methyl, and

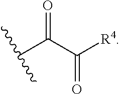

In some cases of Formulae (XXII), (XXIIa), and (XXIIb), each instance of X¹ is independently selected from the group consisting of —H and

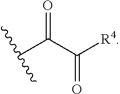

In some cases of Formulae (XXII), (XXIIa), and (XXIIb), at most two instances of X¹ are —H and the remaining instances of X¹ are

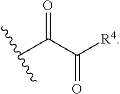

In some cases of Formulae (XXII), (XXIIa), and (XXIIb), at most one instance of X¹ is-H and the remaining instances of X¹ are

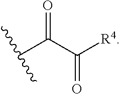

In some cases of Formulae (XXII), (XXIIa), and (XXIIb), one, two, or three instances of X¹ are

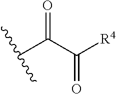

and the remaining instances are —H. In some cases of Formulae (XXII), (XXIIa), and (XXIIb), 1 or 2 instances of X¹ are

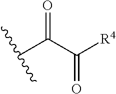

and the remaining instances are —H. In some cases of Formulae (XXII), (XXIIa), and (XXIIb), one instance of X¹ is

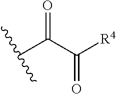

and the remaining instances are —H.

In some cases of Formulae (XXII), (XXIIa), and (XXIIb), subscript j is an integer from 1 to 10. In some cases of Formulae (XXII), (XXIIa), and (XXIIb), subscript j is an integer from 0 to 6. In some cases of Formulae (XXII), (XXIIa), and (XXIIb), subscript j is an integer from 1 to 6. In some cases of Formulae (XXII), (XXIIa), and (XXIIb), subscript j is an integer from 1 to 4. In some cases of Formulae (XXII), (XXIIa), and (XXIIb), subscript j is an integer from 1 to 3. In some cases of Formulae (XXII), (XXIIa), and (XXIIb), subscript j is 0. In some cases of Formulae (XXII), (XXIIa), and (XXIIb), subscript j is 1. In some cases of Formulae (XXII), (XXIIa), and (XXIIb), subscript j is 2. In some cases, subscript j is 3.

In some cases of Formulae (XXII), (XXIIa), and (XXIIb), each instance of R⁴ is independently selected from the group consisting of non-proline, non-alanine, and non-glycine natural amino acid sidechains. In some cases of Formulae (XXII), (XXIIa), and (XXIIb), each instance of R⁴ is independently selected from the group consisting of non-proline, non-alanine, and non-glycine proteinogenic amino acid sidechains. In some cases of Formulae (XXII), (XXIIa), and (XXIIb), each instance of R⁴ is independently selected from the group consisting of essential amino acid sidechains. In some cases of Formulae (XXII), (XXIIa), and (XXIIb), each instance of R⁴ is identical. In some cases of Formulae (XXII), (XXIIa), and (XXIIb), at least two instances of R⁴ are not identical.

### Glyceride Keto Esters

In certain cases, the compounds of the present disclosure include glycerol backbones. The glycerol backbone of glyceride keto acid esters with amino acid side chains may facilitate delivery of the amino acid sidechains. Glycerol is an inert primary alcohol suitable for synthesis with an alpha-keto acid to form the disclosed compounds. Glycerol may be hydrolyzed in the stomach to release the alpha-keto acid forms of the amino acids for subsequent metabolism. Further, since up to three amino acid sidechain groups may be linked to each glyceride, fewer osmotically active molecules will be delivered with each amino acid. Examples of glyceride keto ester compounds consistent with the present disclosure include Formula (XVI)-Formula (XX), detailed further herein.

In some embodiments, a glyceride keto ester of the present disclosure may comprise a compound of Formula (XVI), wherein R¹ is any amino acid sidechain except proline, R² is any amino acid sidechain except proline, and R³ is any amino acid sidechain except proline. In some cases, R¹ is any natural amino acid sidechain except proline, R² is any natural amino acid sidechain except proline, and R³ is any natural amino acid sidechain except proline. In some cases, R¹ is any proteinogenic amino acid sidechain except proline, R² is any proteinogenic amino acid sidechain except proline, and R³ is any proteinogenic amino acid sidechain except proline. In some cases, R¹ is any essential amino acid sidechain, R² is any essential amino acid sidechain, and R³ is any essential amino acid sidechain. In some cases of Formula (XVI), R¹, R², and R³ are identical. In some cases of Formula (XVI), R¹, R², and R³ are not identical.

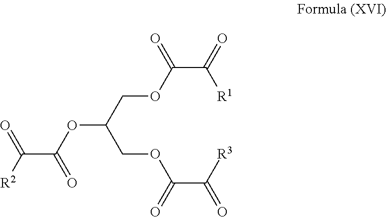

In some embodiments, a glyceride keto ester of the present disclosure may comprise a compound of Formula (XVII), wherein R¹ is any amino acid sidechain except proline and R² is any amino acid sidechain except proline. In some cases, R¹ is any natural amino acid sidechain except proline and R² is any natural amino acid sidechain except proline. In some cases, R¹ is any proteinogenic amino acid sidechain except proline and R² is any proteinogenic amino acid sidechain except proline. In some cases, R¹ is any essential amino acid sidechain, and R² is any essential amino acid sidechain. In some cases of Formula (XVII), R¹ and R² are identical. In some cases of Formula (XVII), R¹ and R² are different.

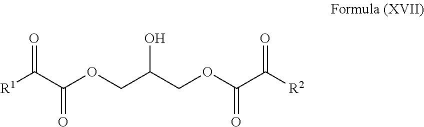

In some embodiments, a glyceride keto ester of the present disclosure may comprise a compound of Formula (XVIII), wherein R¹ is any amino acid sidechain except proline and R² is any amino acid sidechain except proline. In some cases, R¹ is any natural amino acid sidechain except proline and R² is any natural amino acid sidechain except proline. In some cases, R¹ is any proteinogenic amino acid sidechain except proline and R² is any proteinogenic amino acid sidechain except proline. In some cases, R¹ is any essential amino acid sidechain, and R² is any essential amino acid sidechain. In some cases of Formula (XVIII), R¹ and R² are identical. In some cases of Formula (XVIII), R¹ and R² are different.

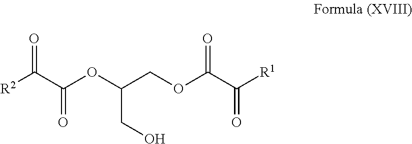

In some embodiments, a glyceride keto ester of the present disclosure may comprise a compound of Formula (XIX), wherein R¹ is any amino acid sidechain except proline. In some cases, R¹ is any natural amino acid sidechain except proline. In some cases, R¹ is any proteinogenic amino acid sidechain except proline. In some cases, R¹ is any essential amino acid sidechain.

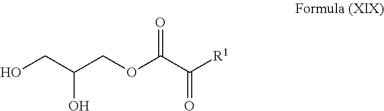

In some embodiments, a glyceride keto ester of the present disclosure may comprise a compound of Formula (XX), wherein R¹ is any amino acid sidechain except proline. In some cases, R¹ is any natural amino acid sidechain except proline. In some cases, R¹ is any proteinogenic amino acid sidechain except proline. In some cases, R¹ is any essential amino acid sidechain.

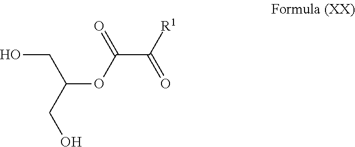

An amino acid sidechain of the present disclosure (e.g., an amino acid sidechain included in a compound of any one of Formula (XVI)-Formula (XXII)) may be a sidechain of a natural amino acid. While only 21 amino acids have been identified as proteinogenic in eukaryotes, many of the approximately 500 amino acids identified in nature are active metabolized in humans, rendering them as valuable nutrients. Furthermore, many non-proteinogenic amino acids are direct precursors to proteinogenic amino acids, which can make them effective sources for proteinogenic amino acids, and useful for treating specific amino acid deficiencies. Examples of natural, non-proteinogenic amino acids include citrulline, ornithine, and arginosuccinate, which are intermediates in the urea cycle; 1-3,4-dihydroxyphenylalanine (DOPA), which is a downstream oxidation product of tyrosine and a precursor to the neurotransmitter dopamine; and numerous hydroxylated amino acids, including 3-hydroxyproline and 5-hydroxylysine, which are typically formed post-translationally but which can be funneled into amino acid biosynthesis pathways. Non-limiting examples of natural amino acid sidechains consistent with the present disclosure include proteinogenic amino acid side chains (e.g., those outlined in TABLE 1, TABLE 2, and TABLE 3),

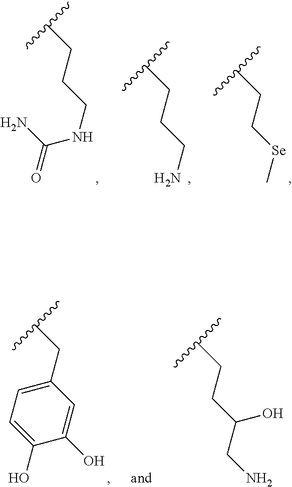

(wherein [symbol] is the point of connection to the polyol backbone).

An amino acid sidechain of the present disclosure (e.g., an amino acid sidechain included in a compound of any one of Formula (XVI)-Formula (XXII)) can be a non-proline proteinogenic amino acid sidechain. In some cases, the non-proline proteinogenic amino acid sidechain is selected from the group consisting of:

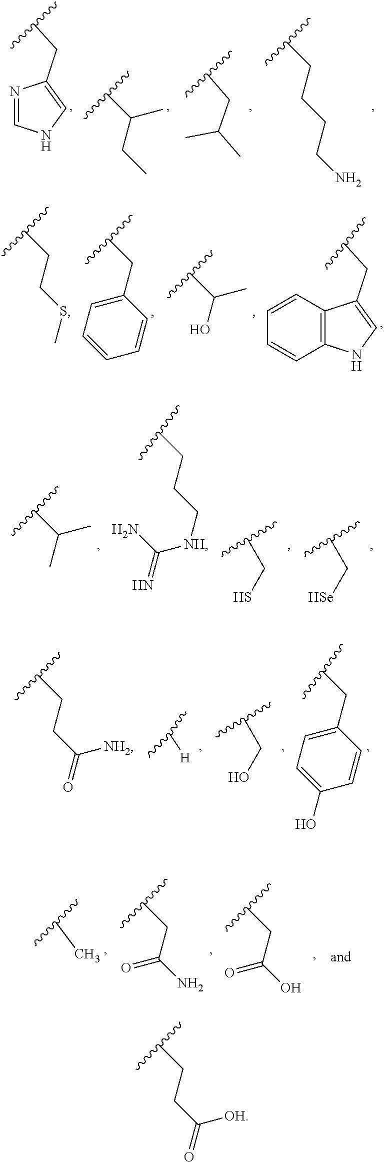

In some cases, the non-proline proteinogenic amino acid sidechain is selected from the group consisting of:

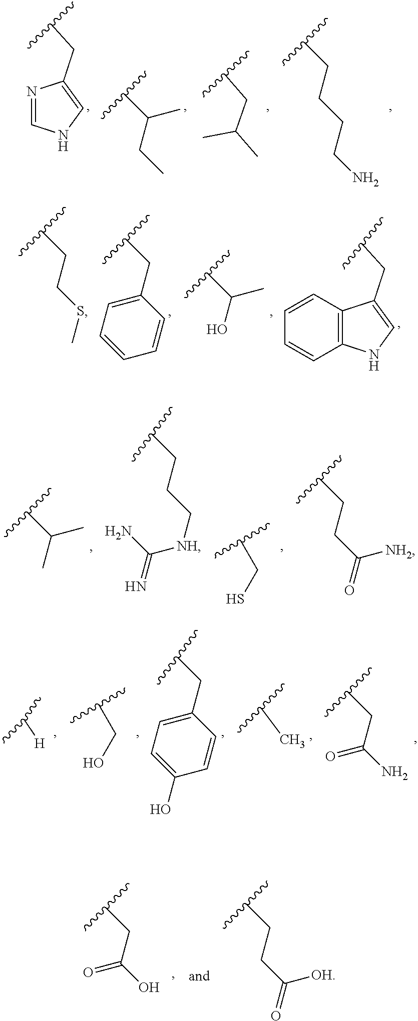

(e.g., does not include selenocysteine).

An amino acid sidechain of the present disclosure (e.g., an amino acid sidechain included in a compound of any one of Formula (XVI)-Formula (XXII)) may be an essential amino acid sidechain. For example, an essential amino acid sidechain may comprise a histidine sidechain, an isoleucine sidechain, a leucine sidechain, a lysine sidechain, a methionine sidechain, a phenylalanine sidechain, a threonine sidechain, a tryptophan sidechain, or a valine sidechain. Examples of essential amino acid sidechains that may be included in a glyceride keto ester of the present disclosure are provided in TABLE 1 (wherein [symbol] is the point of connection to the polyol backbone).

An amino acid sidechain of the present disclosure (e.g., an amino acid sidechain included in a compound of any one of Formula (XVI)-Formula (XXII)) may be a quasi-essential amino acid sidechain. As used herein, the term “quasi-essential amino acids” can refer to the set amino acids which includes arginine, cysteine, glutamine, glycine, serine, and tyrosine, and can be used interchangeably with the term “conditionally essential amino acids.” For example, a quasi-essential amino acid sidechain may comprise an arginine sidechain, a cysteine sidechain, a glutamine sidechain, a tyrosine sidechain, a glycine sidechain, or a serine sidechain. Examples of quasi-essential amino acid sidechains that may be included in a glyceride keto ester of the present disclosure are provided in TABLE 2 (wherein [symbol] is the point of connection to the polyol backbone).

An amino acid sidechain of the present disclosure (e.g., an amino acid sidechain included in a compound of any one of Formula (XVI)-Formula (XXII)) may be a non-essential amino acid sidechain. For example, a non-essential amino acid sidechain may comprise an alanine sidechain, an asparagine sidechain, an aspartate sidechain, or a glutamate sidechain. Examples of non-essential amino acid sidechains that may be included in a glyceride keto ester of the present disclosure are provided in TABLE 3 (wherein [symbol] is the point of connection to the polyol backbone).

While the amino acid sidechains provided in TABLE 1, TABLE 2, and TABLE 3 are shown in their nonionized forms, a polyol keto ester (e.g., a glyceride keto ester) of the present disclosure may include an ionized form of the amino acid (e.g., a protonated or deprotonated amino acid). For example, a polyol keto ester may comprise a deprotonated glutamic acid sidechain or aspartic acid sidechain, or a protonated arginine or lysine side chain.

In some embodiments, R¹ of Formula (XVI) may comprise a non-proline, non-glycine, non-alanine, natural amino acid sidechain. In some embodiments, R¹ of Formula (XVI) may comprise a non-proline, non-alanine, non-glycine proteinogenic amino acid sidechain. In some embodiments, R¹ of Formula (XVI) may comprise an essential amino acid sidechain (e.g., a histidine sidechain, an isoleucine sidechain, a leucine sidechain, a lysine sidechain, a methionine sidechain, a phenylalanine sidechain, a threonine sidechain, a tryptophan sidechain, or a valine sidechain). In some embodiments, R¹ of Formula (XVI) may comprise a quasi-essential amino acid sidechain (e.g., an arginine sidechain, a cysteine sidechain, a glutamine sidechain, a tyrosine sidechain, a glycine sidechain, or a serine sidechain). In some embodiments, R¹ of Formula (XVI) may comprise a non-essential amino acid sidechain (e.g., an alanine sidechain, an asparagine sidechain, an aspartate sidechain, or a glutamate sidechain).

In some embodiments, R² of Formula (XVI) may comprise a non-proline, non-alanine, non-glycine natural amino acid sidechain. In some embodiments, R² of Formula (XVI) may comprise a non-proline, non-alanine, non-glycine proteinogenic amino acid sidechain. In some embodiments, R² of Formula (XVI) may comprise an essential amino acid sidechain (e.g., a histidine sidechain, an isoleucine sidechain, a leucine sidechain, a lysine sidechain, a methionine sidechain, a phenylalanine sidechain, a threonine sidechain, a tryptophan sidechain, or a valine sidechain). In some embodiments, R² of Formula (XVI) may comprise a quasi-essential amino acid sidechain (e.g., an arginine sidechain, a cysteine sidechain, a glutamine sidechain, a tyrosine sidechain, a glycine sidechain, or a serine sidechain). In some embodiments, R² of Formula (XVI) may comprise a non-essential amino acid sidechain (e.g., an alanine sidechain, an asparagine sidechain, an aspartate sidechain, or a glutamate sidechain).

In some embodiments, R³ of Formula (XVI) may comprise a non-proline, non-alanine, non-glycine natural amino acid sidechain. In some embodiments, R³ of Formula (XVI) may comprise a non-proline, non-alanine, non-glycine proteinogenic amino acid sidechain. In some embodiments, R³ of Formula (XVI) may comprise an essential amino acid sidechain (e.g., a histidine sidechain, an isoleucine sidechain, a leucine sidechain, a lysine sidechain, a methionine sidechain, a phenylalanine sidechain, a threonine sidechain, a tryptophan sidechain, or a valine sidechain). In some embodiments, R³ of Formula (XVI) may comprise a quasi-essential amino acid sidechain (e.g., an arginine sidechain, a cysteine sidechain, a glutamine sidechain, a tyrosine sidechain, a glycine sidechain, or a serine sidechain). In some embodiments, R³ of Formula (XVI) may comprise a non-essential amino acid sidechain (e.g., an alanine sidechain, an asparagine sidechain, an aspartate sidechain, or a glutamate sidechain).

In some embodiments, R¹ of Formula (XVII) may comprise a non-proline, non-alanine, non-glycine natural amino acid sidechain. In some embodiments, R¹ of Formula (XVII) may comprise a non-proline, non-alanine, non-glycine proteinogenic amino acid sidechain. In some embodiments, R¹ of Formula (XVII) may comprise an essential amino acid sidechain (e.g., a histidine sidechain, an isoleucine sidechain, a leucine sidechain, a lysine sidechain, a methionine sidechain, a phenylalanine sidechain, a threonine sidechain, a tryptophan sidechain, or a valine sidechain). In some embodiments, R¹ of Formula (XVII) may comprise a quasi-essential amino acid sidechain (e.g., an arginine sidechain, a cysteine sidechain, a glutamine sidechain, a tyrosine sidechain, a glycine sidechain, or a serine sidechain). In some embodiments, R¹ of Formula (XVII) may comprise a non-essential amino acid sidechain (e.g., an alanine sidechain, an asparagine sidechain, an aspartate sidechain, or a glutamate sidechain).

In some embodiments, R² of Formula (XVII) may comprise a non-proline, non-alanine, non-glycine natural amino acid sidechain. In some embodiments, R² of Formula (XVII) may comprise a non-proline, non-alanine, non-glycine proteinogenic amino acid sidechain. In some embodiments, R² of Formula (XVII) may comprise an essential amino acid sidechain (e.g., a histidine sidechain, an isoleucine sidechain, a leucine sidechain, a lysine sidechain, a methionine sidechain, a phenylalanine sidechain, a threonine sidechain, a tryptophan sidechain, or a valine sidechain). In some embodiments, R² of Formula (XVII) may comprise a quasi-essential amino acid sidechain (e.g., an arginine sidechain, a cysteine sidechain, a glutamine sidechain, a tyrosine sidechain, a glycine sidechain, or a serine sidechain). In some embodiments, R² of Formula (XVII) may comprise a non-essential amino acid sidechain (e.g., an alanine sidechain, an asparagine sidechain, an aspartate sidechain, or a glutamate sidechain).

In some embodiments, R¹ of Formula (XVIII) may comprise a non-proline, non-alanine, non-glycine natural amino acid sidechain. In some embodiments, R¹ of Formula (XVIII) may comprise a non-proline, non-alanine, non-glycine proteinogenic amino acid sidechain. In some embodiments, R¹ of Formula (XVIII) may comprise an essential amino acid sidechain (e.g., a histidine sidechain, an isoleucine sidechain, a leucine sidechain, a lysine sidechain, a methionine sidechain, a phenylalanine sidechain, a threonine sidechain, a tryptophan sidechain, or a valine sidechain). In some embodiments, R¹ of Formula (XVIII) may comprise a quasi-essential amino acid sidechain (e.g., an arginine sidechain, a cysteine sidechain, a glutamine sidechain, a tyrosine sidechain, a glycine sidechain, or a serine sidechain). In some embodiments, R¹ of Formula (XVIII) may comprise a non-essential amino acid sidechain (e.g., an alanine sidechain, an asparagine sidechain, an aspartate sidechain, or a glutamate sidechain).

In some embodiments, R² of Formula (XVIII) may comprise a non-proline, non-alanine, non-glycine natural amino acid sidechain. In some embodiments, R² of Formula (XVIII) may comprise a non-proline, non-alanine, non-glycine proteinogenic amino acid sidechain. In some embodiments, R² of Formula (XVIII) may comprise an essential amino acid sidechain (e.g., a histidine sidechain, an isoleucine sidechain, a leucine sidechain, a lysine sidechain, a methionine sidechain, a phenylalanine sidechain, a threonine sidechain, a tryptophan sidechain, or a valine sidechain). In some embodiments, R² of Formula (XVIII) may comprise a quasi-essential amino acid sidechain (e.g., an arginine sidechain, a cysteine sidechain, a glutamine sidechain, a tyrosine sidechain, a glycine sidechain, or a serine sidechain). In some embodiments, R² of Formula (XVIII) may comprise a non-essential amino acid sidechain (e.g., an alanine sidechain, an asparagine sidechain, an aspartate sidechain, or a glutamate sidechain).

In some embodiments, R¹ of Formula (XIX) may comprise a non-proline, non-alanine, non-glycine natural amino acid sidechain. In some embodiments, R¹ of Formula (XIX) may comprise a non-proline, non-alanine, non-glycine proteinogenic amino acid sidechain. In some embodiments, R¹ of Formula (XIX) comprises an essential amino acid sidechain (e.g., a histidine sidechain, an isoleucine sidechain, a leucine sidechain, a lysine sidechain, a methionine sidechain, a phenylalanine sidechain, a threonine sidechain, a tryptophan sidechain, or a valine sidechain). In some embodiments, R¹ of Formula (XIX) comprises a quasi-essential amino acid sidechain (e.g., an arginine sidechain, a cysteine sidechain, a glutamine sidechain, a tyrosine sidechain, a glycine sidechain, or a serine sidechain). In some embodiments, R¹ of Formula (XIX) comprises a non-essential amino acid sidechain (e.g., an alanine sidechain, an asparagine sidechain, an aspartate sidechain, or a glutamate sidechain).

In some embodiments, R¹ of Formula (XX) comprises a non-proline, non-alanine, non-glycine natural amino acid sidechain. In some embodiments, R¹ of Formula (XX) comprises a non-proline, non-alanine, non-glycine proteinogenic amino acid sidechain. In some embodiments, R¹ of Formula (XX) comprises an essential amino acid sidechain (e.g., a histidine sidechain, an isoleucine sidechain, a leucine sidechain, a lysine sidechain, a methionine sidechain, a phenylalanine sidechain, a threonine sidechain, a tryptophan sidechain, or a valine sidechain). In some embodiments, R¹ of Formula (XX) comprises a quasi-essential amino acid sidechain (e.g., an arginine sidechain, a cysteine sidechain, a glutamine sidechain, a tyrosine sidechain, a glycine sidechain, or a serine sidechain). In some embodiments, R¹ of Formula (XX) comprises a non-essential amino acid sidechain (e.g., an alanine sidechain, an asparagine sidechain, an aspartate sidechain, or a glutamate sidechain).

### Consumable Compositions of Polyol Keto Esters

The present disclosure provides consumable compositions comprising one or more polyol keto esters (e.g., compounds of Formula (XVI)-Formula (XXII)). In some cases, the composition comprises a polyol keto ester described herein. In some cases, the consumable composition is a medical grade food. In some cases, the consumable composition is a medical grade composition. In some cases, the consumable composition is a dietary supplement.

In some cases, the consumable composition comprises of a liquid, a solid, a colloid, a gel, or a combination thereof. In some cases, the consumable composition comprises a food or a beverage. For example, the consumable composition (e.g., a medical grade food) can comprise a sports drink, a bar, a cereal, a gel, a gelatin, a gummy, a cracker, a spread, a chip, a granola, a liquid, a tablet, a powder, a suspension, or a combination thereof. In some embodiments, the food may be portioned into a single serving or a plurality of servings. The food can provide one or more amino acids in the form of a polyol keto ester (e.g., a glyceride keto ester of Formula (XVI)-Formula (XX)), and may further provide additional nutrients, such as vitamins, minerals, fats, carbohydrates, proteins, or free amino acids.

The consumable composition can comprise a compound of any one of Formula (XVI)-Formula (XXII). The consumable composition can comprise a plurality of compounds of any one of Formula (XVI)-Formula (XXII). The consumable composition can comprise a plurality of compounds of Formula (XVI)-Formula (XXII). In some embodiments, the consumable composition (e.g., a medical food) may include a mixture of compounds of Formula (XVI)-Formula (XXII) (e.g., multiple polyol keto ester compounds) comprising one or more natural amino acid sidechains except proline, or one more proteinogenic amino acid sidechains except proline, one or more essential amino acid sidechains, one or more quasi-essential amino acid sidechains except proline, one or more non-essential amino acid sidechains, or combinations thereof. In some embodiments, the consumable composition (e.g., a medical food) may include a mixture of polyol keto ester compounds comprising all essential amino acid sidechains. In some embodiments, the consumable compositions (e.g., a medical food) may include a mixture of polyol keto ester compounds comprising all essential amino acid sidechains and all quasi-essential amino acid sidechains except proline. In some embodiments, the consumable compositions (e.g., a medical food) may include a mixture of polyol keto ester compounds comprising all essential amino acid sidechains, all quasi-essential amino acid sidechains except proline, and all non-essential amino acid sidechains.

In some cases, the consumable composition (e.g., a medical grade food) comprises two or more compounds of Formula (XVI)-Formula (XXII), such as a mixture of Formula (XVIII) and Formula (XIX). In some such cases, the mixture can be stochastic, for example a combination of Formulas (XVI)-(XXII) with a statistical distribution based on the ratio of polyols and keto acids used for synthesis. In other cases, the mixture can be non-stochastic, instead reflecting admixture of multiple compounds of Formula (XVI)-Formula (XXII) in controlled ratios, or synthesis of multiple compounds with controlled ratios of backbone (e.g., polyol and sugar) and keto acid (e.g., a collection of Formula (XVI)-Formula (XXII) synthesized with a 5:5:5:4:1 mixture of

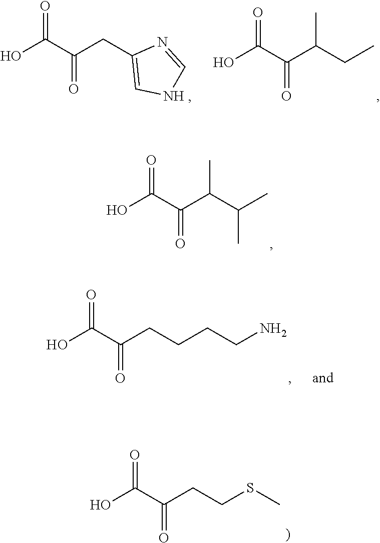

moieties. In such cases, the distributions of Formula (XVI)-Formula (XXII) can be stochastic, but the ratios of amino acid side chains and backbones can be controlled through reagent stoichiometry. In some cases, the consumable composition comprises approximately equal amounts of two compounds (e.g., from a 5:4 ratio to a 4:5 ratio of Formula (XVIII) and Formula (XIX) compounds, or from an 11:10 ratio to a 10:11 ratio of Formula (XVIII) and Formula (XIX) compounds).

In some cases, the consumable composition comprises a plurality of compounds of Formula (XVI) comprising all non-proline natural amino acid side chains (e.g., a first compound of Formula (XVI) comprising alanine side chains, a second compound of Formula (XVI) comprising histidine side chains, etc.). In some cases, the consumable composition comprises a plurality of compounds of Formula (XVII) comprising all non-proline natural amino acid side chains. In some cases, the consumable composition comprises a plurality of compounds of Formula (XVIII) comprising all non-proline natural amino acid side chains. In some cases, the consumable composition comprises a plurality of compounds of Formula (XIX) comprising all non-proline natural amino acid side chains. In some cases, the consumable composition comprises a plurality of compounds of Formula (XX) comprising all non-proline natural amino acid side chains. In some cases, the consumable composition comprises a plurality of compounds of Formula (XXI) comprising all non-proline natural amino acid side chains. In some cases, the consumable composition comprises a plurality of compounds of Formula (XXII) comprising all non-proline natural amino acid side chains.

In some cases, the consumable composition comprises a plurality of compounds of Formula (XVI) comprising all essential amino acid side chains. In some cases, the consumable composition comprises a plurality of compounds of Formula (XVII) comprising all essential amino acid side chains. In some cases, the consumable composition comprises a plurality of compounds of Formula (XVIII) comprising all essential amino acid side chains. In some cases, the consumable composition comprises a plurality of compounds of Formula (XIX) comprising all essential amino acid side chains. In some cases, the consumable composition comprises a plurality of compounds of Formula (XX) comprising all essential amino acid side chains. In some cases, the consumable composition comprises a plurality of compounds of Formula (XXI) comprising all essential amino acid side chains. In some cases, the consumable composition comprises a plurality of compounds of Formula (XXII) comprising all essential amino acid side chains.

In some cases, the consumable composition comprises a plurality of compounds of Formula (XVI) comprising at least 3 essential amino acid side chains. In some cases, the consumable composition comprises a plurality of compounds of Formula (XVII) comprising at least 3 essential amino acid side chains. In some cases, the consumable composition comprises a plurality of compounds of Formula (XVIII) comprising at least 3 essential amino acid side chains. In some cases, the consumable composition comprises a plurality of compounds of Formula (XIX) comprising at least 3 essential amino acid side chains. In some cases, the consumable composition comprises a plurality of compounds of Formula (XX) comprising at least 3 essential amino acid side chains. In some cases, the consumable composition comprises a plurality of compounds of Formula (XXI) comprising at least 3 essential amino acid side chains. In some cases, the consumable composition comprises a plurality of compounds of Formula (XXII) comprising at least 3 essential amino acid side chains.

### Standards for Microbial and Elemental Contamination

The compositions described herein (e.g., a consumable composition comprising a chitosan and one or more alpha-keto acid analogues of amino acids or a consumable composition comprising one or more polyol keto esters) may be substantially free of microbial contamination. Microbial load of a composition of the present disclosure may be quantified by any method known in the art. For example, microbial load may be quantified by total aerobic plate count (TAC). Total aerobic plate count may be used an estimation of the total viable aerobic bacteria present in a sample of raw material, in-process material, or finished product. A composition, or a precursor or intermediate of the composition, may be analyzed in accordance with U.S. Pharmacopeial Convention (USP) Guidelines (e.g., USP Guidelines, Chapter <61>, Microbial Limits Test).

In some embodiments, upper limits of total aerobic plate colonies for a composition of the present disclosure (e.g., a consumable composition comprising a chitosan and one or more alpha-keto acid analogues of amino acids or a consumable composition comprising one or more polyol keto esters) may correspond to an alert level of 1000 colony forming units (cfu) per mL, an action level of 10,000 cfu per mL, or an upper limit of 20,000 cfu per mL. A composition of the present disclosure may comprise total aerobic plate colonies of no more than 1000 cfu per mL, no more than 10,000 cfu per mL, or no more than 20,000 cfu per mL.

In some embodiments, if the water activity of a composition (e.g., a solid medical food) is below 0.85, quantification of total aerobic plate count may be avoided. In some embodiments, if the water activity of a composition (e.g., a solid medical food) is below 0.75, microbiological testing of the composition may be avoided.

Microbial standards and testing procedures are further described in the publication, “Microbial Bioburden on Oral Solid dosage Forms,” by Jose E. Martinez, Pharmaceutical Technology, February 2002, pages 58 to 70, which is herein incorporated herein by reference.

In some embodiments, a composition of the present disclosure (e.g., a medical food) may have an elemental contamination below a threshold level. A consumable composition (e.g., a medical food) of the present disclosure may have a Cd content of less than 5 μg per daily serving. A consumable composition (e.g., a medical food) of the present disclosure may have a Pb content of less than 5 μg per daily serving. A consumable composition of the present disclosure may have an As content of less than 15 μg per daily serving. A consumable composition of the present disclosure may have a Hg content of less than 30 μg per daily serving. A consumable composition of the present disclosure may have a Co content of less than 50 μg per daily serving. A consumable composition of the present disclosure may have a V content of less than 100 μg per daily serving. A consumable composition of the present disclosure may have a Ni content of less than 200 μg per daily serving. A consumable composition of the present disclosure may have a Tl content of less than 8 μg per daily serving. A consumable composition of the present disclosure may have an Au content of less than 300 μg per daily serving. A consumable composition of the present disclosure may have a Pd content of less than 100 μg per daily serving. A consumable composition of the present disclosure may have an Ir content of less than 100 μg per daily serving. A consumable composition of the present disclosure may have an Os content of less than 100 μg per daily serving. A consumable composition of the present disclosure may have a Rh content of less than 100 μg per daily serving. A consumable composition of the present disclosure may have a Ru content of less than 100 μg per daily serving. A consumable composition of the present disclosure may have a Se content of less than 150 μg per daily serving. A consumable composition of the present disclosure may have an Ag content of less than 150 μg per daily serving. A consumable composition of the present disclosure may have a Pt content of less than 100 μg per daily serving. A consumable composition of the present disclosure may have a Li content of less than 550 μg per daily serving. A consumable composition of the present disclosure may have a Sb content of less than 1200 μg per daily serving. A consumable composition of the present disclosure may have a Ba content of less than 1400 μg per daily serving. A consumable composition of the present disclosure may have a Mo content of less than 3000 μg per daily serving. A consumable composition of the present disclosure may have a Cu content of less than 3000 μg per daily serving. A consumable composition of the present disclosure may have a Sn content of less than 6000 μg per daily serving. A consumable composition of the present disclosure may have a Cr content of less than 11000 μg per daily serving.

A consumable composition (e.g., a medical food) of the present disclosure may have a Cd content of less than 0.5 μg per g. A consumable composition of the present disclosure may have a Pb content of less than 0.5 μg per g. A consumable composition of the present disclosure may have an As content of less than 1.5 μg per g. A consumable composition of the present disclosure may have a Hg content of less than 3 μg per g. A consumable composition of the present disclosure may have a Co content of less than 5 μg per g. A consumable composition of the present disclosure may have a V content of less than 10 μg per g. A consumable composition of the present disclosure may have a Ni content of less than 20 μg per g. A consumable composition of the present disclosure may have a Tl content of less than 0.8 μg per g. A consumable composition of the present disclosure may have an Au content of less than 30 μg per g. A consumable composition of the present disclosure may have a Pd content of less than 10 μg per g. A consumable composition of the present disclosure may have an Ir content of less than 10 μg per g. A consumable composition of the present disclosure may have an Os content of less than 10 μg per g. A consumable composition of the present disclosure may have a Rh content of less than 10 μg per g. A consumable composition of the present disclosure may have a Ru content of less than 10 μg per g. A consumable composition of the present disclosure may have a Se content of less than 15 μg per g. A consumable composition of the present disclosure may have an Ag content of less than 15 μg per g. A consumable composition of the present disclosure may have a Pt content of less than 10 μg per g. A consumable composition of the present disclosure may have a Li content of less than 55 μg per g. A consumable composition of the present disclosure may have a Sb content of less than 120 μg per g. A consumable composition of the present disclosure may have a Ba content of less than 140 μg per g. A consumable composition of the present disclosure may have a Mo content of less than 300 μg per g. A consumable composition of the present disclosure may have a Cu content of less than 300 μg per g. A consumable composition of the present disclosure may have a Sn content of less than 600 μg per g. A consumable composition of the present disclosure may have a Cr content of less than 1100 μg per g.

A consumable composition (e.g., a medical food) of the present disclosure may have a Cd content of less than 0.5 μg per g. A consumable composition of the present disclosure may have a Pb content of less than 10 μg per g. A consumable composition of the present disclosure may have an As content of less than 3 μg per g. A consumable composition of the present disclosure may have a Hg content of less than 1 μg per g. A consumable composition of the present disclosure may have a Co content of less than 200 μg per g. A consumable composition of the present disclosure may have a total V content of less than 0.2, less than 0.6, less than 0.9, or less than 1.8 mg per serving. A consumable composition of the present disclosure may have a Ni content of less than 200 μg per g. A consumable composition of the present disclosure may have an Au content of less than 6, less than 15, less than 30, or less than 60 μg per serving. A consumable composition of the present disclosure may have a Pd content of less than 0.2, less than 0.5, less than 1, or less than 2 μg per serving. A consumable composition of the present disclosure may have a Se content of less than 50, less than 100, less than 200, or less than 400 μg per serving. A consumable composition of the present disclosure may have an Ag content of less than 17 ng per g. A consumable composition of the present disclosure may have a Pt content of less than 100 μg per g. A consumable composition of the present disclosure may have a Ba content of less than 0.35, less than 0.7, less than 1.5, or less than 3.5 mg per serving. A consumable composition of the present disclosure may have an Mo content of less than 200, less than 500, less than 1000, or less than 2000 mg per serving. A consumable composition of the present disclosure may have a Cu content of less than 1, less than 2, less than 5, or less than 10 g per serving. A consumable composition of the present disclosure may have a Sn content of less than 5, less than 10, less than 25, less than 50, or less than 100 mg per serving. A consumable composition of the present disclosure can have a Cr content of less than 50, less than 100, less than 200, less than 500, or less than 1000 mg per serving.

### Methods of Use

The present disclosure provides methods for administering the disclosed compositions. The disclosed compositions may be administered to a subject as a protein source since the compositions disclosed herein comprise alpha-keto acids that may be used in amino acid and protein synthesis. The compositions disclosed herein may result in less hepatic stress or metabolic burden on the kidneys compared to other protein sources (e.g., amino acid compositions) or supplements (e.g., alpha-keto acid salt compositions). For example, administering a composition of the present disclosure (e.g., comprising alpha-keto acids) may result in less hepatic stress or metabolic burden on the kidneys than administering a comparable amount of an amino acid composition or an alpha-keto acid salt composition. A comparable amount of an amino acid composition may be an amount with an equal molar amount of amino acid side chains as compared to the molar amount of amino acid side chains in a composition of the present disclosure (e.g., comprising alpha-keto acids). A comparable amount of an alpha-keto acid salt composition may be an amount with an equal molar amount of alpha-keto acids as compared to the molar amount of alpha-keto acids in a composition of the present disclosure (e.g., comprising alpha-keto acids). Hepatic stress or metabolic burden on the kidneys refers to any condition resulting in stress or burden of the kidneys (e.g., stress or burden caused by excess salt or nitrogen). Hepatic stress or metabolic burden of the kidneys may result from a subject's dietary intake of excess salt content or nitrogen content. Hepatic stress (e.g., metabolic burden on the kidneys) may be increased by protein (e.g., amino acid) intake due to the nitrogen content from the amino group of amino acids. Hepatic stress (e.g., metabolic burden on the kidneys) may be increased by alpha keto acid salt administration due to the salt cation (e.g., Na²⁺, Ca²⁺, or Mg²⁺) content. Accordingly, there is a need for protein administration with decreased hepatic stress (e.g., metabolic burden on the kidneys). The administration of the compositions described herein (e.g., biopolymer keto acid compositions) for protein administration may result in a decrease in hepatic stress (e.g., metabolic burden on the kidneys) as compared to other routes of protein administration (e.g., amino acid administration or alpha keto acid salt administration). The decrease in hepatic stress (e.g., metabolic burden on the kidneys) from the administration of the compositions herein (e.g., biopolymer keto acid compositions) as compared to other routes of protein administration (e.g., amino acid administration or alpha keto acid salt administration) may be measured by comparing markers of kidney function. Markers of kidney function may be measured by the blood urea nitrogen (BUN) level, the urine-creatinine ratio (uACR), the estimated glomerular filtration rate (eGFR), or a combination thereof. The blood urea nitrogen (BUN) level of a subject may be measured by a serum BUN test, for example, as provided in the Center for Disease Control Laboratory Procedure Manual Beckman Synchron LX20 Method for Blood Urea Nitrogen (BUN) measurement in refrigerated serum. The creatinine level of a subject may be measured by a serum test, for example, as provided in the Center for Disease Control Laboratory Procedure Manual Roche Cobas 6000 (c501 module) for creatinine measurement in serum.

In some embodiments, a subject has a higher than normal blood urea nitrogen level as measured by a serum blood urea nitrogen (BUN) test, measured as provided in the Center for Disease Control Laboratory Procedure Manual Beckman Synchron LX20 Method for Blood Urea Nitrogen (BUN) measurement in refrigerated serum, prior to administration of the composition. In some embodiments, the decrease of nitrogen by-products in a subject is measured by a serum blood urea nitrogen (BUN) test, measured as provided in the Center for Disease Control Laboratory Procedure Manual Beckman Synchron LX20 Method for Blood Urea Nitrogen (BUN) measurement in refrigerated serum, as compared to administering a comparable amount of an amino acid composition to the subject.

In some embodiments, a composition may be administered orally, topically, transdermally, rectally, intravenously, intra-arterially, intra-ovarianly, vaginally, parenterally, or via inhalation. For example, a composition of the present disclosure may be ingested orally. The composition may provide a dietary protein source for subjects with or at risk of kidney disease. A consumable composition (e.g., a medical food) of the present disclosure may be consumed as a dietary supplement or a supplement for anabolic enhancement. A consumable composition (e.g., a medical food) of the present disclosure may be administered under the guidance of a medical professional as a dietary supplement. In various aspects the consumable composition disclosed herein may be provided to subjects with low blood protein levels to increase their blood protein. Since many of the disclosed compositions do not contain the amino acid amino group, their administration can lead to lower blood ammonia buildup than comparable compositions with proteins or free amino acids. Since the compositions as described herein comprise alpha-keto acid analogues of amino acids, the compositions comprise protein and are considered to be a protein supplement for a subject. The compositions as described herein may be more beneficial as protein supplements as compared to other protein supplements since they comprise alpha-keto acid analogues of amino acids which do not contain the amino group of the amino acid. In some embodiments, the compositions as described herein result in less nitrogen by-products per the amount of protein provided in a subject as compared to the nitrogen by-products per the amount of protein provided in other protein supplement compositions (e.g., compositions comprising amino acids). In some embodiments, administering the composition to the subject results in a decrease of nitrogen by-products in the subject as compared to administering protein comprising amino acids to the subject. In some embodiments, the nitrogen by-products comprise urea, uric acid, creatinine, ammonia, or a combination thereof. In some embodiments, the decrease of nitrogen by-products is measured by a blood urea nitrogen test or a creatinine test.

Alpha-keto acids may also be administered to or taken by a subject to increase protein levels. Alpha-keto acids may be provided as the salt form and can induce unwanted side effects from the salt. Also, alpha-keto acids provided as a salt may cause an unwanted increase in a salt-level of a subject. The compositions as described herein may be more beneficial as protein supplements as compared to other alpha-keto acid salt supplements since they are ionically bound to chitosan instead of a salt.

The administration of the compositions described herein (e.g., biopolymer keto acid compositions) for protein administration may result in a decrease in salt (e.g., Na²⁺, Ca²⁺, or Mg²⁺) intake per kg of protein administered as compared to other routes of protein administration (e.g., amino acid administration or alpha keto acid salt administration). In some embodiments, the compositions as described herein result in a decreased salt level per the amount of protein provided in a subject as compared to the salt level per the amount of protein provided in other protein supplement compositions (e.g., compositions comprising alpha-keto acid salts). In some embodiments, administering the composition to the subject results in a decrease of a level of a salt in the subject as compared to administering protein comprising alpha-keto acid salts to the subject. In some embodiments, the decrease of a level of a salt is measured by a urine test measuring the level of the salt. In some embodiments, the salt comprises sodium, potassium, or chloride. The salt level of a subject may be measured by a urine test measuring sodium, potassium, and chloride, for example, by the protocol provided in the Center of Disease Control Laboratory Procedure Manual, Roche Ion-Selective Electrode Method for measuring sodium, potassium, and chloride in urine (method number 4047.03).

In some embodiments, administering the compositions as described herein to a subject results in a decrease of a level of a salt in the subject as measured by a salt urine test by the protocol provided in the Center of Disease Control Laboratory Procedure Manual, Roche Ion-Selective Electrode Method for measuring sodium, potassium, and chloride in urine (method number 4047.03), as compared to administering a comparable amount of an alpha-keto acid salt composition to the subject.

A consumable composition (e.g., a medical food) of the present disclosure may be provided as a dietary supplement for a patient with an anabolic need. A subject (e.g., a patient) may be in an anabolic need due to a condition or due to a medication being administered. In some embodiments, an anabolic need may be diagnosed by a low protein level in a subject. In some embodiments, an anabolic need is a need of protein and may require protein supplementation.

In some embodiments, the subject may have a higher-than-normal blood urea nitrogen level or creatinine level. In some embodiments, the higher-than-normal blood urea nitrogen level in caused by dehydration, burns, medicines, a high protein diet, or age. The consumable compositions of the present disclosure may be provided to a patient with a condition that is treated with anabolic steroids. For example, patients with severe burn injuries may be treated with anabolic steroids to improve muscle protein metabolism and may be administered consumable compositions of the present disclosure to increase their protein intake. Other conditions that may be treated with anabolic steroids and supplemented with consumable compositions described herein include primary male hypogonadism, hypogonadotropic hypogonadism, delayed puberty, gonadotropin and luteinizing hormone deficiency, breast cancer, endometriosis, or osteoporosis. Anabolic steroids may also be administered to patients for bone marrow stimulation in patients with leukemia, aplastic anemia, kidney failure, growth failure, stimulation of appetite, or stimulation of muscle mass in patients. The consumable compositions of the present disclosure may be provided in conjunction with an anabolic steroid to provide adequate protein for building muscle mass. The compositions of the present disclosure may be provided in conjunction with danazol, testosterone, trenbolone, oxymetholone, fluoxymesterone, methyltestosterone, methandrostenolone, nandrolone, stanozolol, boldenone, and oxandrolone. The composition may be used to treat or prevent a kidney disorder. For example, the composition may provide a dietary protein source which does not increase the concentration of ammonia, urea, or inorganic cations in the blood following administration, and which may therefore be amenable for administration to kidney disease patients. In some embodiments, the composition is administered to a subject having or at risk of having chronic kidney disease, Fabry disease, cystinosis, glomerulonephritis, IgA nephropathy, lupus nephritis, atypical hemolytic uremic syndrome, polycystic kidney disease, diabetic kidney disease, or uremia. In some embodiments, the composition is administered to a subject that is at risk of developing kidney disease. In some cases, the subject has a kidney disease risk factor selected from diabetes, high blood pressure, heart disease, obesity, a family history of chronic kidney disease, inherited kidney disorders, past incidences of kidney damage, and old age. In some embodiments, the composition is administered to a subject in need of a low ammonia and high protein diet. In some embodiments, the composition is administered to a subject irrespective of health status or dietary restrictions.

The composition may be used to treat a low protein level in a subject. The compositions herein comprise alpha-keto acids that may be used for the synthesis of amino acids and protein in the body thereby supplementing and providing protein in a diet of a subject. The low protein level in a subject may be diagnosed by a blood protein test. The subject's protein level may increase after administration of any of the compositions as described herein. The compositions as described herein may be used to increase a protein level in a subject. Compositions as described herein may be used to increase a protein level in a subject. A protein level in a subject may be measured by a blood serum albumin test, for example, as provided in the Center for Disease Control Laboratory Procedure Manual Roche Cobas 6000 (c501) Protocol for measuring albumin in serum.

In some embodiments, the present disclosure provides a method of administering protein to a subject, the method comprising: administering a composition to the subject comprising: a polysaccharide, and an alpha-keto acid composition, wherein administering the composition to the subject increases a protein level in the subject as measured by a serum albumin test, as provided in the Center for Disease Control Laboratory Procedure Manual Roche Cobas 6000 (c501) Protocol for measuring albumin in serum.

In some embodiments, the present disclosure provides a method of treating a subject in need thereof, the method comprising: administering a composition to the subject comprising: a polysaccharide, and an alpha-keto acid composition, and increasing a protein level in the subject as measured by a serum albumin test, thereby treating the subject, as provided in the Center for Disease Control Laboratory Procedure Manual Roche Cobas 6000 (c501) Protocol for measuring albumin in serum.

The composition may be used to treat a condition wherein a symptom of the condition is impacted kidney or renal function. Conditions that may impact kidney or renal function include blood or fluid loss, use of blood pressure medications, heart attacks, heart disease, infections, use of aspirin, ibuprofen, naproxen sodium, acetaminophen, or related drugs, liver failure, allergic reactions, severe burns, or severe hydration. Diseases, conditions, and agents that may damage the kidneys and lead to impaired kidney or renal function include: blood clots in and around the kidneys, cholesterol deposits that block blood flow in the kidneys, glomerulonephritis, hemolytic uremic syndrome, infection, COVID-19, lupus, medications, chemotherapy drugs, antibiotics, dyes used during imaging tests, scleroderma, thrombotic thrombocytopenia purpura, toxins, alcohol, heavy metals, stimulants, muscle tissue breakdown, or breakdown of tumor cells. Disease, conditions, and agents that may block the passage of urine and lead to impaired kidney or renal function include bladder cancer, blood clots in the urinary tract, cervical cancer, colon cancer, enlarged prostate, kidney stones, nerve damage, or prostate cancer. Conditions and diseases that increase a risk for kidney disease and lead to impaired kidney or renal function include hospitalization, advanced age, blockages in the blood vessels in the arms or legs, diabetes, high blood pressure, heart failure, kidney diseases, liver diseases, or cancers and their treatments.

Administration of the compositions described herein may delay a subject's need for dialysis. Administration of the compositions described herein may decrease the frequency of a subject's need for dialysis.

As used herein, the singular forms “a,” “an” and “the” include plural referents unless the context clearly dictates otherwise.

As used herein, the terms “about” and “approximately,” in reference to a number, is used herein to include numbers that fall within a range of 10%, 5%, or 1% in either direction (greater than or less than) the number unless otherwise stated or otherwise evident from the context (except where such number would exceed 100% of a possible value).

A composition can be administered alone or in combination with other treatments, either simultaneously or sequentially dependent upon the condition to be treated.

As used herein, the term “subject” broadly refers to any animal, including but not limited to, human and non-human animals (e.g., dogs, cats, cows, horses, sheep, pigs, poultry, fish, crustaceans, etc.).

As used herein, the term “effective amount” refers to the amount of a composition sufficient to effect beneficial or desired results. An effective amount can be administered in one or more administrations, applications or dosages and is not intended to be limited to a particular formulation or administration route.

As used herein, the term “therapeutically effective amount” is an amount that is effective to ameliorate a symptom of a disease. A therapeutically effective amount can be a “prophylactically effective amount” as prophylaxis can be considered therapy.

As used herein, the terms “administration” and “administering” refer to the act of giving a drug, prodrug, or other agent, or therapeutic treatment to a subject or in vivo, in vitro, or ex vivo cells, tissues, and organs. Exemplary routes of administration to the human body can be through space under the arachnoid membrane of the brain or spinal cord (intrathecal), the eyes (ophthalmic), mouth (oral), skin (topical or transdermal), nose (nasal), lungs (inhalant), oral mucosa (buccal or lingual), ear, rectal, vaginal, by injection (e.g., intravenously, subcutaneously, intratumorally, intra-peritoneally, etc.) and the like.

As used herein, the term “treatment” means an approach to obtaining a beneficial or intended clinical result. The beneficial or intended clinical result can include alleviation of symptoms, a reduction in the severity of the disease, inhibiting an underlying cause of a disease or condition, steadying diseases in a non-advanced state, delaying the progress of a disease, and/or improvement or alleviation of disease conditions.

As used herein, the term “pharmaceutical composition” refers to the combination of an active ingredient with a carrier, inert or active, making the composition especially suitable for therapeutic or diagnostic use in vitro, in vivo or ex vivo.

The terms “pharmaceutically acceptable” or “pharmacologically acceptable,” as used herein, refer to compositions that do not substantially produce adverse reactions, e.g., toxic, allergic, or immunological reactions, when administered to a subject.

As used herein, the term “pharmaceutically acceptable carrier” refers to any of the standard pharmaceutical carriers including, but not limited to, phosphate buffered saline solution, water, emulsions (e.g., such as an oil/water or water/oil emulsions), glycerol, liquid polyethylene glycols, aprotic solvents such as dimethylsulfoxide, N-methylpyrrolidone and mixtures thereof, and various types of wetting agents, solubilizing agents, anti-oxidants, bulking agents, protein carriers such as albumins, any and all solvents, dispersion media, coatings, sodium lauryl sulfate, isotonic and absorption delaying agents, disintegrants (e.g., potato starch or sodium starch glycolate), and the like. The compositions also can include stabilizers and preservatives. For examples of carriers, stabilizers, and adjuvants, see, e.g., Martin, Remington's Pharmaceutical Sciences, 21^{st} Ed., Mack Publ. Co., Easton, Pa. (2005), incorporated herein by reference in its entirety.

As used herein, the term “medical food” refers to a food which is formulated to be consumed or administered enterally under the supervision of a physician and which is intended for the specific dietary management of a disease or condition for which distinctive nutritional requirements, based on recognized scientific principles, are established by medical evaluation.

As used herein, the term “dietary supplement” refers to a product intended for oral consumption that contains a compound or mixture of compounds intended to supplement a diet (e.g., a complete diet or a diet deficient in one or more nutrients), and can include vitamins, minerals, extracts (e.g., herbal extracts), concentrates (e.g., fruit, vegetable, or bone marrow concentrates). Dietary supplements can also be extracts or concentrates, and may be formulated as a liquid, a solid, a powder, a gel, a colloid, a suspension, or a combination thereof.

All terms, chemical names, expressions, and designations have their usual meanings which are well-known to those skilled in the art. When a group of substituents is disclosed herein, it is understood that all individual members of that group and all subgroups, including any isomers, enantiomers, and diastereomers of the group members, are disclosed separately. When a Markush group or other grouping is used herein, all individual members of the group and all combinations and subcombinations possible of the group are intended to be individually included in the disclosure. When a compound is described herein such that a particular isomer, enantiomer or diastereomer of the compound is not specified, for example, in a formula or in a chemical name, that description is intended to include each isomers and enantiomer of the compound described individually or in any combination. Additionally, unless otherwise specified, all isotopic variants of compounds disclosed herein are intended to be encompassed by the disclosure. Specific names of compounds are intended to be exemplary, as it is known that one of ordinary skill in the art can name the same compounds differently.

As used herein, the term “group” may refer to a reactive functional group of a chemical compound. Groups of the present compounds refer to an atom or a collection of atoms that are a part of the compound. Groups of the present disclosure may be attached to other atoms of the compound via one or more covalent bonds. Groups may also be characterized with respect to their valence state. The present disclosure includes groups characterized as monovalent, divalent, trivalent, etc. valence states.

As used herein, the term “substituted” refers to a compound (e.g., an alkyl chain) wherein a hydrogen is replaced by another reactive functional group or atom, as described herein.

As used herein, a broken line in a chemical structure can be used to indicate a bond to the rest of the molecule. For example, [symbol] in

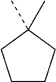

is used to designate the 1-position as the point of attachment of 1-methylcyclopentate to the rest of the molecule. Alternatively,

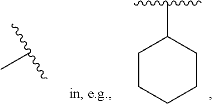

can be used to indicate that the given moiety, the cyclohexyl moiety in this example, is attached to a molecule via the bond that is “capped” with the wavy line.

The following definitions are used, unless otherwise described: halo is fluoro, chloro, bromo, or iodo. Alkyl, alkoxy, alkenyl, alkynyl, etc. denote both straight and branched groups; but reference to an individual radical such as propyl embraces only the straight chain radical, a branched chain isomer such as isopropyl being specifically referred to. Aryl denotes a phenyl radical or an ortho-fused bicyclic carbocyclic radical having about nine to ten ring atoms in which at least one ring is aromatic. Heteroaryl encompasses a radical of a monocyclic aromatic ring containing five or six ring atoms consisting of carbon and one to four heteroatoms each selected from the group consisting of non-peroxide oxygen, sulfur, and N(X) wherein X is absent or is H, O, (C₁-C₄)alkyl, phenyl or benzyl, as well as a radical of an ortho-fused bicyclic heterocycle of about eight to ten ring atoms comprising one to four heteroatoms each selected from the group consisting of non-peroxide oxygen, sulfur, and N(X).

It will be appreciated by those skilled in the art that compounds of the disclosure having a chiral center may exist in and be isolated in optically active and racemic forms. Some compounds may exhibit polymorphism. It is to be understood that the present disclosure encompasses any racemic, optically-active, polymorphic, or stereoisomeric form, or mixtures thereof, of a compound of the disclosure, which possess the useful properties described herein, it being well known in the art how to prepare optically active forms (for example, by resolution of the racemic form by recrystallization techniques, by synthesis from optically-active starting materials, by chiral synthesis, or by chromatographic separation using a chiral stationary phase.

Specific values listed below for radicals, substituents, and ranges, are for illustration only; they do not exclude other defined values or other values within defined ranges for the radicals and substituents.

Specifically, (C₁-C₆)alkyl can be methyl, ethyl, propyl, isopropyl, butyl, iso-butyl, sec-butyl, pentyl, 3-pentyl, or hexyl; (C₃-C₆) cycloalkyl can be cyclopropyl, cyclobutyl, cyclopentyl, or cyclohexyl; (C₃-C₆) cycloalkyl(C₁-C₆)alkyl can be cyclopropylmethyl, cyclobutylmethyl, cyclopentylmethyl, cyclohexylmethyl, 2-cyclopropylethyl, 2-cyclobutylethyl, 2-cyclopentylethyl, or 2-cyclohexylethyl; (C₁-C₆)alkoxy can be methoxy, ethoxy, propoxy, isopropoxy, butoxy, iso-butoxy, sec-butoxy, pentoxy, 3-pentoxy, or hexyloxy; (C₂-C₆)alkenyl can be vinyl, allyl, 1-propenyl, 2-propenyl, 1-butenyl, 2-butenyl, 3-butenyl, 1,-pentenyl, 2-pentenyl, 3-pentenyl, 4-pentenyl, 1-hexenyl, 2-hexenyl, 3-hexenyl, 4-hexenyl, or 5-hexenyl; (C₂-C₆)alkynyl can be ethynyl, 1-propynyl, 2-propynyl, 1-butynyl, 2-butynyl, 3-butynyl, 1-pentynyl, 2-pentynyl, 3-pentynyl, 4-pentynyl, 1-hexynyl, 2-hexynyl, 3-hexynyl, 4-hexynyl, or 5-hexynyl; (C₁-C₆)alkanoyl can be acetyl, propanoyl or butanoyl; (C₁-C₆)alkoxycarbonyl can be methoxycarbonyl, ethoxycarbonyl, propoxycarbonyl, isopropoxycarbonyl, butoxycarbonyl, pentoxycarbonyl, or hexyloxycarbonyl; (C₂-C₆)alkanoyloxy can be acetoxy, propanoyloxy, butanoyloxy, isobutanoyloxy, pentanoyloxy, or hexanoyloxy; aryl can be phenyl, indenyl, or naphthyl; and heteroaryl can be furyl, imidazolyl, triazolyl, triazinyl, oxazoyl, isoxazoyl, thiazolyl, isothiazoyl, pyrazolyl, pyrrolyl, pyrazinyl, tetrazolyl, pyridyl, (or its N-oxide), thienyl, pyrimidinyl (or its N-oxide), indolyl, isoquinolyl (or its N-oxide) or quinolyl (or its N-oxide).

The term “alkyl” refers to an unsubstituted straight chain or branched, saturated hydrocarbon having the indicated number of carbon atoms (e.g., “C₁-C₄ alkyl,” “C₁-C₆ alkyl,” “C₁-C₈ alkyl,” or “C₁-C₁₀” alkyl have from 1 to 4, to 6, 1 to 8, or 1 to 10 carbon atoms, respectively) and is derived by the removal of one hydrogen atom from the parent alkane. Representative straight chain “C₁-C₈ alkyl” groups include, but are not limited to, methyl, ethyl, n-propyl, n-butyl, n-pentyl, n-hexyl, n-heptyl and n-octyl; while branched C₁-C₈ alkyls include, but are not limited to, isopropyl, sec-butyl, isobutyl, tert-butyl, isopentyl, and 2-methylbutyl.

The term “alkylene” refers to a bivalent unsubstituted saturated branched or straight chain hydrocarbon of the stated number of carbon atoms (e.g., a C₁-C₆ alkylene has from 1 to 6 carbon atoms) and having two monovalent centers derived by the removal of two hydrogen atoms from the same or two different carbon atoms of the parent alkane. Alkylene groups can be substituted with 1-6 fluoro groups, for example, on the carbon backbone (as —CHF— or —CF₂—) or on terminal carbons of straight chain or branched alkylenes (such as —CHF₂ or —CF₃). Alkylene groups include but are not limited to: methylene (—CH₂—), ethylene (—CH₂CH₂—), n-propylene (—CH₂CH₂CH₂—), n-propylene (—CH₂CH₂CH₂—), n-butylene (—CH₂CH₂CH₂CH₂—), difluoro-methylene (—CF₂—), tetrafluoroethylene (—CF₂CF₂—), and the like.

The term “alkenyl” refers to an unsubstituted straight chain or branched, hydrocarbon having at least one carbon-carbon double bond and the indicated number of carbon atoms (e.g., “C₂-C₈ alkenyl” or “C₂-C₁₀” alkenyl have from 2 to 8 or 2 to 10 carbon atoms, respectively). When the number of carbon atoms is not indicated, the alkenyl group has from 2 to 6 carbon atoms.

The term “heteroalkyl” refers to a stable straight or branched chain saturated hydrocarbon having the stated number of total atoms and at least one (e.g., 1 to 15) heteroatom selected from the group consisting of O, N, Si and S. The carbon and heteroatoms of the heteroalkyl group can be oxidized (e.g., to form ketones, N-oxides, sulfones, and the like) and the nitrogen atoms can be quaternized. The heteroatom(s) can be placed at any interior position of the heteroalkyl group and/or at any terminus of the heteroalkyl group, including termini of branched heteroalkyl groups), and/or at the position at which the heteroalkyl group is attached to the remainder of the molecule. Heteroalkyl groups can be substituted with 1-6 fluoro groups, for example, on the carbon backbone (as —CHF— or —CF₂—) or on terminal carbons of straight chain or branched heteroalkyls (such as —CHF₂ or —CF₃). Examples of heteroalkyl groups include, but are not limited to, —CH₂—CH₂—O—CH₃, —CH₂—CH₂—NH—CH₃, —CH₂—CH₂—N(CH₃)₂, —C(═O)—NH—CH₂—CH₂—NH—CH₃, —C(═O)—N(CH₃)—CH₂—CH₂—N(CH₃)₂, —C(═O)—NH—CH₂—CH₂—NH—C(═O)—CH₂—CH₃, —C(═O)—N(CH₃)—CH₂—CH₂—N(CH₃)—C(═O)—CH₂—CH₃, —O—CH₂—CH₂—CH₂—NH(CH₃), —O—CH₂—CH₂—CH₂—N(CH₃)₂, —O—CH₂—CH₂—CH₂—NH—C(═O)—CH₂—CH₃, —O—CH₂—CH₂—CH₂—N(CH₃)—C(═O)—CH₂—CH₃, —CH₂—CH₂—CH₂—NH(CH₃), —O—CH₂—CH₂—CH₂—N(CH₃)₂, —CH₂—CH₂—CH₂—NH—C(═O)—CH₂—CH₃, —CH₂—CH₂—CH₂—N(CH₃)—C(═O)—CH₂—CH₃, —CH₂—S—CH₂—CH₃, —CH₂—CH₂—S(O)—CH₃, —NH—CH₂—CH₂—NH—C(═O)—CH₂—CH₃, —CH₂—CH₂—S(O)₂—CH₃, —CH₂—CH₂—O—CF₃, and —Si(CH₃)₃. Up to two heteroatoms may be consecutive, such as, for example, —CH₂—NH—OCH₃ and —CH₂—O—Si(CH₃)₃. A terminal polyethylene glycol (PEG) moiety is a type of heteroalkyl group.

The term “alkynyl” refers to an unsubstituted straight chain or branched, hydrocarbon having at least one carbon-carbon triple bond and the indicated number of carbon atoms (e.g., “C₂-C₈ alkynyl” or “C₂-C₁₀” alkynyl have from 2 to 8 or 2 to 10 carbon atoms, respectively). When the number of carbon atoms is not indicated, the alkynyl group has from 2 to 6 carbon atoms.

The term “acyl” refers to an alkyl, haloalkyl, alkenyl, alkynyl, aryl cycloalkyl, heteroaryl, or heterocyclyl group, as defined herein, connected to the remainder of the compound by a C—O (carbonyl) group.

The term “carboxamido” refers to a —C(═O)NRR′ group, wherein R and R′ are independently selected from the group consisting of hydrogen, alkyl, alkenyl, alkynyl, aryl cycloalkyl, heteroaryl, and heterocyclyl, as defined herein.

The term “heteroalkylene” refers to a bivalent unsubstituted straight or branched group derived from heteroalkyl (e.g., as defined herein). Examples of heteroalkylene groups include, but are not limited to, —CH₂—CH₂—O—CH₂—, —CH₂—CH₂—O—CF₂—, —CH₂—CH₂—NH—CH₂—, —C(═O)—NH—CH₂—CH₂—NH—CH₂—C(═O)—N(CH₃)—CH₂—CH₂—N(CH₃)—CH₂—, —C(═O)—NH—CH₂—CH₂—NH—C(═O)—CH₂—CH₂—, —C(═O)—N(CH₃)—CH₂—CH₂—N(CH₃)—C(═O)—CH₂—CH₂—, —O—CH₂—CH₂—CH₂—NH—CH₂—, —O—CH₂—CH₂—CH₂—N(CH₃)—CH₂—, —O—CH₂—CH₂—CH₂—NH—C(═O)—CH₂—CH₂—, —O—CH₂—CH₂—CH₂—N(CH₃)—C(═O)—CH₂—CH₂—, —CH₂—CH₂—CH₂—NH—CH₂—, —CH₂—CH₂—CH₂—N(CH₃)—CH₂—, —CH₂—CH₂—CH₂—NH—C(═O)—CH₂—CH₂—, —CH₂—CH₂—CH₂—N(CH₃)—C(═O)—CH₂—CH₂—, —CH₂—CH₂—NH—C(═O)—, —CH₂—CH₂—N(CH₃)—CH₂—, —CH₂—CH₂—N⁺(CH₃)₂—, —NH—CH₂—CH₂(NH₂)—CH₂—, and —NH—CH₂—CH₂(NHCH₃)—CH₂—. A bivalent polyethylene glycol (PEG) moiety is a type of heteroalkylene group.

The term “alkoxy” refers to an alkyl group, as defined herein, which is attached to a molecule via an oxygen atom. For example, alkoxy groups include, but are not limited to methoxy, ethoxy, n-propoxy, iso-propoxy, n-butoxy, sec-butoxy, tert-butoxy, n-pentoxy and n-hexoxy.

The term “alkylthio” refers to an alkyl group, as defined herein, which is attached to a molecule via a sulfur atom. For example, alkythio groups include, but are not limited to thiomethyl, thioethyl, thio-n-propyl, thio-iso-propyl, and the like.

The term “haloalkyl” refers to an unsubstituted straight chain or branched, saturated hydrocarbon having the indicated number of carbon atoms (e.g., “C₁-C₄ alkyl,” “C₁-C₆ alkyl,” “C₁-C₈ alkyl,” or “C₁-C₁₀” alkyl have from 1 to 4, to 6, 1 to 8, or 1 to 10 carbon atoms, respectively) wherein at least one hydrogen atom of the alkyl group is replaced by a halogen (e.g., fluoro, chloro, bromo, or iodo). When the number of carbon atoms is not indicated, the haloalkyl group has from 1 to 6 carbon atoms. Representative C₁₋₆ haloalkyl groups include, but are not limited to, trifluoromethyl, 2,2,2-trifluoroethyl, and 1-chloroisopropyl.

The term “cycloalkyl” refers to a cyclic, saturated, or partially unsaturated hydrocarbon having the indicated number of carbon atoms (e.g., “C₃₋₈ cycloalkyl” or “C₃₋₆” cycloalkyl have from 3 to 8 or 3 to 6 carbon atoms, respectively). When the number of carbon atoms is not indicated, the cycloalkyl group has from 3 to 6 carbon atoms. Cycloalkyl groups include bridged, fused, and spiro ring systems, and bridged bicyclic systems where one ring is aromatic and the other is unsaturated. Representative “C₃₋₆ cycloalkyl” groups include, cyclopropyl, cyclobutyl, cyclopentyl, and cyclohexyl.

The term “aryl” refers to an unsubstituted monovalent carbocyclic aromatic hydrocarbon group of 6-10 carbon atoms derived by the removal of one hydrogen atom from a single carbon atom of a parent aromatic ring system. Aryl groups include, but are not limited to, phenyl, naphthyl, anthracenyl, biphenyl, and the like.

The term “heterocycle” refers to a saturated or partially unsaturated ring or a multiple condensed ring system, including bridged, fused, and spiro ring systems. Heterocycles can be described by the total number of atoms in the ring system, for example a 3-10 membered heterocycle has 3 to 10 total ring atoms. The term includes single saturated or partially unsaturated rings (e.g., 3, 4, 5, 6 or 7-membered rings) from about 1 to 6 carbon atoms and from about 1 to 3 heteroatoms selected from the group consisting of oxygen, nitrogen and sulfur in the ring. The ring may be substituted with one or more (e.g., 1, 2 or 3) oxo groups and the sulfur and nitrogen atoms may also be present in their oxidized forms. Such rings include but are not limited to azetidinyl, tetrahydrofuranyl and piperidinyl. The term “heterocycle” also includes multiple condensed ring systems (e.g., ring systems comprising 2, 3 or 4 rings) wherein a single heterocycle ring (as defined above) can be condensed with one or more heterocycles (e.g., decahydronapthyridinyl), carbocycles (e.g., decahydroquinolyl) or aryls. The rings of a multiple condensed ring system can be connected to each other via fused, spiro and bridged bonds when allowed by valency requirements. It is to be understood that the point of attachment of a multiple condensed ring system (as defined above for a heterocycle) can be at any position of the multiple condensed ring system including a heterocycle, aryl and carbocycle portion of the ring. It is also to be understood that the point of attachment for a heterocycle or heterocycle multiple condensed ring system can be at any suitable atom of the heterocycle or heterocycle multiple condensed ring system including a carbon atom and a heteroatom (e.g., a nitrogen). Exemplary heterocycles include, but are not limited to aziridinyl, azetidinyl, pyrrolidinyl, piperidinyl, homopiperidinyl, morpholinyl, thiomorpholinyl, piperazinyl, tetrahydrofuranyl, dihydrooxazolyl, tetrahydropyranyl, tetrahydrothiopyranyl, 1,2,3,4-tetrahydroquinolyl, benzoxazinyl, dihydrooxazolyl, chromanyl, 1,2-dihydropyridinyl, 2,3-dihydrobenzofuranyl, 1,3-benzodioxolyl, and 1,4-benzodioxanyl.

The term “heteroaryl” refers to an aromatic hydrocarbon ring system with at least one heteroatom within a single ring or within a fused ring system, selected from the group consisting of O, N and S. The ring or ring system has 4n+2 electrons in a conjugated π system where all atoms contributing to the conjugated π system are in the same plane. In some embodiments, heteroaryl groups have 5-10 total ring atoms and 1, 2, or 3 heteroatoms (referred to as a “5-10 membered heteroaryl”). Heteroaryl groups include, but are not limited to, imidazole, triazole, thiophene, furan, pyrrole, benzimidazole, pyrazole, pyrazine, pyridine, pyrimidine, and indole.

The term “hydroxyl” refers to an —OH group. The term “cyano” refers to a —CN group. The term “carboxy” refers to a —C(═O)OH group. The term “oxo” refers to a ═O group.

The term “alkanoyl” refers to an alkyl group, as defined herein, connected to the remainder of the molecule by a —C(═O) group. Exemplary alkanoyl groups include, but are not limited to acetyl, n-propanoyl, and n-butanoyl.

The term “alkanoyloxy” refers to an alkyl group, as defined herein, connected to the remainder of the molecule by an —OC(═O) group. Exemplary alkanoyloxy groups include, but are not limited to acetoxy, n-propanoyloxy, and n-butanoyloxy.

The term “alkoxycarbonyl” refers to an alkoxy group, as defined herein, connected to a C(═O)-alkyl group via the oxygen atom of the alkoxy (i.e., an alkyl ester group).

The terms “arylalkyl” and “cycloalkylalkyl” refer to an aryl group or a cycloalkyl group (as defined herein) connected to the remainder of the molecule by an alkyl group, as defined herein. Exemplary arylalkyl groups include but are not limited to benzyl and phenethyl. Exemplary cycloalkylalkyl groups include, but are not limited to cyclopropylmethyl, cyclobutylmethyl, cyclopentylethyl, and cyclohexylethyl.

### Numbered Embodiments

The following embodiments recite non-limiting permutations of combinations of features disclosed herein. Other permutations of combinations of features are also contemplated. In particular, each of these numbered embodiments is contemplated as depending from or relating to every previous or subsequent numbered embodiment, independent of their order as listed. 1. A composition comprising a compound of Formula (XVI),

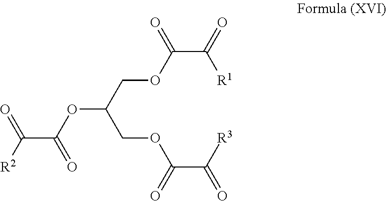

wherein R¹, R², and R³ comprise an amino acid sidechain each independently selected from the group consisting of

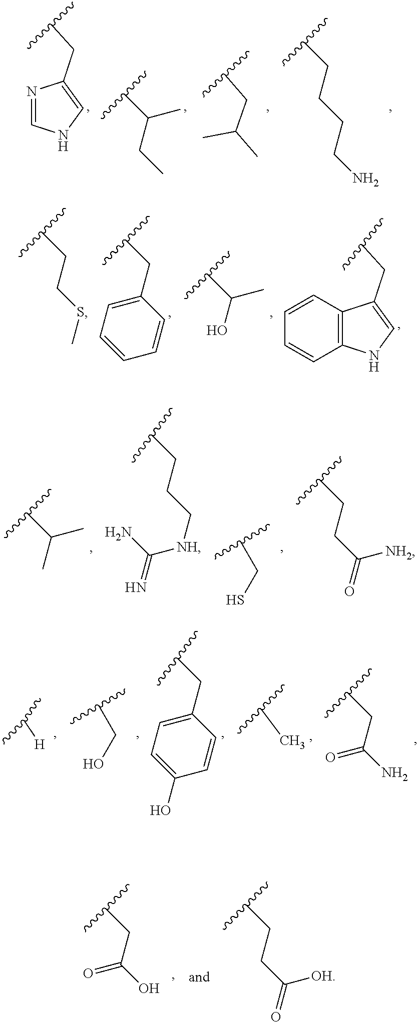

2. The composition of embodiment 1, wherein R¹, R², and R³ comprise an amino acid sidechain each independently selected from the group consisting of

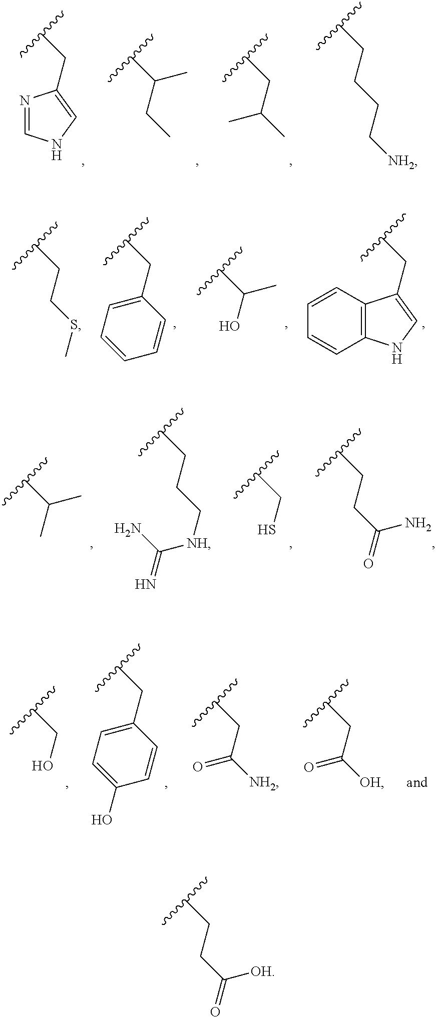

3. The composition of embodiment 1 or embodiment 2, wherein R¹, R², and R³ comprise an amino acid sidechain each independently selected from the group consisting of

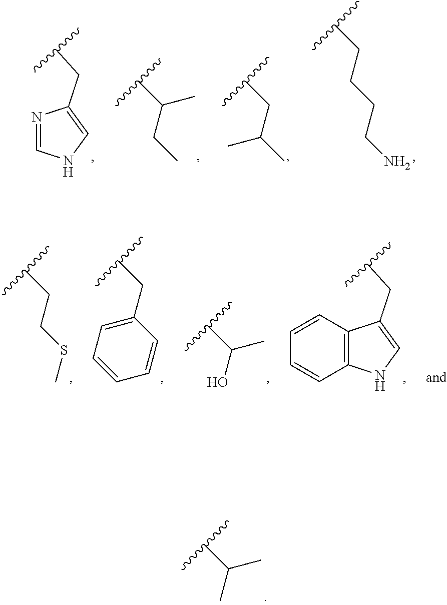

4. The composition of any one of embodiments 1-3, wherein R¹, R², and R³ are the same. 5. The composition of any one of embodiments 1-3, wherein R¹, R², and R³ are different. 6. The composition of any one of embodiments 1-5, wherein one or more of R¹, R², and R³ is

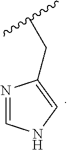 [SMILES: *Cc1c[nH]cn1]

7. The composition of any one of embodiments 1-6, wherein one or more of R¹, R², and R³ is

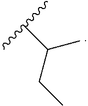 [SMILES: *C(C)CC]

8. The composition of any one of embodiments 1-7, wherein one or more of R¹, R², and R³ is

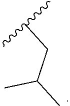 [SMILES: *CC(C)C]

9. The composition of any one of embodiments 1-8, wherein one or more of R¹, R², and R³ is

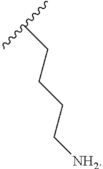 [SMILES: *CCCCN]

10. The composition of any one of embodiments 1-9, wherein one or more of R¹, R², and R³ is

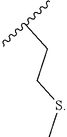 [SMILES: *CCSC]

11. The composition of any one of embodiments 1-10, wherein one or more of R¹, R², and R³ is

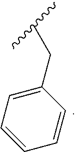 [SMILES: *Cc1ccccc1]

12. The composition of any one of embodiments 1-11, wherein one or more of R¹, R², and R³ is

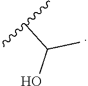 [SMILES: *C(C)O]

13. The composition of any one of embodiments 1-12, wherein one or more of R¹, R², and R³ is

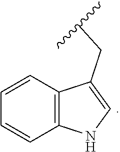 [SMILES: *Cc1c[nH]c2ccccc12]

14. The composition of any one of embodiments 1-13, wherein one or more of R¹, R², and R³ is

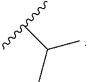 [SMILES: *C(C)C]

15. The composition of any one of embodiments 1-14, wherein one or more of R¹, R², and R³ is

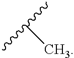

16. The composition of any one of embodiments 1-15, wherein one or more of R¹, R², and R³ is

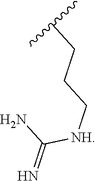 [SMILES: *CCCNC(=N)N]

17. The composition of any one of embodiments 1-16, wherein one or more of R¹, R², and R³ is

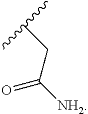 [SMILES: *CC(N)=O]

18. The composition of any one of embodiments 1-17, wherein one or more of R¹, R², and R³ is

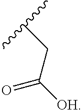 [SMILES: *CC(=O)O]

19. The composition of any one of embodiments 1-18, wherein one or more of R¹, R², and R³ is

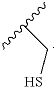 [SMILES: *CS]

20. The composition of any one of embodiments 1-19, wherein one or more of R¹, R², and R³ is

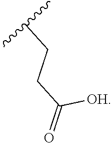 [SMILES: *CCC(=O)O]

21. The composition of any one of embodiments 1-20, wherein one or more of R¹, R², and R³ is

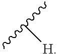

22. The composition of any one of embodiments 1-21, wherein one or more of R¹, R², and R³ is

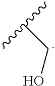 [SMILES: *CO]

23. The composition of any one of embodiments 1-22, wherein one or more of R¹, R², and R³ is

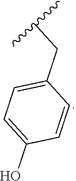 [SMILES: *Cc1ccc(O)cc1]

24. The composition of any one of embodiments 1-23, wherein one or more of R¹, R², and R³ is

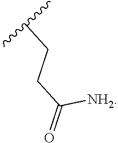 [SMILES: *CCC(N)=O]

25. A composition comprising a compound of Formula (XVII),

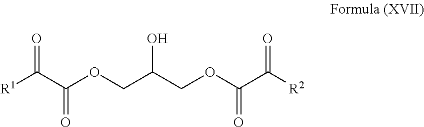

wherein R¹ and R² comprise an amino acid sidechain each independently selected from the group consisting of

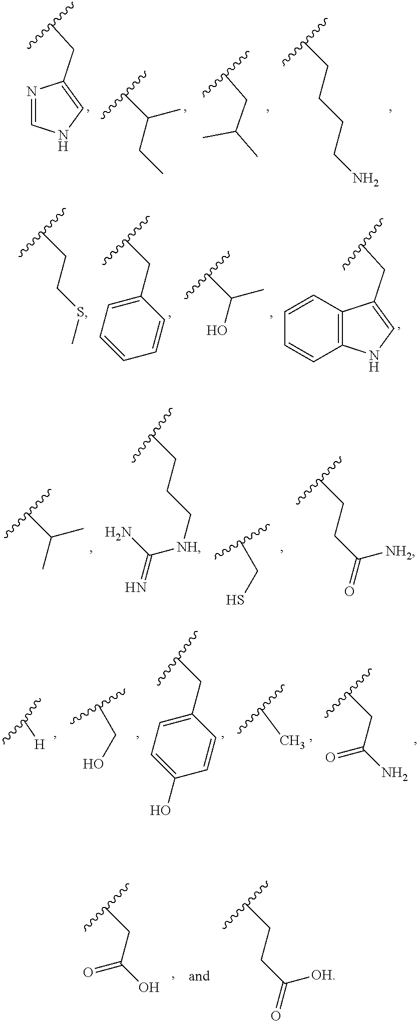

26. The composition of embodiment 25, wherein R¹ and R² comprise an amino acid sidechain each independently selected from the group consisting of

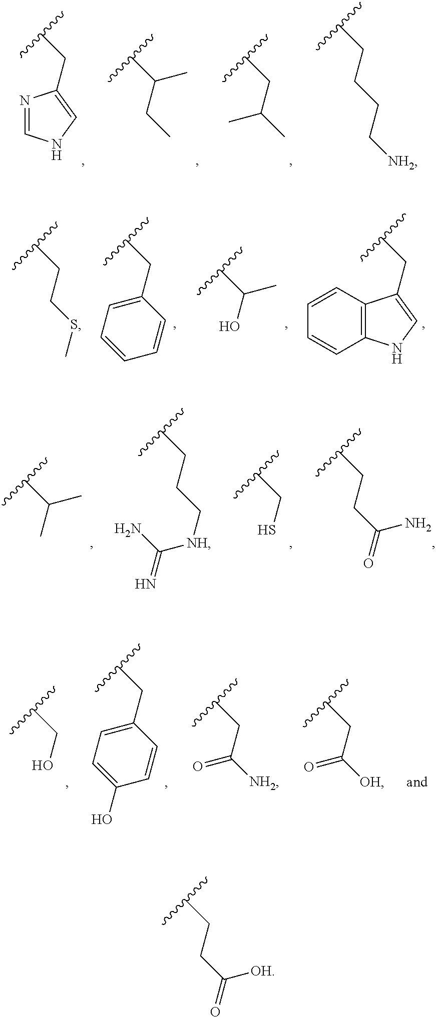

27. The composition of embodiment 25 or embodiment 26, wherein R¹ and R² comprise an amino acid sidechain each independently selected from the group consisting of

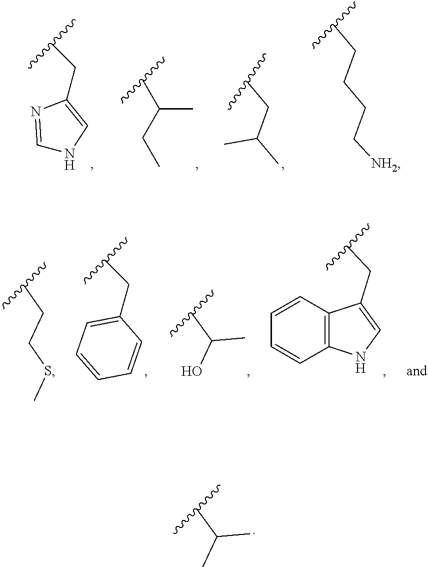

28. The composition of any one of embodiments 25-27, wherein R¹ and R² are the same. 29. The composition of any one of embodiments 25-27, wherein R¹ and R² are different. 30. The composition of any one of embodiments 25-29, wherein one or both of R¹ and R² is

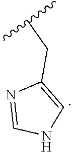 [SMILES: *Cc1c[nH]cn1]

31. The composition of embodiments 25-30, wherein one or both of R¹ and R² is

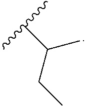 [SMILES: *C(C)CC]

32. The composition of any one of embodiments 25-31, wherein one or both of R¹ and R² is

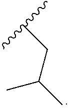 [SMILES: *CC(C)C]

33. The composition of any one of embodiments 25-32, wherein one or both of R¹ and R² is

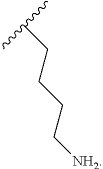 [SMILES: *CCCCN]

34. The composition of any one of embodiments 25-33, wherein one or both of R¹ and R² is

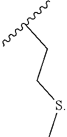 [SMILES: *CCSC]

35. The composition of any one of embodiments 25-34, wherein one or both of R¹ and R² is

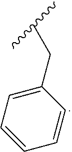 [SMILES: *Cc1ccccc1]

36. The composition of any one of embodiments 25-35, wherein one or both of R¹ and R² is

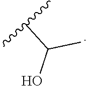 [SMILES: *C(C)O]

37. The composition of any one of embodiments 25-36, wherein one or both of R¹ and R² is

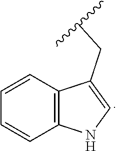 [SMILES: *Cc1c[nH]c2ccccc12]

38. The composition of any one of embodiments 25-37, wherein one or both of R¹ and R² is

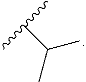 [SMILES: *C(C)C]

39. The composition of any one of embodiments 25-38, wherein one or both of R¹ and R² is

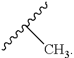

40. The composition of any one of embodiments 25-39, wherein one or both of R¹ and R² is

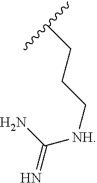 [SMILES: *CCCNC(=N)N]

41. The composition of any one of embodiments 25-40, wherein one or both of R¹ and R² is

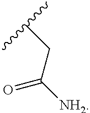 [SMILES: *CC(N)=O]

42. The composition of any one of embodiments 25-41, wherein one or both of R¹ and R² is

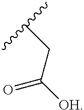 [SMILES: *CC(=O)O]

43. The composition of any one of embodiments 25-42, wherein one or both of R¹ and R² is

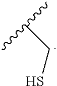 [SMILES: *CS]

44. The composition of any one of embodiments 25-43, wherein one or both of R¹ and R² is

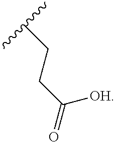 [SMILES: *CCC(=O)O]

45. The composition of any one of embodiments 25-44, wherein one or both of R¹ and R² is

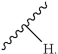

46. The composition of any one of embodiments 25-45, wherein one or both of R¹ and R² is

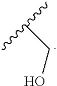 [SMILES: *CO]

47. The composition of any one of embodiments 25-46, wherein one or both of R¹ and R² is

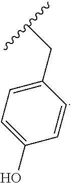 [SMILES: *Cc1ccc(O)cc1]

48. The composition of any one of embodiments 25-47, wherein one or both of R¹ and R² is

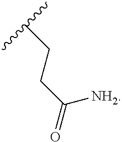 [SMILES: *CCC(N)=O]

49. A composition comprising a compound of Formula (XVIII),

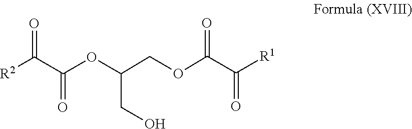

wherein R¹ and R² comprise an amino acid sidechain each independently selected from the group consisting of

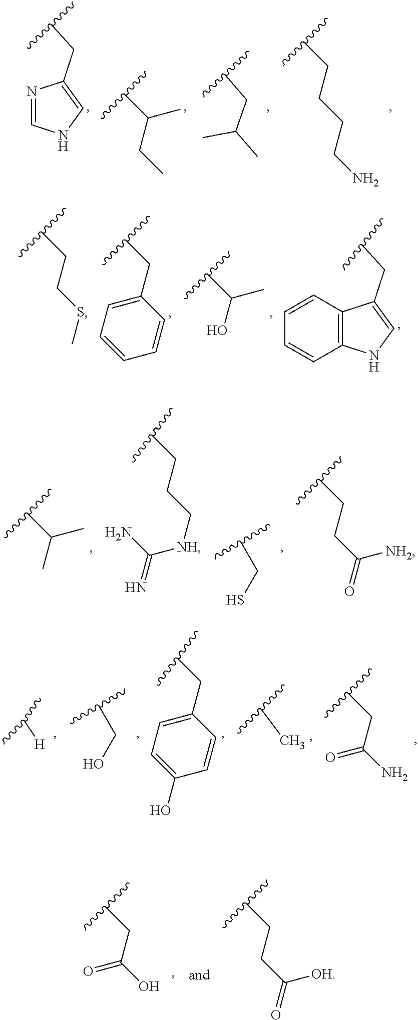

50. The composition of embodiment 49, wherein R¹ and R² comprise an amino acid sidechain each independently selected from the group consisting of

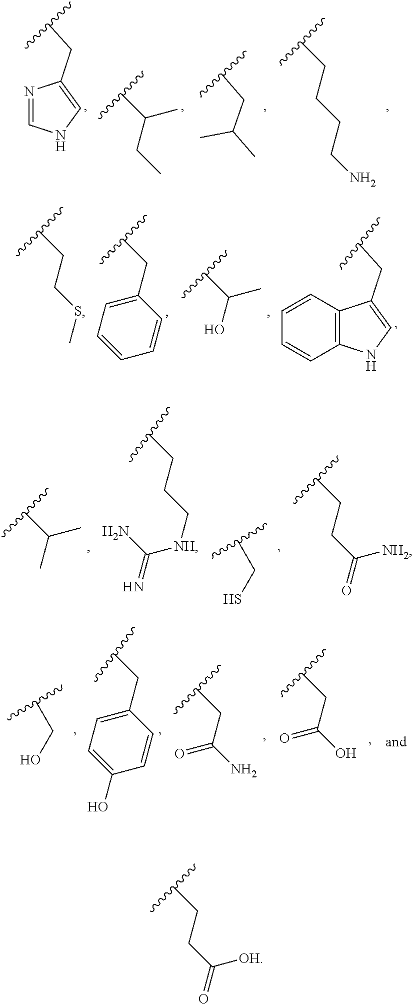

51. The composition of embodiment 49 or embodiment 50, wherein R¹ and R² comprise an amino acid sidechain each independently selected from the group consisting of

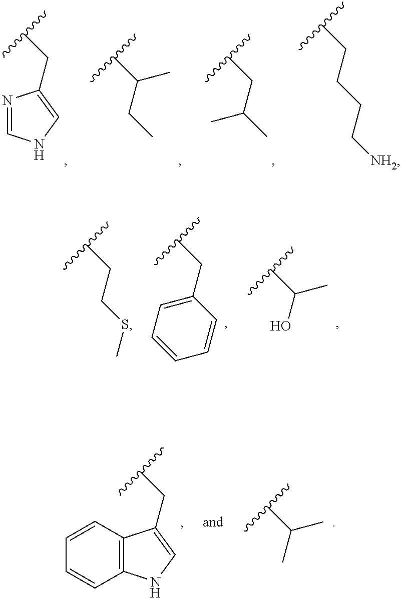

52. The composition of embodiment 50 or embodiment 51, wherein R¹ and R² are the same. 53. The composition of embodiment 50 or embodiment 51, wherein R¹ and R² are different. 54. The composition of any one of embodiments 50-53, wherein one or both of R¹ and R² is

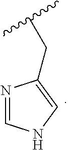 [SMILES: *Cc1c[nH]cn1]

55. The composition of any one of embodiments 50-54, wherein one or both of R¹ and R² is

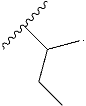 [SMILES: *C(C)CC]

56. The composition of any one of embodiments 50-55, wherein one or both of R¹ and R² is

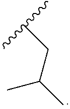 [SMILES: *CC(C)C]

57. The composition of any one of embodiments 50-56, wherein one or both of R¹ and R² is

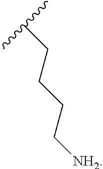 [SMILES: *CCCCN]

58. The composition of any one of embodiments 50-57, wherein one or both of R¹ and R² is

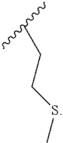 [SMILES: *CCSC]

59. The composition of any one of embodiments 50-58, wherein one or both of R¹ and R² is

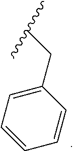 [SMILES: *Cc1ccccc1]

60. The composition of any one of embodiments 50-59, wherein one or both of R¹ and R² is

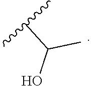 [SMILES: *C(C)O]

61. The composition of any one of embodiments 50-60, wherein one or both of R¹ and R² is

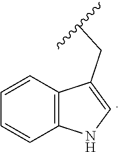 [SMILES: *Cc1c[nH]c2ccccc12]

62. The composition of any one of embodiments 50-61, wherein one or both of R¹ and R² is

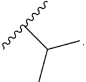 [SMILES: *C(C)C]

63. The composition of any one of embodiments 50-61, wherein one or both of R¹ and R² is

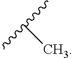

64. The composition of any one of embodiments 50-63, wherein one or both of R¹ and R² is

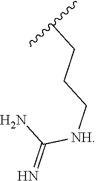 [SMILES: *CCCNC(=N)N]

65. The composition of any one of embodiments 50-64, wherein one or both of R¹ and R² is

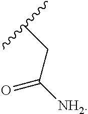 [SMILES: *CC(N)=O]

66. The composition of any one of embodiments 50-65, wherein one or both of R¹ and R² is

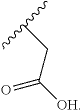 [SMILES: *CC(=O)O]

67. The composition of any one of embodiments 50-66, wherein one or both of R¹ and R² is

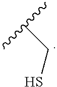 [SMILES: *CS]

68. The composition of any one of embodiments 50-67, wherein one or both of R¹ and R² is

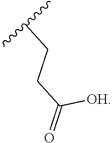 [SMILES: *CCC(=O)O]

69. The composition of any one of embodiments 50-68, wherein one or both of R¹ and R² is

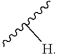

70. The composition of any one of embodiments 50-69, wherein one or both of R¹ and R² is

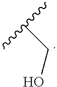 [SMILES: *CO]

71. The composition of any one of embodiments 50-70, wherein one or both of R¹ and R² is

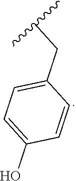 [SMILES: *Cc1ccc(O)cc1]

72. The composition of any one of embodiments 50-71 wherein one or both of R¹ and R² is

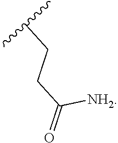 [SMILES: *CCC(N)=O]

73. A composition comprising a compound of Formula (XIX),

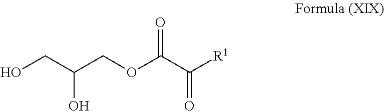

wherein R¹ comprises an amino acid sidechain selected from the group consisting of

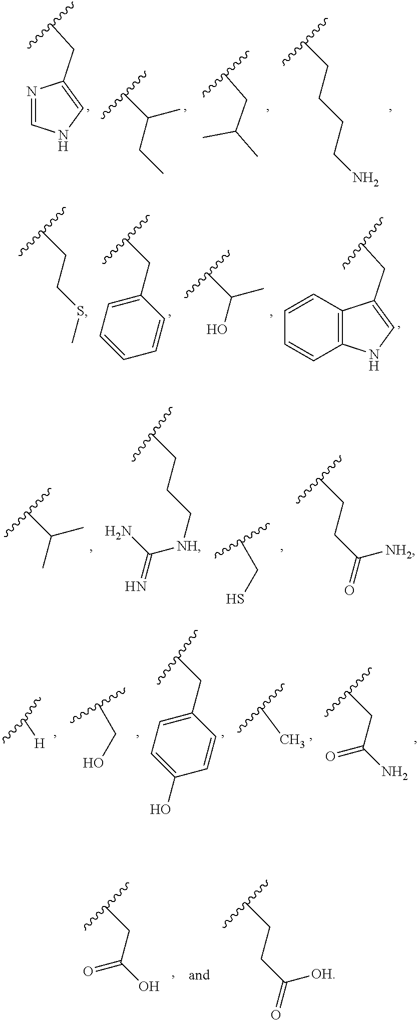

74. A composition comprising a compound of Formula (XX),

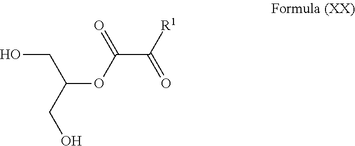

wherein R¹ comprises an amino acid sidechain selected from the group consisting of

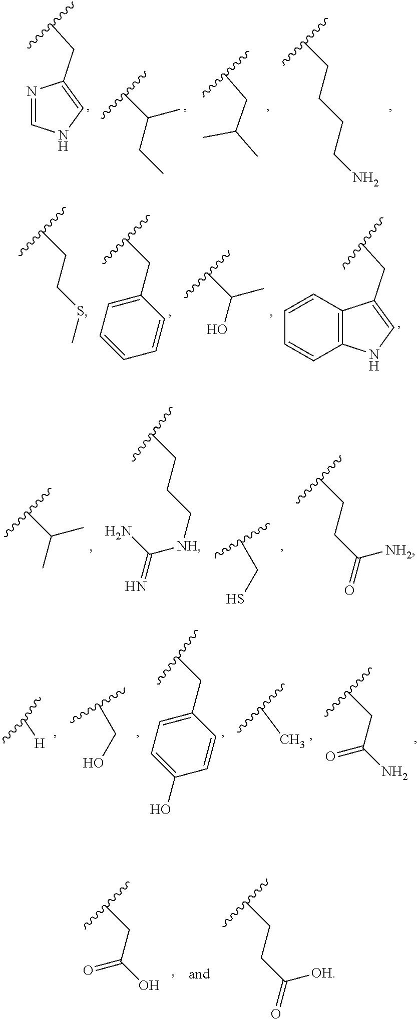

75. The composition of embodiment 73 or embodiment 74, wherein R¹ comprises an amino acid sidechain selected from the group consisting of

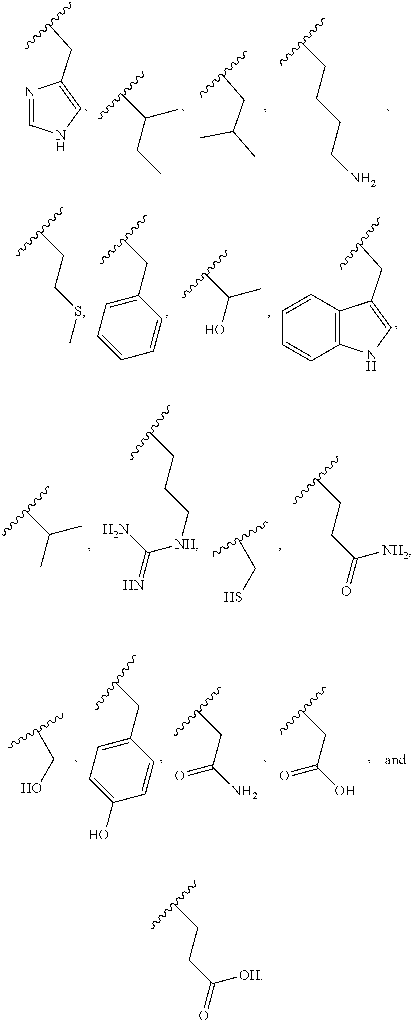

76. The composition of any one of embodiments 73-75, wherein R¹ comprises an amino acid sidechain selected from the group consisting of

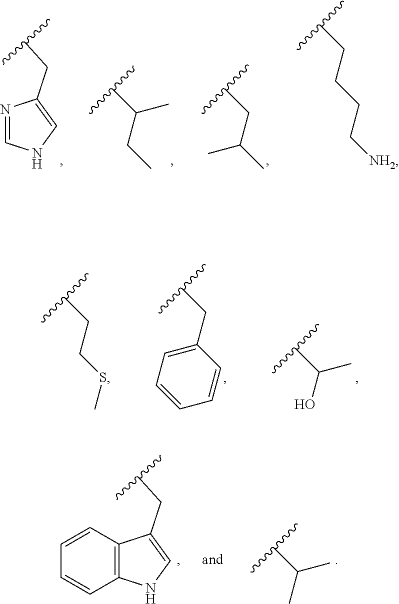

77. The composition of any one of embodiments 73-76, wherein R¹ is

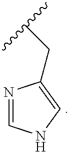 [SMILES: *Cc1c[nH]cn1]

78. The composition of any one of embodiments 73-76, wherein R¹ is

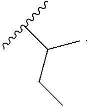 [SMILES: *C(C)CC]

79. The composition of any one of embodiments 73-76, wherein R¹ is

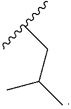 [SMILES: *CC(C)C]

80. The composition of any one of embodiments 73-76, wherein R¹ is

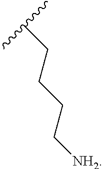 [SMILES: *CCCCN]

81. The composition of any one of embodiments 73-76, wherein R¹ is

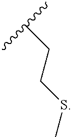 [SMILES: *CCSC]

82. The composition of any one of embodiments 73-76, wherein R¹ is

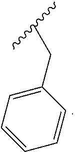 [SMILES: *Cc1ccccc1]

83. The composition of any one of embodiments 73-76, wherein R¹ is

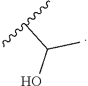 [SMILES: *C(C)O]

84. The composition of any one of embodiments 73-76, wherein R¹ is

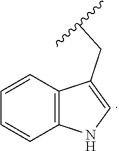 [SMILES: *Cc1c[nH]c2ccccc12]

85. The composition of any one of embodiments 73-76, wherein R¹ is

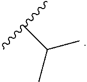 [SMILES: *C(C)C]

86. The composition of embodiment 73 or 74, wherein R¹ is

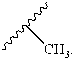

87. The composition of any one of embodiments 73-75, wherein R¹ is

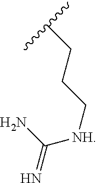 [SMILES: *CCCNC(=N)N]

88. The composition of any one of embodiments 73-75, wherein R¹ is

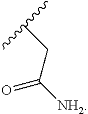 [SMILES: *CC(N)=O]

89. The composition of any one of embodiments 73-75, wherein R¹ is

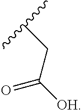 [SMILES: *CC(=O)O]

90. The composition of any one of embodiments 73-75, wherein R¹ is

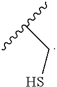 [SMILES: *CS]

91. The composition of any one of embodiments 73-75, wherein R¹ is

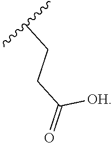 [SMILES: *CCC(=O)O]

92. The composition of embodiment 73 or embodiment 74, wherein R¹ is

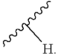

93. The composition of any one of embodiments 73-75, wherein R¹ is

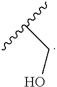 [SMILES: *CO]

94. The composition of any one of embodiments 73-75, wherein R¹ is

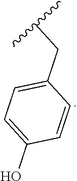 [SMILES: *Cc1ccc(O)cc1]

95. The composition of any one of embodiments 73-75, wherein R¹ is

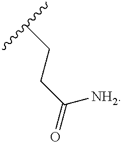 [SMILES: *CCC(N)=O]

96. A composition comprising a compound of Formula (XXI):

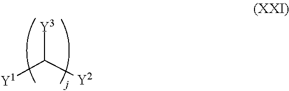

wherein: Y¹ and Y² are each independently selected from the group consisting of —CH₂Y³, —C(═O) H, —C(═O)OX₂, and —C(═O)NX²X³; each instance of Y³ is each independently selected from the group consisting of —H, —OX¹, —NX²X³, —C(═O)OX₂, and —C(═O)NX²X³; each instance of X¹ is independently selected from the group consisting of —H, —C₁₋₃ alkyl, —C(═O)CH₃, —C(═O)OX₂, —C(═O)NX²X³, —PO₃²⁻, —SO₃⁻, a C₄₋₉ monosaccharide, a C₈₋₁₈ disaccharide, and

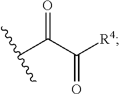

each instance of X² and X³ is independently selected from the group consisting of —H and —C₁-C₃ alkyl; each instance of R⁴ is independently selected from the group consisting of non-proline, non-alanine, and non-glycine natural amino acid sidechains, subscript j is an integer from 0 to 23; and at least one instance of Y¹, Y², and Y³ is —OX¹ of which X¹ is

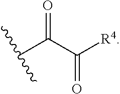

97. The composition of embodiment 96, wherein Y¹ and Y² are each —CH₂Y³. 98. The composition of embodiment 96 or embodiment 97, wherein at most one instance of Y³ is —C(═O)OX₂ and the remaining instances of Y³ are —OX¹. 99. The composition of embodiment 96 or 97, wherein the compound of Formula (XXI) is a compound of Formula (XXIa):

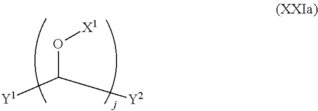

wherein: Y¹ and Y² are each independently selected from the group consisting of —CH₂Y³, —C(═O) H, —C(═O)OX₂, and —C(═O)NX²X³; each instance of Y³ is each independently selected from the group consisting of —H, —OX¹, —NX²X³, —C(═O)OX₂, and —C(═O)NX²X³; each instance of X¹ is independently selected from the group consisting of —H, —C₁₋₃ alkyl, —C(═O)CH₃, —C(═O)OX₂, —C(═O)NX²X³, —PO₃²⁻, —SO₃⁻, a C₄₋₉ monosaccharide, a C₈₋₁₈ disaccharide, and

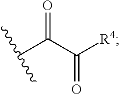

each instance of X² and X³ is independently selected from the group consisting of —H and —C₁-C₃ alkyl; each instance of R⁴ is independently selected from the group consisting of non-proline, non-alanine, and non-glycine natural amino acid sidechains, subscript j is an integer from 0 to 23; and at least one instance of X¹ is

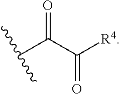

100. The composition of any one of embodiments 96-99, wherein at most one instance of X¹ is —PO₃²⁻, —SO₃⁻, a C₄₋₉ monosaccharide, or a C₈₋₁₈ disaccharide, and the remaining instances are each independently selected from the
group consisting of —H, C₁₋₃ alkyl, and

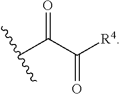

101. The composition of any one of embodiments 96-100, wherein each instance of X¹ is independently selected from the group consisting of —H and

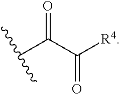

102. The composition of any one of embodiments 96-101, wherein subscript j is an integer from 1 to 6. 103. The composition of any one of embodiments 96-102, wherein each instance of R⁴ is independently selected from the group consisting of

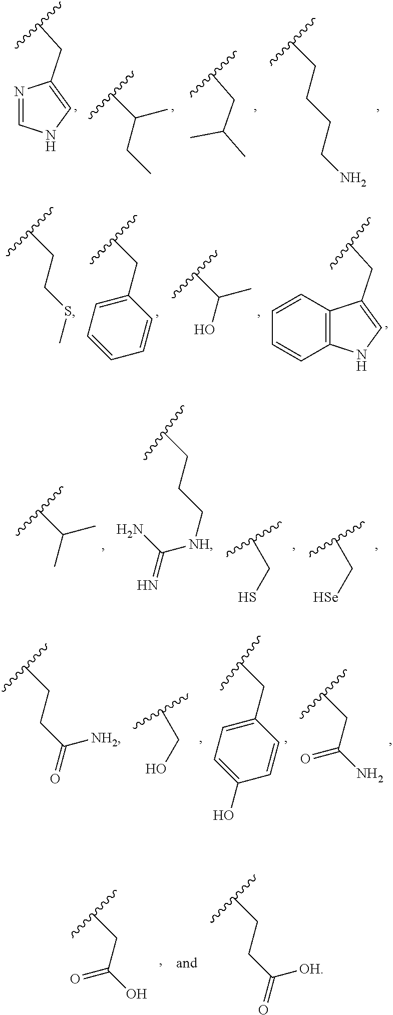

104. The composition of any one of embodiments 96-103, wherein each instance of R⁴ is independently selected from the group consisting of

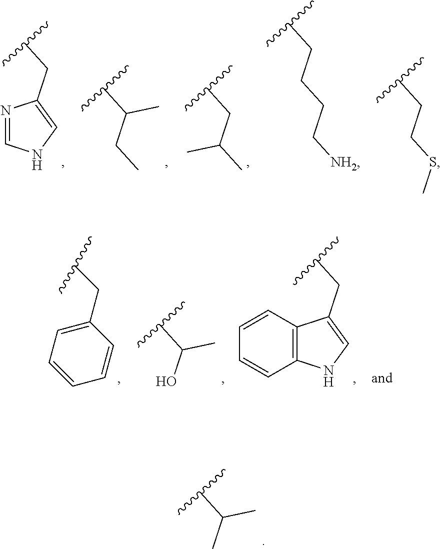

105. A composition comprising a compound of Formula (XXII):

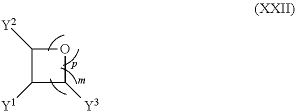

wherein: Y¹ is selected from the group consisting of —H, —OX¹, —C(═O)OX₂, —C(═O)NX²x³; Y² is selected from the group consisting of —H, —OX¹, —CH₂OX¹, —C(═O)OX₂, —C(═O)NX²X³, and

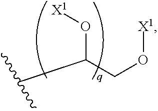

wherein subscript q is an integer from 1 to 3 and [symbol] denotes a point of attachment of Y² to the remainder of Formula (XXII); each instance of Y³ is each independently selected from the group consisting of —H, —OX¹, —C(═O)OX₂, and —C(═O)NX²X³; each instance of X¹ is independently selected from the group consisting of —H, —C₁₋₃ alkyl, —PO₃²⁻, —SO₃⁻, a C₄₋₉ monosaccharide, a C₈₋₁₈ disaccharide, and

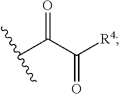

each instance of X² and X³ is independently selected from the group consisting of —H and C₁₋₃ alkyl; each instance of R⁴ is independently selected from the group consisting of non-proline, non-alanine, and non-glycine natural amino acid sidechains, and subscript m is an integer from 1 to 23, and subscript p is 0 or 1. 106. The composition of embodiment 105, wherein subscript m is an integer from 2 to 5. 107. The composition of embodiment 105 or embodiment 106, wherein Y¹ is selected from the group consisting of —H and —OX¹. 108. The composition of any one of embodiments 105-107, wherein subscript p is 0 and Y² is —OX¹. 109. The composition of any one of embodiments 105-108, wherein each instance of Y³ is each independently selected from the group consisting of —H and —OX¹. 110. The composition of any one of embodiments 105-109, wherein the compound of Formula (XXII) is a compound of Formula (XXIIa):

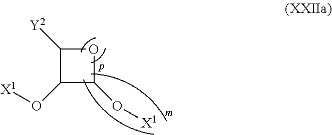

wherein: Y² is selected from the group consisting of —OX¹, —CH₂OX¹, and

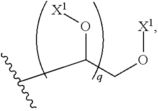

wherein subscript q is an integer from 1 to 3 and [symbol] denotes a point of attachment of Y² to the remainder of Formula (XXII); each instance of X¹ is independently selected from the group consisting of —H, —C₁₋₃ alkyl, —PO₃²⁻, —SO₃⁻, a C₄₋₉ monosaccharide, a C₈₋₁₈ disaccharide, and

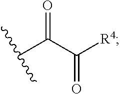

each instance of X² and X³ is independently selected from the group consisting of —H and C₁₋₃ alkyl; each instance of R⁴ is independently selected from the group consisting of non-proline, non-alanine, and non-glycine natural amino acid sidechains, subscript m is an integer from 1 to 23; and subscript p is 0 or 1. 111. The composition of any one of embodiments 105-110, wherein one, two, or three instances of X¹ are

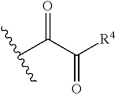

and the remaining instances are —H. 112. The composition of any one of embodiments 105-111, wherein at most one instance of X¹ is —PO₃²⁻, —SO₃⁻, a C₄₋₉ monosaccharide, or a C₈₋₁₈ disaccharide, and the remaining instances are each independently selected from the group consisting of —H and

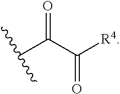

113. The composition of any one of embodiments 105-112, wherein each instance of R⁴ is independently selected from the group consisting of

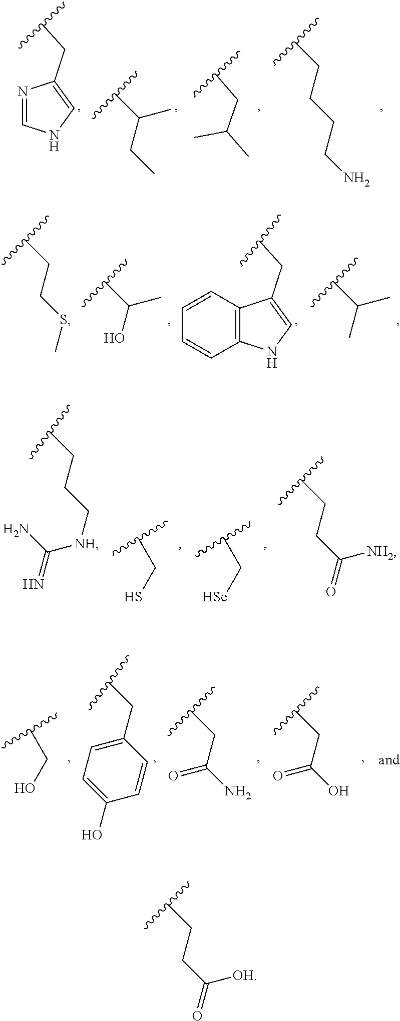

114. The composition of any one of embodiments 105-113, wherein each instance of R⁴ is independently selected from the group consisting of

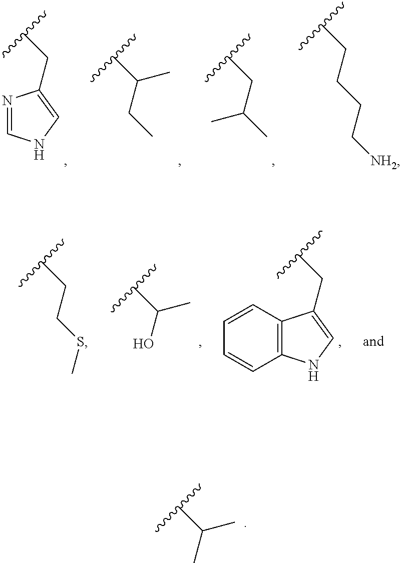

115. A consumable composition comprising the composition of any one of embodiments 1-114. 116. The consumable composition of embodiment 115, wherein the consumable composition comprises a solid, a liquid, a colloid, a gel, or a combination thereof. 117. The consumable composition of embodiment 116, wherein the consumable composition is a medical grade food. 118. A medical food comprising the composition of any one of embodiments 1-114 and a food or beverage. 119. The medical food of any one of embodiment 118, wherein the medical food comprises a food. 120. The medical food of any one of embodiments 118-119, wherein the medical food comprises a beverage. 121. The medical food of any one of embodiments 118-120, wherein the medical food is a solid. 122. The medical food of any one of embodiments 118-121, wherein the medical grade food is a liquid. 123. The medical food of any one of embodiments 118-122, wherein the medical grade food is gelatinous. 124. The medical food of any one of embodiments 118-123, wherein the medical grade food is a beverage, a bar, a cereal, a sports drink, a gel, a gelatin, a gelatinous gummy, a cracker, a chip, a puff, a granola cereal, a granola bar, a tablet, a powder, or an additive. 125. The medical food of any one of embodiments 118-124, comprising a Cd content of less than 5 μg per daily serving or of less than 0.5 μg per g. 126. The medical food of any one of embodiments 118-125, comprising a Pb content of less than 5 μg per daily serving or less than 0.5 μg per g. 127. The medical food of any one of embodiments 118-126, comprising an As content of less than 15 μg per daily serving or less than 1.5 μg per g. 128. The medical food of any one of embodiments 118-127, comprising a Hg content of less than 30 μg per daily serving or less than 3 μg per g. 129. The medical food of any one of embodiments 118-128, comprising a Co content of less than 50 μg per daily serving or less than 5 μg per g. 130. The medical food of any one of embodiments 118-129, comprising a V content of less than 100 μg per daily serving or less than 10 μg per g. 131. The medical food of any one of embodiments 118-130, comprising a Ni content of less than 200 μg per daily serving or less than 20 μg per g. 132. The medical food of any one of embodiments 118-131, comprising a Tl content of less than 8 μg per daily serving or less than 0.8 μg per g. 133. The medical food of any one of embodiments 118-132, comprising an Au content of less than 300 μg per daily serving or less than 30 μg per g. 134. The medical food of any one of embodiments 118-133, comprising a Pd content of less than 100 μg per daily serving or less than 10 μg per g. 135. The medical food of any one of embodiments 118-134, comprising an Ir content of less than 100 μg per daily serving or less than 10 μg per g. 136. The medical food of any one of embodiments 118-135, comprising an Os content of less than 100 μg per daily serving or less than 10 μg per g. 137. The medical food of any one of embodiments 118-136, comprising a Rh content of less than 100 μg per daily serving or less than 10 μg per g. 138. The medical food of any one of embodiments 118-137, comprising a Ru content of less than 100 μg per daily serving or less than 10 μg per g. 139. The medical food of any one of embodiments 118-138, comprising a Se content of less than 150 μg per daily serving or less than 15 μg per g. 140. The medical food of any one of embodiments 118-139, comprising an Ag content of less than 150 μg per daily serving or less than 15 μg per g. 141. The medical food of any one of embodiments 118-140, comprising a Pt content of less than 100 μg per daily serving or less than 10 μg per g. 142. The medical food of any one of embodiments 118-141, comprising a Li content of less than 550 μg per daily serving or less than 55 μg per g. 143. The medical food of any one of embodiments 118-142, comprising a Sb content of less than 1200 μg per daily serving or less than 120 μg per g. 144. The medical food of any one of embodiments 118-143, comprising a Ba content of less than 1400 μg per daily serving or less than 140 μg per g. 145. The medical food of any one of embodiments 118-144, comprising a Mo content of less than 3000 μg per daily serving or less than 300 μg per g. 146. The medical food of any one of embodiments 118-145, comprising a Cu content of less than 3000 μg per daily serving or less than 300 μg per g. 147. The medical food of any one of embodiments 118-146, comprising a Sn content of less than 6000 μg per daily serving or less than 600 μg per g. 148. The medical food of any one of embodiments 118-147, comprising a Cr content of less than 11000 μg per daily serving or less than 1100 μg per g. 149. The medical food of any one of embodiments 118-148, comprising a microbial plate count of no more than 1000 colony forming per mL, no more than 10,000 colony forming per mL, or no more than 20,000 colony forming per mL. 150. A method of treating a subject in need thereof, the method comprising administering to the subject the composition of any one of embodiments 1-114, the consumable composition of any one of embodiments 115-117, or the medical food of any one of embodiments 118-149. 151. The method of embodiment 150, wherein the subject has or is at risk of having a kidney disease. 152. The method of embodiment 151, wherein the kidney disease is chronic kidney disease, Fabry disease, cystinosis, glomerulonephritis, IgA nephropathy, lupus nephritis, atypical hemolytic uremic syndrome, polycystic kidney disease, diabetic kidney disease, or uremia.

### Additional Numbered Embodiments

The following embodiments recite non-limiting permutations of combinations of features disclosed herein. Other permutations of combinations of features are also contemplated. In particular, each of these numbered embodiments is contemplated as depending from or relating to every previous or subsequent numbered embodiment, independent of their order as listed. 1. A composition comprising a polysaccharide and an alpha-keto acid, 2. A composition comprising a polysaccharide and an alpha-keto acid analogue of an essential amino acid. 3. A composition comprising a polysaccharide non-covalently coupled to an alpha-keto acid, 4. The composition of any one of embodiments 1-3, wherein the polysaccharide is a starch, a cellulose, an amino polysaccharide, an alginate, a carrageenan, a chitin, a chondroitin sulfate, a dextran, a galactomannan, a glycogen, a hyaluronic acid, a glycogen, a galactogen, an inulin, an arabinoxylan, or a pectin. 5. The composition of any one of embodiments 1-4, wherein the polysaccharide is an amino polysaccharide. 6. The composition of embodiment 5, wherein the amino polysaccharide comprises an amino cellulose, an amino dextran, an amino galactose, an amino arabinose, a chitin, a chitosan, or a combination thereof. 7. The composition of any one of embodiments 1-4, wherein the polysaccharide is a chitosan. 8. The composition of embodiment 7, wherein the chitosan comprises Formula (I):

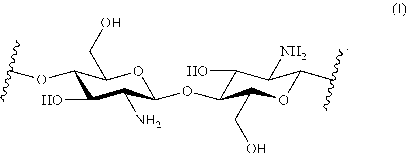

9. The composition of any one of embodiments 1-8 wherein the alpha-keto acid comprises:

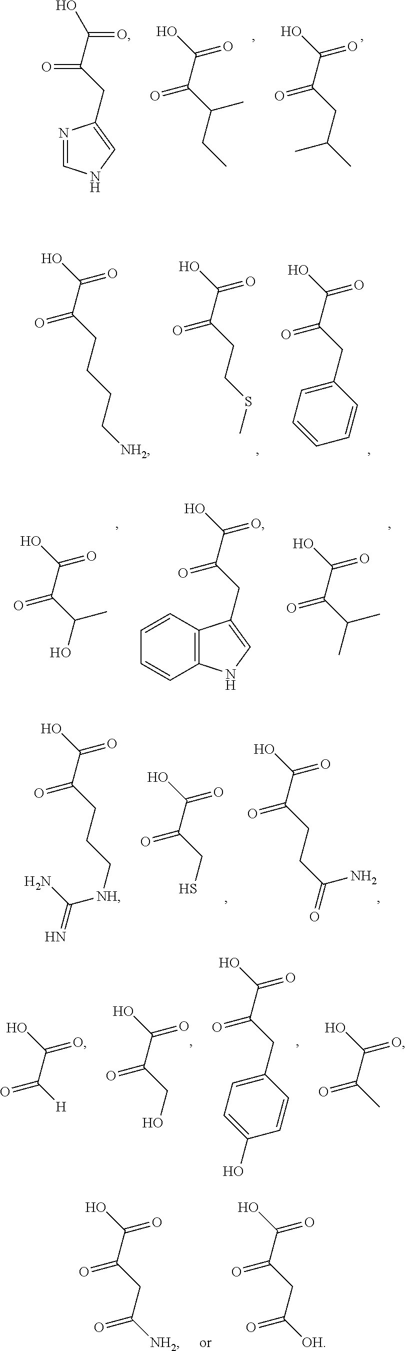

10. The composition of any one of embodiments 1-9, wherein the alpha-keto acid comprises an alpha-keto acid analogue of an essential amino acid. 11. The composition of embodiment 10, wherein the alpha-keto acid analogue of an essential amino acid comprises:

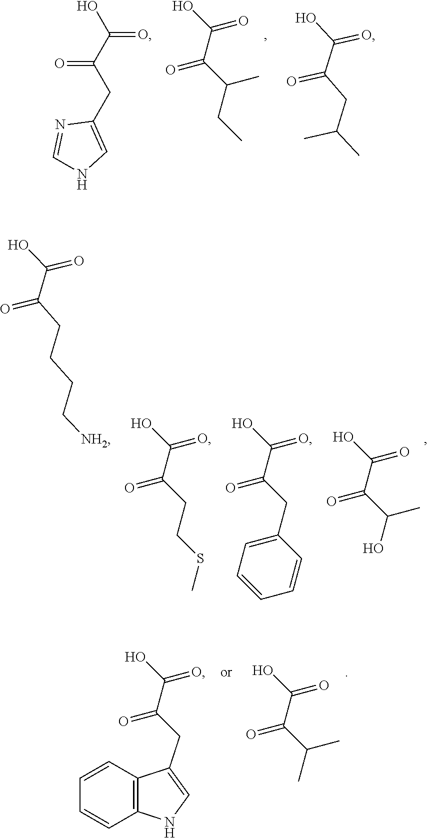

12. The composition of any one of embodiments 1-11, wherein the alpha-keto acid is neutrally charged at pH 7. 13. The composition of any one of embodiments 1-11, wherein the alpha-keto acid is negatively charged at pH 7. 14. The composition of any one of embodiments 1-13, wherein the polysaccharide is neutrally charged at pH 7. 15. The composition of any one of embodiments 1-13, wherein the polysaccharide is positively charged at pH 7. 16. The composition of any one of embodiments 6-15, wherein the chitosan is neutrally charged at pH 7. 17. The composition of any one of embodiments 6-15, wherein the chitosan is positively charged at pH 7. 18. The composition of any one of embodiments 1-17, wherein the polysaccharide is non-covalently coupled to the alpha-keto acid. 19. The composition of any one of embodiments 6-18, wherein the chitosan is ionically coupled to an alpha-keto acid through a positive charge on the chitosan and a negative charge on the alpha-keto acid. 20. The composition of any one of embodiments 6-19, wherein the chitosan is coupled to the alpha-keto acid through hydrogen bonding interactions. 21. The composition of any one of embodiments 6-20, wherein the chitosan is covalently coupled to the alpha-keto acid. 22. The composition of any one of embodiments 1-21, wherein the alpha-keto acid is covalently coupled to the polysaccharide as an amide. 23. The composition of any one of embodiments 1-22, wherein the alpha-keto acid is covalently coupled to the polysaccharide as an alpha-keto amide. 24. The composition of any one of embodiments 6-23, wherein a plurality of alpha-keto acids is coupled to the chitosan. 25. The composition of embodiment 24, wherein the plurality of alpha-keto acids comprises from 2 to 100,000, from 2 to 10,000, from 2 to 1000, from 10 to 100,000, from 10 to 10,000, from 10 to 1000, from 100 to 100,000, from 100 to 10,000, or from 100 to 1000 alpha-keto acids. 26. The composition of embodiment 24 or embodiment 25, wherein between about 50% and 99.9%, about 60% and 99%, about 70% and 99%, about 80% and 99.9%, or about 90% and 99.9% of the plurality of alpha-keto acids are non-covalently coupled to the chitosan. 27. The composition of any one of embodiments 24-26, wherein between about 10% and 50%, about 20% and 60%, about 30% and 70%, about 40% and 80%, or about 50% and 90% of the plurality alpha-keto acids are covalently coupled to the chitosan. 28. The composition of any one of embodiments 24-27, wherein the plurality of alpha-keto acids comprises a first alpha-keto acid covalently coupled to the chitosan a second alpha-keto acid non-covalently coupled to the chitosan. 29. The composition of any one of embodiments 1-28, wherein the composition comprises Formula (VI):

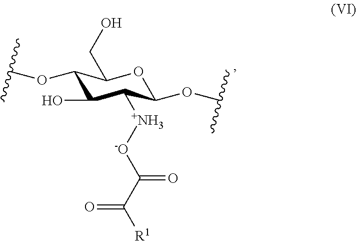

wherein each R¹ is independently a sidechain of a natural amino acid. 30. The composition of any one of embodiments 1-29, wherein the composition comprises a chitosan cation and an alpha-keto acid anion. 31. The composition of any one of embodiments 1-30, wherein the composition comprises Formula (VIII):

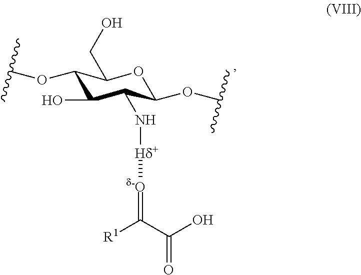

wherein each R¹ is independently a sidechain of a natural amino acid. 32. The composition of any one of embodiments 1-31, wherein the composition comprises Formula (XII),

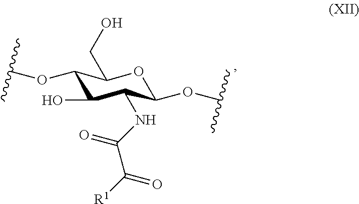

wherein each R¹ is independently a sidechain of a natural amino acid. 33. The composition of any one of embodiments 1-32, wherein each R¹ is independently:

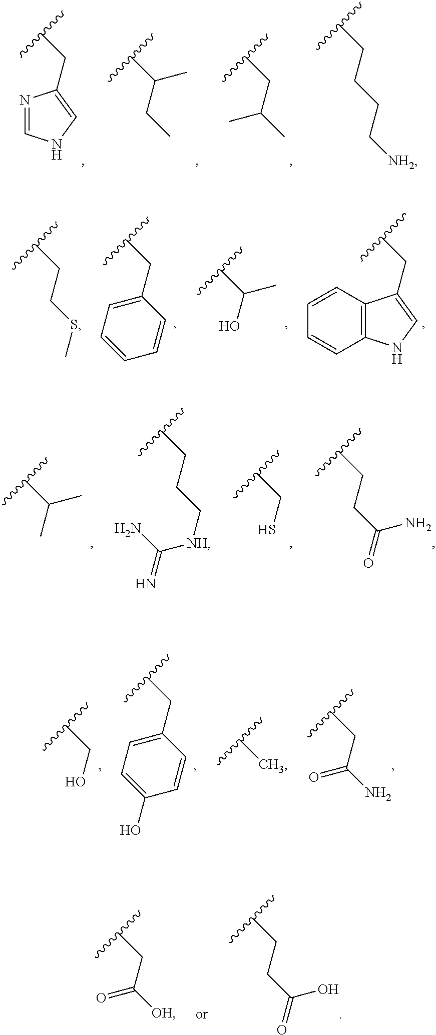

34. The composition of any one of embodiments 1-33, wherein each R¹ is independently:

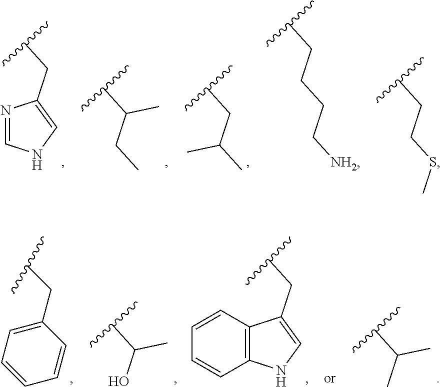

35. The composition of any one of embodiments 1-34, wherein R¹ comprises:

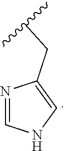 [SMILES: *Cc1c[nH]cn1]

36. The composition of any one of embodiments 1-35, wherein R¹ comprises:

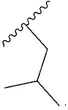 [SMILES: *CC(C)C]

37. The composition of any one of embodiments 1-36, wherein R¹ comprises:

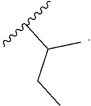 [SMILES: *C(C)CC]

38. The composition of any one of embodiments 1-37, wherein R¹ comprises:

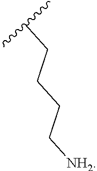 [SMILES: *CCCCN]

39. The composition of any one of embodiments 1-38, wherein R¹ comprises:

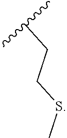 [SMILES: *CCSC]

40. The composition of any one of embodiments 1-39, wherein R¹ comprises:

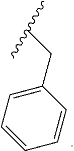 [SMILES: *Cc1ccccc1]

41. The composition of any one of embodiments 1-40, wherein R¹ comprises:

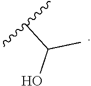 [SMILES: *C(C)O]

42. The composition of any one of embodiments 1-41, wherein R¹ comprises:

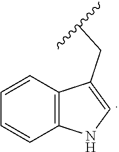 [SMILES: *Cc1c[nH]c2ccccc12]

43. The composition of any one of embodiments 1-42, wherein R¹ comprises:

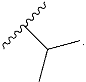 [SMILES: *C(C)C]

44. The composition of any one of embodiments 1-43, wherein R¹ comprises:

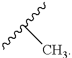

45. The composition of any one of embodiments 1-44, wherein R¹ comprises:

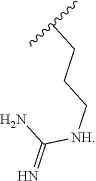 [SMILES: *CCCNC(=N)N]

46. The composition of any one of embodiments 1-45, wherein R¹ comprises:

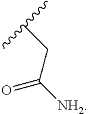 [SMILES: *CC(N)=O]

47. The composition of any one of embodiments 1-46, wherein R¹ comprises:

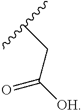 [SMILES: *CC(=O)O]

48. The composition of any one of embodiments 1-47, wherein R¹ comprises:

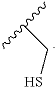 [SMILES: *CS]

49. The composition of any one of embodiments 1-48, wherein R¹ comprises:

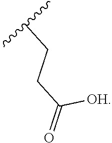 [SMILES: *CCC(=O)O]

50. The composition of any one of embodiments 1-49, wherein R¹ comprises:

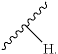

51. The composition of any one of embodiments 1-50, wherein R¹ comprises:

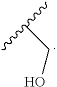 [SMILES: *CO]

52. The composition of any one of embodiments 1-51, wherein R¹ comprises:

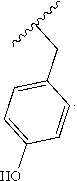 [SMILES: *Cc1ccc(O)cc1]

53. The composition of any one of embodiments 1-52, wherein R¹ comprises:

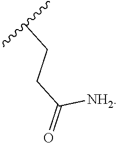 [SMILES: *CCC(N)=O]

54. The composition of any one of embodiments 6-53, wherein not more than 50%, not more than 45%, not more than 40%, not more than 35%, not more than 30%, not more than 25%, not more than 20%, not more than 15%, not more than 10%, or not more than 5% of amines of the chitosan are acetylated. 55. The composition of any one of embodiments 6-54, wherein the chitosan comprises a copolymer. 56. The composition of embodiment 55, wherein the copolymer is a linear copolymer, a block copolymer, an alternating copolymer, a periodic copolymer, a statistical copolymer, a stereoblock copolymer, a gradient copolymer, branched copolymer, a graft copolymer, a start copolymer, or a combination thereof. 57. The composition of embodiment 55 or embodiment 56, wherein the copolymer comprises Formula (I), Formula (II), Formula (III), Formula (IV), or combinations thereof:

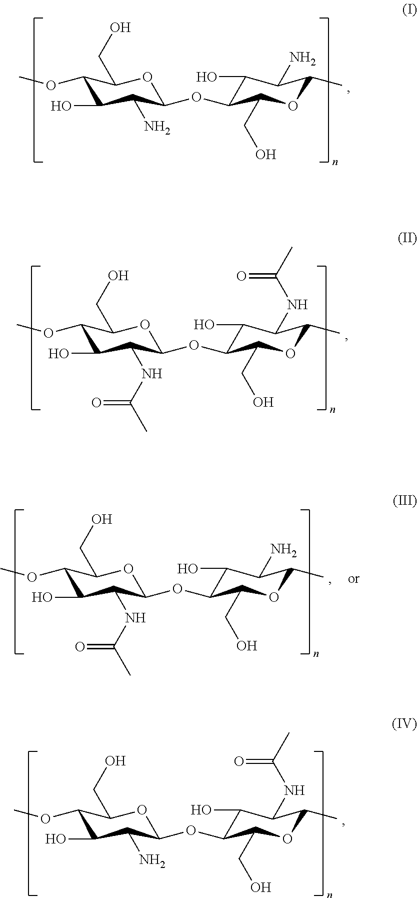

wherein each instance of n is independently an integer. 58. The composition of embodiment 57, wherein each instance of n is independently an integer of from 1 to 1000. 59. The composition of embodiment 57 or embodiment 58, wherein the chitosan comprises two or more of Formula (VI), Formula (VIII), Formula (X), Formula (XII), or Formula (XIV). 60. The composition of any one of embodiments 57-59, wherein the chitosan comprises a ratio of Formula (VI) to Formula (VIII) of from 10:1 to 1:10, from 100:1 to 10:1, or from 1:10 to 1:100. 61. The composition of any one of embodiments 6-60, wherein the alpha-keto acid is non-stochastically distributed about the chitosan. 62. The composition of any one of embodiments 6-61, wherein a stoichiometric ratio of the chitosan amine and the alpha-keto acid is from 1:1 to 1:10, from 1:1 to 1:100, from 1:1 to 1:1000, from 1:1 to 1:10,000, or from 1:1 to 1:100,000. 63. The composition of any one of embodiments 6-62, wherein a weight ratio of the chitosan to the alpha-keto acid is from 1:1 to 1:10, from 1:1 to 1:100, from 1:1 to 1:1000, from 1:1 to 1:10,000, or from 1:1 to 1:100,000. 64. The composition of any one of embodiments 1-63, further comprising a food component. 65. The composition of embodiment 64, formulated as a medical food. 66. The composition of embodiment 65, wherein the medical food is a beverage, a drink mix, or a solid food. 67. The composition of any one of embodiments 1-63, further comprising a pharmaceutically acceptable excipient. 68. The composition of embodiment 67, formulated for administration orally, topically, transdermally, rectally, intravenously, intra-arterially, intra-peritoneally, parenterally, or via inhalation. 69. A consumable formulation comprising the composition of any one of embodiments 1-68. 70. The consumable formulation of embodiment 69, wherein between about 30% and 90%, between about 50% and 80%, or between about 60% and 95% of dry weight of the consumable formulation is carbohydrates, fats, protein, or a combination thereof. 71. The consumable formulation of embodiment 69 or embodiment 70, wherein the consumable formulation is formulated as a liquid, a solid, a colloid, a gel, or a combination thereof. 72. The consumable formulation of any one of embodiments 69-71, wherein the consumable formulation is a medical grade food. 73. A medical food comprising the composition of any one of embodiments 1-66. 74. The medical food of embodiment 73, wherein the medical food is formulated as a beverage, a drink mix, or a solid food. 75. The medical food of embodiment 73 or embodiment 74, wherein the medical food is formulated as a solid food. 76. The medical food of embodiment 73 or embodiment 74, wherein the medical food is formulated as a beverage. 77. The medical food of any one of embodiments 73-76, wherein the medical food is a solid. 78. The medical food of any one of embodiments 73-76, wherein the medical food is a liquid. 79. The medical food of any one of embodiments 73-76, wherein the medical food is gelatinous. 80. The medical food of any one of embodiments 73-79, wherein the medical food is formulated as a beverage, a bar, a cereal, a sports drink, a gel, a gelatin, a gelatinous gummy, a cracker, a chip, a puff, a granola cereal, a granola bar, a tablet, a powder, or an additive. 81. The medical food of any one of embodiments 73-80, comprising a Cd content of less than 5 μg per daily serving or of less than 0.5 μg per g. 82. The medical food of any one of embodiments 73-81, comprising a Pb content of less than 5 μg per daily serving or less than 0.5 μg per g. 83. The medical food of any one of embodiments 73-82, comprising an As content of less than 15 μg per daily serving or less than 1.5 μg per g. 84. The medical food of any one of embodiments 73-83, comprising a Hg content of less than 30 μg per daily serving or less than 3 μg per g. 85. The medical food of any one of embodiments 73-84, comprising a Co content of less than 50 μg per daily serving or less than 5 μg per g. 86. The medical food of any one of embodiments 73-85, comprising a V content of less than 100 μg per daily serving or less than 10 μg per g. 87. The medical food of any one of embodiments 73-86, comprising a Ni content of less than 200 μg per daily serving or less than 20 μg per g. 88. The medical food of any one of embodiments 73-87, comprising a TI content of less than 8 μg per daily serving or less than 0.8 μg per g. 89. The medical food of any one of embodiments 73-88, comprising an Au content of less than 300 μg per daily serving or less than 30 μg per g. 90. The medical food of any one of embodiments 73-89, comprising a Pd content of less than 100 μg per daily serving or less than 10 μg per g. 91. The medical food of any one of embodiments 73-90, comprising an Ir content of less than 100 μg per daily serving or less than 10 μg per g. 92. The medical food of any one of embodiments 73-91, comprising an Os content of less than 100 μg per daily serving or less than 10 μg per g. 93. The medical food of any one of embodiments 73-92, comprising a Rh content of less than 100 μg per daily serving or less than 10 μg per g. 94. The medical food of any one of embodiments 73-93, comprising a Ru content of less than 100 μg per daily serving or less than 10 μg per g. 95. The medical food of any one of embodiments 73-94, comprising a Se content of less than 150 μg per daily serving or less than 15 μg per g. 96. The medical food of any one of embodiments 73-95, comprising an Ag content of less than 150 μg per daily serving or less than 15 μg per g. 97. The medical food of any one of embodiments 73-96, comprising a Pt content of less than 100 μg per daily serving or less than 10 μg per g. 98. The medical food of any one of embodiments 73-97, comprising a Li content of less than 550 μg per daily serving or less than 55 μg per g. 99. The medical food of any one of embodiments 73-98, comprising a Sb content of less than 1200 μg per daily serving or less than 120 μg per g. 100. The medical food of any one of embodiments 73-99, comprising a Ba content of less than 1400 μg per daily serving or less than 140 μg per g. 101. The medical food of any one of embodiments 73-100, comprising a Mo content of less than 3000 μg per daily serving or less than 300 μg per g. 102. The medical food of any one of embodiments 73-101, comprising a Cu content of less than 3000 μg per daily serving or less than 300 μg per g. 103. The medical food of any one of embodiments 73-102, comprising a Sn content of less than 6000 μg per daily serving or less than 600 μg per g. 104. The medical food of any one of embodiments 73-103, comprising a Cr content of less than 11000 μg per daily serving or less than 1100 μg per g. 105. The medical food of any one of embodiments 73-104, comprising a microbial plate count of no more than 1000 colony forming per mL, no more than 10,000 colony forming per mL, or no more than 20,000 colony forming per mL. 106. A method of increasing a protein level in a subject in need thereof, the method comprising administering to the subject the composition of any one of embodiments 1-68, the consumable formulation of any one of embodiments 69-72, or the medical food of any one of embodiments 73-105, thereby increasing the protein level in the subject. 107. The method of embodiment 106, wherein the subject does not have kidney disease. 108. The method of embodiment 106 or embodiment 107, wherein the subject is at risk of developing kidney disease. 109. The method of any one of embodiments 106-108, wherein the subject has a condition that puts the subject at risk of kidney disease. 110. The method of embodiment 109, wherein the condition is diabetes, high blood pressure, heart disease, obesity, a family history of chronic kidney disease, inherited kidney disorders, past damage to the kidneys, or old age. 111. The method of embodiment 106, wherein the subject has a kidney disease. 112. A method of treating disease in a subject in need thereof, the method comprising administering to the subject the composition of any one of embodiments 1-68, the consumable formulation of any one of embodiments 69-72, or the medical food of any one of embodiments 73-105, thereby treating the disease in the subject. 113. The method of embodiment 112, wherein the subject has kidney disease. 114. The method of embodiment 112 or embodiment 113, wherein the subject is at risk of developing kidney disease. 115. The method of embodiment 113 or embodiment 114, wherein the kidney disease is chronic kidney disease, Fabry disease, cystinosis, glomerulonephritis, IgA nephropathy, lupus nephritis, atypical hemolytic uremic syndrome, polycystic kidney disease, diabetic kidney disease, or uremia. 116. The method of any one of embodiments 112-115, wherein the subject is in an anabolic need.

### EXAMPLES

The invention is further illustrated by the following non-limiting examples.

### Example 1

Chitosan Compositions with Valine and Isoleucine Alpha-Keto Acids

This example describes the production of a composition comprising a chitosan and an alpha-keto acid. First, 250 mg of chitosan powder was suspended in 10 mL of a solvent mixture of 10% water in acetonitrile (80 mL/g) under vigorous stirring conditions. The stirred suspension of chitosan was then treated with keto acid compositions as follows. The chitosan was treated with either a molar equivalent of a single keto acid or a molar equivalent of a mixture of two keto acids. The single keto acid was made as a solution containing 3-methyl-2-oxobutanoic acid (alpha-keto acid analogue of valine) in 10 mL of the 10% water in acetonitrile (80 mL/g) solvent mixture. The mixture of two keto acids was made as a 1:1 mixture of 3-methyl-2-oxo-butanoic acid (alpha-keto acid analogue of valine) and 3-methyl-2-oxopentanoic acid (alpha-keto acid analogue of isoleucine) in 10 mL of the 10% water in acetonitrile (80 mL/g) solvent mixture. After the additions of the keto acid compositions to the chitosan, the chitosan powder immediately became more granular in appearance. The suspension was stirred overnight and then was filtered. The resulting solids were washed with acetonitrile and dried under dry nitrogen flow. The obtained product was the modified chitosan as a grey granular solid.

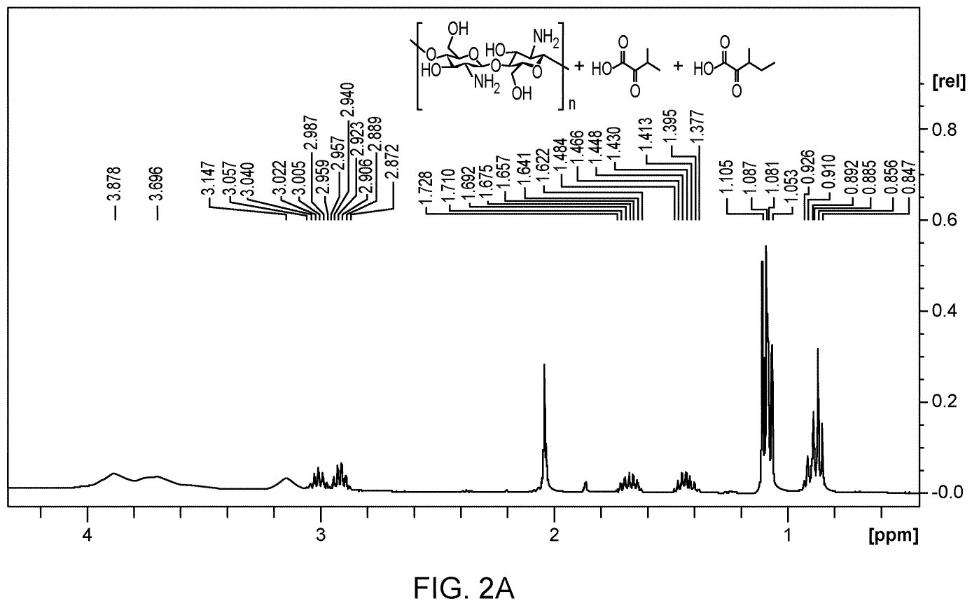

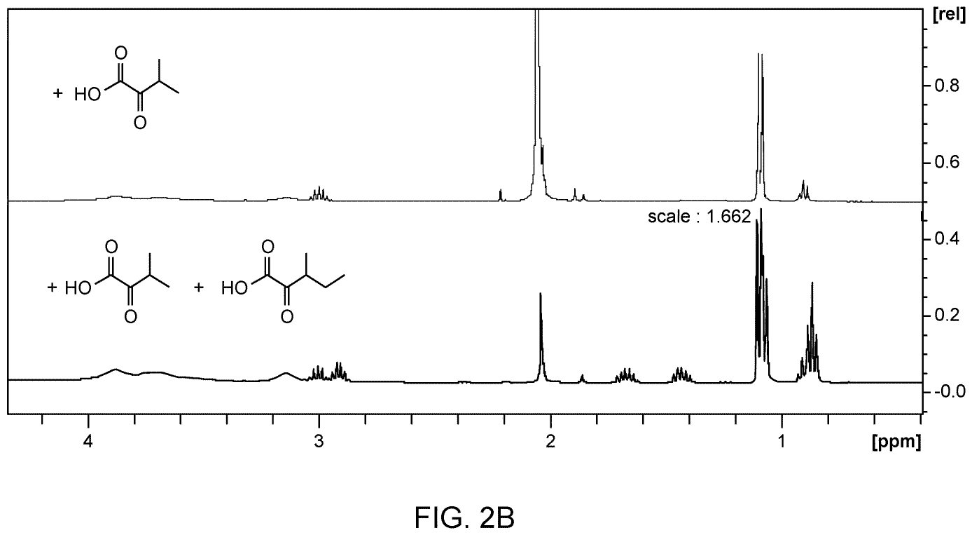

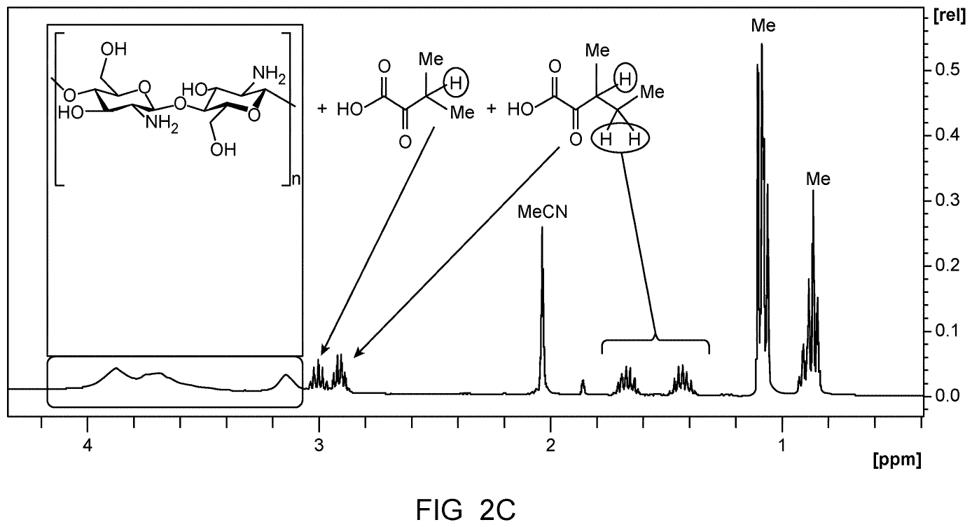

The chitosan alpha-keto acid compositions were characterized with proton NMR (¹H NMR) by dissolving about 5 mg of the product in 0.75 mL of deuterated water (D₂O) with 1% of deuterated acetic acid (CD₃CO₂D). The proton NMR spectrum of the chitosan composition with two keto acids is shown in FIG. 2A; H¹ NMR (D₂O+1% CD₃CO₂D, 400 MHz): δ 0.847, 0.866, 0.885, 0.892, 0.910, 0.926, 1.063, 1.081, 1.087, 1.105, 1.377, 1.395, 1.413, 1.430, 1.448, 1.466, 1.484, 1.622, 1.641, 1.657, 1.675, 1.692, 1.710, 1.728, 2.872, 2.889, 2.906, 2.923, 2.940, 2.957, 2.969, 2.987, 3.005, 3.022, 3.040, 3.057, 3.147, 3.696, and 3.878. The proton NMR peaks were assigned to each keto acids by overlaying the single keto acid chitosan composition with the double keto acid chitosan composition, as shown in FIG. 2B. The assigned signals for each keto acid in the double keto acid chitosan composition are detailed in FIG. 2C. As shown in FIG. 2C, the signals from the hydrogen on the tertiary carbon of 3-methyl-2-oxobutanoic acid (alpha-keto acid analogue of valine) are shown to be around 3 ppm and the signals from the hydrogen on the tertiary carbon of 3-methyl-2-oxopentanoic acid (alpha-keto acid analogue of isoleucine) are around 2.9 ppm with the signals from hydrogens on the secondary carbon of 3-methyl-2-oxopentanoic acid (alpha-keto acid analogue of isoleucine) around 1.15-1.8 ppm. Further shown in FIG. 2C, the signal from the protons on the chitosan are in the range of about 3.1-4.1 ppm. The high contrast in peak shape between the chitosan signals and the keto acids may suggest that the keto acids are disassociated from the polymer in the low pH aqueous NMR solution. The disassociation also supports the ionic mode of interaction between the chitosan polymer and the keto acids. There may also be non-specific modes of interaction between the chitosan polymer and the keto acids such as adsorption of the keto acids to the surface of the polymer or sequestration of the keto acids in the microscopic structure of the polymer (e.g., the pores of the solid product).

This procedure is repeated for the alpha-keto acid analogues of each essential amino acid including histidine, isoleucine, leucine, lysine, methionine, phenylalanine, threonine, tryptophan, and valine. Specifically, to the chitosan solution, a molar equivalent is added of a mixture of the following alpha-keto acids: 3-(1H-imidazol-4-yl)-2-oxopropanoic acid (alpha-keto acid analogue of histidine), 3-methyl-2-oxopentanoic acid (alpha-keto acid analogue of isoleucine), 4-methyl-2-oxopentanoic acid (alpha-keto acid analogue of leucine), 6-amino-2-oxohexanoic acid (alpha-keto acid analogue of lysine), 4-(methylthio)-2-oxobutanoic acid (alpha-keto acid analogue of methionine), 2-oxo-3-phenylpropanoic acid (alpha-keto acid analogue of phenylalanine), 3-hydroxy-2-oxobutanoic acid (alpha-keto acid analogue of threonine), 3-(1H-indol-3-yl)-2-oxopropanoic acid (alpha-keto acid analogue of tryptophan), and 3-methyl-2-oxobutanoic acid (alpha-keto acid analogue of valine). The product is then characterized by proton NMR.

### Example 2

### Chitosan Alpha-Keto Acids Compositions

A solution with a known concentration of chitosan is made in a solvent mixture of water in acetonitrile under vigorous stirring conditions. Increasing quantities of the alpha-keto acids are incrementally added to this solution under stirring. The alpha-keto acids are added in ratios comparable to the amino acid content of human albumin protein or the recommended daily requirement, as displayed in TABLE 4.

TABLE 4
Amino Acid Composition of Human Albumin Protein and Daily Requirement
of Amino Acids

| | Weight | Amount in | Amount in | | kg for | Daily |
|---|---|---|---|---|---|---|
| | total | 100 g albumin | 100 g albumin | | animal | requirement |
| Amino Acid | (g/mol) | moles (g) | moles (mg) | % of total | studies | (mg per kg) |
| Tryptophan | 408.46 | 0.24 | 244.8 | 34.0 | 0.306184431 | 5 |
| Glycine | 975.91 | 0.10 | 102.5 | 14.2 | 0.128151256 | 22 |
| Methionine | 1044.47 | 0.10 | 95.7 | 13.3 | 0.119739287 | 19 |
| Isoleucine | 1180.53 | 0.08 | 84.7 | 11.8 | 0.105938936 | 19 |
| Histidine | 2482.40 | 0.04 | 40.3 | 5.6 | 0.050380314 | 14 |
| Tyrosine | 3442.61 | 0.03 | 29.0 | 4.0 | 0.036328278 | 100 |
| Threonine | 3454.48 | 0.03 | 28.9 | 4.0 | 0.03620345 | 20 |
| Arginine | 4703.40 | 0.02 | 21.3 | 3.0 | 0.026590146 | 55 |
| Valine | 5037.45 | 0.02 | 19.9 | 2.8 | 0.024826865 | 24 |
| Phenylalanine | 5781.65 | 0.02 | 17.3 | 2.4 | 0.021631211 | 33 |
| Leucine | 8394.88 | 0.01 | 11.9 | 1.7 | 0.014897663 | 42 |
| Cysteine | 8410.50 | 0.01 | 11.9 | 1.7 | 0.014869995 | 4 |
| Lysine | 8771.40 | 0.01 | 11.4 | 1.6 | 0.014258168 | 30 |
| Total | | | 719.6 | 100.0 | 0.9 | |

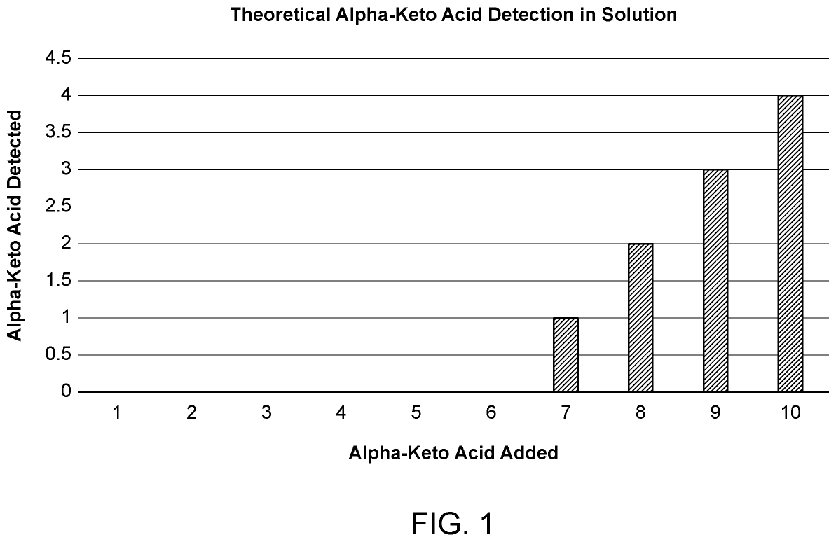

Following 15 additional minutes of stirring, a portion of the solution is collected and filtered, generating a supernatant containing alpha-keto acid-bound chitosan. The concentration of alpha-keto acid in the supernatant is determined by high-performance liquid chromatography (HPLC). A theoretical plot of alpha-keto acid detection as a function of alpha-keto acid added is provided as FIG. 1, showing that alpha-keto acid detection may increase as more alpha-keto acid is added to the solution. The chitosan formula is Formula I as shown below.

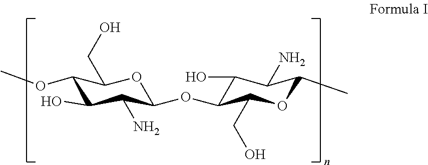

The expected binding can be determined by treating chitosan as a polymer of conjugated glucosamine and monitoring the molecular weight (MW) of the composition during alpha-keto acid association.

### Example 3

### Synthesis of Chitosan Keto Acid Compositions

This example describes the synthesis of various chitosan keto acid compositions and characteristics of the compositions. Compositions of chitosan and keto acids were synthesized by combining a chitosan solution and a mixture of keto acids and other chemical components. Each sample synthesized is provided in TABLE 5. As noted in the “Description” column of TABLE 5, samples were synthesized with various percentages of chitosan (CS) (e.g., 0.5%, 1%, 3%, 6%, and 8%), the keto acid mixture (KA), and other chemical components including tripoly phosphate (TPP), sodium alginate (alginate and ALG), (hydroxypropyl)methyl cellulose phthalate (HPMCP), polysorbate 80 (PS80), Poloxamer 188 (P188), polyethylene glycol 3350 (PEG3350), N-(carbonyl-methoxypolyethylene glycol-2000)-1,2-distearoyl-sn-glycero-3-phosphoethanol amine sodium salt (DSPE-PEG), citrate (Cit), magnesium stearate (MgSt), and mannitol.

As noted below in TABLE 5 in the “Conclusions” column, each of the synthetic methods of the chitosan keto acid compositions resulted in different properties of the compositions such as keto acid binding properties of the compositions. Samples 69, 70, 75, 80, 81, and 82 were then further characterized for keto acid binding as described in EXAMPLE 4.

TABLE 5
Summary of Keto Acid Chitosan Sample Formulations

| Sample | Description | Comments | Conclusions |
|---|---|---|---|
| 1 | 1% CS + KA + TPP | | Requires further optimization |
| 2 | 3% CS + KA + TPP | | The TPP addition after KAs showed |
| | | | slightly higher decrease in KA in the |
| | | | supernatant. |
| 3 | 3% CS + TPP + KA | | Phe KA and Trp KA showed a greater |
| | | | degree of decrease in KA in the |
| | | | supernatant. |
| 4 | 3% CS + KA + TPP + | | Alginate had higher decrease of KAs: |
| | alginate | | His, Ile, and Leu, in the supernatant. |
| 5 | 3% CS + KA + TPP + | | Alginate had higher decrease in KA: |
| | HPMCP | | His, Ile, and Leu, in the supernatant. |
| 6 | 3% CS + KA + TPP | TPP added dropwise (2 | |
| | | hr) | |
| 7 | 3% CS + KA + TPP | TPP added dropwise (2 | Decrease in KA in the supernatant |
| | | hr) | greater than 45% |
| 8 | 3% CS + KA + TPP | TPP added dropwise (2 | Heating did not increase the % decrease |
| | | hr), heated | in KA in the supernatant. |
| 9 | 3% CS + KA + TPP + | TPP added dropwise (2 | PS80 showed a slight decrease of KA in |
| | PS80 | hr), heated | in the supernatant when compared to |
| | | | P188 and PEG3350. |
| 10 | 3% CS + KA + TPP + | TPP added dropwise (2 | PS80 showed a slight decrease of KA in |
| | P188 | hr), heated | supernatant when compared to P188 |
| | | | and PEG3350. |
| 11 | 3% CS + KA + TPP + | TPP added dropwise (2 | PS80 showed a slight crease of KA in |
| | PEG3350 | hr), heated | supernatant when compared to P188 |
| | | | and PEG3350. |
| 12 | 3% CS + KA + TPP | KAs added directly | Lyophilization enhanced the decrease of |
| | | (without additional | KA in the supernatant: Phe and Trp. |
| | | solvent) | |
| 13 | 3% CS + KA + TPP | KAs added directly (with | This had lower decrease of KA in the |
| | | no Ethanol) | supernatant than the lyophilized |
| | | | experiment. |
| 14 | 3% CS + KA + alginate | KAs added directly | Alginate had a higher decrease of KA in |
| | | (without additional | the supernatant: Gly, Ile, and Leu when |
| | | solvent) | compared to HPMCP (Sample 15) and |
| | | | DSPE-PEG (Sample 16). |
| 15 | 3% CS + KA + HPMCP | KAs added directly | HPMCP had lower decrease of KA in |
| | | (without additional | the supernatant than alginate. |
| | | solvent) | |
| 16 | 3% CS + KA + | KAs added directly | DSPE-PEG had lower decrease of KA |
| | DSPE-PEG | (without additional | in the supernatant than alginate. |
| | | solvent) | |
| 17 | 3% CS + KA + | KAs added directly | TPP addition after 2nd polymer |
| | alginate + TPP | (without additional | addition, hindered the decrease of KA |
| | | solvent) | in the supernatant. |
| 18 | 3% CS + KA + | KAs added directly | TPP addition after 2nd polymer |
| | HPMCP + TPP | (without additional | addition, hindered the decrease of KA |
| | | solvent) | in the supernatant. |
| 19 | 3% CS + KA + | KAs added directly | TPP addition after 2nd polymer |
| | DSPE-PEG + TPP | (without additional | addition, hindered the decrease of KA |
| | | solvent) | in the supernatant. |
| 20 | 1% CS + KA + TPP | KAs added directly | 6% CS and 8% CS showed an |
| | | (without additional | improvement in the decrease of KA in |
| | | solvent) | the supernatant of KAs: Trp and Phe. |
| 21 | 3% CS + KA + TPP | KAs added directly | 6% CS and 8% CS showed an |
| | | (without additional | improvement in the decrease of KA in |
| | | solvent) | the supernatant of KAs: Trp and Phe. |
| 22 | 6% CS + KA + TPP | KAs added directly | 6% CS and 8% CS showed an |
| | | (without additional | improvement in the decrease of KA in |
| | | solvent) | the supernatant of KAs: Trp and Phe. |
| 23 | 8% CS + KA + TPP | KAs added directly | 6% CS and 8% CS showed an |
| | | (without additional | improvement in the decrease of KA in |
| | | solvent) | the supernatant of KAs: Trp and Phe. |
| 24 | 3% CS + KA + TPP | KAs added directly | Only Phe KA and Trp KA bound. |
| | (2 hr) | (without additional | |
| | | solvent) | |
| 25 | Lyophilization of | KAs added directly | Greater than 10% bound KAs. |
| | Sample 24 | (without additional | |
| | | solvent) | |
| 26 | 3% CS + KA + Cit (2 | KAs added directly | Had microparticles, but Ile KA and Leu |
| | hr) | (without additional | KA did not bind. |
| | | solvent) | |
| 27 | Lyophilization of | KAs added directly | Bound KAs range = 19-83%. |
| | Sample 26 | (without additional | |
| | | solvent) | |
| 28 | 3% CS + KA + 0.5% | KAs added directly | Bound KAs range = 49-99%. |
| | alginate | (without additional | |
| | | solvent) | |
| 29 | Lyophilization of | KAs added directly | Bound KAs range = 34-99%. |
| | Sample 28 | (without additional | |
| | | solvent) | |
| 30 | 6% CS + KA + 10% | KAs added directly | Formulations with 1% alginate |
| | TPP + 1% alginate | (without additional | had >70% KAs bound. |
| | | solvent) | |
| 31 | Lyophilization of | KAs added directly | Formulations with 1% alginate |
| | Sample 30 | (without additional | had >70% KAs bound. |
| | | solvent) | |
| 32 | 6% CS + KA + 10% TPP | KAs added directly | Ile KA had less binding with the |
| | | (without additional | lyophilized formulation. |
| | | solvent) | |
| 33 | Lyophilization of | KAs added directly | Ile KA had less binding with the |
| | Sample 32 | (without additional | lyophilized formulation. |
| | | solvent) | |
| 34 | 6% CS + KA + 10% | KAs added directly | Formulations with 1% alginate |
| | Cit + 1% alginate | (without additional | had >70% KAs bound. |
| | | solvent) | |
| 35 | Lyophilization of | KAs added directly | Formulations with 1% alginate |
| | Sample 34 | (without additional | had >70% KAs bound. |
| | | solvent) | |
| 36 | 6% CS + KA + 10% Cit | KAs added directly | Ile KA had less binding with the |
| | | (without additional | lyophilized formulation. |
| | | solvent) | |
| 37 | Lyophilization of | KAs added directly | Ile KA had less binding with the |
| | Sample 36 | (without additional | lyophilized formulation. |
| | | solvent) | |
| 38 | 1% CS + KA + 5% TPP + | KAs added directly | Formulations with 1% alginate |
| | 5% Cit + 1% alginate | (without additional | had >70% KAs bound. |
| | | solvent) | |
| 39 | Lyophilization of | KAs added directly | Formulations with 1% alginate |
| | Sample 38 | (without additional | had >70% KAs bound. |
| | | solvent) | |
| 40 | 1% CS + KA + 5% TPP + | KAs added directly | This formulation had lower KA binding |
| | 5% Cit | (without additional | than 6% CS with either TPP or Cit. |
| | | solvent) | |
| 41 | Lyophilization of | KAs added directly | This formulation had lower KA binding |
| | Sample 40 | (without additional | than 6% CS with either TPP or Cit. |
| | | solvent) | |
| 69 | 0.5% CS + | | Compositions used for binding |
| | 0.1% Mg ST + | | characterization as described in |
| | 1% ALG | | EXAMPLE 4 |
| | (5 mL) | | |
| 70 | 1% CS + | | Compositions used for binding |
| | 5% TPP + | | characterization as described in |
| | 0.2% Mg ST + | | EXAMPLE 4 |
| | 1% ALG (5 mL) | | |
| 75 | Lyophilization of | | Compositions used for binding |
| | Sample 69 | | characterization as described in |
| | | | EXAMPLE 4 |
| 80 | 0.5% CS + | pH was increased with | Compositions used for binding |
| | 0.1% Mg ST + | NaOH before addition of | EXAMPLE 4 and lyophilized |
| | 1% ALG (5 mL) | MgSt; solution was | characterization as described in |
| | | sonicated before and after | formulations as described in |
| | | pH adjustment; solution | EXAMPLE 5 |
| | | was sonicated after the | |
| | | last addition (ALG) | |
| 81 | 0.5% CS + | pH was increased with | characterization as described in |
| | 0.025% DSPE-PEG + | NaOH before addition of | formulations as described in |
| | 0.1% Mg | MgSt; solution was | Compositions used for binding |
| | ST + | sonicated before and after | EXAMPLE 4 and lyophilized |
| | 1% ALG | pH adjustment; solution | EXAMPLE 5 |
| | (5 mL) | was sonicated after the | |
| | | last addition (ALG) | |
| 82 | 0.5% CS + | pH was increased with | Compositions used for binding |
| | 0.025% DSPE-PEG + | NaOH before addition of | characterization as described in |
| | 5% mannitol | MgSt; solution was | EXAMPLE 4 and lyophilized |
| | (5 mL) | sonicated before and after | formulations as described in |
| | | pH adjustment; solution | EXAMPLE 5 |
| | | was sonicated after the | |
| | | last addition (mannitol) | |

As shown in TABLE 5, Samples 80, 81, and 82 were synthesized with 0.5% chitosan (CS). Samples 80, 81, and 82 were also synthesized with a keto acid (KA) mixture with concentrations relevant to keto acid concentrations desirable for an in vivo study and provided below in TABLE 6. Samples 80, 81, and 82 were synthesized by combining a 0.5% chitosan (CS) solution with a keto acid (KA) solution with the concentrations in TABLE 6.

TABLE 6
Concentration of Keto Acids for In Vivo Study and Formulations

| | Amount required in | Volume per day per | Concentration |
|---|---|---|---|
| Keto Acid | 500 g rat (mg) | rat (mL) | (mg/mL) |
| Tryptophan | 2 | 2.5 | 0.8 |
| Glycine | 11 | 2.5 | 4.4 |
| Methionine | 3.75 | 2.5 | 1.5 |
| Isoleucine | 10 | 2.5 | 14.0 |
| Histidine | 5 | 2.5 | 2.0 |
| Valine | 13 | 2.5 | 5.2 |
| Phenylalanine | 6.25 | 2.5 | 2.5 |
| Leucine | 19.5 | 12.5 | 7.8 |
| Cysteine | 2 | 2.5 | 0.8 |
| Tyrosine | 16.25 | 2.5 | 2.5 |

Sample 80 was synthesized by first combining the 0.5% chitosan (CS) solution with a keto acid (KA) solution and then increasing the pH of the solution with sodium hydroxide (NaOH) before the addition of 0.1% magnesium stearate (MgSt). Sample 80 was sonicated before and after the pH adjustment. Sample 80 was also sonicated before the last addition, i.e., before the addition of 1% alginate. Sample 80 was then formulated into liquid or lyophilized formulations to test the keto acid (KA) binding properties of the composition.

Sample 81 was synthesized by first combining the 0.5% chitosan (CS) solution with a keto acid (KA) solution and a 0.025% solution of DSPE-PEG. The pH of Sample 81 was then increased with sodium hydroxide (NaOH) before the addition of 0.1% magnesium stearate (MgSt). Sample 81 was sonicated before and after the pH adjustment. Sample 81 was also sonicated before the last addition, i.e., before the addition of 1% alginate. Sample 81 was then formulated into liquid or lyophilized formulations to test the keto acid (KA) binding properties of the composition.

Sample 82 was synthesized by first combining the 0.5% chitosan (CS) solution with a keto acid (KA) solution and a 0.025% solution of DSPE-PEG. The pH of Sample 82 was then increased with sodium hydroxide (NaOH). Sample 82 was sonicated before and after the pH adjustment. Sample 82 was also sonicated before the last addition, i.e., before the addition of 5% mannitol. Sample 82 was then formulated into liquid or lyophilized formulations to test the keto acid (KA) binding properties of the composition.

Samples 80-82 did not include the histidine keto acid analogue in the keto acid (KA) mixture.

### Example 4

### Characterization of Percent Bound Keto Acid of Chitosan Keto Acid Compositions

This example describes the characterization of the chitosan keto acid compositions synthesized in EXAMPLE 3. Specifically, Samples 70, 69, 75, and 80-82 synthesized as described in EXAMPLE 3 were evaluated for keto acid (KA) binding capabilities and compared. Keto acid (KA) binding was evaluated using high performance liquid chromatography (HPLC). The percent keto acid (KA) was evaluated by comparing the HPLC spectrum of each of the samples to the HPLC spectrum of a keto acid control mixture (control) to evaluate how much keto acid (KA) remained in solution (un-bound to the chitosan). The percent bound keto acid (KA) to the chitosan compositions was then calculated by the difference of 100% and the percent of keto acid (KA) unbound as determined by HPLC.

The HPLC parameters used are provided in TABLE 7.

TABLE 7
HPLC Method Parameters

| Parameter | Value |
|---|---|
| Column | Thermo Scientific Acclaim mixed- |
| | mode WAX-1, 3 μm, 2.1 × 150 mm |
| | (070089) |
| Mobile Phase | MPA: 6 mM sodium phosphate, pH = 6.0 |
| | MPB: 6 mM sodium phosphate, |
| | pH = 6.0 and 75% acetonitrile |
| Flow rate | 0.35 mL/min |
| Column temperature | 30° C. |
| Autosampler | 5° C. |
| temperature | |
| Detector | 210 nm, 220 nm, 250 nm, 280 nm, |
| | and reference of 360 nm |
| Gradient | 0 to 90% B over 45 minutes |
| Injection volume | 0.25 μL |

The percent keto acid (KA) bound was measured for sample 70 and is provided in TABLE 8. As shown in TABLE 8, Sample 70 which comprises 1% chitosan (CS), 5% tripoly phosphate (TPP), 0.2% magnesium stearate (MgSt), and 1% alginate (ALG) in a 5 ml volume, showed properties of keto acid binding for His, Val, Met, Ile, Tyr, Leu, Phe, and Trp keto acid analogues as measured by the absorbance at 210 nm and 280 nm.

TABLE 8
Percent Keto Acid Binding of Sample 70

| | | | | Sample 70 |
|---|---|---|---|---|
| | | | | measured binding |
| | | Sample 70 | Sample 70 | (subtracted blank |
| KA Analogue | Control | (210 nm) | (280 nm) | response) |
| His | 0.0% | 78.9% | 79.3% | 78.9% |
| Gly | 0.0% | 0.0% | | 0.0% |
| Val | 0.0% | 24.5% | | 24.5% |
| Met | 0.0% | 39.2% | | 39.2% |
| Ile | 0.0% | 41.5% | | 41.5% |
| Tyr | 0.0% | 58.3% | 58.3% | 58.3% |
| Leu | 0.0% | 37.8% | | 37.8% |
| Phe | 0.0% | 34.0% | 34.9% | 70.7% |
| Trp | 0.0% | 100.0% | 99.3% | 100.0% |

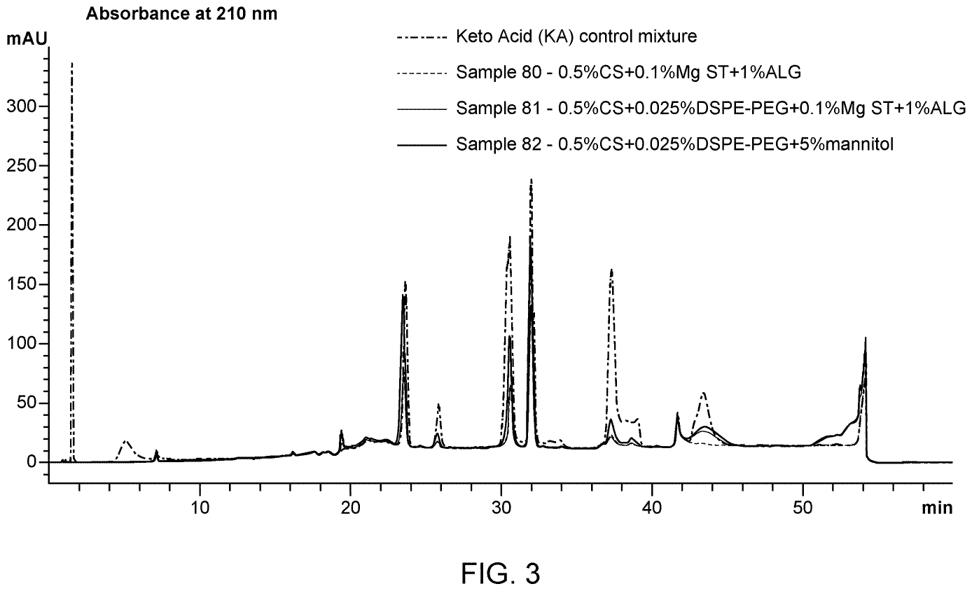

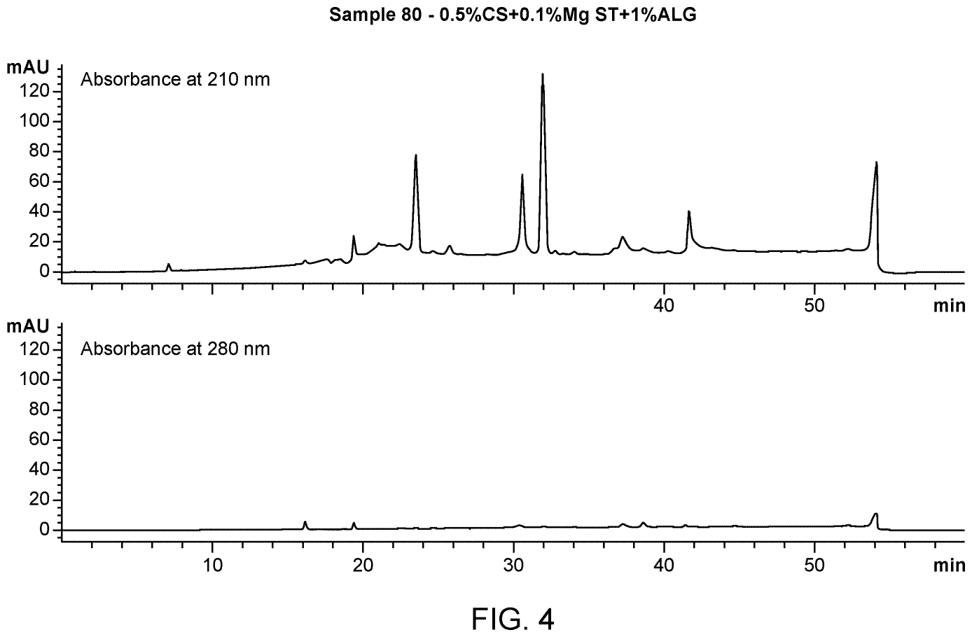

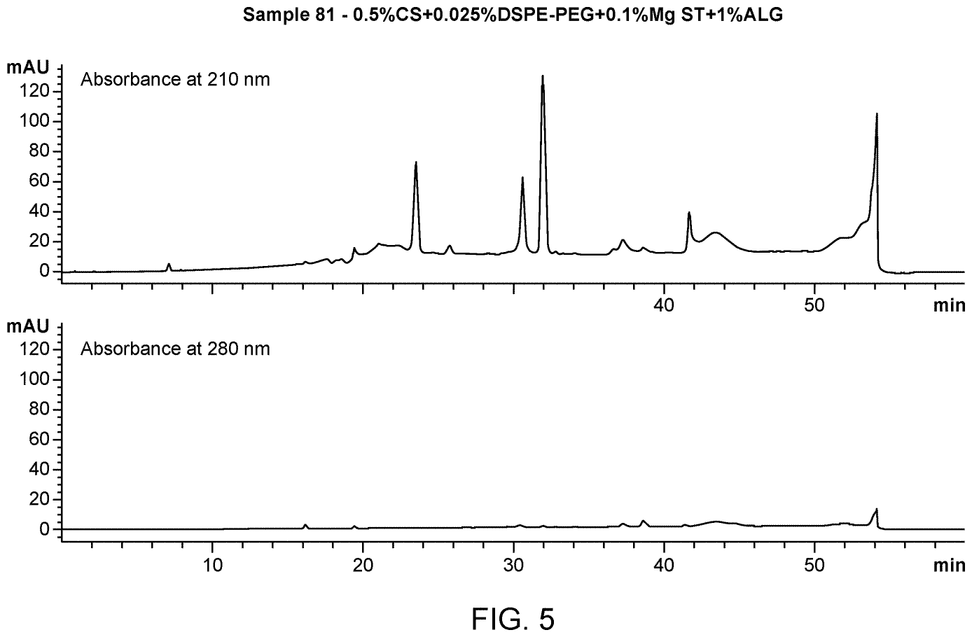

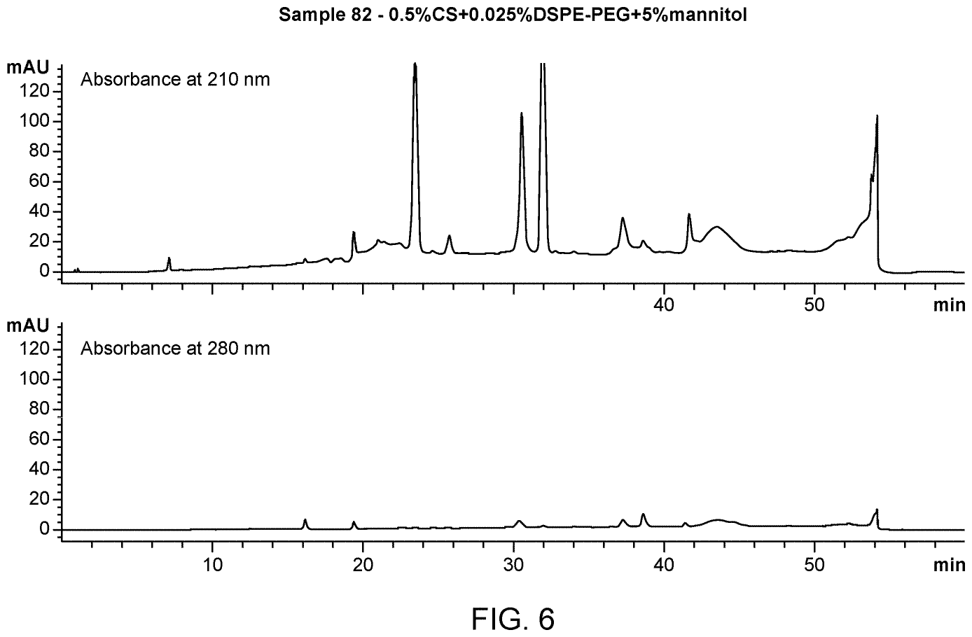

An overlay of the keto acid (KA) spectra for the keto acid control mixture, Sample 80, Sample 81, and Sample 82 is provided in FIG. 3, of the absorbance measured at 210 nm. The individual HPLC spectrum for Sample 80 is provided in FIG. 4, including the absorbance measured at 210 nm (top) and the absorbance measured at 280 nm (bottom). The individual HPLC spectrum for Sample 81 is provided in FIG. 5, including the absorbance measured at 210 nm (top) and the absorbance measured at 280 nm (bottom). The individual HPLC spectrum for Sample 82 is provided in FIG. 6, including the absorbance measured at 210 nm (top) and the absorbance measured at 280 nm (bottom).

The percent binding of keto acid (KA) analogues for Samples 80-82 in a liquid formulation is provided in TABLE 10 with a comparison to the keto acid (KA) analogue binding of Sample 69. As shown in TABLE 10, binding of the liquid formulation (prior to lyophilization) was comparable between Sample 80 and Sample 81 and had increased keto acid (KA) analogue binding from Sample 82. Further Sample 80 and Sample 81 also had increased keto acid (KA) analogue binding than Sample 69 other than Gly and Ile demonstrating that the synthetic method of Sample 80 and Sample 81 achieved favorable keto acid (KA) analogue properties of the chitosan keto acid compositions.

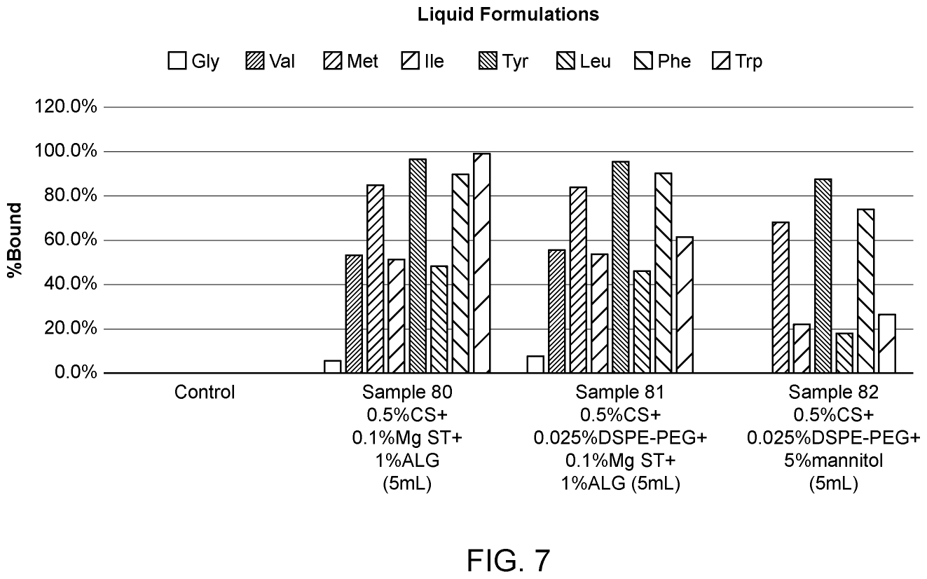

The percent binding of keto acid (KA) analogues is also shown in FIG. 7 for the liquid formulations of Sample 80, Sample 81, and Sample 82.

TABLE 10
Calculated Percent Binding Results in Liquid Formulations

| KA | | | | | |
|---|---|---|---|---|---|
| analogue | Control | Sample 69 | Sample 80 | Sample 81 | Sample 82 |
| His | 0.0% | 95.6% | N/A | N/A | N/A |
| Gly | 0.0% | 55.4% | 5.9% | 7.8% | 0.0% |
| Val | 0.0% | 10.1% | 53.3% | 55.3% | 0.0% |
| Met | 0.0% | 52.7% | 85.1% | 84.3% | 68.0% |
| Ile | 0.0% | 67.4% | 51.5% | 53.6% | 22.0% |
| Tyr | 0.0% | 80.7% | 96.4% | 95.8% | 87.6% |
| Leu | 0.0% | 6.7% | 48.7% | 46.1% | 18.1% |
| Phe | 0.0% | 43.1% | 89.5% | 90.3% | 74.0% |
| Trp | 0.0% | 94.0% | 99.1% | 61.9% | 26.6% |

The percent binding of keto acid (KA) analogues for Samples 80-82 in a lyophilized formulation is provided in TABLE 11 with a comparison to the keto acid (KA) analogue binding of Sample 75 (a lyophilization of Sample 69). As shown in TABLE 11, the lyophilized formulations of Sample 80 and Sample 81 and had increased keto acid (KA) analogue binding from the lyophilized formulation of Sample 82. Further, Sample 80 and Sample 81 also had comparable or decreased keto acid (KA) analogue binding than the corresponding liquid formulations of Sample 80 and Sample 81.

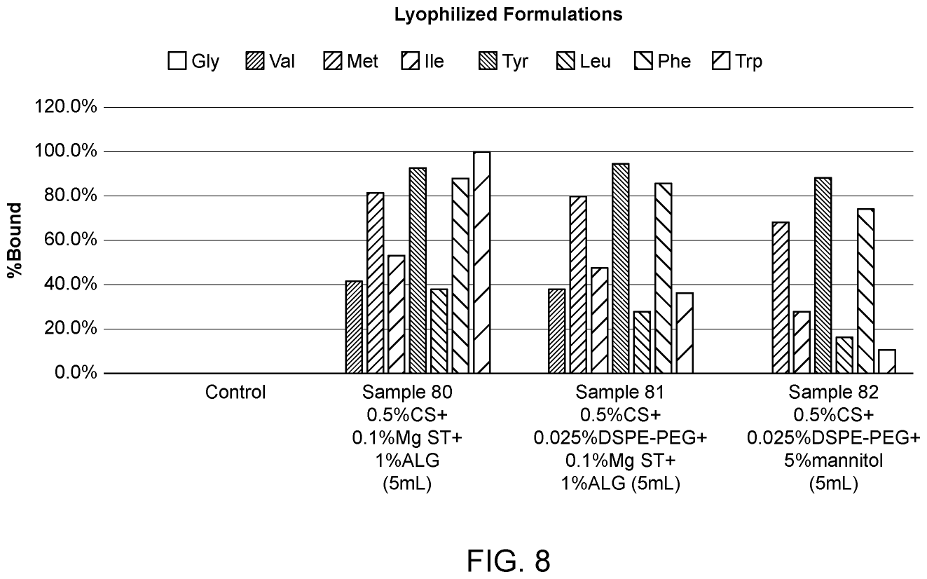

The percent binding of keto acid (KA) analogues is also shown in FIG. 8 for the lyophilized formulations of Sample 80, Sample 81, and Sample 82.

TABLE 11
Calculated Percent Binding Results in Lyophilized Formulations

| KA | | | | | |
|---|---|---|---|---|---|
| analogue | Control | Sample 75 | Sample 80 | Sample 81 | Sample 82 |
| His | 0.0% | 99.2% | N/A | N/A | N/A |
| Gly | 0.0% | 68.7% | 0.0% | 0.0% | 0.0% |
| Val | 0.0% | 67.8% | 41.7% | 38.2% | 0.0% |
| Met | 0.0% | 90.5% | 81.7% | 79.5% | 68.1% |
| Ile | 0.0% | 76.3% | 53.4% | 47.7% | 28.1% |
| Tyr | 0.0% | 92.7% | 92.2% | 94.4% | 88.0% |
| Leu | 0.0% | 68.3% | 37.8% | 28.1% | 16.3% |
| Phe | 0.0% | 81.4% | 87.9% | 85.8% | 74.1% |
| Trp | 0.0% | 88.1% | 100.0% | 36.5% | 10.8% |

### Example 5

### Formulation of Chitosan Keto Acid Compositions

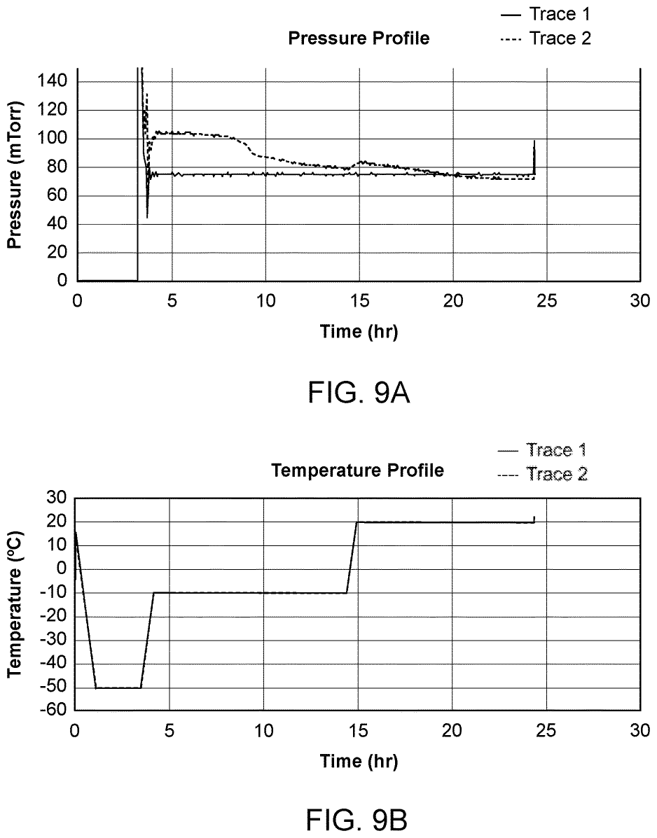

This example describes the formulation of the chitosan keto acid compositions synthesized in EXAMPLE 3 and characterized in EXAMPLE 4. The chitosan keto acid compositions of Sample 80, Sample 81, and Sample 82 were lyophilized producing a lyophilized chitosan keto acid formulation. The lyophilization parameters used for making lyophilized formulations of Sample 80, Sample 81, and Sample 82 are provided in TABLE 12. The pressure and temperature change over the time of the lyophilization cycle are shown in FIG. 9A and FIG. 9B, respectively. The pressure profile in FIG. 9A includes the set lyophilizer pressure (“Trace 1”) as well as the measured Pirani pressure (“Trace 2”). The temperature profile in FIG. 9B includes the set temperature in the lyophilizer (“Trace 1”) and the measured temperature in the lyophilizer (“Trace 2”). The Pirani pressure is higher than the set pressure when there is moisture in the system and will decrease to the set pressure once the moisture is removed. Accordingly, as shown in FIG. 9A and FIG. 9B, the primary drying of the sample was complete at −10° C. and ready for the secondary drying step that was performed at 20° C.

TABLE 12
Lyophilization Method Parameters

| Step | Ramp or Hold Temperature | Time (hr) |
|---|---|---|
| Freezing | Ramp 1: from ambient to −50° C. | 1.1 |
| | at 1° C./min | |
| | Hold at −50° C. | 2.4 |
| Primary | Ramp 2: from −50° C. | 0.7 |
| Drying | to −10° C. at 1° C./min | |
| | Hold at −10° C. | 10.2 |
| Secondary | Ramp 3: from −10° C. | 0.5 |
| Drying | to 20° C. at 1° C./min | |
| | Hold at 20° C. | 9.4 |

| | |
|---|---|
| Total Cycle Time (hr) | 24.3 |

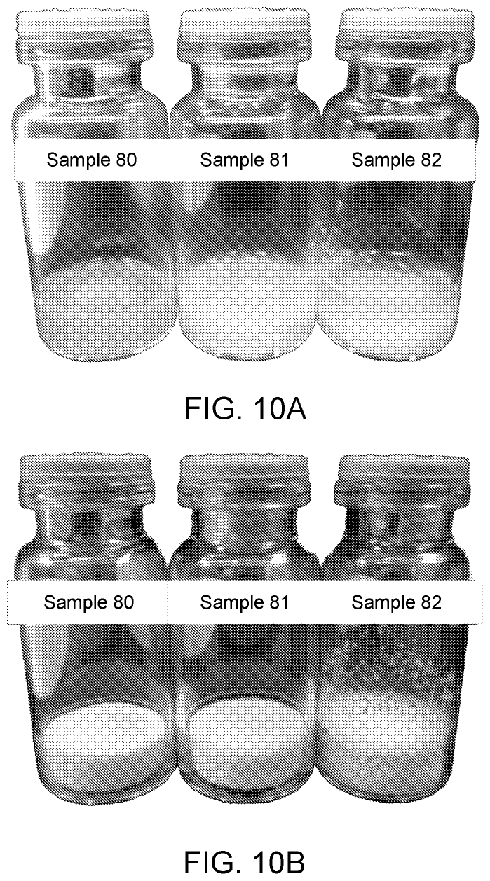

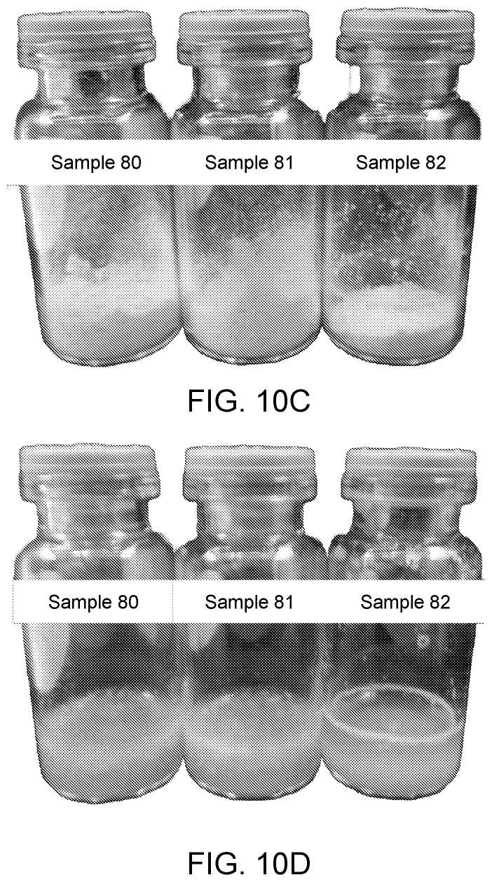

The lyophilized formulations of Sample 80, Sample 81, and Sample 82 were characterized before and after the lyophilization cycle and as well as after grinding and reconstitution. The appearance of Sample 80, Sample 81, and Sample 82 was observed before lyophilization (FIG. 10A), after lyophilization (FIG. 10B), after grinding (FIG. 10C), and after reconstitution (FIG. 10D). As shown in FIG. 10A, before lyophilization, Sample 80 and Sample 81 were gels and Sample 82 had solids at the bottom of the vial. As shown in FIG. 10B, after lyophilization, Sample 80 and Sample 81 produced a solid cake. As shown in FIG. 10C, the lyophilized samples were easily ground into a powder. As shown in FIG. 10D, after reconstitution, Sample 80 and Sample 81 produced a gel and Sample 82 had solid at the bottom of the vial. A lyophilized powder was also produced from the chitosan and keto acid (KA) analogue compositions by lyophilizing the liquid formulation in a lyophilization tray and then grind up the resulting lyophilized cakes. The lyophilization formulation of the chitosan and keto acid (KA) analogue compositions resulted in a cake that was readily ground into a free-flowing powder suitable for the use in the medical grade food or medical food compositions described herein.

### Example 6

### Toxicity Testing of Formulations of Chitosan Keto Acid Compositions

This example describes toxicity testing of the lyophilized formulations of chitosan keto acid compositions as described in EXAMPLE 5. The lyophilized formulations of Sample 80 and Sample 81 are ground to a free-flowing powder. The free-flowing powder is formulated into a medical grade food and administered to an animal subject. The toxicity is measured of the medical grade food in the animal subject to evaluate its safety for a medical grade food composition and medical food compositions to be administered to human subjects.

### Example 7

### Medical Grade Food Compositions Comprising Chitosan Alpha-Keto Acids

This example describes a composition of a medical grade food that comprises chitosan alpha-keto acids. A chitosan alpha-keto acid compositions is prepared as described in EXAMPLE 1-EXAMPLE 4 or formulated as described in EXAMPLE 5. Each chitosan alpha-keto acid has a chitosan polymer backbone that is ionically bonded to an alpha-keto acid. The alpha-keto acids are alpha-keto acid analogues of essential amino acids. The chitosan alpha-keto acids of the essential amino acids are prepared as a medical food composition such that the medical food provides a source for all the essential amino acids. The chitosan alpha-keto acid compositions and formulations are combined in solid form with an electrolyte to produce a medical food in the form of an electrolyte containing powder. The electrolyte containing powder is suitable for mixing with water to form a beverage that may be consumed by a subject in need of essential amino acids.

### Example 8

### Medical Grade Food Compositions Comprising Glyceride Keto Esters

This example describes a composition of a medical grade food that comprises glyceride keto ester compounds. Each glyceride keto ester compound in the composition is a tri-glyceride compound having groups R¹, R², and R³ amino acid sidechains, a di-glyceride compound having groups R¹ and R² amino acid sidechains, or a mono-glyceride having a group R¹ amino acid sidechain. Each tri-, di-, or mono-glyceride compound comprises a glycerol backbone wherein one or more primary hydroxyl groups are covalently linked to the alpha-keto acid form of the amino acids, thereby forming a keto ester functional group. All essential amino acid sidechains are represented in the R¹, R², and R³ amino acid sidechains of the glyceride keto ester compounds in the medical grade food such that the medical food provides a source for all the essential amino acids. The glyceride keto ester compounds are combined in solid form into an electrolyte containing powder suitable for mixing with water, forming a medical food in the form of a beverage.

### Example 9

Use of Medical Food Composition for Treatment of Low Blood Protein Levels in a Patient with Kidney Disease

This example describes the use of a medical food for treatment of low blood protein levels in a patient with kidney disease. The patient with kidney disease is protein deficient. The patient is administered the medical food comprising a chitosan alpha-keto acid composition including all essential amino acid sidechains, such as the medical food described in EXAMPLE 7 or EXAMPLE 8. Administration of the medical food increases the patient's protein levels, thereby treating the low blood protein in the patient.

### Example 10

### Use of Medical Food Composition for Administration of Low-Nitrogen Protein for a Diabetic Subject

This example describes the use of a medical food for increasing blood amino acid levels without increasing blood ammonia levels for a subject with diabetes. The subject with diabetes is protein deficient. The subject is administered the medical food which includes a chitosan alpha-keto acid composition with all essential amino acid sidechains, such as the medical food described in EXAMPLE 7 or EXAMPLE 8. Administration of the medical food increases the subject's blood amino acid levels but does not increase their blood ammonia levels.

While preferred embodiments of the present invention have been shown and described herein, it will be apparent to those skilled in the art that such embodiments are provided by way of example only. Numerous variations, changes, and substitutions will now occur to those skilled in the art without departing from the invention. It should be understood that various alternatives to the embodiments of the invention described herein may be employed in practicing the invention. It is intended that the following claims define the scope of the invention and that methods and structures within the scope of these claims and their equivalents be covered thereby.

What is claimed is:

1. A composition comprising:
a polysaccharide, and
an alpha-keto acid composition comprising one or more of an alpha-keto acid analogue of an amino acid.

2. The composition of claim 1, wherein the alpha-keto acid composition comprises one or more of an alpha-keto acid analogue of an essential amino acid.

3. The composition of claim 1 or claim 2, wherein the polysaccharide comprises a starch, a cellulose, an amino polysaccharide, an alginate, a carrageenan, a chitin, a chondroitin sulfate, a dextran, a galactomannan, a glycogen, a hyaluronic acid, a galactogen, an inulin, an arabinoxylan, or a pectin.

4. The composition of any one of claims 1-3, wherein the polysaccharide comprises an amino polysaccharide.

5. The composition of claim 4, wherein the amino polysaccharide comprises an amino cellulose, an amino dextran, an amino galactose, an amino arabinose, a chitin, a chitosan, or a combination thereof.

6. The composition of any one of claims 1-5, wherein the polysaccharide comprises a chitosan.

7. The composition of claim 6, wherein the chitosan comprises Formula (I):

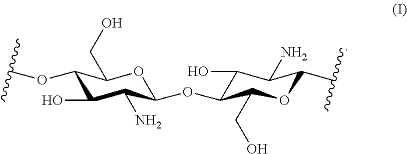

8. The composition of any one of claims 1-7, wherein the polysaccharide comprises a positive charge at a neutral pH.

9. The composition of claim 8, wherein the positive charge comprises a positively charged amino group.

10. The composition of any one of claims 1-9, wherein the one or more of the alpha-keto acid analogue of the amino acid comprises a negative charge at a neutral pH.

11. The composition of claim 10, wherein the negative charge comprises a negatively charged carboxylate group.

12. The composition of any one of claims 1-11, wherein the one or more of the alpha-keto acid analogue of the amino acid comprises one or more of: 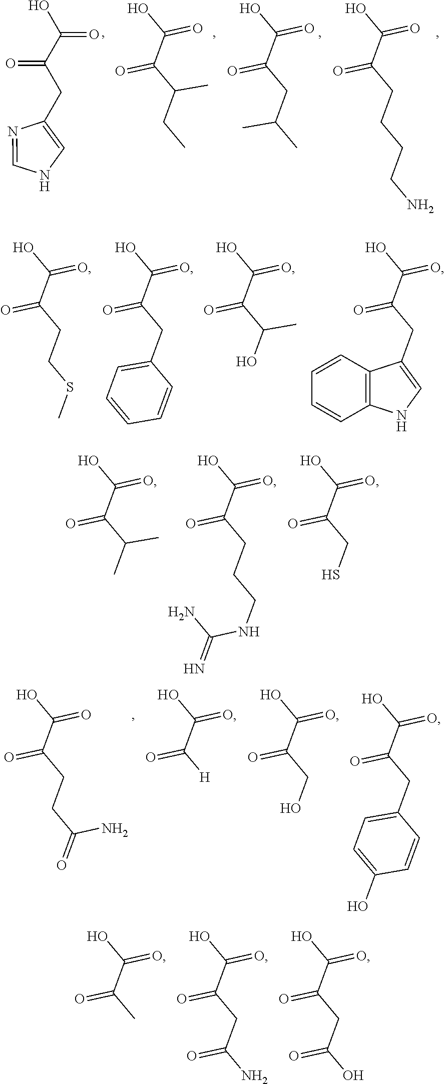
or a stereoisomer or pharmaceutically acceptable salt thereof.

13. The composition of any one of claims 1-12, wherein the one or more of the alpha-keto acid analogue of the amino acid comprises one or more of: 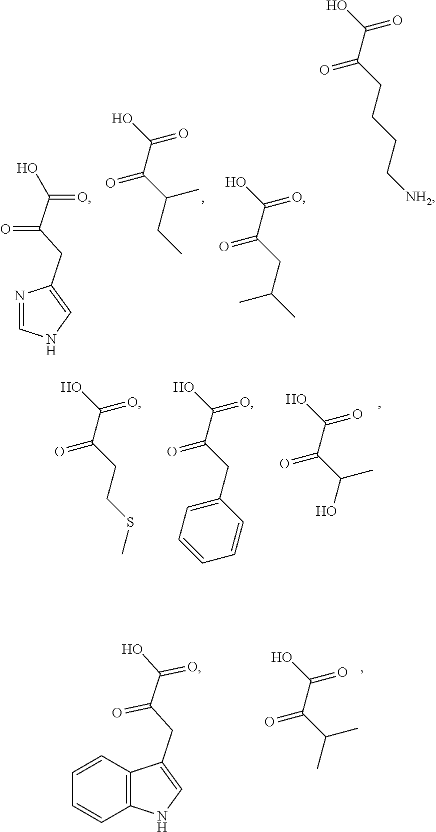
or a stereoisomer or pharmaceutically acceptable salt thereof.

14. The composition of any one of claims 1-13, comprising an ionic coupling between the polysaccharide and the one or more of the alpha-keto acid analogue of the amino acid.

15. The composition of claim 14, wherein the ionic coupling comprises an ionic bond between a positively charged amino group and a negatively charged carboxylate group.

16. The composition of any one of claims 1-15, wherein at least 10% and no more than 99% of the alpha-keto acid composition is bound to the polysaccharide.

17. The composition of any one of claims 1-16, wherein at least 15% and no more than 99%, at least 20% and no more than 99%, at least 25% and no more than 99%, at least 30% and no more than 99%, at least 35% and no more than 99%, at least 40% and no more than 99%, at least 45% and no more than 99%, at least 50% and no more than 99%, at least 55% and no more than 99%, at least 60% and no more than 99%, at least 65% and no more than 99%, at least 70% and no more than 99%, at least 75% and no more than 99%, at least 80% and no more than 99%, at least 85% and no more than 99%, at least 90% and no more than 99%, or at least 95% and no more than 99% of the alpha-keto acid composition is bound to the polysaccharide.

18. The composition of any one of claims 1-17, wherein the composition comprises Formula (VI): 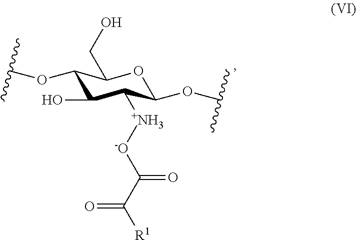
or a stereoisomer or pharmaceutically acceptable salt thereof,
wherein each R¹ is independently a sidechain of a natural amino acid.

19. The composition of claim 18, wherein each R¹ is independently:

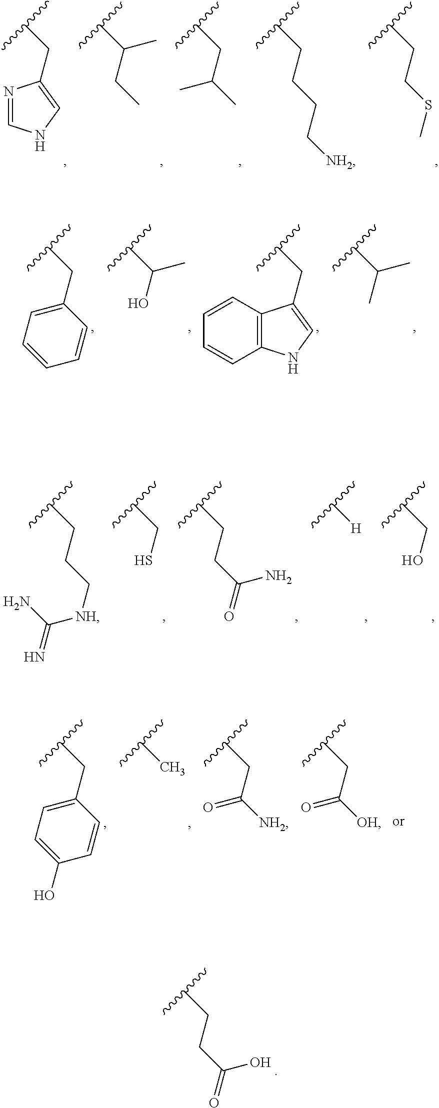

20. The composition of claim 18 or claim 19, wherein each R¹ is independently:

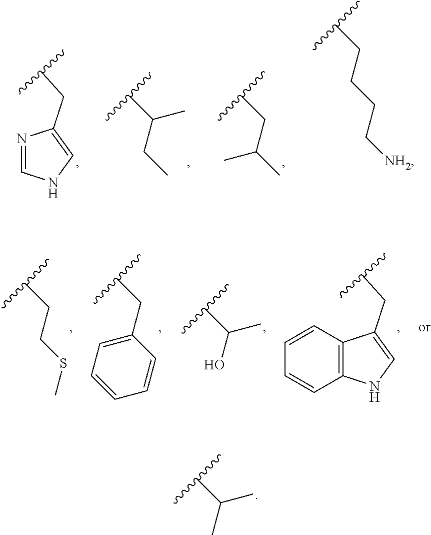

21. The composition of any one of claims 18-20, wherein R¹ comprises:

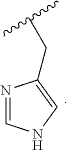 [SMILES: *Cc1c[nH]cn1]

22. The composition of any one of claims 18-21, wherein R¹ comprises:

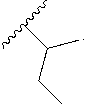 [SMILES: *C(C)CC]

23. The composition of any one of claims 18-22, wherein R¹ comprises:

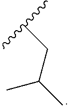 [SMILES: *CC(C)C]

24. The composition of any one of claims 18-23, wherein R¹ comprises:

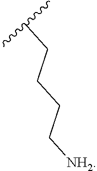 [SMILES: *CCCCN]

25. The composition of any one of claims 18-24, wherein R¹ comprises:

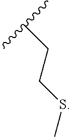 [SMILES: *CCSC]

26. The composition of any one of claims 18-25, wherein R¹ comprises:

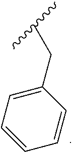 [SMILES: *Cc1ccccc1]

27. The composition of any one of claims 18-26, wherein R¹ comprises:

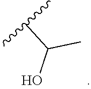 [SMILES: *C(C)O]

28. The composition of any one of claims 18-27, wherein R¹ comprises:

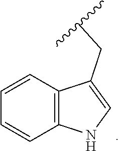 [SMILES: *Cc1c[nH]c2ccccc12]

29. The composition of any one of claims 18-28, wherein R¹ comprises:

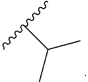 [SMILES: *C(C)C]

30. The composition of any one of claims 18-29, wherein R¹ comprises:

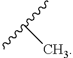

31. The composition of any one of claims 18-30, wherein R¹ comprises:

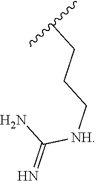 [SMILES: *CCCNC(=N)N]

32. The composition of any one of claims 18-31, wherein R¹ comprises:

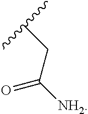 [SMILES: *CC(N)=O]

33. The composition of any one of claims 18-32, wherein R¹ comprises:

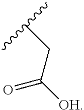 [SMILES: *CC(=O)O]

34. The composition of any one of claims 18-33, wherein R¹ comprises:

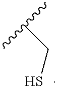 [SMILES: *CS]

35. The composition of any one of claims 18-34, wherein R¹ comprises:

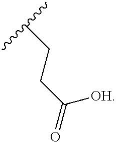 [SMILES: *CCC(=O)O]

36. The composition of any one of claims 18-35, wherein R¹ comprises:

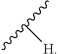

37. The composition of any one of claims 18-36, wherein R¹ comprises:

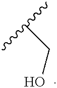 [SMILES: *CO]

38. The composition of any one of claims 18-37, wherein R¹ comprises:

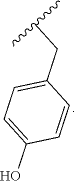 [SMILES: *Cc1ccc(O)cc1]

39. The composition of any one of claims 18-38, wherein R¹ comprises:

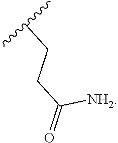 [SMILES: *CCC(N)=O]

40. The composition of any one of claims 6-39, wherein the chitosan is not more than 50%, not more than 45%, not more than 40%, not more than 35%, not more than 30%, not more than 25%, not more than 20%, not more than 15%, not more than 10%, or not more than 5% acetylated.

41. The composition of any one of claims 6-40, wherein the chitosan comprises a copolymer.

42. The composition of claim 41, wherein the copolymer comprises a linear copolymer, a block copolymer, an alternating copolymer, a periodic copolymer, a statistical copolymer, a stereoblock copolymer, a gradient copolymer, branched copolymer, a graft copolymer, a start copolymer, or a combination thereof.

43. The composition of any one of claims 6-42, wherein a stoichiometric ratio of the chitosan to the alpha-keto acid composition is from 1:1 to 1:10, from 1:1 to 1:100, from 1:1 to 1:1000, from 1:1 to 1:10,000, or from 1:1 to 1:100,000.

44. The composition of any one of claims 6-43, wherein a weight ratio of the chitosan to the alpha-keto acid composition is from 1:1 to 1:10, from 1:1 to 1:100, from 1:1 to 1:1000, from 1:1 to 1:10,000, or from 1:1 to 1:100,000.

45. The composition of any one of claims 1-44, wherein the alpha-keto acid composition comprises at least 0.01 mg/ml and no more than 20 mg/ml of the one or more of the alpha-keto acid analogue of the amino acid.

46. The composition of any one of claims 1-45, wherein the alpha-keto acid composition comprises at least 0.01 mg/ml and no more than 20 mg/ml, at least 0.1 mg/ml and no more than 20 mg/ml, at least 0.1 mg/ml and no more than 15 mg/ml, at least 0.1 mg/ml and no more than 10 mg/ml, or at least 0.5 mg/ml and no more than 10 mg/ml of the one or more of the alpha-keto acid analogue of the amino acid.

47. A pharmaceutical composition comprising the composition of any one of claims 1-46 and a pharmaceutically acceptable diluent or excipient.

48. The pharmaceutical composition of claim 47, wherein the pharmaceutical composition is formulated for oral, topical, transdermal, rectal, intravenous, intra-arterial, intra-peritoneal, parenteral, or inhalation administration.

49. A medical food composition comprising the composition of any one of claims 1-46 or the pharmaceutical composition of claim 47 or claim 48 and a food component.

50. The medical food composition of claim 49, wherein the medical food composition is a liquid, a solid, a colloid, a gel, or a combination thereof.

51. The medical food composition of claim 49 or claim 50, wherein the medical food composition is formulated as a beverage, a drink mix, a solid food, a bar, a cereal, a sports drink, a gel, a gelatin, a gelatinous gummy, a cracker, a chip, a puff, a granola cereal, a granola bar, a tablet, a powder, or an additive.

52. The medical food composition of any one of claims 49-51, further comprising:
a) a Cd content of less than 5 μg per daily serving or of less than 0.5 μg per g; and/or
b) a Pb content of less than 5 μg per daily serving or less than 0.5 μg per g; and/or
c) an As content of less than 15 μg per daily serving or less than 1.5 μg per g; and/or
d) a Hg content of less than 30 μg per daily serving or less than 3 μg per g; and/or
e) a Co content of less than 50 μg per daily serving or less than 5 μg per g; and/or
f) a V content of less than 100 μg per daily serving or less than 10 μg per g; and/or
g) a Ni content of less than 200 μg per daily serving or less than 20 μg per g; and/or
h) a Tl content of less than 8 μg per daily serving or less than 0.8 μg per g; and/or
i) an Au content of less than 300 μg per daily serving or less than 30 μg per g; and/or
j) a Pd content of less than 100 μg per daily serving or less than 10 μg per g; and/or
k) an Ir content of less than 100 μg per daily serving or less than 10 μg per g; and/or
l) an Os content of less than 100 μg per daily serving or less than 10 μg per g; and/or
m) a Rh content of less than 100 μg per daily serving or less than 10 μg per g; and/or
n) a Ru content of less than 100 μg per daily serving or less than 10 μg per g; and/or
o) a Se content of less than 150 μg per daily serving or less than 15 μg per g; and/or
p) an Ag content of less than 150 μg per daily serving or less than 15 μg per g; and/or
q) a Pt content of less than 100 μg per daily serving or less than 10 μg per g; and/or
r) a Li content of less than 550 μg per daily serving or less than 55 μg per g; and/or
s) a Sb content of less than 1200 μg per daily serving or less than 120 μg per g; and/or
t) a Ba content of less than 1400 μg per daily serving or less than 140 μg per g; and/or
u) a Mo content of less than 3000 μg per daily serving or less than 300 μg per g; and/or
v) a Cu content of less than 3000 μg per daily serving or less than 300 μg per g; and/or
w) a Sn content of less than 6000 μg per daily serving or less than 600 μg per g; and/or
x) a Cr content of less than 11000 μg per daily serving or less than 1100 μg per g; or
any combination thereof.

53. The medical food composition of any one of claims 49-52, comprising a microbial plate count of no more than 1000 colony forming per mL, no more than 10,000 colony forming per mL, or no more than 20,000 colony forming per mL.

54. A method of administering protein to a subject, the method comprising:
administering a composition to the subject comprising:
a polysaccharide, and
an alpha-keto acid composition,
wherein administering the composition to the subject increases a protein level in the subject as measured by a serum albumin test.

55. A method of treating a subject in need thereof, the method comprising:
administering a composition to the subject comprising:
a polysaccharide, and
an alpha-keto acid composition, and
increasing a protein level in the subject as measured by a serum albumin test, thereby treating the subject.

56. The method of claim 54 or claim 55, wherein the composition comprises the composition of any one of claims 1-46, the pharmaceutical composition of claim 47 or claim 48, or the medical food composition of any one of claims 49-53.

57. The method of any one of claims 54-56, wherein the polysaccharide comprises chitosan.

58. The method of any one of claims 54-57, wherein the alpha-keto acid composition comprises one or more of an alpha-keto acid analogue of an amino acid.

59. The method of claim 54-58, wherein the alpha-keto acid composition comprises one or more of an alpha-keto acid analogue of an essential amino acid.

60. The method of any one of claims 54-59, wherein the subject has a higher than normal blood urea nitrogen level as measured by a serum blood urea nitrogen (BUN) test prior to administration of the composition.

61. The method of claim 60, wherein the higher than normal blood urea nitrogen level is caused by dehydration, burns, medicines, a high protein diet, or age.

62. The method of any one of claims 54-61, wherein administering the composition to the subject results in a decrease of nitrogen by-products in the subject as compared to administering a comparable amount of an amino acid composition to the subject.

63. The method of claim 62, wherein the nitrogen by-products comprise urea, uric acid, creatinine, ammonia, or a combination thereof.

64. The method of claim 62 or claim 63, wherein the decrease of nitrogen by-products is measured by a serum blood urea nitrogen (BUN) test.

65. The method of any one of claims 54-64, wherein administering the composition to the subject results in a decrease of a level of a salt in the subject as measured by a salt urine test as compared to administering a comparable amount of an alpha-keto acid salt composition to the subject.

66. The method of claim 65, wherein the decrease of the level of the salt is measured by a urine test measuring sodium, potassium, and chloride.

67. The method of claim 65 or claim 66, wherein the salt comprises sodium, calcium, or magnesium.

68. The method of any one of claims 54-67, wherein the subject does not have a kidney disease.

69. The method of any one of claims 54-68, wherein the subject is at risk of developing a kidney disease.

70. The method of any one of claims 54-69, wherein the subject has a condition that puts the subject at risk of a kidney disease.

71. The method of claim 70, wherein the condition is diabetes, high blood pressure, heart disease, obesity, a family history of chronic kidney disease, inherited kidney disorders, past damage to the kidneys, or old age.

72. The method of any one of claims 54-71, wherein the subject has a kidney disease.

73. The method of any one of claims 68-70 or claim 72, wherein the kidney disease is chronic kidney disease, Fabry disease, cystinosis, glomerulonephritis, IgA nephropathy, lupus nephritis, atypical hemolytic uremic syndrome, polycystic kidney disease, diabetic kidney disease, or uremia.

74. The method of any one of claims 54-73, wherein the subject is in an anabolic need.

75. The method of claim 74, wherein the anabolic need is characterized by a low protein-level as measured by a serum albumin test in the subject.

76. A method of synthesizing a chitosan keto acid composition comprising:
combining a chitosan composition and an alpha-keto acid composition,
adjusting the pH to promote binding of the alpha-keto acid composition to the chitosan composition,
binding the alpha-keto acid composition to the chitosan composition thereby forming the chitosan keto acid composition.

77. The method of claim 76, further comprising lyophilizing the chitosan keto acid composition.

78. The method of claim 76 or claim 77, wherein the chitosan composition binds at least 15% and no more than 99%, at least 20% and no more than 99%, at least 25% and no more than 99%, at least 30% and no more than 99%, at least 35% and no more than 99%, at least 40% and no more than 99%, at least 45% and no more than 99%, at least 50% and no more than 99%, at least 55% and no more than 99%, at least 60% and no more than 99%, at least 65% and no more than 99%, at least 70% and no more than 99%, at least 75% and no more than 99%, at least 80% and no more than 99%, at least 85% and no more than 99%, at least 90% and no more than 99%, or at least 95% and no more than 99% of the alpha-keto acid composition.

79. The method of any one of claims 76-78, wherein the alpha-keto acid composition comprises one or more of an alpha-keto acid analogue of an essential amino acid.

80. The method of any one of claims 76-79, wherein the alpha-keto acid composition comprises at least 0.01 mg/ml and no more than 20 mg/ml, at least 0.1 mg/ml and no more than 20 mg/ml, at least 0.1 mg/ml and no more than 15 mg/ml, at least 0.1 mg/ml and no more than 10 mg/ml, or at least 0.5 mg/ml and no more than 10 mg/ml of one or more alpha-keto acid analogues of an amino acid.
